SEQUENCE LISTING (123 sequences)

SEQ ID NO: 1 - DNA, 21 bp, Hepatitis C virus
ggcatgcaagcttaattaatt

SEQ ID NO: 2 - DNA, 68 bp, Hepatitis C virus
ccggggaggcctgcacgtgatcgagggcagacaccatcaccaccatcactaatagttaattaactgca

SEQ ID NO: 3 - DNA, 642 bp, Hepatitis C virus
  CDS 1..639
  mat_peptide 1..636
atgcccggttgctctttctctatcttcctcttggctttactgtcctgtctgaccattccagcttccgcttatgaggtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccaccacgacaatacgacgccacgtcgatttgctcgttggggcggctgctctctgttccgctatgtacgtgggggatctctgcggatctgtcttcctcgtctcccagctgttcaccatctcgcctcgccggcatgagacggtgcaggactgcaattgctcaatctatcccggccacataacaggtcaccgtatggcttgggatatgatgatgaactggtcgcctacaacggccctggtggtatcgcagctgctccggatcccacaagctgtcgtggacatggtggcgggggcccattggggagtcctggcgggcctcgcctactattccatggtggggaactgggctaaggttttgattgtgatgctactctttgctctctaatag

SEQ ID NO: 4 - protein, 212 aa, Hepatitis C virus
MPGCSFSIFLLALLSCLTIPASAYEVRNVSGMYHVTNDCSNSSIVYEAADMIMHTPGCVPCVRENNSSRCWVALTPTLAARNASVPTTTIRRHVDLLVGAAALCSAMYVGDLCGSVFLVSQLFTISPRRHETVQDCNCSIYPGHITGHRMAWDMMMNWSPTTALVVSQLLRIPQAVVDMVAGAHWGVLAGLAYYSMVGNWAKVLIVMLLFAL

SEQ ID NO: 5 - DNA, 795 bp, Hepatitis C virus
  CDS 1..792
  mat_peptide 1..789
atgttgggtaaggtcatcgatacccttacatgcggcttcgccgacctcgtggggtacattccgctcgtcggcgcccccctagggggcgctgccagggccctggcgcatggcgtccgggttctggaggacggcgtgaactatgcaacagggaatttgcccggttgctctttctctatcttcctcttggctttgctgtcctgtctgaccgttccagcttccgcttatgaagtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccaccacgacaatacgacgccacgtcgatttgctcgttggggcggctgctttctgttccgctatgtacgtgggggacctctgcggatctgtcttcctcgtctcccagctgttcaccatctcgcctcgccggcatgagacggtgcaggactgcaattgctcaatctatcccggccacataacgggtcaccgtatggcttgggatatgatgatgaactggtcgcctacaacggccctggtggtatcgcagctgctccggatcccacaagctgtcgtggacatggtggcgggggcccattggggagtcctggcgggtctcgcctactattccatggtggggaactgggctaaggttttgattgtgatgctactctttgctccctaatag

SEQ ID NO: 6 - protein, 263 aa, Hepatitis C virus
MLGKVIDTLTCGFADLVGYIPLVGAPLGGAARALAHGVRVLEDGVNYATGNLPGCSFSIFLLALLSCLTVPASAYEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNWSPTTALVVSQLLRIPQAVVDMVAGAHWGVLAGLAYYSMVGNWAKVLIVMLLFAP

SEQ ID NO: 7 - DNA, 633 bp, Hepatitis C virus
  CDS 1..630
  mat_peptide 1..627
atgttgggtaaggtcatcgatacccttacgtgcggcttcgccgacctcatggggtacattccgctcgtcggcgcccccctagggggtgctgccagagccctggcgcatggcgtccgggttctggaagacggcgtgaactatgcaacagggaatttgcctggttgctctttctctatcttcctcttggctttactgtcctgtctgaccattccagcttccgcttatgaggtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccactacgacaatacgacgccacgtcgatttgctcgttggggcggctgctttctgttccgctatgtacgtgggggatctctgcggatctgtcttcctcgtctcccagctgttcaccatctcgcctcgccggcatgagacggtgcaggactgcaattgctcaatctatcccggccacataacaggtcaccgtatggcttgggatatgatgatgaactggtaatag

SEQ ID NO: 8 - protein, 209 aa, Hepatitis C virus
MLGKVIDTLTCGFADLMGYIPLVGAPLGGAARALAHGVRVLEDGVNYATGNLPGCSFSIFLLALLSCLTIPASAYEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNW

SEQ ID NO: 9 - DNA, 483 bp, Hepatitis C virus
  CDS 1..480
  mat_peptide 1..477
atgcccggttgctctttctctatcttcctcttggccctgctgtcctgtctgaccataccagcttccgcttatgaagtgcgcaacgtgtccggggtgtaccatgtcacgaacgactgctccaactcaagcatagtgtatgaggcagcggacatgatcatgcacacccccgggtgcgtgccctgcgttcgggagggcaactcctcccgttgctgggtggcgctcactcccacgctcgcggccaggaacgccagcgtccccacaacgacaatacgacgccacgtcgatttgctcgttggggctgctgctttctgttccgctatgtacgtgggggatctctgcggatctgttttccttgtttcccagctgttcaccttctcacctcgccggcatcaaacagtacaggactgcaactgctcaatctatcccggccatgtatcaggtcaccgcatggcttgggatatgatgatgaactggtcctaatag

SEQ ID NO: 10 - protein, 159 aa, Hepatitis C virus
MPGCSFSIFLLALLSCLTIPASAYEVRNVSGVYHVTNDCSNSSIVYEAADMIMHTPGCVPCVREGNSSRCWVALTPTLAARNASVPTTTIRRHVDLLVGAAAFCSAMYVGDLCGSVFLVSQLFTFSPRRHQTVQDCNCSIYPGHVSGHRMAWDMMMNWS

SEQ ID NO: 11 - DNA, 480 bp, Hepatitis C virus
  CDS 1..477
  mat_peptide 1..474
atgtccggttgctctttctctatcttcctcttggccctgctgtcctgtctgaccataccagcttccgcttatgaagtgcgcaacgtgtccggggtgtaccatgtcacgaacgactgctccaactcaagcatagtgtatgaggcagcggacatgatcatgcacacccccgggtgcgtgccctgcgttcgggagggcaactcctcccgttgctgggtggcgctcactcccacgctcgcggccaggaacgccagcgtccccacaacgacaatacgacgccacgtcgatttgctcgttggggctgctgctttctgttccgctatgtacgtgggggatctctgcggatctgttttccttgtttcccagctgttcaccttctcacctcgccggcatcaaacagtacaggactgcaactgctcaatctatcccggccatgtatcaggtcaccgcatggcttgggatatgatgatgaactggtaatag

SEQ ID NO: 12 - protein, 158 aa, Hepatitis C virus
MSGCSFSIFLLALLSCLTIPASAYEVRNVSGVYHVTNDCSNSSIVYEAADMIMHTPGCVPCVREGNSSRCWVALTPTLAARNASVPTTTIRRHVDLLVGAAAFCSAMYVGDLCGSVFLVSQLFTFSPRRHQTVQDCNCSIYPGHVSGHRMAWDMMMNW

SEQ ID NO: 13 - DNA, 636 bp, Hepatitis C virus
  CDS 1..633
  mat_peptide 1..630
atgctgggtaaggccatcgatacccttacgtgcggcttcgccgacctcgtggggtacattccgctcgtcggcgcccccctagggggcgctgccagggccctggcgcatggcgtccgggttctggaagacggcgtgaactatgcaacagggaatttgcctggttgctctttctctatcttcctcttggctttactgtcctgtctaaccattccagcttccgcttacgaggtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcggctaggaacgccagcatccccactacaacaatacgacgccacgtcgatttgctcgttggggcggctgctttctgttccgctatgtacgtgggggatctctgcggatctgtcttcctcgtctcccagctgttcaccatctcgcctcgccggcatgagacggtgcaggactgcaattgctcaatctatcccggccacataacgggtcaccgtatggcttgggatatgatgatgaactggtactaatag

SEQ ID NO: 14 - protein, 210 aa, Hepatitis C virus
MLGKAIDTLTCGFADLVGYIPLVGAPLGGAARALAHGVRVLEDGVNYATGNLPGCSFSIFLLALLSCLTIPASAYEVRNVSGMYHVTNDCSNSSIVYEAADMIMHTPGCVPCVRENNSSRCWVALTPTLAARNASIPTTTIRRHVDLLVGAAAFCSAMYVGDLCGSVFLVSQLFTISPRRHETVQDCNCSIYPGHITGHRMAWDMMMNWY

SEQ ID NO: 15 - DNA, 26 bp, Hepatitis C virus
atgcccggttgctctttctctatctt

SEQ ID NO: 16 - DNA, 26 bp, Hepatitis C virus
atgttgggtaaggtcatcgataccct

SEQ ID NO: 17 - DNA, 30 bp, Hepatitis C virus
  misc_feature: /note=“antisense”
ctattaggaccagttcatcatcatatccca

SEQ ID NO: 18 - DNA, 27 bp, Hepatitis C virus
ctattaccagttcatcatcatatccca

SEQ ID NO: 19 - DNA, 36 bp, Hepatitis C virus
atacgacgccacgtcgattcccagctgttcaccatc

SEQ ID NO: 20 - DNA, 36 bp, Hepatitis C virus
  misc_feature: /note=“antisense”
gatggtgaacagctgggaatcgacgtggcgtcgtat

SEQ ID NO: 21 - DNA, 723 bp, Hepatitis C virus
  CDS 1..720
  mat_peptide 1..717
atgttgggtaaggtcatcgatacccttacatgcggcttcgccgacctcgtggggtacattccgctcgtcggcgcccccctagggggcgctgccagggccctggcgcatggcgtccgggttctggaggacggcgtgaactatgcaacagggaatttgcccggttgctctttctctatcttcctcttggctttgctgtcctgtctgaccgttccagcttccgcttatgaagtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccaccacgacaatacgacgccacgtcgattcccagctgttcaccatctcgcctcgccggcatgagacggtgcaggactgcaattgctcaatctatcccggccacataacgggtcaccgtatggcttgggatatgatgatgaactggtcgcctacaacggccctggtggtatcgcagctgctccggatcccacaagctgtcgtggacatggtggcgggggcccattggggagtcctggcgggtctcgcctactattccatggtggggaactgggctaaggttttgattgtgatgctactctttgctccctaatag

SEQ ID NO: 22 - protein, 239 aa, Hepatitis C virus
MLGKVIDTLTCGFADLVGYIPLVGAPLGGAARALAHGVRVLEDGVNYATGNLPGCSFSIFLLALLSCLTVPASAYEVRNVSGMYHVTNDCSNSSIVYEAADMIMHTPGCVPCVRENNSSRCWVALTPTLAARNASVPTTTIRRHVDSQLFTISPRRHETVQDCNCSIYPGHITGHRMAWDMMMNWSPTTALVVSQLLRIPQAVVDMVAGAHWGVLAGLAYYSMVGNWAKVLIVMLLFAP

SEQ ID NO: 23 - DNA, 561 bp, Hepatitis C virus
  CDS 1..558
  mat_peptide 1..555
atgttgggtaaggtcatcgatacccttacatgcggcttcgccgacctcgtggggtacattccgctcgtcggcgcccccctagggggcgctgccagggccctggcgcatggcgtccgggttctggaggacggcgtgaactatgcaacagggaatttgcccggttgctctttctctatcttcctcttggctttgctgtcctgtctgaccgttccagcttccgcttatgaagtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccaccacgacaatacgacgccacgtcgattcccagctgttcaccatctcgcctcgccggcatgagacggtgcaggactgcaattgctcaatctatcccggccacataacgggtcaccgtatggcttgggatatgatgatgaactggtaatag

SEQ ID NO: 24 - protein, 185 aa, Hepatitis C virus
MLGKVIDTLTCGFADLVGYIPLVGAPLGGAARALAHGVRVLEDGVNYATGNLPGCSFSIFLLALLSCLTVPASAYEVRNVSGMYHVTNDCSNSSIVYEAADMIMHTPGCVPCVRENNSSRCWVALTPTLAARNASVPTTTIRRHVDSQLFTISPRRHETVQDCNCSIYPGHITGHRMAWDMMMNW

SEQ ID NO: 25 - DNA, 606 bp, Hepatitis C virus
  CDS 1..603
  mat_peptide 1..600
atgttgggtaaggtcatcgatacccttacatgcggcttcgccgacctcgtggggtacattccgctcgtcggcgcccccctagggggcgctgccagggccctggcgcatggcgtccgggttctggaggacggcgtgaactatgcaacagggaatttgcccggttgctctttctctatcttcctcttggctttgctgtcctgtctgaccgttccagcttccgcttatgaagtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccaccacgacaatacgacgccacgtcgattcccagctgttcaccatctcgcctcgccggcatgagacggtgcaggactgcaattgctcaatctatcccggccacataacgggtcaccgtatggcttgggatatgatgatgaactggtcgcctacaacggccctggtggtatcgcagctgctccggatcctctaatag

SEQ ID NO: 26 - protein, 200 aa, Hepatitis C virus
MLGKVIDTLTCGFADLVGYIPLVGAPLGGAARALAHGVRVLEDGVNYATGNLPGCSFSIFLLALLSCLTVPASAYEVRNVSGMYHVTNDCSNSSIVYEAADMIMHTPGCVPCVRENNSSRCWVALTPTLAARNASVPTTTIRRHVDSQLFTISPRRHETVQDCNCSIYPGHITGHRMAWDMMMNWSPTTALVVSQLLRIL

SEQ ID NO: 27 - DNA, 636 bp, Hepatitis C virus
  CDS 1..633
  mat_peptide 1..630
atgttgggtaaggtcatcgatacccttacatgcggcttcgccgacctcgtggggtacattccgctcgtcggcgcccccctagggggcgctgccagggccctggcgcatggcgtccgggttctggaggacggcgtgaactatgcaacagggaatttgcccggttgctctttctctatcttcctcttggctttgctgtcctgtctgaccgttccagcttccgcttatgaagtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccaccacgacaatacgacgccacgtcgattcccagctgttcaccatctcgcctcgccggcatgagacggtgcaggactgcaattgctcaatctatcccggccacataacgggtcaccgtatggcttgggatatgatgatgaactggtcgcctacaacggccctggtggtatcgcagctgctccggatcgtgatcgagggcagacaccatcaccaccatcactaatag

SEQ ID NO: 28 - protein, 210 aa, Hepatitis C virus
MLGKVIDTLTCGFADLVGYIPLVGAPLGGAARALAHGVRVLEDGVNYATGNLPGCSFSIFLLALLSCLTVPASAYEVRNVSGMYHVTNDCSNSSIVYEAADMIMHTPGCVPCVRENNSSRCWVALTPTLAARNASVPTTTIRRHVDSQLFTISPRRHETVQDCNCSIYPGHITGHRMAWDMMMNWSPTTALVVSQLLRIVIEGRHHHHHH

SEQ ID NO: 29 - DNA, 630 bp, Hepatitis C virus
  CDS 1..627
  mat_peptide 1..624
atgggtaaggtcatcgatacccttacgtgcggattcgccgatctcatggggtacatcccgctcgtcggcgctcccgtaggaggcgtcgcaagagcccttgcgcatggcgtgagggcccttgaagacgggataaatttcgcaacagggaatttgcccggttgctccttttctattttccttctcgctctgttctcttgcttaattcatccagcagctagtctagagtggcggaatacgtctggcctctatgtccttaccaacgactgttccaatagcagtattgtgtacgaggccgatgacgttattctgcacacacccggctgcataccttgtgtccaggacggcaatacatccacgtgctggaccccagtgacacctacagtggcagtcaagtacgtcggagcaaccaccgcttcgatacgcagtcatgtggacctattagtgggcgcggccacgatgtgctctgcgctctacgtgggtgacatgtgtggggctgtcttcctcgtgggacaagccttcacgttcagacctcgtcgccatcaaacggtccagacctgtaactgctcgctgtacccaggccatctttcaggacatcgaatggcttgggatatgatgatgaactggtaatag

SEQ ID NO: 30 - protein, 208 aa, Hepatitis C virus
MGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCIPCVQDGNTSTCWTPVTPTVAVKYVGATTASIRSHVDLLVGAATMCSALYVGDMCGAVFLVGQAFTFRPRRHQTVQTCNCSLYPGHLSGHRMAWDMMMNW

SEQ ID NO: 31 - DNA, 630 bp, Hepatitis C virus
  CDS 1..627
  mat_peptide 1..624
atgggtaaggtcatcgataccctaacgtgcggattcgccgatctcatggggtatatcccgctcgtaggcggccccattgggggcgtcgcaagggctctcgcacacggtgtgagggtccttgaggacggggtaaactatgcaacagggaatttacccggttgctctttctctatctttattcttgctcttctctcgtgtctgaccgttccggcctctgcagttccctaccgaaatgcctctgggatttatcatgttaccaatgattgcccaaactcttccatagtctatgaggcagataacctgatcctacacgcacctggttgcgtgccttgtgtcatgacaggtaatgtgagtagatgctgggtccaaattacccctacactgtcagccccgagcctcggagcagtcacggctcctcttcggagagccgttgactacctagcgggaggggctgccctctgctccgcgttatacgtaggagacgcgtgtggggcactattcttggtaggccaaatgttcacctataggcctcgccagcacgctacggtgcagaactgcaactgttccatttacagtggccatgttaccggccaccggatggcatgggatatgatgatgaactggtaatag

SEQ ID NO: 32 - protein, 208 aa, Hepatitis C virus
MGKVIDTLTCGFADLMGYIPLVGGPIGGVARALAHGVRVLEDGVNYATGNLPGCSFSIFILALLSCLTVPASAVPYRNASGIYHVTNDCPNSSIVYEADNLILHAPGCVPCVMTGNVSRCWVQITPTLSAPSLGAVTAPLRRAVDYLAGGAALCSALYVGDACGALFLVGQMFTYRPRQHATVQNCNCSIYSGHVTGHRMAWDMMMNW

SEQ ID NO: 33 - DNA, 23 bp, Hepatitis C virus
tgggatatgatgatgaactggtc

SEQ ID NO: 34 - DNA, 30 bp, Hepatitis C virus
ctattatggtggtaagccacagagcaggag

SEQ ID NO: 35 - DNA, 1476 bp, Hepatitis C virus
  CDS 1..1473
  mat_peptide 1..1470
tgggatatgatgatgaactggtcgcctacaacggccctggtggtatcgcagctgctccggatcccacaagctgtcgtggacatggtggcgggggcccattggggagtcctggcgggcctcgcctactattccatggtggggaactgggctaaggttttggttgtgatgctactctttgccggcgtcgacgggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacctacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagcccgctgctgctgtctacaacagagtggcagatactgccctgttccttcaccaccctgccggccctatccaccggcctgatccacctccatcagaacatcgtggacgtgcaatacctgtacggtgtagggtcggcggttgtctcccttgtcatcaaatgggagtatgtcctgttgctcttccttctcctggcagacgcgcgcatctgcgcctgcttatggatgatgctgctgatagctcaagctgaggccgccttagagaacctggtggtcctcaatgcggcggccgtggccggggcgcatggcactctttccttccttgtgttcttctgtgctgcctggtacatcaagggcaggctggtccctggtgcggcatacgccttctatggcgtgtggccgctgctcctgcttctgctggccttaccaccacgagcttatgcctagtaa

SEQ ID NO: 36 - protein, 490 aa, Hepatitis C virus
WDMMMNWSPTTALVVSQLLRIPQAVVDMVAGAHWGVLAGLAYYSMVGNWAKVLVVMLLFAGVDGHTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTEWQILPCSFTTLPALSTGLIHLHQNIVDVQYLYGVGSAVVSLVIKWEYVLLLFLLLADARICACLWMMLLIAQAEAALENLVVLNAAAVAGAHGTLSFLVFFCAAWYIKGRLVPGAAYAFYGVWPLLLLLLALPPRAYA

SEQ ID NO: 37 - DNA, 1021 bp, Hepatitis C virus
  CDS 2..1018
  mat_peptide 2..1015
gatcccacaagctgtcgtggacatggtggcgggggcccattggggagtcctggcgggcctcgcctactattccatggtggggaactgggctaaggttttggttgtgatgctactctttgccggcgtcgacgggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacctacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagcccgctgctgctgtctacaacagagtggcagagtggcagagcttaatta

SEQ ID NO: 38 - protein, 338 aa, Hepatitis C virus
IPQAVVDMVAGAHWGVLAGLAYYSMVGNWAKVLVVMLLFAGVDGHTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTEWQSGRA

SEQ ID NO: 39 - DNA, 1034 bp, Hepatitis C virus
  CDS 2..1032
  mat_peptide 2..1029
gatcccacaagctgtcgtggacatggtggcgggggcccattggggagtcctggcgggcctcgcctactattccatggtggggaactgggctaaggttttggttgtgatgctactctttgccggcgtcgacgggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacctacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagcccgctgctgctgtctacaacaggtgatcgagggcagacaccatcaccaccatcactaatag

SEQ ID NO: 40 - protein, 343 aa, Hepatitis C virus
IPQAVVDMVAGAHWGVLAGLAYYSMVGNWAKVLVVMLLFAGVDGHTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTGDRGQTPSPPSL

SEQ ID NO: 41 - DNA, 945 bp, Hepatitis C virus
  CDS 1..942
  mat_peptide 1..939
atggtggggaactgggctaaggttttggttgtgatgctactctttgccggcgtcgacgggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacctacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagcccgctgctgctgtctacaacagagtggcagagcttaattaattag

SEQ ID NO: 42 - protein, 314 aa, Hepatitis C virus
MVGNWAKVLVVMLLFAGVDGHTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTEWQSLIN

SEQ ID NO: 43 - DNA, 961 bp, Hepatitis C virus
  CDS 1..958
  mat_peptide 1..955
atggtggggaactgggctaaggttttggttgtgatgctactctttgccggcgtcgacgggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacctacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagcccgctgctgctgtctacaacaggtgatcgagggcagacaccatcaccaccatcactaatag

SEQ ID NO: 44 - protein, 319 aa, Hepatitis C virus
MVGNWAKVLVVMLLFAGVDGHTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTGDRGQTPSPPSL

SEQ ID NO: 45 - DNA, 1395 bp, Hepatitis C virus
  CDS 1..1392
  mat_peptide 1..1389
atggtggcgggggcccattggggagtcctggcgggcctcgcctactattccatggtggggaactgggctaaggttttggttgtgatgctactctttgccggcgtcgacgggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacctacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagcccgctgctgctgtctacaacagagtggcagatactgccctgttccttcaccaccctgccggccctatccaccggcctgatccacctccatcagaacatcgtggacgtgcaatacctgtacggtgtagggtcggcggttgtctcccttgtcatcaaatgggagtatgtcctgttgctcttccttctcctggcagacgcgcgcatctgcgcctgcttatggatgatgctgctgatagctcaagctgaggccgccttagagaacctggtggtcctcaatgcggcggccgtggccggggcgcatggcactctttccttccttgtgttcttctgtgctgcctggtacatcaagggcaggctggtccctggtgcggcatacgccttctatggcgtgtggccgctgctcctgcttctgctggccttaccaccacgagcttatgcctagtaa

SEQ ID NO: 46 - protein, 463 aa, Hepatitis C virus
MVAGAHWGVLAGLAYYSMVGNWAKVLVVMLLFAGVDGHTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTEWQILPCSFTTLPALSTGLIHLHQNIVDVQYLYGVGSAVVSLVIKWEYVLLLFLLLADARICACLWMMLLIAQAEAALENLVVLNAAAVAGAHGTLSFLVFFCAAWYIKGRLVPGAAYAFYGVWPLLLLLLALPPRAYA

SEQ ID NO: 47 - DNA, 2082 bp, Hepatitis C virus
  CDS 1..2079
  mat_peptide 1..2076
aatttgggtaaggtcatcgatacccttacatgcggcttcgccgacctcgtggggtacattccgctcgtcggcgcccccctagggggcgctgccagggccctggcgcatggcgtccgggttctggaggacggcgtgaactatgcaacagggaatttgcccggttgctctttctctatcttcctcttggctttgctgtcctgtctgaccgttccagcttccgcttatgaagtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccaccacgacaatacgacgccacgtcgatttgctcgttggggcggctgctttctgttccgctatgtacgtgggggacctctgcggatctgtcttcctcgtctcccagctgttcaccatctcgcctcgccggcatgagacggtgcaggactgcaattgctcaatctatcccggccacataacgggtcaccgtatggcttgggatatgatgatgaactggtcgcctacaacggccctggtggtatcgcagctgctccggatcccacaagctgtcgtggacatggtggcgggggcccattggggagtcctggcgggcctcgcctactattccatggtggggaactgggctaaggttttggttgtgatgctactctttgccggcgtcgacgggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacctacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagcccgctgctgctgtctacaacagagtggcagatactgccctgttccttcaccaccctgccggccctatccaccggcctgatccacctccatcagaacatcgtggacgtgcaatacctgtacggtgtagggtcggcggttgtctcccttgtcatcaaatgggagtatgtcctgttgctcttccttctcctggcagacgcgcgcatctgcgcctgcttatggatgatgctgctgatagctcaagctgaggccgccttagagaacctggtggtcctcaatgcggcggccgtggccggggcgcatggcactctttccttccttgtgttcttctgtgctgcctggtacatcaagggcaggctggtccctggtgcggcatacgccttctatggcgtgtggccgctgctcctgcttctgctggccttaccaccacgagcttatgcctagtaa

SEQ ID NO: 48 - protein, 692 aa, Hepatitis C virus
NLGKVIDTLTCGFADLVGYIPLVGAPLGGAARALAHGVRVLEDGVNYATGNLPGCSFSIFLLALLSCLTVPASAYEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNWSPTTALVVSQLLRIPQAVVDMVAGAHWGVLAGLAYYSMVGNWAKVLVVMLLFAGVDGHTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTEWQILPCSFTTLPALSTGLIHLHQNIVDVQYLYGVGSAVVSLVIKWEYVLLLFLLLADARICACLWMMLLIAQAEAALENLVVLNAAAVAGAHGTLSFLVFFCAAWYIKGRLVPGAAYAFYGVWPLLLLLLALPPRAYA

SEQ ID NO: 49 - DNA, 2433 bp, Hepatitis C virus
  CDS 1..2430
  mat_peptide 1..2427
atgagcacgaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcaacctcgtgggaggcgacaacctatccccaaggctcgccgacccgagggtagggcctgggctcagcccgggtacccttggcccctctatggcaatgagggcatggggtgggcaggatggctcctgtcaccccgcggctctcggcctagttggggccctacagacccccggcgtaggtcgcgtaatttgggtaaggtcatcgatacccttacatgcggcttcgccgacctcgtggggtacattccgctcgtcggcgcccccctagggggcgctgccagggccctggcgcatggcgtccgggttctggaggacggcgtgaactatgcaacagggaatttgcccggttgctctttctctatcttcctcttggctttgctgtcctgtctgaccgttccagcttccgcttatgaagtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccaccacgacaatacgacgccacgtcgatttgctcgttggggcggctgctttctgttccgctatgtacgtgggggacctctgcggatctgtcttcctcgtctcccagctgttcaccatctcgcctcgccggcatgagacggtgcaggactgcaattgctcaatctatcccggccacataacgggtcaccgtatggcttgggatatgatgatgaactggtcgcctacaacggccctggtggtatcgcagctgctccggatcccacaagctgtcgtggacatggtggcgggggcccattggggagtcctggcgggcctcgcctactattccatggtggggaactgggctaaggttttggttgtgatgctactctttgccggcgtcgacgggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacctacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagcccgctgctgctgtctacaacagagtggcagatactgccctgttccttcaccaccctgccggccctatccaccggcctgatccacctccatcagaacatcgtggacgtgcaatacctgtacggtgtagggtcggcggttgtctcccttgtcatcaaatgggagtatgtcctgttgctcttccttctcctggcagacgcgcgcatctgcgcctgcttatggatgatgctgctgatagctcaagctgaggccgccttagagaacctggtggtcctcaatgcggcggccgtggccggggcgcatggcactctttccttccttgtgttcttctgtgctgcctggtacatcaagggcaggctggtccctggtgcggcatacgccttctatggcgtgtggccgctgctcctgcttctgctggccttaccaccacgagcttatgcctagtaa

SEQ ID NO: 50 - protein, 809 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGMGWAGWLLSPRGSRPSWGPTDPRRRSRNLGKVIDTLTCGFADLVGYIPLVGAPLGGAARALAHGVRVLEDGVNYATGNLPGCSFSIFLLALLSCLTVPASAYEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNWSPTTALVVSQLLRIPQAVVDMVAGAHWGVLAGLAYYSMVGNWAKVLVVMLLFAGVDGHTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTEWQILPCSFTTLPALSTGLIHLHQNIVDVQYLYGVGSAVVSLVIKWEYVLLLFLLLADARICACLWMMLLIAQAEAALENLVVLNAAAVAGAHGTLSFLVFFCAAWYIKGRLVPGAAYAFYGVWPLLLLLLALPPRAYA

SEQ ID NO: 51 - protein, 17 aa, Hepatitis C virus
SNSSEAADMIMHTPGCV

SEQ ID NO: 52 - protein, 22 aa, Hepatitis C virus
GGITGHRMAWDMMMNWSPTTAL

SEQ ID NO: 53 - protein, 37 aa, Hepatitis C virus
YEVRNVSGIYHVTNDCSNSSIVYEAADMIMHTPGCGK

SEQ ID NO: 54 - protein, 25 aa, Hepatitis C virus
GGTPTVATRDGKLPATQLRRHIDLL

SEQ ID NO: 55 - protein, 25 aa, Hepatitis C virus
GGTPTLAARDASVPTTTIRRHVDLL

SEQ ID NO: 56 - protein, 20 aa, Hepatitis C virus
LLSCLTVPASAYQVRNSTGL

SEQ ID NO: 57 - protein, 20 aa, Hepatitis C virus
QVRNSTGLYHVTNDCPNSSI

SEQ ID NO: 58 - protein, 20 aa, Hepatitis C virus
NDCPNSSIVYEAHDAILHTP

SEQ ID NO: 59 - protein, 20 aa, Hepatitis C virus
SNSSIVYEAADMIMHTPGCV

SEQ ID NO: 60 - protein, 19 aa, Hepatitis C virus
HDAILHTPGVPCVREGNVS

SEQ ID NO: 61 - protein, 20 aa, Hepatitis C virus
CVREGNVSRCWVAMTPTVAT

SEQ ID NO: 62 - protein, 20 aa, Hepatitis C virus
AMTPTVATRDGKLPATQLRR

SEQ ID NO: 63 - protein, 20 aa, Hepatitis C virus
LPATQLRRHIDLLVGSATLC

SEQ ID NO: 64 - protein, 20 aa, Hepatitis C virus
LVGSATLCSALYVGDLCGSV

SEQ ID NO: 65 - protein, 20 aa, Hepatitis C virus
QLFTFSPRRHWTTQGCNCSI

SEQ ID NO: 66 - protein, 20 aa, Hepatitis C virus
TQGCNCSIYPGHITGHRMAW

SEQ ID NO: 67 - protein, 20 aa, Hepatitis C virus
ITGHRMAWDMMMNWSPTAAL

SEQ ID NO: 68 - protein, 20 aa, Hepatitis C virus
NWSPTAALVMAQLLRIPQAI

SEQ ID NO: 69 - protein, 20 aa, Hepatitis C virus
LLRIPQAILDMIAGAHWGVL

SEQ ID NO: 70 - protein, 20 aa, Hepatitis C virus
AGAHWGVLAGIAYFSMVGNM

SEQ ID NO: 71 - protein, 20 aa, Hepatitis C virus
VVLLLFAGVDAETIVSGGQA

SEQ ID NO: 72 - protein, 20 aa, Hepatitis C virus
SGLVSLFTPGAKQNIQLINT

SEQ ID NO: 73 - protein, 20 aa, Hepatitis C virus
QNIQLINTNGQWHINSTALN

SEQ ID NO: 74 - protein, 21 aa, Hepatitis C virus
LNCNESLNTGWWLAGLIYQHK

SEQ ID NO: 75 - protein, 20 aa, Hepatitis C virus
AGLIYQHKFNSSGCPERLAS

SEQ ID NO: 76 - protein, 20 aa, Hepatitis C virus
GCPERLASCRPLTDFDQGWG

SEQ ID NO: 77 - protein, 20 aa, Hepatitis C virus
TDFDQGWGPISYANGSGPDQ

SEQ ID NO: 78 - protein, 20 aa, Hepatitis C virus
ANGSGPDQRPYCWHYPPKPC

SEQ ID NO: 79 - protein, 20 aa, Hepatitis C virus
WHYPPKPCGIVPAKSVCGPV

SEQ ID NO: 80 - protein, 20 aa, Hepatitis C virus
AKSVCGPVYCFTPSPVVVGT

SEQ ID NO: 81 - protein, 20 aa, Hepatitis C virus
PSPVVVGTTDRSGAPTYSWG

SEQ ID NO: 82 - protein, 20 aa, Hepatitis C virus
GAPTYSWGENDTDVFVLNNT

SEQ ID NO: 83 - protein, 20 aa, Hepatitis C virus
GNWFGCTWMNSTGFTKVCGA

SEQ ID NO: 84 - protein, 20 aa, Hepatitis C virus
GFTKVCGAPPVCIGGAGNNT

SEQ ID NO: 85 - protein, 19 aa, Hepatitis C virus
IGGAGNNTLHCPTDCRKHP

SEQ ID NO: 86 - protein, 20 aa, Hepatitis C virus
TDCFRKHPDATYSRCGSGPW

SEQ ID NO: 87 - protein, 20 aa, Hepatitis C virus
SRCGSGPWITPRCLVDYPYR

SEQ ID NO: 88 - protein, 20 aa, Hepatitis C virus
CLVDYPYRLWHYPCTINYTI

SEQ ID NO: 89 - protein, 20 aa, Hepatitis C virus
PCTINYTIFKIRMYVGGVEH

SEQ ID NO: 90 - protein, 20 aa, Hepatitis C virus
MYVGGVEHRLEAACNWTPGE

SEQ ID NO: 91 - protein, 20 aa, Hepatitis C virus
ACNWTPGERCDLEDRDRSEL

SEQ ID NO: 92 - protein, 20 aa, Hepatitis C virus
EDRDRSELSPLLLTTTQWQV

SEQ ID NO: 93 - protein, 9 aa, Hepatitis C virus
YQVRNSTGL

SEQ ID NO: 94 - DNA, 29 bp, Hepatitis C virus
  misc_feature: /note=“antisense”
acgtccgtacgttcgaattaattaatcga

SEQ ID NO: 95 - DNA, 60 bp, Hepatitis C virus
  misc_feature: /note=“antisense”
cctccggacgtgcactagctcccgtctgtggtagtggtggtagtgattatcaattaattg

SEQ ID NO: 96 - DNA, 19 bp, Hepatitis C virus
gtttaaccactgcatgatg

SEQ ID NO: 97 - DNA, 20 bp, Hepatitis C virus
gtcccatcgagtgcggctac

SEQ ID NO: 98 - DNA, 45 bp, Hepatitis C virus
cgtgacatggtacattccggacacttggcgcacttcataagcgga

SEQ ID NO: 99 - DNA, 42 bp, Hepatitis C virus
tgcctcatacacaatggagctctgggacgagtcgttcgtgac

SEQ ID NO: 100 - DNA, 42 bp, Hepatitis C virus
tacccagcagcgggagctctgttgctcccgaacgcagggcac

SEQ ID NO: 101 - DNA, 42 bp, Hepatitis C virus
tgtcgtggtggggacggaggcctgcctagctgcgagcgtggg

SEQ ID NO: 102 - DNA, 48 bp, Hepatitis C virus
cgttatgtggcccgggtagattgagcactggcagtcctgcaccgtctc

SEQ ID NO: 103 - DNA, 42 bp, Hepatitis C virus
cagggccgttctaggcctccactgcatcatcatatcccaagc

SEQ ID NO: 104 - DNA, 26 bp, Hepatitis C virus
ccggaatgtaccatgtcacgaacgac

SEQ ID NO: 105 - DNA, 24 bp, Hepatitis C virus
gctccattgtgtatgaggcagcgg

SEQ ID NO: 106 - DNA, 23 bp, Hepatitis C virus
gagctcccgctgctgggtagcgc

SEQ ID NO: 107 - DNA, 25 bp, Hepatitis C virus
cctccgtccccaccacgacaatacg

SEQ ID NO: 108 - DNA, 27 bp, Hepatitis C virus
ctacccgggccacataacgggtcaccg

SEQ ID NO: 109 - DNA, 24 bp, Hepatitis C virus
ggaggcctacaacggccctggtgg

SEQ ID NO: 110 - DNA, 22 bp, Hepatitis C virus
ttctatcgattaaatagaattc

SEQ ID NO: 111 - DNA, 23 bp, Hepatitis C virus
gccatacgctcacagccgatccc

SEQ ID NO: 112 - protein, 20 aa, Hepatitis C virus
YEVRNVSGIYHVTNDCSNSS

SEQ ID NO: 113 - protein, 20 aa, Hepatitis C virus
TNDCSNSSIVYEAADMIMHT

SEQ ID NO: 114 - protein, 20 aa, Hepatitis C virus
AADMIMHTPGCVPCVRENNS

SEQ ID NO: 115 - protein, 20 aa, Hepatitis C virus
PCVRENNSSRCWVALTPTLA

SEQ ID NO: 116 - protein, 20 aa, Hepatitis C virus
VALTPTLAARNASVPTTTIR

SEQ ID NO: 117 - protein, 20 aa, Hepatitis C virus
SVPTTTIRRHVDLLVGAAAF

SEQ ID NO: 118 - protein, 20 aa, Hepatitis C virus
LLVGAAAFCSAMYVGDLCGS

SEQ ID NO: 119 - protein, 20 aa, Hepatitis C virus
YVGDLCGSVFLVSQLFTISP

SEQ ID NO: 120 - protein, 20 aa, Hepatitis C virus
SQLFTISPRRHETVQDCNCS

SEQ ID NO: 121 - protein, 20 aa, Hepatitis C virus
TVQDCNCSIYPGHITGHRMA

SEQ ID NO: 122 - protein, 20 aa, Hepatitis C virus
HITGHRMAWDMMMNWSPTTA

SEQ ID NO: 123 - protein, 135 aa, Artificial Sequence
  Description of Artificial SequenceHCV E1s protein (amino acids 192-326 )
YEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNW

(19) United States
(12) Patent Application Publication
(10) Pub. No.: US 2004/0126395 A1
(43) Pub. Date: Jul. 1, 2004

(54) Purified hepatitis C virus envelope proteins for diagnostic and therapeutic use

(76) Inventors: Geert Maertens, Brugge (BE); Erik Depla, Destelbergen (BE); Fons Bosman, Opwijk (BE)

Correspondence Address: NIXON & VANDERHYE, PC, 1100 N GLEBE ROAD, 8TH FLOOR, ARLINGTON, VA, 22201-4714, US

(21) Appl. No.: 10/321,798

(22) Filed: Dec. 18, 2002

Related U.S. Application Data
(60) Provisional application No. 60/418,358, filed on Oct. 16, 2002.
(60) Provisional application No. 60/453,708, filed on Dec. 18, 2001.

(51) Int. Cl. A61K 39/29

(52) U.S. Cl. 424/228.1

(57) ABSTRACT

The present invention relates to a method for purifying recombinant HCV single or specific oligomeric envelope proteins selected from the group consisting of E1 and/or E2 and/or E1/E2, characterized in that upon lysing the transformed host cells to isolate the recombinantly expressed protein a disulphide bond cleavage or reduction step is carried out with a disulphide bond cleavage agent. The present invention also relates to a composition isolated by such a method. The present invention also relates to the diagnostic and therapeutic application of these compositions. Furthermore, the invention relates to the use of HCV E1 protein and peptides for prognosing and monitoring the clinical effectiveness and/or clinical outcome of HCV treatment.

U.S. Provisional Patent Application No. 60/418,358, filed Oct. 16, 2002, and U.S. Patent application Ser. No. 10/020,510 (now 60/___,___), filed Dec. 18, 2001 All references cited herein are incorporated in their entirety by reference.

### FIELD OF THE INVENTION

The present invention relates to the general fields of recombinant protein expression, purification of recombinant proteins, synthetic peptides, diagnosis of HCV infection, prophylactic treatment against HCV infection and to the prognosis/monitoring of the clinical efficiency of treatment of an individual with chronic hepatitis, or the prognosis/monitoring of natural disease.

More particularly, the present invention relates to purification methods for hepatitis C virus envelope proteins, the use in diagnosis, prophylaxis or therapy of HCV envelope proteins purified according to the methods described in the present invention, the use of single or specific oligomeric E1 and/or E2 and/or E1/E2 envelope proteins in assays for monitoring disease, and/or diagnosis of disease, and/or treatment of disease. The invention also relates to epitopes of the E1 and/or E2 envelope proteins and monoclonal antibodies thereto, as well their use in diagnosis, prophylaxis or treatment.

### BACKGROUND OF THE INVENTION

The E2 protein purified from cell lysates according to the methods described in the present invention reacts with approximately 95% of patient sera. This reactivity is similar to the reactivity obtained with E2 secreted from CHO cells (Spaete et al., 1992). However, the intracellularly expressed form of E2 may more closely resemble the native viral envelope protein because it contains high mannose carbohydrate motifs, whereas the E2 protein secreted from CHO cells is further modified with galactose and sialic acid sugar moieties. When the aminoterminal half of E2 is expressed in the baculovirus system, only about 13 to 21% of sera from several patient groups can be detected (Inoue et al., 1992). After expression of E2 from E. coli, the reactivity of HCV sera was even lower and ranged from 14 (Yokosuka et al., 1992) to 17% (Mita et al., 1992). About 75% of HCV sera (and 95% of chronic patients) are anti-E1 positive using the purified, vaccinia-expressed recombinant E1 protein of the present invention, in sharp contrast with the results of Kohara et al. (1992) and Hsu et al. (1993). Kohara et al. used a vaccinia-virus expressed E1 protein and detected anti-E1 antibodies in 7 to 23% of patients, while Hsu et al. only detected 14/50 (28%) sera using baculovirus-expressed E1.

These results show that not only a good expression system but also a good purification protocol are required to reach a high reactivity of the envelope proteins with human patient sera. This can be obtained using the proper expression system and/or purification protocols of the present invention which guarantee the conservation of the natural folding of the protein and the purification protocols of the present invention which guarantee the elimination of contaminating proteins and which preserve the conformation, and thus the reactivity of the HCV envelope proteins. The amounts of purified HCV envelope protein needed for diagnostic screening assays are in the range of grams per year. For vaccine purposes, even higher amounts of envelope protein would be needed. Therefore, the vaccinia virus system may be used for selecting the best expression constructs and for limited upscaling, and large-scale expression and purification of single or specific oligomeric envelope proteins containing high-mannose carbohydrates may be achieved when expressed from several yeast strains. In the case of hepatitis B for example, manufacturing of HBsAg from mammalian cells was much more costly compared with yeast-derived hepatitis B vaccines.

Clinical Importance of Necro-Inflammation and Fibrosis in HCV Infection.

The natural history of liver disease after HCV infection does vary significantly from patient to patient. About 20% of the acutely infected persons are able to resolve infection spontaneously, while 80% of infected persons progresses to a chronic infection. Chronic infection results in an ongoing inflammation and/or necrosis (=necro-inflammation) in the liver which can be diagnosed by histological analysis of a liver biopsy or which can be diagnosed using a surrogate marker such as the presence of the liver enzyme ALT in serum. This chronic infection increases the risk for development of fibrosis which can lead to development of cirrhosis and ultimately liver carcinoma. Many data suggest that the ongoing necro-inflammation drives progression to fibrosis and cirrhosis. It is estimated that up to 20% of HCV chronic carriers may develop cirrhosis over a time period of about 20 years and that of those with cirrhosis between 1 to 4%/year is at risk to develop liver carcinoma. (Lauer and Walker 2001, Shiffman 1999). Both cirrhosis and liver carcinoma are end-stage liver diseases for which the treatment options are limited to liver transplantation. Consequently, the most important aim of therapy for HCV is to reduce the risk of development of end-stage liver disease by reducing liver necro-inflammation and/or reducing fibrosis progression. For the documentation and/or diagnosis of liver damage several scoring systems have been developed for histological interpretation of a liver biopsy. These scoring systems may combine inflammation, necrosis and fibrosis in a single score such as the Histology Activity Index (HAI). Other scoring systems have separated the scores for necro-inflammation (=grading) from the one for fibrosis/cirrhosis (=staging). These systems include the system proposed by Ishak or the Metavir scoring system. A review of these scoring systems was published by Lefkowitch in 1997.

It has been shown in several studies that treatment with interferon, and more recently treatment with interferon combined with ribavirin and most recently treatment with pegylated interferon with or without ribavirin does change the natural history of HCV and halts further progression of liver fibrosis especially in those patients with a sustained viral response (Schvarcz et al. 1999, Shiffman 1999, Reichard et al. 1999, Poynard et al. 2002). The reduction of the risk for hepatocarcinogenesis in persons with sustained virological and even with sustained biochemical response has also been documented (Takimoto et al. 2002).

For persons without sustained virological response to interferon based therapy a maintenance interferon therapy may be helpful to prevent histological progression, but this only in a subset of patients (Alric et al. 2001).

Thus, many patients who do not respond to interferon based therapies or who are excluded from these therapies for several reasons (this may mount up to 70% of patients referred to clinic, Falck-Ytter et al. 2002), remain without a therapeutic option today to reduce liver necro-inflammation and/or reduce the progression of fibrosis in order to avoid end-stage liver disease.

Aims of the Invention

It is an aim of the present invention to provide a new purification method for recombinantly expressed E1 and/or E2 and/or E1/E2 proteins such that said recombinant proteins are directly usable for diagnostic and vaccine purposes as single or specific oligomeric recombinant proteins free from contaminants instead of aggregates.

It is another aim of the present invention to provide compositions comprising purified (single or specific oligomeric) recombinant E1 and/or E2 and/or E1/E2 glycoproteins comprising conformational epitopes from the E1 and/or E2 domains of HCV.

It is yet another aim of the present invention to provide novel recombinant vector constructs for recombinantly expressing E and/or E2 and/or E1/E2 proteins, as well as host cells transformed with said vector constructs.

It is also an aim of the present invention to provide a method for producing and purifying recombinant HCV E1 and/or E2 and/or E1/E2 proteins.

It is also an aim of the present invention to provide diagnostic and immunogenic uses of the recombinant HCV E1 and/or E2 and/or E1/E2 proteins of the present invention, as well as to provide kits for diagnostic use, vaccines or therapeutics comprising any of the recombinant HCV E1 and/or E2 and/or E1/E2 proteins of the present invention.

It is further an aim of the present invention to provide for a new use of E1, E2, and/or E1/E2 proteins, or suitable parts thereof, for monitoring/prognosing the response to treatment of patients (e.g. with interferon) suffering from HCV infection.

It is also an aim of the present invention to provide for the use of the recombinant E1, E2, and/or E1/E2 proteins of the present invention in HCV screening and confirmatory antibody tests.

It is also an aim of the present invention to provide E1 and/or E2 peptides which can be used for diagnosis of HCV infection and for raising antibodies. Such peptides may also be used to isolate human monoclonal antibodies.

It is also an aim of the present invention to provide monoclonal antibodies, more particularly human monoclonal antibodies or mouse monoclonal antibodies which are humanized, which react specifically with E1 and/or E2 epitopes, either comprised in peptides or conformational epitopes comprised in recombinant proteins.

It is also an aim of the present invention to provide possible uses of anti-E1 or anti-E2 monoclonal antibodies for HCV antigen detection or for therapy of chronic HCV infection.

It is also an aim of the present invention to provide kits for monitoring/prognosing the response to treatment (e.g. with interferon) of patients suffering from HCV infection or monitoring/prognosing the outcome of the disease.

All the aims of the present invention are considered to have been met by the embodiments as set out below.

Definitions

The following definitions serve to illustrate the different terms and expressions used in the present invention.

The term ‘hepatitis C virus single envelope protein’ refers to a polypeptide or an analogue thereof (e.g. mimotopes) comprising an amino acid sequence (and/or amino acid analogues) defining at least one HCV epitope of either the E1 or the E2 region. These single envelope proteins in the broad sense of the word may be both monomeric or homo-oligomeric forms of recombinantly expressed envelope proteins. Typically, the sequences defining the epitope correspond to the amino acid sequence of either the E1 or the E2 region of HCV (either identically or via substitution of analogues of the native amino acid residue that do not destroy the epitope). In general, the epitope-defining sequence will be 3 or more amino acids in length, more typically, 5 or more amino acids in length, more typically 8 or more amino acids in length, and even more typically 10 or more amino acids in length. With respect to conformational epitopes, the length of the epitope-defining sequence can be subject to wide variations, since it is believed that these epitopes are formed by the three-dimensional shape of the antigen (e.g. folding). Thus, the amino acids defining the epitope can be relatively few in number, but widely dispersed along the length of the molecule being brought into the correct epitope conformation via folding. The portions of the antigen between the residues defining the epitope may not be critical to the conformational structure of the epitope. For example, deletion or substitution of these intervening sequences may not affect the conformational epitope provided sequences critical to epitope conformation are maintained (e.g. cysteines involved in disulfide bonding, glycosylation sites, etc.). A conformational epitope may also be formed by 2 or more essential regions of subunits of a homooligomer or heterooligomer.

The HCV antigens of the present invention comprise conformational epitopes from the E1 and/or E2 (envelope) domains of HCV. The E1 domain, which is believed to correspond to the viral envelope protein, is currently estimated to span amino acids 192-383 of the HCV polyprotein (Hijikata et al., 1991). Upon expression in a mammalian system (glycosylated), it is believed to have an approximate molecular weight of 35 kDa as determined via SDS-PAGE. The E2 protein, previously called NS1, is believed to span amino acids 384-809 or 384-746 (Grakoui et al., 1993) of the HCV polyprotein and to also be an envelope protein. Upon expression in a vaccinia system (glycosylated), it is believed to have an apparent gel molecular weight of about 72 kDa. It is understood that these protein endpoints are approximations (e.g. the carboxy terminal end of E2 could lie somewhere in the 730-820 amino acid region, e.g. ending at amino acid 730, 735, 740, 742, 744, 745, preferably 746, 747, 748, 750, 760, 770, 780, 790, 800, 809, 810, 820). The E2 protein may also be expressed together with the E1, P7 (aa 747-809), NS2 (aa 810-1026), NS4A (aa 1658-1711) or NS4B (aa 1712-1972). Expression together with these other HCV proteins may be important for obtaining the correct protein folding.

It is also understood that the isolates used in the examples section of the present invention were not intended to limit the scope of the invention and that any HCV isolate from type 1, 2, 3, 4, 5, 6, 7, 8, 9, 10 or any other new genotype of HCV is a suitable source of E1 and/or E2 sequence for the practice of the present invention.

The E1 and E2 antigens used in the present invention may be full-length viral proteins, substantially full-length versions thereof, or functional fragments thereof (e.g. fragments which are not missing sequence essential to the formation or retention of an epitope). Furthermore, the HCV antigens of the present invention can also include other sequences that do not block or prevent the formation of the conformational epitope of interest. The presence or absence of a conformational epitope can be readily determined though screening the antigen of interest with an antibody (polyclonal serum or monoclonal to the conformational epitope) and comparing its reactivity to that of a denatured version of the antigen which retains only linear epitopes (if any). In such screening using polyclonal antibodies, it may be advantageous to adsorb the polyclonal serum first with the denatured antigen and see if it retains antibodies to the antigen of interest.

The HCV antigens of the present invention can be made by any recombinant method that provides the epitope of intrest. For example, recombinant intracellular expression in mammalian or insect cells is a preferred method to provide glycosylated E1 and/or E2 antigens in ‘native’ conformation as is the case for the natural HCV antigens. Yeast cells and mutant yeast strains (e.g. mnn 9 mutant (Kniskern et al., 1994) or glycosylation mutants derived by means of vanadate resistence selection (Ballou et al., 1991)) may be ideally suited for production of secreted high-mannose-type sugars; whereas proteins secreted from mammalian cells may contain modifications including galactose or sialic acids which may be undesirable for certain diagnostic or vaccine applications. However, it may also be possible and sufficient for certain applications, as it is known for proteins, to express the antigen in other recombinant hosts (such as E. coli) and renature the protein after recovery.

The term ‘fusion polypeptide’ intends a polypeptide in which the HCV antigen(s) are part of a single continuous chain of amino acids, which chain does not occur in nature. The HCV antigens may be connected directly to each other by peptide bonds or be separated by intervening amino acid sequences. The fusion polypeptides may also contain amino acid sequences exogenous to HCV.

The term ‘solid phase’ intends a solid body to which the individual HCV antigens or the fusion polypeptide comprised of HCV antigens are bound covalently or by noncovalent means such as hydrophobic adsorption.

The term ‘biological sample’ intends a fluid or tissue of a mammalian individual (e.g. an anthropoid, a human) that commonly contains antibodies produced by the individual, more particularly antibodies against HCV. The fluid or tissue may also contain HCV antigen. Such components are known in the art and include, without limitation, blood, plasma, serum, urine, spinal fluid, lymph fluid, secretions of the respiratory, intestinal or genitourinary tracts, tears, saliva, milk, white blood cells and myelomas. Body components include biological liquids. The term ‘biological liquid’ refers to a fluid obtained from an organism. Some biological fluids are used as a source of other products, such as clotting factors (e.g. Factor VIII;C), serum albumin, growth hormone and the like. In such cases, it is important that the source of biological fluid be free of contamination by virus such as HCV.

The term ‘immunologically reactive’ means that the antigen in question will react specifically with anti-HCV antibodies present in a body component from an HCV infected individual.

The term ‘immune complex’ intends the combination formed when an antibody binds to an epitope on an antigen.

‘E1’ as used herein refers to a protein or polypeptide expressed within the first 400 amino acids of an HCV polyprotein, sometimes referred to as the E, ENV or S protein. In its natural form it is a 35 kDa glycoprotein which is found in strong association with membranes. In most natural HCV strains, the E1 protein is encoded in the viral polyprotein following the C (core) protein. The E1 protein extends from approximately amino acid (aa) 192 to about aa 383 of the full-length polyprotein.

The term ‘E1’ as used herein also includes analogs and truncated forms that are immunologically cross-reactive with natural E1, and includes E1 proteins of genotypes 1, 2, 3, 4, 5, 6, 7, 8, 9, 10, or any other newly identified HCV type or subtype.

‘E2’ as used herein refers to a protein or polypeptide expressed within the first 900 amino acids of an HCV polyprotein, sometimes referred to as the NS 1 protein. In its natural form it is a 72 kDa glycoprotein that is found in strong association with membranes. In most natural HCV strains, the E2 protein is encoded in the viral polyprotein following the E1 protein. The E2 protein extends from approximately amino acid position 384 to amino acid position 746, another form of E2 extends to amino acid position 809. The term ‘E2’ as used herein also includes analogs and truncated forms that are immunologically cross-reactive with natural E2. For example, insertions of multiple codons between codon 383 and 384, as well as deletions of amino acids 384-387 have been reported by Kato et al. (1992).

‘E1/E2’ as used herein refers to an oligomeric form of envelope proteins containing at least one E1 component and at least one E2 component.

The term ‘specific oligomeric’ E1 and/or E2 and/or E1/E2 envelope proteins refers to all possible oligomeric forms of recombinantly expressed E1 and/or E2 envelope proteins which are not aggregates. E1 and/or E2 specific oligomeric envelope proteins are also referred to as homo-oligomeric E1 or E2 envelope proteins (see below).

The term ‘single or specific oligomeric’ E1 and/or E2 and/or E1/E2 envelope proteins refers to single monomeric E1 or E2 proteins (single in the strict sense of the word) as well as specific oligomeric E1 and/or E2 and/or E1/E2 recombinantly expressed proteins. These single or specific oligomeric envelope proteins according to the present invention can be further defined by the following formula (E1)ₓ(E2)_{y} wherein x can be a number between 0 and 100, and y can be a number between o and 100, provided that x and y are not both 0. With x=1 and y=0 said envelope proteins include monomeric E1.

The term ‘homo-oligomer’ as used herein refers to a complex of E1 and/or E2 containing more than one E1 or E2 monomer, e.g. E1/E1 dimers, E1/E1/E1 trimers or E1/E1/E1/E1 tetramers and E2/E2 dimers, E2/E2/E2 trimers or E2/E2/E2/E2 tetramers, E1 pentamers and hexamers, E2 pentamers and hexamers or any higher-order homo-oligomers of E1 or E2 are all ‘homo-oligomers’ within the scope of this definition. The oligomers may contain one, two, or several different monomers of E1 or E2 obtained from different types or subtypes of hepatitis C virus including for example those described in an international application published under WO 94/25601 and European application No. 94870166.9 both by the present applicants. Such mixed oligomers are still homo-oligomers within the scope of this invention, and may allow more universal diagnosis, prophylaxis or treatment of HCV.

The term ‘purified’ as applied to proteins herein refers to a composition wherein the desired protein comprises at least 35% of the total protein component in the composition. The desired protein preferably comprises at least 40%, more preferably at least about 50%, more preferably at least about 60%, still more preferably at least about 70%, even more preferably at least about 80%, even more preferably at least about 90%, and most preferably at least about 95% of the total protein component. The composition may contain other compounds such as carbohydrates, salts, lipids, solvents, and the like, withouth affecting the determination of the percentage purity as used herein. An ‘isolated’ HCV protein intends an HCV protein composition that is at least 35% pure.

The term ‘essentially purified proteins’ refers to proteins purified such that they can be used for in vitro diagnostic methods and as a therapeutic compound. These proteins are substantially free from cellular proteins, vector-derived proteins or other HCV viral components. Usually these proteins are purified to homogeneity (at least 80% pure, preferably, 90%, more preferably 95%, more preferably 97%, more preferably 98%, more preferably 99%, even more preferably 99.5%, and most preferably the contaminating proteins should be undetectable by conventional methods like SDS-PAGE and silver staining.

The term ‘recombinantly expressed’ used within the context of the present invention refers to the fact that the proteins of the present invention are produced by recombinant expression methods be it in prokaryotes, or lower or higher eukaryotes as discussed in detail below.

The term ‘lower eukaryote’ refers to host cells such as yeast, fungi and the like. Lower eukaryotes are generally (but not necessarily) unicellular. Preferred lower eukaryotes are yeasts, particularly species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts.

The term ‘prokaryotes’ refers to hosts such as E. coli, Lactobacillus, Lactococcus, Salmonella, Streptococcus, Bacillus subtilis or Streptomyces. Also these hosts are contemplated within the present invention.

The term ‘higher eukaryote’ refers to host cells derived from higher animals, such as mammals, reptiles, insects, and the like. Presently preferred higher eukaryote host cells are derived from Chinese hamster (e.g. CHO), monkey (e.g. COS and Vero cells), baby hamster kidney (BHK), pig kidney (PK15), rabbit kidney 13 cells (RK13), the human osteosarcoma cell line 143 B, the human cell line HeLa and human hepatoma cell lines like Hep G2, and insect cell lines (e.g. Spodoptera frugiperda). The host cells may be provided in suspension or flask cultures, tissue cultures, organ cultures and the like. Alternatively the host cells may also be transgenic animals.

The term ‘polypeptide’ refers to a polymer of amino acids and does not refer to a specific length of the product; thus, peptides, oligopeptides, and proteins are included within the definition of polypeptide. This term also does not refer to or exclude post-expression modifications of the polypeptide, for example, glycosylations, acetylations, phosphorylations and the like. Included within the definition are, for example, polypeptides containing one or more analogues of an amino acid (including, for example, unnatural amino acids, PNA, etc.), polypeptides with substituted linkages, as well as other modifications known in the art, both naturally occurring and non-naturally occurring.

The term ‘recombinant polynucleotide or nucleic acid’ intends a polynucleotide or nucleic acid of genomic, CDNA, semisynthetic, or synthetic origin which, by virtue of its origin or manipulation: (1) is not associated with all or a portion of a polynucleotide with which it is associated in nature, (2) is linked to a polynucleotide other than that to which it is linked in nature, or (3) does not occur in nature.

The term ‘recombinant host cells’, ‘host cells’, ‘cells’, ‘cell lines’, ‘cell cultures’, and other such terms denoting microorganisms or higher eukaryotic cell lines cultured as unicellular entities refer to cells which can be or have been, used as recipients for a recombinant vector or other transfer polynucleotide, and include the progeny of the original cell which has been transfected. It is understood that the progeny of a single parental cell may not necessarily be completely identical in morphology or in genomic or total DNA complement as the original parent, due to natural, accidental, or deliberate mutation.

The term ‘replicon’ is any genetic element, e.g., a plasmid, a chromosome, a virus, a cosmid, etc., that behaves as an autonomous unit of polynucleotide replication within a cell; i.e., capable of replication under its own control.

The term ‘vector’ is a replicon further comprising sequences providing replication and/or expression of a desired open reading frame.

The term ‘control sequence’ refers to polynucleotide sequences which are necessary to effect the expression of coding sequences to which they are ligated. The nature of such control sequences differs depending upon the host organism; in prokaryotes, such control sequences generally include promoter, ribosomal binding site, and terminators; in eukaryotes, generally, such control sequences include promoters, terminators and, in some instances, enhancers. The term ‘control sequences’ is intended to include, at a minimum, all components whose presence is necessary for expression, and may also include additional components whose presence is advantageous, for example, leader sequences which govem secretion.

The term ‘promoter’ is a nucleotide sequence which is comprised of consensus sequences which allow the binding of RNA polymerase to the DNA template in a manner such that mRNA production initiates at the normal transcription initiation site for the adjacent structural gene.

The expression ‘operably linked’ refers to a juxtaposition wherein the components so described are in a relationship permitting them to function in their intended manner. A control sequence ‘operably linked’ to a coding sequence is ligated in such a way that expression of the coding sequence is achieved under conditions compatible with the control sequences.

An ‘open reading frame’ (ORF) is a region of a polynucleotide sequence which encodes a polypeptide and does not contain stop codons; this region may represent a portion of a coding sequence or a total coding sequence.

A ‘coding sequence’ is a polynucleotide sequence which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include but is not limited to mRNA, DNA (including cDNA), and recombinant polynucleotide sequences.

As used herein, ‘epitope’ or ‘antigenic determinant’ means an amino acid sequence that is immunoreactive. Generally an epitope consists of at least 3 to 4 amino acids, and more usually, consists of at least 5 or 6 amino acids, sometimes the epitope consists of about 7 to 8, or even about 10 amino acids. As used herein, an epitope of a designated polypeptide denotes epitopes with the same amino acid sequence as the epitope in the designated polypeptide, and immunologic equivalents thereof. Such equivalents also include strain, subtype (=genotype), or type(group)-specific variants, e.g. of the currently known sequences or strains belonging to genotypes 1a, 1b, 1c, 1d, 1e, 1f, 2a, 2b, 2c, 2d, 2e, 2f, 2g, 2h, 2i, 3a, 3b, 3c, 3d, 3e, 3f, 3g, 4a, 4b, 4c, 4d, 4e, 4f, 4g, 4h, 4i, 4j, 4k, 41, 5a, 5b, 6a, 6b, 6c, 7a, 7b, 7c, 8a, 8b, 9a, 9b, 10a, or any other newly defined HCV (sub)type. It is to be understood that the amino acids constituting the epitope need not be part of a linear sequence, but may be interspersed by any number of amino acids, thus forming a conformational epitope.

The term ‘immunogenic’ refers to the ability of a substance to cause a humoral and/or cellular response, whether alone or when linked to a carrier, in the presence or absence of an adjuvant. ‘Neutralization’ refers to an immune response that blocks the infectivity, either partially or fully, of an infectious agent. A ‘vaccine’ is an immunogenic composition capable of eliciting protection against HCV, whether partial or complete. A vaccine may also be useful for treatment of an individual, in which case it is called a therapeutic vaccine.

The term ‘therapeutic’ refers to a composition capable of treating HCV infection.

The term ‘effective amount’ refers to an amount of epitope-bearing polypeptide sufficient to induce an immunogenic response in the individual to which it is administered, or to otherwise detectably immunoreact in its intended system (e.g., immunoassay). Preferably, the effective amount is sufficient to effect treatment, as defined above. The exact amount necessary will vary according to the application. For vaccine applications or for the generation of polyclonal antiserum/antibodies, for example, the effective amount may vary depending on the species, age, and general condition of the individual, the severity of the condition being treated, the particular polypeptide selected and its mode of administration, etc. It is also believed that effective amounts will be found within a relatively large, non-critical range. An appropriate effective amount can be readily determined using only routine experimentation. Preferred ranges of E1 and/or E2 and/or E1/E2 single or specific oligomeric envelope proteins for prophylaxis of HCV disease are 0.01 to 100 μg/dose, preferably 0.1 to 50 μg/dose. Several doses may be needed per individual in order to achieve a sufficient immune response and subsequent protection against HCV disease.

### DETAILED DESCRIPTION OF THE INVENTION

More -particularly, the present invention contemplates a method for isolating or purifying recombinant HCV single or specific oligomeric envelope protein selected from the group consisting of E1 and/or E2 and/or E1/E2, characterized in that upon lysing the transformed host cells to isolate the recombinantly expressed protein a disulphide bond cleavage or reduction step is carried out with a disculphide bond cleaving agent.

The essence of these ‘single or specific oligomeric’ envelope proteins of the invention is that they are free from contaminating proteins and that they are not disulphide bond linked with contaminants.

The proteins according to the present invention are recombinantly expressed in lower or higher eukaryotic cells or in prokaryotes. The recombinant proteins of the present invention are preferably glycosylated and may contain high-mannose-type, hybrid, or complex glycosylations. Preferentially said proteins are expressed from mammalian cell lines as discussed in detail in the Examples section, or in yeast such as in mutant yeast strains also as detailed in the Examples section.

The proteins according to the present invention may be secreted or expressed within components of the cell, such as the ER or the Golgi Apparatus. Preferably, however, the proteins of the present invention bear high-mannose-type glycosylations and are retained in the ER or Golgi Apparatus of mammalian cells or are retained in or secreted from yeast cells, preferably secreted from yeast mutant strains such as the mnn9 mutant (Kniskem et al., 1994), or from mutants that have been selected by means of vanadate resistence (Ballou et al., 1991).

Upon expression of HCV envelope proteins, the present inventors could show that some of the free thiol groups of cysteines not involved in intra- or inter-molecular disulphide bridges, react with cysteines of host or expression-system-derived (e.g. vaccinia) proteins or of other HCV envelope proteins (single or oligomeric), and form aspecific intermolecular bridges. This results in the formation of ‘aggregates’ of HCV envelope proteins together with contaminating proteins. It was also shown in WO 92/08734 that ‘aggregates’ were obtained after purification, but it was not described which protein interactions were involved. In patent application WO 92/08734, recombinant E1/E2 protein expressed with the vaccinia virus system were partially purified as aggregates and only found to be 70% pure, rendering the purified aggregates not useful for diagnostic, prophylactic or therapeutic purposes.

Therefore, a major aim of the present invention resides in the separation of single or specific-oligomeric HCV envelope proteins from contaminating proteins, and to use the purified proteins (>95% pure) for diagnostic, prophylactic and therapeutic purposes. To those purposes, the present inventors have been able to provide evidence that aggregated protein complexes (‘aggregates’) are formed on the basis of disulphide bridges and non-covalent protein-protein interactions. The present invention thus provides a means for selectively cleaving the disulphide bonds under specific conditions and for separating the cleaved proteins from contaminating proteins which greatly interfere with diagnostic, prophylactic and therapeutic applications. The free thiol groups may be blocked (reversibly or irreversibly) in order to prevent the reformation of disulphide bridges, or may be left to oxidize and oligomerize with other envelope proteins (see definition homo-oligomer). It is to be understood that such protein oligomers are essentially different from the ‘aggregates’ described in WO 92/08734 and WO 94/01778, since the level of contaminating proteins is undetectable.

Said Disuphide Bond Cleavage May also be Achieved by:

(1) performic acid oxidation by means of cysteic acid in which case the cysteine residues are modified into cysteic acid (Moore et al., 1963).

(2) Sulfitolysis (R-S-S-R→2 R-SO⁻₃) for example by means of sulphite (SO²⁻₃) together with a proper oxidant such as Cu²⁺ in which case the cysteine is modified into S-sulpho-cysteine (Bailey and Cole, 1959).

(3) Reduction by means of mercaptans, such as dithiotreitol (DDT), β-mercapto-ethanol, cysteine, glutathione Red, ε-mercapto-ethylamine, or thioglycollic acid, of which DTT and β-mercapto-ethanol are commonly used (Cleland, 1964), is the preferred method of this invention because the method can be performed in a water environment and because the cysteine remains unmodified.

(4) Reduction by means of a phosphine (e.g. BU₃P) (Ruegg and Rudinger, 1977).

All these compounds are thus to be regarded as agents or means for cleaving disulphide bonds according to the present invention.

Said disulphide bond cleavage (or reducing) step of the present invention is preferably a partial disulphide bond cleavage (reducing) step (carried out under partial cleavage or reducing conditions).

A preferred disulphide bond cleavage or reducing agent according to the present invention is dithiothreitol (DTT). Partial reduction is obtained by using a low concentration of said reducing agent, i.e. for DTT for example in the concentration range of about 0.1 to about 50 mM, preferably about 0.1 to about 20 mM, preferably about 0.5 to about 10 mM, preferably more than 1 mM, more than 2 mM or more than 5 mM, more preferably about 1.5 mM, about 2.0 mM, about 2.5 mM, about 5 mM or about 7.5 mM.

Said disulphide bond cleavage step may also be carried out in the presence of a suitable detergent (as an example of a means for cleaving disulphide bonds or in combination with a cleaving agent) able to dissociate the expressed proteins, such as DecylPEG, EMPIGEN-BB, NP-40, sodium cholate, Triton X-100.

Said reduction or cleavage step (preferably a partial reduction or cleavage step) is carried out preferably in in the presence of (with) a detergent. A preferred detergent according to the present invention is Empigen-BB. The amount of detergent used is preferably in the range of 1 to 10%, preferably more than 3%, more preferably about 3.5% of a detergent such as Empigen-BB.

A particularly preferred method for obtaining disulphide bond cleavage employs a combination of a classical disulphide bond cleavage agent as detailed above and a detergent (also as detailed above). As contemplated in the Examples section, the particular combination of a low concentration of DTT (1.5 to 7.5 mM) and about 3.5% of Empigen-BB is proven to be a particularly preferred combination of reducing agent and detergent for the purification of recombinantly expressed E1 and E2 proteins. Upon gelfiltration chromatography, said partial reduction is shown to result in the production of possibly dimeric E1 protein and separation of this E1 protein from contaminating proteins that cause false reactivity upon use in immunoassays.

It is, however, to be understood that also any other combination of any reducing agent known in the art with any detergent or other means known in the art to make the cysteines better accessible is also within the scope of the present invention, insofar as said combination reaches the same goal of disulphide bridge cleavage as the preferred combination examplified in the present invention.

Apart from reducing the disulphide bonds, a disulphide bond cleaving means according to the present invention may also include any disulphide bridge exchanging agents (competitive agent being either organic or proteinaeous, see for instance Creighton, 1988) known in the art which allows the following type of reaction to occur:

R1 S−S R2+R3 SH→R1 S−S R3+R2 SH

R1, R2: compounds of protein aggregates

R3 SH: competitive agent (organic, proteinaeous)

The term ‘disulphide bridge exchanging agent’ is to be interpretated as including disulphide bond reforming as well as disulphide bond blocking agents.

The present invention also relates to methods for purifying or isolating HCV single or specific oligomeric envelelope proteins as set out above further including the use of any SH group blocking or binding reagent known in the art such as chosen from the following list:

Glutathion

5,5′-dithiobis-(2-nitrobenzoic acid) or bis-(3-carboxy4-nitrophenyl)-disulphide (DTNB or Ellman's reagent) (Elmann, 1959)

N-ethylmaleimide (NEM; Benesch et al., 1956)

N-(4-dimethylamino-3,5-dinitrophenyl) maleimide or Tuppy's maleimide which provides a color to the protein

P-chloromercuribenzoate (Grassetti et al., 1969)

4-vinylpyridine (Friedman and Krull, 1969) can be liberated after reaction by acid hydrolysis

acrylonitrile, can be liberated after reaction by acid hydrolysis (Weil and Seibles, 1961)

NEM-biotin (e.g. obtained from Sigma B1267)

2,2′-dithiopyridine (Grassetti and Murray, 1967)

4,4′-dithiopyridine (Grassetti and Murray, 1967)

6,6′-dithiodinicontinic acid (DTDNA; Brown and Cunnigham, 1970)

2,2′-dithiobis-(5′-nitropyridine) (DTNP; U.S. Pat. No. 3,597,160) or other dithiobis (heterocyclic derivative) compounds (Grassetti and Murray, 1969)

A survey of the publications cited shows that often different reagents for sulphydryl groups will react with varying numbers of thiol groups of the same protein or enzyme molecule. One may conclude that this variation in reactivity of the thiol groups is due to the steric environment of these groups, such as the shape of the molecule and the surrounding groups of atoms and their charges, as well as to the size, shape and charge of the reagent molecule or ion. Frequently the presence of adequate concentrations of denaturants such as sodium dodecylsulfate, urea or guanidine hydrochoride will cause sufficient unfolding of the protein molecule to permit equal access to all of the reagents for thiol groups. By varying the concentration of denaturant, the degree of unfolding can be controlled and in this way thiol groups with different degrees of reactivity may be revealed. Although up to date most of the work reported has been done with p-chloromercuribenzoate, N-ethylmaleimide and DTNB, it is likely that the other more recently developed reagents may prove equally useful. Because of their varying structures, it seems likely, in fact, that they may respond differently to changes in the steric environment of the thiol groups.

Alternatively, conditions such as low pH (preferably lower than pH 6) for preventing free SH groups from oxidizing and thus preventing the formation of large intermolecular aggregates upon recombinant expression and purification of E1 and E2 (envelope) proteins are also within the scope of the present invention.

A preferred SH group blocking reagent according to the present invention is N-ethylmaleimide (NEM). Said SH group blocking reagent may be administrated during lysis of the recombinant host cells and after the above-mentioned partial reduction process or after any other process for cleaving disulphide bridges. Said SH group blocking reagent may also be modified with any group capable of providing a detectable label and/or any group aiding in the immobilization of said recombinant protein to a solid substrate, e.g. biotinylated NEM.

Methods for cleaving cysteine bridges and blocking free cysteines have also been described in Darbre (1987), Means and Feeney (1971), and by Wong (1993).

A method to purify single or specific oligomeric recombinant E1 and/or E2 and/or E1/E2 proteins according to the present invention as defined above is further characterized as comprising the following steps:

lysing recombinant E1 and/or E2 and/or E1/E2 expressing host cells, preferably in the presence of an SH group blocking agent, such as N-ethylmaleimide (NEM), and possibly a suitable detergent, preferably Empigen-BB,

recovering said HCV envelope protein by affinity purification for instance by means lectin-chromatography, such as lentil-lectin chromatography, or immunoaffinity chromatography using anti-E1 and/or anti-E2 specific monoclonal antibodies, followed by,

reduction or cleavage of disulphide bonds with a disulphide bond cleaving agent, such as DTT, preferably also in the presence of an SH group blocking agent, such as NEM or Biotin-NEM, and,

recovering the reduced HCV E1 and/or E2 and/or E1/E2 envelope proteins for instance by gelfiltration (size exclusion chromatography or molecular sieving) and possibly also by an additional Ni²⁺-IMAC chromatography and desalting step.

It is to be understood that the above-mentioned recovery steps may also be carried out using any other suitable technique known by the person skilled in the art.

Preferred lectin-chromatography systems include Galanthus nivalis agglutinin (GNA)

chromatography, or Lens culinaris agglutinin (LCA) (lentil) lectin chromatography as illustrated in the Examples section. Other useful lectins include those recognizing high-mannose type sugars, such as Narcissus pseudonarcissus agglutinin (NPA), Pisum sativum agglutinin (PSA), or Allium ursinum agglutinin (AUA).

Preferably said method is usable to purify single or specific oligomeric HCV envelope protein produced intracellularly as detailed above.

For secreted E1 or E2 or E1/E2 oligomers, lectins binding complex sugars such as Ricinus communis agglutinin I (RCA I), are preferred lectins.

The present invention more particularly contemplates essentially purified recombinant HCV single or specific oligomeric envelope proteins, selected from the group consisting of E1 and/or E2 and/or E1/E2, characterized as being isolated or purified by a method as defined above.

The present invention more particularly relates to the purification or isolation of recombinant envelope proteins which are expressed from recombinant mammalian cells such as vaccinia.

The present invention also relates to the purification or isolation of recombinant envelope proteins which are expressed from recombinant yeast cells.

The present invention equally relates to the purification or isolation of recombinant envelope proteins which are expressed from recombinant bacterial (prokaryotic) cells.

The present invention also contemplates a recombinant vector comprising a vector sequence, an appropriate prokaryotic, eukaryotic or viral or synthetic promoter sequence followed by a nucleotide sequence allowing the expression of the single or specific oligomeric E1 and/or E2 and/or E1/E2 of the invention.

Particularly, the present invention contemplates a recombinant vector comprising a vector sequence, an appropriate prokaryotic, eukaryotic or viral or synthetic promoter sequence followed by a nucleotide sequence allowing the expression of the single E1 or E1 of the invention.

Particularly, the present invention contemplates a recombinant vector comprising a vector sequence, an appropriate prokaryotic, eukaryotic or viral or synthetic promoter sequence followed by a nucleotide sequence allowing the expression of the single E1 or E2 of the invention.

The segment of the HCV cDNA encoding the desired E1 and/or E2 sequence inserted into the vector sequence may be attached to a signal sequence. Said signal sequence may be that from a non-HCV source, e.g. the IgG or tissue plasminogen activator (tpa) leader sequence for expression in mammalian cells, or the α-mating factor sequence for expression into yeast cells, but particularly preferred constructs according to the present invention contain signal sequences appearing in the HCV genome before the respective start points of the E1 and E2 proteins. The segment of the HCV cDNA encoding the desired E1 and/or E2 sequence inserted into the vector may also include deletions e.g. of the hydrophobic domain(s) as illustrated in the examples section, or of the E2 hypervariable region I.

More particularly, the recombinant vectors according to the present invention encompass a nucleic acid having an HCV cDNA segment encoding the polyprotein starting in the region between amino acid positions 1 and 192 and ending in the region between positions 250 and 400 of the HCV polyprotein, more preferably ending in the region between positions 250 and 341, even more preferably ending in the region between positions 290 and 341 for expression of the HCV single E1 protein. Most preferably, the present recombinant vector encompasses a recombinant nucleic acid having a HCV cDNA seqment encoding part of the HCV polyprotein starting in the region between positions 117 and 192, and ending at any position in the region between positions 263 and 326, for expression of HCV single E1 protein. Also within the scope of the present invention are forms that have the first hydrophobic domain deleted (positions 264 to 293 plus or minus 8 amino acids), or forms to which a 5′-terminal ATG codon and a 3′-terminal stop codon has been added, or forms which have a factor Xa cleavage site and/or 3 to 10, preferably 6 Histidine codons have been added.

More particularly, the recombinant vectors according to the present invention encompass a nucleic acid having an HCV cDNA segment encoding the polyprotein starting in the region between amino acid positions 290 and 406 and ending in the region between positions 600 and 820 of the HCV polyprotein, more preferably starting in the region between positions 322 and 406, even more preferably starting in the region between positions 347 and 406, even still more preferably starting in the region between positions 364 and 406 for expression of the HCV single E2 protein. Most preferably, the present recombinant vector encompasses a recombinant nucleic acid having a HCV cDNA seqment encoding the polyprotein starting in the region between positions 290 and 406, and ending at any position of positions 623, 650, 661, 673, 710, 715, 720, 746 or 809, for expression of HCV single E2 protein. Also within the scope of the present invention are forms to which a 5′-terminal ATG codon and a 3′-terminal stop codon has been added, or forms which have a factor Xa cleavage site and/or 3 to 10, preferably 6 Histidine codons have been added.

A variety of vectors may be used to obtain recombinant expression of HCV single or specific oligomeric envelope proteins of the present invention. Lower eukaryotes such as yeasts and glycosylation mutant strains are typically transformed with plasmids, or are transformed with a recombinant virus. The vectors may replicate within the host independently, or may integrate into the host cell genome.

Higher eukaryotes may be transformed with vectors, or may be infected with a recombinant virus, for example a recombinant vaccinia virus. Techniques and vectors for the insertion of foreign DNA into vaccinia virus are well known in the art, and utilize, for example homologous recombination. A wide variety of viral promoter sequences, possibly terminator sequences and poly(A)-addition sequences, possibly enhancer sequences and possibly amplification sequences, all required for the mammalian expression, are available in the art. Vaccinia is particularly preferred since vaccinia halts the expression of host cell proteins. Vaccinia is also very much preferred since it allows the expression of E1 and E2 proteins of HCV in cells or individuals which are immunized with the live recombinant vaccinia virus. For vaccination of humans the avipox and Ankara Modified Virus (AMV) are particularly useful vectors.

Also known are insect expression transfer vectors derived from baculovirus Autographa californica nuclear polyhedrosis virus (AcNPV), which is a helper-independent viral expression vector. Expression vectors derived from this system usually use the strong viral polyhedrin gene promoter to drive the expression of heterologous genes. Different vectors as well as methods for the introduction of heterologous DNA into the desired site of baculovirus are available to the man skilled in the art for baculovirus expression. Also different signals for posttranslational modification recognized by insect cells are known in the art.

Also included within the scope of the present invention is a method for producing purified recombinant single or specific oligomeric HCV E1 or E2 or E1/E2 proteins, wherein the cysteine residues involved in aggregates formation are replaced at the level of the nucleic acid sequence by other residues such that aggregate formation is prevented. The recombinant proteins expressed by recombinant vectors caarying such a mutated E1 and/or E2 protein encoding nucleic acid are also within the scope of the present invention.

The present invention also relates to recombinant E1 and/or E2 and/or E1/E2 proteins characterized in that at least one of their glycosylation sites has been removed and are consequently termed glycosylation mutants. As explained in the Examples section, different glycosylation mutants may be desired to diagnose (screening, confirmation, prognosis, etc.) and prevent HCV disease according to the patient in question. An E2 protein glycosylation mutant lacking the GLY4 has for instance been found to improve the reactivity of certain sera in diagnosis. These glycosylation mutants are preferably purified according to the method disclosed in the present invention. Also contemplated within the present invention are recombinant vectors carrying the nucleic acid insert encoding such a E1 and/or E2 and/or E1/E2 glycosylation mutant as well as host cells tranformed with such a recombinant vector.

The present invention also relates to recombinant vectors including a polynucleotide which also forms part of the present invention. The present invention relates more particularly to the recombinant nucleic acids as represented in SEQ ID NO 3, 5, 7, 9, 11, 13, 21, 23, 25, 27, 29, 31, 35, 37, 39, 41, 43, 45, 47 and 49, or parts thereof.

The present invention also contemplates host cells transformed with a recombinant vector as defined above, wherein said vector comprises a nucleotide sequence encoding HCV E1 and/or E2 and/or E1/E2 protein as defined above in addition to a regulatory sequence operably linked to said HCV E1 and/or E2 and/or E1/E2 sequence and capable of regulating the expression of said HCV E1 and/or E2 and/or E1/E2 protein.

Eukaryotic hosts include lower and higher eukaryotic hosts as described in the definitions section. Lower eukaryotic hosts include yeast cells well known in the art. Higher eukaryotic hosts mainly include mammalian cell lines known in the art and include many immortalized cell lines available from the ATCC, inluding HeLa cells, Chinese hamster ovary (CHO) cells, Baby hamster kidney (BHK) cells, PK15, RK13 and a number of other cell lines.

The present invention relates particularly to a recombinant E1 and/or E2 and/or E1/E2 protein expressed by a host cell as defined above containing a recombinany vector as defined above. These recombinant proteins are particularly purified according to the method of the present invention.

A preferred method for isolating or purifying HCV envelope proteins as defined above is further characterized as comprising at least the following steps:

growing a host cell as defined above transformed with a recombinant vector according to the present invention or with a known recombinant vector expressing E1 and/or E2 and/or E1/E2 HCV envelope proteins in a suitable culture medium,

causing expression of said vector sequence as defined above under suitable conditions, and,

lysing said transformed host cells, preferably in the presence of a SH group blocking agent, such as N-ethylmaleimide (NEM), and possibly a suitable detergent, preferably Empigen-BB,

recovering said HCV envelope protein by affinity purification such as by means of lectin-chromatography or immunoaffinity chromatography using anti-E1 and/or anti-E2 specific monoclonal antibodies, with said lectin being preferably lentil-lectin or GNA, followed by,

incubation of the eluate of the previous step with a disulphide bond cleavage means, such as DTT, preferably followed by incubation with an SH group blocking agent, such as NEM or Biotin-NEM, and,

isolating the HCV single or specific oligomeric E1 and/or E2 and/or E1/E2 proteins such as by means of gelfiltration and possibly also by a subsequent Ni²⁺-IMAC chromatography followed by a desalting step.

As a result of the above-mentioned proces, E1 and/or E2 and/or E1/E2 proteins may be produced in a form which elute differently from the large aggregates containing vector-derived components and/or cell components in the void volume of the gelfiltration column or the IMAC column as illustrated in the Examples section. The disulphide bridge cleavage step advantageously also eliminates the false reactivity due to the presence of host and/or expression-system-derived proteins. The presence of NEM and a suitable detergent during lysis of the cells may already partly or even completely prevent the aggregation between the HCV envelope proteins and contaminants.

N²⁺-IMAC chromatography followed by a desalting step is preferably used for contructs bearing a (His)₆ as described by Janknecht et al., 1991, and Hochuli et al., 1988.

The present invention also relates to a method for producing monoclonal antibodies in small animals such as mice or rats, as well as a method for screening and isolating human B-cells that recognize anti-HCV antibodies, using the HCV single or specific oligomeric envelope proteins of the present invention.

The present invention further relates to a composition comprising at least one of the following E1 peptides as listed in Table 3 and described elsewhere herein:

E1-31 (SEQ ID NO:56) spanning amino acids 181 to 200 of the Core/E1 V1 region,

E1-33 (SEQ ID NO:57) spanning amino acids 193 to 212 of the E1 region,

E1-35 (SEQ ID NO:58) spanning amino acids 205 to 224 of the E1 V2 region (epitope B),

E1-35A (SEQ ID NO:59) spanning amino acids 208 to 227 of the E1 V2 region (epitope B),

1bE1 (SEQ ID NO:53) spanning amino acids 192 to 228 of E1 regions (V1, C1, and V2 regions (containing epitope B)),

E1-51 (SEQ ID NO:66) spanning amino acids 301 to 320 of the E1 region,

E1-53 (SEQ ID NO:67) spanning amino acids 313 to 332 of the E1 C4 region (epitope A),

E1-55 (SEQ ID NO:68) spanning amino acids 325 to 344 of the E1 region,

The present invention also relates to a composition comprising at least one of the following E2 peptides as listed in Table 3:

Env 67 or E2-67 (SEQ ID NO:72) spanning amino acid positions 397 to 416 of the

E2 region (epitope A, recognized by monoclonal antibody 2F10H10, see FIG. 19),

Env 69 or E2-69 (SEQ ID NO:73) spanning amino acid positions 409 to 428 of the E2 region (epitope A),

Env 23 or E2-23 (SEQ ID NO:86) spanning positions 583 to 602 of the E2 region (epitope E),

Env 25 or E2-25 (SEQ ID NO:87) spanning positions 595 to 614 of the E2 region (epitope E),

Env 27 or E2-27 (SEQ ID NO:88) spanning positions 607 to 626 of the E2 region (epitope E),

Env 17B or E2-17B (SEQ ID NO:83) spanning positions 547 to 566 of the E2 region (epitope D),

Env 13B or E2-13B (SEQ ID NO:82) spanning positions 523 to 542 of the E2 region (epitope C; recognized by monoclonal antibody 16A6E7, see FIG. 19).

The present invention also relates to a composition comprising at least one of the following E2 conformational epitopes:

epitope F recognized by monoclonal antibodies 15C8C1, 12D11 F1 and 8G10D1H9,

epitope G recognized by monoclonal antibody 9G3E6,

epitope H (or C) recognized by monoclonal antibody 10D3C4 and 4H6B2, or,

epitope I recognized by monoclonal antibody 17F2C2.

The present invention also relates to an E1 or E2 specific antibody raised upon immunization with a peptide or protein composition, with said antibody being specifically reactive with any of the polypeptides or peptides as defined above, and with said antibody being preferably a monoclonal antibody.

The present invention also relates to an E1 or E2 specific antibody screened from a variable chain library in plasmids or phages or from a population of human B-cells by means of a process known in the art, with said antibody being reactive with any of the polypeptides or peptides as defined above, and with said antibody being preferably a monoclonal antibody.

The E1 or E2 specific monoclonal antibodies of the invention can be produced by any hybridoma liable to be formed according to classical methods from splenic cells of an animal, particularly from a mouse or rat, immunized against the HCV polypeptides or peptides according to the invention, as defined above on the one hand, and of cells of a myeloma cell line on the other hand, and to be selected by the ability of the hybridoma to produce the monoclonal antibodies recognizing the polypeptides which has been initially used for the immunization of the animals.

The antibodies involved in the invention can be labelled by an appropriate label of the enzymatic, fluorescent, or radioactive type.

The monoclonal antibodies according to this preferred embodiment of the invention may be humanized versions of mouse monoclonal antibodies made by means of recombinant DNA technology, departing from parts of mouse and/or human genomic DNA sequences coding for H and L chains from cDNA or genomic clones coding for H and L chains.

Alternatively the monoclonal antibodies according to this preferred embodiment of the invention may be human monoclonal antibodies. These antibodies according to the present embodiment of the invention can also be derived from human peripheral blood lymphocytes of patients infected with HCV, or vaccinated against HCV. Such human monoclonal antibodies are prepared, for instance, by means of human peripheral blood lymphocytes (PBL) repopulation of severe combined immune deficiency (SCID) mice (for recent review, see Duchosal et al., 1992).

The invention also relates to the use of the proteins or peptides of the invention, for the selection of recombinant antibodies by the process of repertoire cloning (Persson et al., 1991).

Antibodies directed to peptides or single or specific oligomeric envelope proteins derived from a certain genotype may be used as a medicament, more particularly for incorporation into an immunoassay for the detection of HCV genotypes (for detecting the presence of HCV E1 or E2 antigen), for prognosing/monitoring of HCV disease, or as therapeutic agents.

Alternatively, the present invention also relates to the use of any of the above-specified E1 or E2 specific monoclonal antibodies for the preparation of an immunoassay kit for detecting the presence of E1 or E2 antigen in a biological sample, for the preparation of a kit for prognosing/monitoring of HCV disease or for the preparation of a HCV medicament.

The present invention also relates to the a method for in vitro diagnosis or detection of HCV antigen present in a biological sample, comprising at least the following steps:

(i) contacting said biological sample with any of the E1 and/or E2 specific monoclonal antibodies as defined above, preferably in an immobilized form under appropriate conditions which allow the formation of an immune complex,

(ii) removing unbound components,

(iii) incubating the immune complexes formed with heterologous antibodies, which specifically bind to the antibodies present in the sample to be analyzed, with said heterologous antibodies having conjugated to a detectable label under appropriate conditions,

(iv) detecting the presence of said immune complexes visually or mechanically (e.g. by means of densitometry, fluorimetry, colorimetry).

The present invention also relates to a kit for in vitro diagnosis of HCV antigen present in a biological sample, comprising:

at least one monoclonal antibody as defined above, with said antibody being preferentially immobilized on a solid substrate,

a buffer or components necessary for producing the buffer enabling binding reaction between these antibodies and the HCV antigens present in the biological sample,

a means for detecting the immune complexes formed in the preceding binding reaction,

possibly also including an automated scanning and interpretation device for inferring the HCV antigens present in the sample from the observed binding pattern.

The present invention also relates to a composition comprising E1 and/or E2 and/or E1/E2 recombinant HCV proteins purified according to the method of the present invention or a composition comprising at least one peptides as specified above for use as a medicament.

The present invention more particularly relates to a composition comprising at least one of the above-specified envelope peptides or a recombinant envelope protein composition as defined above, for use as a vaccine for immunizing a mammal, preferably humans, against HCV, comprising administering a sufficient amount of the composition possibly accompanied by pharmaceutically acceptable adjuvant(s), to produce an immune response.

More particularly, the present invention relates to the use of any of the compositions as described here above for the preparation of a vaccine as described above.

Also, the present invention relates to a vaccine composition for immunizing a mammal, preferably humans, against HCV, comprising HCV single or specific oligomeric proteins or peptides derived from the E1 and/or the E2 region as described above.

Immunogenic compositions can be prepared according to methods known in the art. The present compositions comprise an immunogenic amount of a recombinant E1 and/or E2 and/or E1/E2 single or specific oligomeric proteins as defined above or E1 or E2 peptides as defined above, usually combined with a pharmaceutically acceptable carrier, preferably further comprising an adjuvant.

The single or specific oligomeric envelope proteins of the present invention, either E1 and/or E2 and/or E1/E2, are expected to provide a particularly useful vaccine antigen, since the formation of antibodies to either E1 or E2 may be more desirable than to the other envelope protein, and since the E2 protein is cross-reactive between HCV types and the E1 protein is type-specific. Cocktails including type 1 E2 protein and E1 proteins derived from several genotypes may be particularly advantageous. Cocktails containing a molar excess of E1 versus E2 or E2 versus E1 may also be particularly useful. Immunogenic compositions may be administered to animals to induce production of antibodies, either to provide a source of antibodies or to induce protective immunity in the animal.

Pharmaceutically acceptable carriers include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers; and inactive virus particles. Such carriers are well known to those of ordinary skill in the art.

Preferred adjuvants to enhance effectiveness of the composition include, but are not limited to: aluminim hydroxide (alum), N-acetyl-muramyl-L-threonyl-D-isoglutamine (thr-MDP) as found in U.S. Pat. No. 4,606,918, N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (MTP-PE) and RIBI, which contains three components extracted from bacteria, monophosphoryl lipid A, trehalose dimycolate, and cell wall skeleton (MPL+TDM+CWS) in a 2% squalene/Tween 80 emulsion. Any of the 3 components MPL, TDM or CWS may also be used alone or combined 2 by 2. Additionally, adjuvants such as Stimulon (Cambridge Bioscience, Worcester, Mass.) or SAF-1 (Syntex) may be used. Further, Complete Freund's Adjuvant (CFA) and Incomplete Freund's Adjuvant (IFA) may be used for non-human applications and research purposes.

The immunogenic compositions typically will contain pharmaceutically acceptable vehicles, such as water, saline, glycerol, ethanol, etc. Additionally, auxiliary substances, such as wetting or emulsifying agents, pH buffering substances, preservatives, and the like, may be included in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect. The E1 and E2 proteins may also be incorporated into Immune Stimulating Complexes together with saponins, for example Quil A (ISCOMS).

Immunogenic compositions used as vaccines comprise a ‘sufficient amount’ or ‘an immunologically effective amount’ of the envelope proteins of the present invention, as well as any other of the above mentioned components, as needed. ‘Immunologically effective amount’, means that the administration of that amount to an individual, either in a single dose or as part of a series, is effective for treatment, as defined above. This amount varies depending upon the health and physical condition of the individual to be treated, the taxonomic group of individual to be treated (e.g. nonhuman primate, primate, etc.), the capacity of the individual's immune system to synthesize antibodies, the degree of protection desired, the formulation of the vaccine, the treating doctor's assessment of the medical situation, the strain of infecting HCV, and other relevant factors. It is expected that the amount will fall in a relatively broad range that can be determined through routine trials. Usually, the amount will vary from 0.01 to 1000 μg/dose, more particularly from 0.1 to 100 μg/dose.

The single or specific oligomeric envelope proteins may also serve as vaccine carriers to present homologous (e.g. T cell epitopes or B cell epitopes from the core, NS2, NS3, NS4 or NS5 regions) or heterologous (non-HCV) haptens, in the same manner as Hepatitis B surface antigen (see European Patent Application 174,444). In this use, envelope proteins provide an immunogenic carrier capable of stimulating an immune response to haptens or antigens conjugated to the aggregate. The antigen may be conjugated either by conventional chemical methods, or may be cloned into the gene encoding E1 and/or E2 at a location corresponding to a hydrophilic region of the protein. Such hydrophylic regions include the V1 region (encompassing amino acid positions 191 to 202), the V2 region (encompassing amino acid positions 213 to 223), the V3 region (encompassing amino acid positions 230 to 242), the V4 region (encompassing amino acid positions 230 to 242), the V5 region (encompassing amino acid positions 294 to 303) and the V6 region (encompassing amino acid positions 329 to 336). Another useful location for insertion of haptens is the hydrophobic region (encompassing approximately amino acid positions 264 to 293). It is shown in the present invention that this region can be deleted without affecting the reactivity of the deleted E1 protein with antisera. Therefore, haptens may be inserted at the site of the deletion.

The immunogenic compositions are conventionally administered parenterally, typically by injection, for example, subcutaneously or intramuscularly. Additional formulations suitable for other methods of administration include oral formulations and suppositories. Dosage treatment may be a single dose schedule or a multiple dose schedule. The vaccine may be administered in conjunction with other immunoregulatory agents.

The present invention also relates to a composition comprising peptides or polypeptides as described above, for in vitro detection of HCV antibodies present in a biological sample.

The present invention also relates to the use of a composition as described above for the preparation of an immunoassay kit for detecting HCV antibodies present in a biological sample.

The present invention also relates to a method for in vitro diagnosis of HCV antibodies present in a biological sample, comprising at least the following steps

(i) contacting said biological sample with a composition comprising any of the envelope peptide or proteins as defined above, preferably in an immobilized form under appropriate conditions which allow the formation of an immune complex, wherein said peptide or protein can be a biotinylated peptide or protein which is covalently bound to a solid substrate by means of streptavidin or avidin complexes,

(ii) removing unbound components,

(iii) incubating the immune complexes formed with heterologous antibodies, with said heterologous antibodies having conjugated to a detectable label under appropriate conditions,

(iv) detecting the presence of said immune complexes visually or mechanically (e.g. by means of densitometry, fluorimetry, colorimetry).

Alternatively, the present invention also relates to competition immunoassay formats in which recombinantly produced purified single or specific oligomeric protein E1 and/or E2 and/or E1/E2 proteins as disclosed above are used in combination with E1 and/or E2 peptides in order to compete for HCV antibodies present in a biological sample.

The present invention also relates to a kit for determining the presence of HCV antibodies, in a biological sample, comprising:

at least one peptide or protein composition as defined above, possibly in combination with other polypeptides or peptides from HCV or other types of HCV, with said peptides or proteins being preferentially immobilized on a solid substrate, more preferably on different microwells of the same ELISA plate, and even more preferentially on one and the same membrane strip,

a buffer or components necessary for producing the buffer enabling binding reaction between these polypeptides or peptides and the antibodies against HCV present in the biological sample,

means for detecting the immune complexes formed in the preceding binding reaction,

possibly also including an automated scanning and interpretation device for inferring the HCV genotypes present in the sample from the observed binding pattern.

The immunoassay methods according to the present invention utilize single or specific oligomeric antigens from the E1 and/or E2 domains that maintain linear (in case of peptides) and conformational epitopes (single or specific oligomeric proteins) recognized by antibodies in the sera from individuals infected with HCV. It is within the scope of the invention to use for instance single or specific oligomeric antigens, dimeric antigens, as well as combinations of single or specific oligomeric antigens. The HCV E1 and E2 antigens of the present invention may be employed in virtually any assay format that employs a known antigen to detect antibodies. Of course, a format that denatures the HCV conformational epitope should be avoided or adapted. A common feature of all of these assays is that the antigen is contacted with the body component suspected of containing HCV antibodies under conditions that permit the antigen to bind to any such antibody present in the component. Such conditions will typically be physiologic temperature, pH and ionic strenght using an excess of antigen. The incubation of the antigen with the specimen is followed by detection of immune complexes comprised of the antigen.

Design of the immunoassays is subject to a great deal of variation, and many formats are known in the art. Protocols may, for example, use solid supports, or immunoprecipitation. Most assays involve the use of labeled antibody or polypeptide; the labels may be, for example, enzymatic, fluorescent, chemiluminescent, radioactive, or dye molecules. Assays which amplify the signals from the immune complex are also known; examples of which are assays which utilize biotin and avidin or streptavidin, and enzyme-labeled and mediated immunoassays, such as ELISA assays.

The immunoassay may be, without limitation, in a heterogeneous or in a homogeneous format, and of a standard or competitive type. In a heterogeneous format, the polypeptide is typically bound to a solid matrix or support to facilitate separation of the sample from the polypeptide after incubation. Examples of solid supports that can be used are nitrocellulose (e.g., in membrane or microtiter well form), polyvinyl chloride (e.g., in sheets or microtiter wells), polystyrene latex (e.g., in beads or microtiter plates, polyvinylidine fluoride (known as Immunolon™), diazotized paper, nylon membranes, activated beads, and Protein A beads. For example, Dynatech Immunolon™ 1 or Immunlon™ 2 microtiter plates or 0.25 inch polystyrene beads (Precision Plastic Ball) can be used in the heterogeneous format. The solid support containing the antigenic polypeptides is typically washed after separating it from the test sample, and prior to detection of bound antibodies. Both standard and competitive formats are know in the art.

In a homogeneous format, the test sample is incubated with, the combination of antigens in solution. For example, it may be under conditions that will precipitate any antigen-antibody complexes which are formed. Both standard and competitive formats for these assays are known in the art.

In a standard format, the amount of HCV antibodies in the antibody-antigen complexes is directly monitored. This may be accomplished by determining whether labeled anti-xenogeneic (e.g. anti-human) antibodies which recognize an epitope on anti-HCV antibodies will bind due to complex formation. In a competitive format, the amount of HCV antibodies in the sample is deduced by monitoring the competitive effect on the binding of a known amount of labeled antibody (or other competing ligand) in the complex.

Complexes formed comprising anti-HCV antibody (or in the case of competitive assays, the amount of competing antibody) are detected by any of a number of known techniques, depending on the format. For example, unlabeled HCV antibodies in the complex may be detected using a conjugate of anti-xenogeneic Ig complexed with a label (e.g. an enzyme label).

In an immunoprecipitation or agglutination assay format the reaction between the HCV antigens and the antibody forms a network that precipitates from the solution or suspension and forms a visible layer or film of precipitate. If no anti-HCV antibody is present in the test specimen, no visible precipitate is formed.

There currently exist three specific types of particle agglutination (PA) assays. These assays are used for the detection of antibodies to various antigens when coated to a support. One type of this assay is the hemagglutination assay using red blood cells (RBCs) that are sensitized by passively adsorbing antigen (or antibody) to the RBC. The addition of specific antigen antibodies present in the body component, if any, causes the RBCs coated with the purified antigen to agglutinate.

To eliminate potential non-specific reactions in the hemagglutination assay, two artificial carriers may be used instead of RBC in the PA. The most common of these are latex particles. However, gelatin particles may also be used. The assays utilizing either of these carriers are based on passive agglutination of the particles coated with purified antigens.

The HCV single or specififc oligomeric E1 and/or E2 and/or E1/E2 antigens of the present invention comprised of conformational epitopes will typically be packaged in the form of a kit for use in these immunoassays. The kit will normally contain in separate containers the native HCV antigen, control antibody formulations (positive and/or negative), labeled antibody when the assay format requires the same and signal generating reagents (e.g. enzyme substrate) if the label does not generate a signal directly. The native HCV antigen may be already bound to a solid matrix or separate with reagents for binding it to the matrix. Instructions (e.g. written, tape, CD-ROM, etc.) for carrying out the assay usually will be included in the kit.

Immunoassays that utilize the native HCV antigen are useftil in screening blood for the preparation of a supply from which potentially infective HCV is lacking. The method for the preparation of the blood supply comprises the following steps. Reacting a body component, preferably blood or a blood component, from the individual donating blood with HCV E1 and/or E2 proteins of the present invention to allow an immunological reaction between HCV antibodies, if any, and the HCV antigen. Detecting whether anti-HCV antibody—HCV antigen complexes are formed as a result of the reacting. Blood contributed to the blood supply is from donors that do not exhibit antibodies to the native HCV antigens, E1 or E2.

In cases of a positive reactivity to the HCV antigen, it is preferable to repeat the immunoassay to lessen the possibility of false positives. For example, in the large scale screening of blood for the production of blood products (e.g. blood transfusion, plasma, Factor VIII, immunoglobulin, etc.) ‘screening’ tests are typically formatted to increase sensitivity (to insure no contaminated blood passes) at the expense of specificity; i.e. the false-positive rate is increased. Thus, it is typical to only defer for further testing those donors who are ‘repeatedly reactive’; i.e. positive in two or more runs of the immunoassay on the donated sample. However, for confirmation of HCV-positivity, the ‘confirmation’ tests are typically formatted to increase specificity (to insure that no false-positive samples are confirmed) at the expense of sensitivity. Therefore the purification method described in the present invention for E1 and E2 will be very advantageous for including single or specific oligomeric envelope proteins into HCV diagnostic assays.

The solid phase selected can include polymeric or glass beads, nitrocellulose, microparticles, microwells of a reaction tray, test tubes and magnetic beads. The signal generating compound can include an enzyme, a luminescent compound, a chromogen, a radioactive element and a chemiluminescent compound. Examples of enzymes include alkaline phosphatase, horseradish peroxidase and beta-galactosidase. Examples of enhancer compounds include biotin, anti-biotin and avidin. Examples of enhancer compounds binding members include biotin, anti-biotin and avidin. In order to block the effects of rheumatoid factor-like substances, the test sample is subjected to conditions sufficient to block the effect of rheumatoid factor-like substances. These conditions comprise contacting the test sample with a quantity of anti-human IgG to form a mixture, and incubating the mixture for a time and under conditions sufficient to form a reaction mixture product substantially free of rheumatoid factor-like substance.

The present invention further contemplates the use of E1 proteins, or parts thereof, more particularly HCV single or specific oligomeric E1 proteins as defined above, for in vitro monitoring HCV disease or prognosing the response to treatment (for instance with Interferon) of patients suffering from HCV infection comprising:

incubating a biological sample from a patient with hepatitis C infection with an E1 protein or a suitable part thereof under conditions allowing the formation of an immunological complex,

removing unbound components,

calculating the anti-E1 titers present in said sample (for example at the start of and/or during the course of (interferon) therapy),

monitoring the natural course of HCV disease, or prognosing the response to treatment of said patient on the basis of the amount anti-E1 titers found in said sample at the start of treatment and/or during the course of treatment.

Patients who show a decrease of 2, 3, 4, 5, 7, 10, 15, or preferably more than 20 times of the initial anti-E1 titers could be concluded to be long-term, sustained responders to HCV therapy, more particularly to interferon therapy. It is illustrated in the Examples section, that an anti-E1 assay may be very useful for prognosing long-term response to IFN treatment, or to treatment of Hepatitis C virus disease in general.

More particularly the following E1 peptides as listed in Table 3 were found to be useful for in vitro monitoring HCV disease or prognosing the response to interferon treatment of patients suffering from HCV infection:

E1-31 (SEQ ID NO:56) spanning amino acids 181 to 200 of the Core/E1 V1 region,

E1-33 (SEQ ID NO:57) spanning amino acids 193 to 212 of the E1 region,

E1-35 (SEQ ID NO:58) spanning amino acids 205 to 224 of the E1 V2 region (epitope B),

E1-35A (SEQ ID NO:59) spanning amino acids 208 to 227 of the E1 V2 region (epitope B),

1bE1 (SEQ ID NO:53) spanning amino acids 192 to 228 of E1 regions (V1, C1, and

V2 regions (containing epitope B)),

E1-51 (SEQ ID NO:66) spanning amino acids 301 to 320 of the E1 region,

E1-53 (SEQ ID NO:67) spanning amino acids 313 to 332 of the E1 C4 region (epitope A),

E1-55 (SEQ ID NO:68) spanning amino acids 325 to 344 of the E1 region.

It is to be understood that smaller fragments of the above-mentioned peptides also fall within the scope of the present invention. Said smaller fragments can be easily prepared by chemical synthesis and can be tested for their ability to be used in an assay as detailed above and in the Examples section.

The present invention also relates to a kit for monitoring HCV disease or prognosing the response to treatment (for instance to interferon) of patients suffering from HCV infection comprising:

at least one E1 protein or E1 peptide, more particularly an E1 protein or E1 peptide as defined above,

a buffer or components necessary for producing the buffer enabling the binding reaction between these proteins or peptides and the anti-E1 antibodies present in a biological sample,

means for detecting the immune complexes formed in the preceding binding reaction,

possibly also an automated scanning and interpretation device for inferring a decrease of anti-E1 titers during the progression of treatment.

It is to be understood that also E2 protein and peptides according to the present invention can be used to a certain degree to monitor/prognose HCV treatment as indicated above for the E1 proteins or peptides because also the anti-E2 levels decrease in comparison to antibodies to the other HCV antigens. It is to be understood, however, that it might be possible to determine certain epitopes in the E2 region which would also be suited for use in an test for monitoring/prognosing HCV disease.

The present invention also relates to a serotyping assay for detecting one or more serological types of HCV present in a biological sample, more particularly for detecting antibodies of the different types of HCV to be detected combined in one assay format, comprising at least the following steps:

(i) contacting the biological sample to be analyzed for the presence of HCV antibodies of one or more serological types, with at least one of the E1 and/or E2 and/or E1/E2 protein compositions or at least one of the E1 or E2 peptide compositions as defined above, preferentially in an immobilized form under appropriate conditions which allow the formation of an immune complex,

(ii) removing unbound components,

(iii) incubating the immune complexes formed with heterologous antibodies, with said heterologous antibodies being conjugated to a detectable label under appropriate conditions,

(iv) detecting the presence of said immune complexes visually or mechanically (e.g. by means of densitometry, fluorimetry, colorimetry) and inferring the presence of one or more HCV serological types present from the observed binding pattern.

It is to be understood that the compositions of proteins or peptides used in this method are recombinantly expressed type-specific envelope proteins or type-specific peptides.

The present invention further relates to a kit for serotyping one or more serological types of HCV present in a biological sample, more particularly for detecting the antibodies to these serological types of HCV comprising:

at least one E1 and/or E2 and/or E1/E2 protein or E1 or E2 peptide, as defined above,

a buffer or components necessary for producing the buffer enabling the binding reaction between these proteins or peptides and the anti-E1 antibodies present in a biological sample,

means for detecting the immune complexes formed in the preceding binding reaction,

possibly also an automated scanning and interpretation device for detecting the presence of one or more serological types present from the observed binding pattern.

The present invention also relates to the use of a peptide or protein composition as defined above, for immobilization on a solid substrate and incorporation into a reversed phase hybridization assay, preferably for immobilization as parallel lines onto a solid support such as a membrane strip, for determining the presence or the genotype of HCV according to a method as defined above. Combination with other type-specific antigens from other HCV polyprotein regions also lies within the scope of the present invention.

The present invention provides a method for purifying recombinant HCV single or specific oligomeric envelope proteins selected from E1 and/or E2 and/or E1/E2 proteins which have been produced by a recombinant process comprising contacting said proteins with a disulphide bond cleavage or reducing agent. The contacting of the method of the invention may be carried out under partial cleavage or reducing conditions. Preferably, the disulphide bond cleavage agent is dithiothreitol (DTT), preferably in a concentration range of 0.1 to 50 mM, preferably 0.1 to 20 mM, more preferably 0.5 to 10 mM. Alternatively, the disulphide bond cleavage agent may be a detergent, such as Empigen-BB (which is a mixture containing N-Docecyl-N,N-dimethylglycine as a major component), preferably at a concentration of 1 to 10%, more preferably at a concentration of 3.5%. Mixtures of detergents, disulphide bond cleavage agents and/or reducing agents may also be used. In one embodiment, disulphide bond reformation is prevented with an SH group blocking agent, such as N-ethylmaleimide (NEM) or a derivative thereof. In a preferred embodiment, the disulphide bond reformation is blocked by use of low pH conditions.

The present invention further provides a method as described herein, further involving the following steps: lysing recombinant E1 and/or E2 and/or E1/E2 expressing host cells, optionally in the presence of an SH blocking agent such as N-ethylmaleimide (NEM); recovering said HCV envelope proteins by affinity purification such as by means of lectin-chromatography, such as lentil-lectin chromatography, or by means of immunoaffinity using anti-E1 and/or anti-E2 specific monoclonal antibodies; reducing or cleaving of the disulfide bonds with a disulphide bond cleaving agent, such as DTT, preferably also in the presence of an SH blocking agent, such as NEM or Biotin-NEM; and, recovering the reduced E1 and/or E2 and/or E1/E2 envelope proteins by gelfiltration and optionally additionally by a subsequent Ni²⁺-IMAC chromatography and desalting step.

The present invention provides a composition containing substantially isolated and/or purified, and/or isolated and/or purified recombinant HCV single or specific oligomeric recombinant envelope proteins selected from E1 and/or E2 and/or E1/E2, which have preferably been isolated from the methods described herein. In a preferred embodiment, the recombinant HCV envelope proteins of the invention have been expressed in recombinant mammalian cells, such as vaccinia, recombinant yeast cells.

The present invention provides a recombinant vector containing a vector sequence, a prokaryotic, eukaryotic or viral promoter sequence and a nucleotide sequence allowing the expression of a single or specific oligomeric E1 and/or E2 and/or E1/E2 protein, in operable combination. In one embodiment, the nucleotide sequence of the vector encodes a single HCV E1 protein starting in the region between amino acid positions 1 and 192 and ending in the region between amino acid positions 250 and 400, more particularly ending in the region between positions 250 and 341, even more preferably ending in the region between position 290 and 341. In another embodiment, the nucleotide sequence of the vector encodes a single HCV E1 protein starting in the region between amino acid positions 117 and 192 and ending in the region between amino acid positions 263 and 400, more particularly ending in the region between positions 250 and 326. In yet another embodiment, the nucleotide sequence of the vector encodes a single HCV E1 protein bearing a deletion of the first hydrophobic domain between positions 264 to 293, plus or minus 8 amino acids. In a further embodiment, the nucleotide sequence of the vector encodes a single HCV E2 protein starting in the region between amino acid positions 290 and 406 and ending in the region between amino acid positions 600 and 820, more particularly starting in the region between positions 322 and 406, even more preferably starting in the region between position 347 and 406 and most preferably starting in the region between positions 364 and 406; and preferably ending at any of amino acid positions 623, 650, 661, 673, 710, 715, 720, 746 or 809. The vector of the present invention, in one embodiment, contains a 5′-terminal ATG codon and a 3′-terminal stop codon operably linked to the nucleotide sequence. The vector further contains, in one embodiment, a nucleotide sequence further containing at a factor Xa cleavage site and/or 3 to 10, preferably 6, histidine codons added 3′-terminally to the coding region. The vector of the present invention optionally contains a nucleotide sequence wherein at least one of the glycosylation sites present in the E1 or E2 proteins has been removed at the nucleic acid level.

The present invention provides a nucleic acid containing any one of SEQ ID NOs: 3, 5, 7, 9, 11, 13, 21, 23, 25, 27, 29, 31, 35, 37, 39, 41, 43, 45, 47 and 49, or parts thereof. The vector of the invention may preferably contain a nucleotide sequence containing a nucleic acid containing any one of SEQ ID NOs: 3, 5, 7, 9, 11, 13, 21, 23, 25, 27, 29, 31, 35, 37, 39, 41, 43, 45, 47 and 49, or parts thereof.

The composition of the present invention further contains recombinant HCV envelope proteins which have been expressed or are the expression product of a vector described herein.

The present invention provides a host cell transformed with at least one recombinant vector as described herein, wherein the vector contains a nucleotide sequence encoding HCV E1 and/or E2 and/or E1/E2 protein as described herein in addition to a regulatory sequence operable in the host cell and capable of regulating expression of the HCV E1 and/or E2 and/or E1/E2 protein. Moreover, the present invention provides a ecombinant E1 and/or E2 and/or E1/E2 protein expressed by a host cell of the invention.

The present invention further provides a method as described herein and containing the following steps: growing a host cell as described herein which has been transformed with a recombinant vector as described herein in a suitable culture medium; causing expression of the vector nucleotide sequence of the vector, as described herein under suitable conditions; lysing the transformed host cells, preferably in the presence of an SH group blocking agent, such as N-ethylmaleimide (NEM); recovering the HCV envelope protein by affinity purification by means of for instance lectin-chromatography or immunoaffinity chromatography using anti-E1 and/or anti-E2 specific monoclonal antibodies, with said lectin being preferably lentil-lectin, followed by, incubation of the eluate of the previous step with a disulphide bond cleavage agent, such as DTT, preferably also in the presence of an SH group blocking agent, such as NEM or Biotin-NEM; and, isolating the HCV single or specific oligomeric E1 and/or E2 and/or E1/E2 proteins by means of gelfiltration and possibly also by means of an additional Ni²⁺-IMAC chromatography and desalting step.

The present invention provides a composition containing at least one of the following E1 and/or E2 peptides:

E1-31 (SEQ ID NO:56) spanning amino acids 181 to 200 of the Core/E1 V1 region,

E1-33 (SEQ ID NO:57) spanning amino acids 193 to 212 of the E1 region,

E1-35 (SEQ ID NO:58) spanning amino acids 205 to 224 of the E1 V2 region (epitope B),

E1-35A (SEQ ID NO:59) spanning amino acids 208 to 227 of the E1 V2 region (epitope B),

1bE1 (SEQ ID NO:53) spanning amino acids 192 to 228 of E1 regions (V1, C1, and V2 regions (containing epitope B),

E1-51 (SEQ ID NO:66) spanning amino acids 301 to 320 of the E1 region,

E1-53 (SEQ ID NO:67) spanning amino acids 313 to 332 of the E1 C4 region (epitope A),

E1-55 (SEQ ID NO:68) spanning amino acids 325 to 344 of the E1 region.

Env 67 or E2-67 (SEQ ID NO:72) spanning amino acid positions 397 to 416 of the E2 region (epitope A),

Env 69 or E2-69 (SEQ ID NO:73) spanning amino acid positions 409 to 428 of the E2 region (epitope A),

Env 23 or E2-23 (SEQ ID NO:86) spanning positions 583 to 602 of the E2 region (epitope E),

Env 25 or E2-25 (SEQ ID NO:87) spanning positions 595 to 614 of the E2 region (epitope E),

Env 27 or E2-27 (SEQ ID NO:88) spanning positions 607 to 626 of the E2 region (epitope E),

Env 17B or E2-17B (SEQ ID NO:83) spanning positions 547 to 566 of the E2 region (epitope D),

Env 13B or E2-13B (SEQ ID NO:82) spanning positions 523 to 542 of the E2 region (epitope C).

The present invention provides a composition containing at least one of the following E2 conformational epitopes:

epitope F recognized by monoclonal antibodies 15C8C1, 12D11F1, and 8G10D1H9,

epitope G recognized by monoclonal antibody 9G3E6,

epitope H (or C) recognized by monoclonal antibodies 10D3C4 and 4H6B2,

epitope I recognized by monoclonal antibody 17F2C2.

The present invention provides an E1 and/or E2 specific monoclonal antibody raised upon immunization with a composition as described herein. The antibodies of the present invention may be used, for example, as a medicament, for incorporation into an immunoassay kit for detecting the presence of HCV E1 or E2 antigen, for prognosis/monitoring of disease or for HCV therapy. The present invention provides for the use of an E1 and/or E2 specific monoclonal antibody as described herein for the preparation of an immunoassay kit for detecting HCV E1 or E2 antigens, for the preparation of a kit for prognosing/monitoring of HCV disease or for the preparation of a HCV medicament.

The present invention provides a method for in vitro diagnosis of HCV antigen present in a biological sample, containing at least the following steps:

(i) contacting said biological sample with an E1 and/or E2 specific monoclonal antibody as described herein, preferably in an immobilized form under appropriate conditions which allow the formation of an immune complex,

(ii) removing unbound components,

(iii) incubating the immune complexes formed with heterologous antibodies, with the heterologous antibodies being conjugated to a detectable label under appropriate conditions,

(iv) detecting the presence of the immune complexes visually or mechanically.

The present invention provides a kit for determining the presence of HCV antigens present in a biological sample, which includes at least the following: at least one E1 and/or E2 specific monoclonal antibody as described herein, preferably in an immobilized form on a solid substrate, a buffer or components necessary for producing the buffer enabling binding reaction between these antibdodies and the HCV antigens present in a biological sample, and optionally a means for detecting the immune complexes formed in the preceding binding reaction.

The composition of the present invention may be provided in the form of a medicament.

The present invention provides a composition, as described herein for use as a vaccine for immunizing a mammal, preferably humans, against HCV, comprising administrating an effective amount of said composition being optionally accompanied by pharmaceutically acceptable adjuvants, to produce an immune response.

The present invention provides a method of using the composition, as described herein, for the preparation of a vaccine for immunizing a mammal, preferably humans, against HCV, comprising administrating an effective amount of said composition, optionally accompanied by pharmaceutically acceptable adjuvants, to produce an immune response.

The present invention provides a vaccine composition for immunzing a mammal, preferably humans, against HCV, which contains an effective amount of a composition containing an E1 and/or E2 containing composition as described herein, optionally also accompanied by pharmaceutically acceptable adjuvants.

The composition of the present invention may be provided in a form for in vitro detection of HCV antibodies present in a biological sample. The present invention also provides a method of preparing an immunoassay kit for detecting HCV antibodies present in a biological sample and a method of detecting HCV antibodies present in a biological sample using the kit of the invention to diagnose HCV antibodies present in a biological sample. Such a method of the present invention includes at least the following steps:

(i) contacting said biological sample with a composition as described herein, preferably in an immobilized form under appropriate conditions which allow the formation of an immune complex with HCV antibodies present in the biological sample,

(ii) removing unbound components,

(iii) incubating the immune complexes formed with heterologous antibodies, with the heterologous antibodies being conjugated to a detectable label under appropriate conditions,

(iv) detecting the presence of the immune complexes visually or mechanically.

The present invention provides a kit for determining the presence of HCV antibodies present in a biological sample, containing: at least one peptide or protein composition as described herein, preferably in an immobilized form on a solid substrate; a buffer or components necessary for producing the buffer enabling binding reaction between these proteins or peptides and the antibodies against HCV present in the biological sample; and, optionally, a means for detecting the immune complexes formed in the preceding binding reaction.

The present invention provides a method of in vitro monitoring HCV disease or diagnosing the response of a patient suffering from HCV infection to treatment, preferably with interferon, the method including: incubating a biological sample from the patient with HCV infection with an E1 protein or a suitable part thereof under conditions allowing the formation of an immunological complex; removing unbound components; calculating the anti-E1 titers present in the sample at the start of and during the course of treatment; monitoring the natural course of HCV disease, or diagnosing the response to treatment of the patient on the basis of the amount anti-E1 titers found in the sample at the start of treatment and/or during the course of treatment.

The present invention provides a kit for monitoring HCV disease or prognosing the response to treatment, particularly with interferon, of patients suffering from HCV infection, wherein the kit contains: at least one E1 protein or E1 peptide, more particularly an E1 protein or E1 peptide as described herein; a buffer or components necessary for producing the buffer enabling the binding reaction between these proteins or peptides and the anti-E1 antibodies present in a biological sample; and optionally, means for detetecting the immune complexes formed in the preceding binding reaction, optionally, also an automated scanning and interpretation device for inferring a decrease of anti-E1 titers during the progression of treatment.

The present invention provides a serotyping assay for detecting one or more serological types of HCV present in a biological sample, more particularly for detecting antibodies of the different types of HCV to be detected combined in one assay format, including at least the following steps: (i) contacting the biological sample to be analyzed for the presence of HCV antibodies of one or more serological types, with at least one of the E1 and/or E2 and/or E/E2 protein compositions as described herein or at least one of the E1 or E2 peptide compositions described herein, preferentially in an immobilized form under appropriate conditions which allow the formation of an immune complex; (ii) removing unbound components; (iii) incubating the immune complexes formed with heterologous antibodies, with the heterologous antibodies being conjugated to a detectable label under appropriate conditions; and optionally, (iv) detecting the presence of said immune complexes visually or mechanically (e.g. by means of densitometry, fluorimetry, colorimetry) and inferring the presence of one or more HCV serological types present from the observed binding pattern.

The present invention provides a kit for serotyping one or more serological types of HCV present in a biological sample, more particularly for detecting the antibodies to these serological types of HCV containing: at least one E1 and/or E2 and/or E1/E2 protein as described herein or an E1 or E2 peptide as described herein; a buffer or components necessary for producing the buffer enabling the binding reaction between these proteins or peptides and the anti-E1 antibodies present in a biological sample; optionally, means for detecting the immune complexes formed in the preceding binding reaction, optionally, also an automated scanning and interpretation device for detecting the presence of one or more serological types present from the observed binding pattern.

The present invention provides a peptide or protein composition as described herein, for immobilization on a solid substrate and incorporation into a reversed phase hybridization assay, preferably for immobilization as parallel lines onto a solid support such as a membrane strip, for determining the presence or the genotype of HCV according to a method as described herein.

The present invention provides a therapeutic vaccine composition, such as a therapeutic HCV vaccine composition, containing or comprising a therapeutically effective amount of: a composition containing at least one purified recombinant HCV single or specific oligomeric recombinant envelope proteins selected from the group consisting of an E1 protein, an E2 protein, a part of said E1 and E2 proteins, an E1/E2 protein complex formed from purified HCV single or specific oligomeric recombinant E1 or E2 proteins or parts thereof, and optionally a pharmaceutically acceptable adjuvant. Another therapeutic vaccine composition, such as a therapeutic HCV vaccine composition, of the invention may be comprising a therapeutically effective amount of a combination of at least two purified HCV single or specific oligomeric recombinant envelope proteins selected from the group consisting of E1 proteins derived from different HCV genotypes or subtypes, E2 proteins derived from different HCV genotypes or subtypes, parts of said E1 and E2 proteins, and E1/E2 protein complexes formed from purified HCV single or specific oligomeric recombinant E1 or E2 proteins, or parts thereof, derived from different HCV genotypes or subtypes; and optionally a pharmaceutically acceptable adjuvant.

The HCV envelope proteins of the vaccine, or more particularly the HCV vaccine, of the present invention are optionally produced by recombinant mammalian cells, by recombinant yeast cells, or by or via a recombinant virus. The invention provides a therapeutic vaccine composition, such as a therapeutic HCV vaccine composition, containing or comprising a therapeutically effective amount of a composition comprising at least one of the following E1 and E2 peptides:

E1-31 (SEQ ID NO:56) spanning amino acids 181 to 200 of the Core/E1 V1 region,

E1-33 (SEQ ID NO:57) spanning amino acids 193 to 212 of the E1 region,

E1-35 (SEQ ID NO:58) spanning amino acids 205 to 224 of the E1 V2 region (epitope B),

E1-35A (SEQ ID NO:59) spanning amino acids 208 to 227 of the E1 V2 region (epitope B),

1bE1 (SEQ ID NO:53) spanning amino acids 192 to 228 of E1 regions V1, C1, and V2 regions (containing epitope B),

E1-51 (SEQ ID NO:66) spanning amino acids 301 to 320 of the E1 region,

E1-53 (SEQ ID NO:67) spanning amino acids 313 to 332 of the E1 C4 region (epitope A),

E1-55 (SEQ ID NO:68) spanning amino acids 325 to 344 of the E1 region,

Env 67 or E2-67 (SEQ ID NO:72) spanning amino acid positions 397 to 418 of the E2 region (epitope A),

Env 69 or E2-69 (SEQ ID NO:73) spanning amino acid positions 409 to 428 of the E2 region (epitope A),

Env 23 or E2-23 (SEQ ID NO:86) spanning positions 583 to 602 of the E2 region (epitope E),

Env 25 or E2-25 (SEQ ID NO:87) spanning positions 595 to 614 of the E2 region (epitope E),

Env 27 or E2-27 (SEQ ID NO:88) spanning positions 607 to 626 of the E2 region (epitope E),

Env 17B or E2-17B (SEQ ID NO:83) spanning positions 547 to 586 of the E2 region (epitope D),

Env 13B or E2-13B (SEQ ID NO:82) spanning positions 523 to 542 of the E2 region (epitope C),

IGP 1626 spanning positions 192-211 of the E1 region (SEQ ID NO:112),

IGP 1627 spanning positions 204-223 of the E1 region (SEQ ID NO:113),

IGP 1628 spanning positions 216-235 of the E1 region (SEQ ID NO:114),

IGP 1629 spanning positions 228-247 of the E1 region (SEQ ID NO:115),

IGP 1630 spanning positions 240-259 of the E1 region (SEQ ID NO:116),

IGP 1631 spanning positions 252-271 of the E1 region (SEQ ID NO:117),

IGP 1632 spanning positions 264-283 of the E1 region (SEQ ID NO:118),

IGP 1633 spanning positions 276-295 of the E1 region (SEQ ID NO:119),

IGP 1634 spanning positions 288-307 of the E1 region (SEQ ID NO:120),

IGP 1635 spanning positions 300-319 of the E1 region (SEQ ID NO:121) and

IGP 1636 spanning positions 312-331 of the E1 region (SEQ ID NO:122); and wherein said peptides may be of recombinant or synthetic origin, and optionally combined with a pharmaceutically acceptable adjuvant.

Any of the above mentioned therapeutic, vaccine compositions may also be considered or regarded as therapeutic HCV vaccine compositions, or as therapeutic compositions or therapeutic HCV compositions, or as compositions or HCV compositions.

The present invention provides a method of treating a mammal, such as a human, infected with HCV comprising administering an effective amount of a composition as described herein, such as the above described vaccines or therapeutic compositions, and optionally, a pharmaceutically acceptable adjuvant. In one embodiment, the composition of the invention is administered in combination with or at a time in conjunction with antiviral therapy, either soon prior to or subsequent to or with administration of the composition of the invention. It will be clear that any of the compositions of the invention, e.g. a therapeutic HCV vaccine composition, can be used for treating a mammal chronically infected with HCV (a “chronic HCV-infected mammal”).

The present invention provides a composition, such as a therapeutic HCV composition or a HCV composition, containing or comprising at least one purified recombinant HCV recombinant envelope proteins selected from the group consisting of an E1 protein and an E2 protein, and optionally an adjuvant. In a preferred embodiment, the composition contains at least one of the following E1 and E2 peptides:

E1-31 (SEQ ID NO:56) spanning amino acids 181 to 200 of the Core/E1 V1 region,

E1-33 (SEQ ID NO:57) spanning amino acids 193 to 212 of the E1 region,

E1-35 (SEQ ID NO:58) spanning amino acids 205 to 224 of the E1 V2 region (epitope B),

E1-35A (SEQ ID NO:59) spanning amino acids 208 to 227 of the E1 V2 region (epitope B),

1bE1 (SEQ ID NO:53) spanning amino acids 192 to 228 of E1 regions V1, C1, and V2 regions (containing epitope B),

E1-51 (SEQ ID NO:66) spanning amino acids 301 to 320 of the E1 region,

E1-53 (SEQ ID NO:67) spanning amino acids 313 to 332 of the E1 C4 region (epitope A),

E1-55 (SEQ ID NO:68) spanning amino acids 325 to 344 of the E1 region,

Env 67 or E2-67 (SEQ ID NO:72) spanning amino acid positions 397 to 418 of the E2 region (epitope A),

Env 69 or E2-69 (SEQ ID NO:73) spanning amino acid positions 409 to 428 of the E2 region (epitope A),

Env 23 or E2-23 (SEQ ID NO:86) spanning positions 583 to 602 of the E2 region (epitope E),

Env 25 or E2-25 (SEQ ID NO:87) spanning positions 595 to 614 of the E2 region (epitope E),

Env 27 or E2-27 (SEQ ID NO:88) spanning positions 607 to 626 of the E2 region (epitope E),

Env 17B or E2-17B (SEQ ID NO:83) spanning positions 547 to 586 of the E2 region (epitope D),

Env 13B or E2-13B (SEQ ID NO:82) spanning positions 523 to 542 of the E2 region (epitope C),

IGP 1626 spanning positions 192-211 of the E1 region (SEQ ID NO:112),

IGP 1627 spanning positions 204-223 of the E1 region (SEQ ID NO:113),

IGP 1628 spanning positions 216-235 of the E1 region (SEQ ID NO:114),

IGP 1629 spanning positions 228-247 of the E1 region (SEQ ID NO:115),

IGP 1630 spanning positions 240-259 of the E1 region (SEQ ID NO:116),

IGP 1631 spanning positions 252-271 of the E1 region (SEQ ID NO:117),

IGP 1632 spanning positions 264-283 of the E1 region (SEQ ID NO:118),

IGP 1633 spanning positions 276-295 of the E1 region (SEQ ID NO:119),

IGP 1634 spanning positions 288-307 of the E1 region (SEQ ID NO:120),

IGP 1635 spanning positions 300-319 of the E1 region (SEQ ID NO:121) and

IGP 1636 spanning positions 312-331 of the E1 region (SEQ ID NO:122), and wherein said peptides may be of recombinant or synthetic origin, and optionally combined with a pharmaceutically acceptable adjuvant.

Another composition, such as a therapeutic HCV composition or HCV composition, of the invention may be comprising a therapeutically effective amount of a combination of at least two purified HCV single or specific oligomeric recombinant envelope proteins selected from the group consisting of E1 proteins derived from different HCV genotypes or subtypes, E2 proteins derived from different HCV genotypes or subtypes, parts of said E1 and E2 proteins, and E1/E2 protein complexes formed from purified HCV single or, specific oligomeric recombinant E1 or E2 proteins, or parts thereof, derived from different HCV genotypes or subtypes; and optionally a pharmaceutically acceptable adjuvant.

The present invention provides a therapeutic composition or therapeutic vaccine composition or composition for inducing HCV-specific antibodies or for stimulating T-cell activity or for stimulating cytokine secretion or cytokine production. The present invention provides a therapeutic HCV composition or therapeutic HCV vaccine composition or HCV composition for inducing HCV-specific antibodies or for stimulating T-cell activity or for stimulating cytokine secretion or cytokine production.

The therapeutic composition according to the present invention, such as a therapeutic HCV vaccine composition or therapeutic HCV composition, may also be therapeutically effective in a HCV carrier infected with a HCV genotype different from the HCV genotype or HCV genotypes from which said E1, E2, or E1/E2 protein complexes are derived.

The recombinant HCV envelope proteins may be produced by recombinant mammalian cells, recombinant HCV envelope proteins are produced by recombinant yeast cells, or recombinant HCV envelope proteins are produced by or via a recombinant virus. The present invention provides a method of treating a mammal, such as a human, infected with HCV including administering an effective amount of a composition described herein, such as a therapeutic HCV vaccine composition, and, optionally, a pharmaceutically acceptable adjuvant. It will be clear that any of the compositions of the invention, such as a therapeutic HCV vaccine composition, can be used for treating a chronic HCV-infected mammal. The present invention provides a therapeutic composition for inducing HCV-specific antibodies, for stimulating T-cell activity or for stimulating cytokine secretion or cytokine production, said composition containing a therapeutic effective amount of a composition containing at least one purified recombinant HCV single or specific oligomeric recombinant envelope protein selected from the group consisting of an E1 protein and an E2 protein; and optionally a pharmaceutically acceptable adjuvant.

In particular, any of the compositions according to this invention (including the vaccine compositions and therapeutic compositions) may comprise recombinant HCV envelope proteins wherein the cysteines of said recombinant HCV envelope proteins are blocked, or may comprise E1 and/or E2 peptides wherein the cysteines of said E1 or E2 peptides are blocked.

In another embodiment of the invention, the compositions (including the vaccine compositions and therapeutic compositions) according to the invention may comprise recombinant HCV envelope proteins which are added to said compositions as viral-like particles (VLPs).

In a further embodiment, a composition of the invention such as a therapeutic HCV vaccine composition may comprise as recombinant HCV E1 envelope protein an E1s protein. More particularly, said E1s protein is defined by SEQ ID NO:123.

Another aspect of the invention relates to an immunogenic composition, in particular a HCV immunogenic composition, comprising a recombinant virus allowing expression of at least one HCV recombinant envelope protein chosen from an E1 protein and/or an E2 protein, and parts of said E1 and E2 proteins; and, optionally, a pharmaceutically acceptable adjuvant.

In yet a further aspect, the invention is envisaging a vaccine composition such as a HCV vaccine composition comprising a recombinant virus allowing expression of at least one HCV recombinant envelope protein chosen from an E1 protein and/or an E2 protein, and parts of said E1 and E2 proteins; and, optionally, a pharmaceutically acceptable adjuvant.

In one embodiment, the above recombinant virus compositions may be effective against a HCV genotype or subtype different from the HCV genotype or subytpe from which said E1 protein and/or E2 protein, or said parts thereof, are derived.

In another embodiment, the above recombinant virus compositions may be used for inducing HCV-specific antibodies or for stimulating T-cell activity or for stimulating cytokine secretion or cytokine production.

In another embodiment to the recombinant virus compositions, said recombinant virus is a vaccinia virus, a recombinant avipox virus or a recombinant Ankara Modified Virus.

Another aspect of the invention relates to a method of treating a mammal infected with HCV comprising administering an effective amount of a recombinant vaccine composition as described above.

The mammal in any of the above aspects of the invention may in particular be a human.

One further aspect of the present invention relates to a method to reduce liver disease in a chronic HCV-infected mammal or human comprising administering a therapeutic vaccine to said mammal or human.

In another aspect, a method to reduce liver fibrosis progression in a chronic HCV-infected mammal or human comprising administering a therapeutic vaccine to said mammal or human is covered.

A further aspect relates to a method to reduce liver fibrosis in a chronic HCV-infected mammal or human comprising administering a therapeutic vaccine to said mammal or human.

A further aspect relates to a method to reduce liver steatosis in a chronic HCV-infected mammal or human comprising administering a therapeutic vaccine to said mammal or human.

Yet another aspect of the invention embodies a method to reduce liver disease by at least 2 points according to the overall Ishak score or Ishak activity score in a chronic HCV-infected mammal or human comprising administering a therapeutic vaccine to said mammal or human.

Another further aspect relates to a method to reduce liver disease or liver fibrosis by at least 1 point according to the Ishak fibrosis score in a chronic HCV-infected mammal or human comprising administering a therapeutic vaccine to said mammal or human.

A further aspect of the invention provides a method to reduce serum ALT levels in a chronic HCV-infected mammal or human comprising administering a therapeutic vaccine to said mammal or human.

Yet another aspect of the invention relates to a method to reduce anti-E1 and/or anti-E2 immunoreactivity in the liver of a chronic HCV-infected mammal or human comprising administering a therapeutic vaccine to said mammal or human.

Furthermore included in the invention is a method for treating a chronic HCV-infected mammal or human wherein said method is comprising administration of multiple doses of any of the compositions of the invention, such as a therapeutic HCV vaccine composition, to said mammal or human and wherein said multiple doses are administrated to said mammal or human separated by a specified time interval. Thus, said plurality of administrations of a composition of the invention to treat a chronic HCV-infected carrier may be separated, e.g., by a time-interval of 4 weeks or less. Thus, said time intervals could be 1 or 1.5 or 2 or 2.5 or 3 or 3.5 or 4 weeks, or could be 1, 2, 3, 4, 5, 6, 7, 8, 9, 10, 11, 12, 13, 14, 15, 16, 17, 18, 19, 20, 21, 22, 23, 24, 25, 26, 27 or 28 days. In particular, said method of treating a chronic HCV-infected mammal or human comprises a plurality of administrations of a composition of the invention, such as a therapeutic HCV vaccine composition, to said mammal or human wherein said administrations are separated by a time interval of 3 weeks. In a further embodiment, said plurality of administrations consists of a first series of at least 5 administrations followed by an administration-free period of at least 12 weeks followed by a second series of at least 3 administrations. In particular, a series of administrations may comprise at least 3, 4, 5, 6, 7, 8, 9, 10, 11, 12, or more administrations. The administration-free period may be at least 6, 7, 8, 9, 10, 11, 12, 13, 14, 15, 16, 17, 18 or more weeks.

It will furthermore be clear that any of the compositions of the invention, such as a therapeutic HCV vaccine composition, can be used for obtaining any of the effects aimed at in the various methods as described above. Said therapeutic vaccine as applied in the methods described above may be any composition of the current invention, such as a therapeutic HCV vaccine composition.

The use of the HCV E1/E2 proteins or parts thereof or E1/E2 peptides as outlined throughout the above description for the manufacture of a composition, HCV composition, vaccine, HCV vaccine, therapeutic vaccine or therapeutic HCV vaccine for use in any of the methods outlined above or used for obtaining any of the effects aimed at in the various methods outlined above is also envisaged by the current invention.

In any of these methods said therapeutic vaccine comprises at least one HCV antigen and, optionally, a pharmaceutically acceptable adjuvant such as alum. In one embodiment thereto said HCV antigen is an E1 or E2 antigen, or an immunogenic part of an E1 or E2 antigen. When referring to the HCV antigen as being an E1s antigen, the E1s antigen maybe defined by SEQ ID NO:123.

With the term “liver disease” is meant in this context any abnormal liver condition caused by infection with the hepatitis C virus including inflammation, fibrosis, cirrhosis, necrosis, necro-inflammation and hepatocellular carcinoma.

With “steatosis” is meant a histological feature of lipid accumulation in the hepatocytes that is indicative of liver involvement in a wide variety of systemic disorders, toxic or drug-induced liver injury, as well as of various specific liver diseases, including hepatitis C infection, Wilson's disease, and galactosemia.

With “reducing liver disease” is meant any stabilization or reduction of the liver disease status. Liver disease can be determined, e.g., by the Knodell scoring system or the Knodell scoring system adapted by Ishak. A reduction of this score by two points is accepted as therapeutically beneficial effect in several studies (see, e.g., studies published after 1996 as indicated in Table 2 of Shiffman 1999).

With “reducing liver fibrosis progression” is meant any slowing down, halting or reverting of the normally expected progression of liver fibrosis. Liver fibrosis progression can be determined, e.g., by the Metavir scoring system. Normal expected progression of liver fibrosis according to this system was published to be an increase of the Metavir score of an untreated chronic HCV patient of approximately 0.133 per year (Poynard et al. 1997). Fibrosis is considered to include any form of fibrosis, e.g. as scored by the Metavir or Ishak system, including perisinusoidal fibrosis.

With the term “HCV antigen” is meant any HCV protein or fragment thereof comprising at least one T cell epitope or B cell epitope.

A further aspect of the current invention provides a method to predict changes in liver disease in a chronic HCV-infected mammal or human, said method comprising:

(i) determining the level of serum anti-E1 antibody level prior to therapeutic vaccination with a HCV vaccine composition comprising an E1 antigen;

(ii) determining the level of serum anti-E1 antibody level after therapeutic vaccination with a HCV vaccine composition comprising an E1 antigen;

(iii) inferring the difference in level of serum anti-E1 antibody level determined in (i) and (ii) and, therefrom, predicting the change in liver disease.

It will be clear to the skilled artisan that a significantly high and positive difference in level of serum anti-E1 antibody, calculated as the value measured in (ii) minus the value measured in (i), would tip the balance in favor of a positive prediction, i.e., a prediction that the degree of liver disease will decrease. It will also be clear that a sustained significantly high and positive difference would add to the positive character of the prediction of decreased liver disease. When said difference is, however, not significantly high, is zero, is negative, or is significantly high and positive at one point in time but is not sustained, this would then tip the balance in favor of a negative prediction, i.e., a prediction that the degree of liver disease will remain unchanged or will increase. A sustained high level of serum anti-E1 antibody could be reached trough additional immunizations either by administering a new series of immunizations after an administration free period or by repeating immunizations with a larger time interval, e.g. 6 weeks, after an initial priming series consisting of administrations with a short time interval, e.g. 3 weeks.

### FIGURE AND TABLE LEGENDS

FIG. 1: Restriction map of plasmid pgpt ATA 18

FIG. 2: Restriction map of plasmid pgs ATA 18

FIG. 3: Restriction map of plasmid pMS 66

FIG. 4: Restriction map of plasmid pv HCV-11A

FIG. 5: Anti-E1 levels in non-responders to IFN treatment

FIG. 6: Anti-E1 levels in responders to IFN treatment

FIG. 7: Anti-E1 levels in patients with complete response to EFN treatment

FIG. 8: Anti-E1 levels in incomplete responders to IFN treatment

FIG. 9: Anti-E2 levels in non-responders to IFN treatment

FIG. 10: Anti-E2 levels in responders to IFN treatment

FIG. 11: Anti-E2 levels in incomplete responders to IFN treatment

FIG. 12: Anti-E2 levels in complete responders to IFN treatment

FIG. 13: Human anti-E1 reactivity competed with peptides

FIG. 14: Competition of reactivity of anti-E1 monoclonal antibodies with peptides

FIG. 15: Anti-E1 (epitope 1) levels in non-responders to IFN treatment

FIG. 16: Anti-E1 (epitope 1) levels in responders to IFN treatment

FIG. 17: Anti-E1 (epitope 2) levels in non-responders to IFN treatment

FIG. 18: Anti-E1 (epitope 2) levels in responders to IFN treatment

FIG. 19: Competition of reactivity of anti-E2 monoclonal antibodies with peptides

FIG. 20: Human anti-E2 reactivity competed with peptides

FIG. 21: Nucleic acid sequences of the present invention. The nucleic acid sequences encoding an E1 or E2 protein according to the present invention may be translated (SEQ ID NO 3 to 13, 21-31, 35 and 41-49 are translated in a reading frame starting from residue number 1, SEQ ID NO 37-39 are translated in a reading frame starting from residue number 2), into the amino acid sequences of the respective E1 or E2 proteins as shown in the sequence listing.

FIG. 22: ELISA results obtained from lentil lectin chromatography eluate fractions of 4 different E1 purifications of cell lysates infected with vvHCV39 (type 1b), vvHCV40 (type 1b), vvHCV62 (type 3a), and vvHCV63 (type 5a).

FIG. 23: Elution profiles obtained from the lentil lectin chromatography of the 4 different E1 constructs on the basis of the values as shown in FIG. 22.

FIG. 24: ELISA results obtained from fractions obtained after gelfiltration chromatography of 4 different E1 purifications of cell lysates infected with vvHCV39 (type 1b), vvHCV40 (type 1b), vvHCV62 (type 3a), and vvHCV63 (type 5a).

FIG. 25: Profiles obtained from purifications of E1 proteins of type 1b (1), type 3a (2), and type 5a (3) (from RK13 cells infected with vvHCV39, vvHCV62, and vvHCV63, respectively; purified on lentil lectin and reduced as in example 5.2-5.3) and a standard (4). The peaks indicated with ‘1’, ‘2’, and ‘3’, represent pure E1 protein peaks (see FIG. 24, E1 reactivity mainly in fractions 26 to 30).

FIG. 26: Silver staining of an SDS-PAGE as described in example 4 of a raw lysate of E1 vvHCV40 (type 1b) (lane 1), pool 1 of the gelfiltration of vVHCV40 representing fractions 10 to 17 as shown in FIG. 25 (lane 2), pool 2 of the gelfiltration of vvHCV40 representing fractions 18 to 25 as shown in FIG. 25 (lane 3), and E1 pool (fractions 26 to 30) (lane 4).

FIG. 27: Streptavidine-alkaline phosphatase blot of the fractions of the gelfiltration of E1 constructs 39 (type 1b) and 62 (type 3a). The proteins were labelled with NEM-biotin. Lane 1: start gelfiltration construct 39, lane 2: fraction 26 construct 39, lane 3: fraction 27 construct 39, lane 4: fraction 28 construct 39, lane 5: fraction 29 construct 39, lane 6: fraction 30 construct 39, lane 7 fraction 31 construct 39, lane 8: molecular weight marker, lane 9: start gelfiltration construct 62, lane 10: fraction 26 construct 62, lane 11: fraction 27 construct 62, lane 12: fraction 28 construct 62, lane 13: fraction 29 construct 62, lane 14: fraction 30 construct 62, lane 15: fraction 31 construct 62.

FIG. 28: Siver staining of an SDS-PAGE gel of the gelfiltration fractions of vvHCV-39 (E1 s, type 1b) and vvHCV-62 (E1 s, type 3a) run under identical conditions as FIG. 26. Lane 1: start gelfiltration construct 39, lane 2: fraction 26 construct 39, lane 3: fraction 27 construct 39, lane 4: fraction 28 construct 39, lane 5: fraction 29 construct 39, lane 6: fraction 30 construct 39, lane 7 fraction 31 construct 39, lane 8: molecular weight marker, lane 9: start gelfiltration construct 62, lane 10: fraction 26 construct 62, lane 11: fraction 27 construct 62, lane 12: fraction 28 construct 62, lane 13: fraction 29 construct 62, lane 14: fraction 30 construct 62, lane 15: fraction 31 construct 62.

FIG. 29: Western Blot analysis with anti-E1 mouse monoclonal antibody 5E1A10 giving a complete overview of the purification procedure. Lane 1: crude lysate, Lane 2: flow through of lentil chromagtography, Lane 3: wash with Empigen BB after lentil chromatography, Lane 4: Eluate of lentil chromatography, Lane 5: Flow through during concentration of the lentil eluate, Lane 6: Pool of E1 after Size Exclusion Chromatography (gelfiltration).

FIG. 30: OD₂₈₀ profile (continuous line) of the lentil lectin chromatography of E2 protein from RK13 cells infected with vvHCV44. The dotted line represents the E2 reactivity as detected by ELISA (as in example 6).

FIG. 31A: OD₂₈₀ profile (continuous line) of the lentil-lectin gelfiltration chromatography E2 protein pool from RK13 cells infected with vvHCV44 in which the E2 pool is applied immediately on the gelfiltration column (non-reduced conditions). The dotted line represents the E2 reactivity as detected by ELISA (as in example 6).

FIG. 31B: OD₂₈₀ profile (continuous line) of , the lentil-lectin gelfiltration chromatography E2 protein pool from RK13 cells infected with vvHCV44 in which the E2 pool was reduced and blocked according to Example 5.3 (reduced conditions). The dotted line represents the E2 reactivity as detected by ELISA (as in example 6).

FIG. 32: Ni²⁺-IMAC chromatography and ELISA reactivity of the E2 protein as expressed from vvHCV44 after gelfiltration under reducing conditions as shown in FIG. 31B.

FIG. 33: Silver staining of an SDS-PAGE of 0.5 μg of purified E2 protein recovered by a 200 mM imidazole elution step (lane 2) and a 30 mM imidazole wash (lane 1) of the Ni²⁺-IMAC chromatography as shown in FIG. 32.

FIG. 34: OD profiles of a desalting step of the purified E2 protein recovered by 200 mM imidazole as shown in FIG. 33, intended to remove imidazole.

FIG. 35A: Antibody levels to the different HCV antigens (Core 1, Core 2, E2HCVR, NS3) for NR and LTR followed during treatment and over a period of 6 to 12 months after treatment determined by means of the LIAscan method. The average values are indicated by the curves with the open squares.

FIG. 35B: Antibody levels to the different HCV antigens (NS4, NS5, E1 and E2) for NR and LTR followed during treatment and over a period of 6 to 12 months after treatment determined by means of the LIAscan method. The average values are indicated by the curve with the open squares.

FIG. 36: Average E1 antibody (E1Ab) and E2 antibody (E2Ab) levels in the LTR and NR groups.

FIG. 37: Averages E1 antibody (E1Ab) levels for non-responders (NR) and long term responders (LTR) for type 1b and type 3a.

FIG. 38: Relative map positions of the anti-E2 monoclonal antibodies.

FIG. 39: Partial deglycosylation of HCV E1 envelope protein. The lysate of vvHCV10A-infected RK 13 cells were incubated with different concentrations of glycosidases according to the manufacturer's instructions. Right panel: Glycopeptidase F (PNGase F). Left panel: Endoglycosidase H (Endo H).

FIG. 40: Partial deglycosylation of HCV E2 envelope proteins. The lysate of vvHCV64-infected (E2) and vvHCV41-infected (E2s)RK13 cells were incubated with different concentrations of Glycopeptidase F (PNGase F) according to the manufacturer's instructions.

FIG. 41: In vitro mutagenesis of HCV E1 glycoproteins. Map of the mutated sequences and the creation of new restriction sites.

FIG. 42A: In vitro mutagenesis of HCV E1 glycoprotein (part 1). First step of PCR amplification.

FIG. 42B: In vitro mutagensis of HCV E1 glycoprotein (part 2). Overlap extension and nested PCR.

FIG. 43: In vitro mutagesesis of HCV E1 glycoproteins. Map of the PCR mutated fragments (GLY-# and OVR-#) synthesized during the first step of amplification.

FIG. 44A: Analysis of E1 glycoprotein mutants by Western blot expressed in HeLa (left) and RK13 (right) cells. Lane 1: wild type VV (vaccinia virus), Lane 2: original E1 protein (vvHCV-10A), Lane 3: E1 mutant Gly-1 (vvHCV-81), Lane 4: E1 mutant Gly-2 (vvHCV-82), Lane 5: E1 mutant Gly-3 (vvHCV-83), Lane 6: E1 mutant Gly-4 (vvHCV-84), Lane 7: E1 mutant Gly-5 (vvHCV-85), Lane 8: E1 mutant Gly-6 (vvHCV-86).

FIG. 44B: Analysis of E1 glycosylation mutant vaccinia viruses by PCR amplification/restriction. Lane 1: E1 (vvHCV-10A), BspE I, Lane 2: E1.GLY-1 (vvHCV-81), BspE I, Lane 4: E1 (vvHCV-10A), Sac I, Lane 5: E1.GLY-2 (vvHCV-82), Sac I, Lane 7: E1 (vvHCV-10A), Sac I, Lane 8: E1.GLY-3 (vvHCV-83), Sac I, Lane 10: E1 (vvHCV-10A), Stu I, Lane 11: E1.GLY-4 (vvHCV-84), Stu I, Lane 13: E1 (vvHCV-10A), Sma I, Lane 14: E1.GLY-5 (vvHCV-85), Sma I, Lane 16: E1 (vvHCV-10A), Stu I, Lane 17: E1.GLY-6 (vvHCV-86), Stu I, Lane 3-6-9-12-15: Low Molecular Weight Marker, pBluescript SK+, Msp I.

FIG. 45: SDS polyacrylamide gel electrophoresis of recombinant E2 expressed in S. cerevisiae. Innoculates were grown in leucine selective medium for 72 hrs. and diluted 1/15 in complete medium. After 10 days of culture at 28° C., medium samples were taken. The equivalent of 200 μl of culture supernatant concentrated by speedvac was loaded on the gel. Two independent transformants were analysed.

FIG. 46: SDS polyacrylamide gel electrophoresis of recombinant E2 expressed in a glycosylation deficient S. cerevisiae mutant. Innoculae were grown in leucine selective medium for 72 hrs. and diluted 1/15 in complete medium. After 10 days of culture at 28° C., medium samples were taken. The equivalent of 350 μl of culture supernatant, concentrated by ion exchange chromatography, was loaded on the gel.

FIG. 47: Profile of chimpanzees and immunization schedule.

FIG. 48: Cellular response after 3 immunizations.

FIG. 49: Evolution of cellular response upon repeated E1 immunizations.

FIG. 50: Cellular response upon NS3 immunizations.

FIG. 51: Stimulation index through week 28. The stimulation index (SI; cellular immune response) was obtained by culturing PBMC (10⁵ cells), drawn from the individuals before immunization (week 0), two weeks after the third immunization (week 8), before the booster immunization (week 26) and two weeks after the booster immunization (week 28), in the presence or absence of 3 μg of recombinant E1s or 2 μg tetanos toxoid and determining the amount of tritiated thymidine incorporated in these cells during a pulse of 18 hours after 5 days of culture. The stimulation index is the ratio of thymidine incorporated in the cells cultured with envelope antigen versus the ones cultured without antigen. Samples of week 0 and 8 were determined in a first assay (A), while the samples of week 26 and 28 were determined in a second assay (B) in which the samples of week 0 were reanalyzed. Results are expressed as the geometric mean stimulation index of all 20 (A, experiment) or 19 (B, experiment) volunteers.

FIG. 52: Cytokine production of PBMCs. PBMC (10⁵ cells), drawn from the individuals before the booster immunization (week 26) and two weeks after the booster immunization (week 28), were cultured in the presence of 3 μg of recombinant E1s (E1) or 2 μg of tetanos toxoid (TT) or no antigen (B1). Cytokines were measured in the supernatant taken after 24 hours (interleukin-5) or after 120 hours (interferon-gamma) by means of ELISA. The stimulation index is the ratio of cytokine measured in the supernatants of cells cultured with envelope antigen versus the ones cultured without antigen. Results are expressed as the geometric mean of pg cytokine/ml secreted of all 19 volunteers. Samples with a cytokine amount below detection limit were assigned the value of the detection limit. Similarly samples with extremely high concentrations of cytokine out of the linear range of the assay were assigned the value of the limit of the linear range of the assay.

FIG. 53: Thymidine incorporation results. The stimulation index (cellular immune response) was obtained by culturing PBMC (3×10⁵ cells), in the presence or absence of peptides and determining the amount of tritiated thymidine incorporated in these cells during a pulse after 5-6 days of culture. The stimulation index is the ratio of thymidine incorporated in the cells cultured with peptide versus the ones cultured without peptide. Results are expressed as individual values for vaccinated persons (top panel) or non vaccinated or controls (lower panel).

FIG. 54: Three-dimensional graph showing for the individual patients (each represented by a dot) the % change in ALT-levels (absolute change from baseline) on the X-axis, the change in serum anti-E1 antibody levels (in mU/mL) on the Y-axis, and the change in Ishak fibrosis score on the Z-axis.

FIG. 55: Three-dimensional graph showing for the individual patients (each represented by a dot) the % change in ALT-levels (absolute change from baseline) on the X-axis, the change in serum anti-E1 antibody levels (in mU/mL) on the Y-axis, and the change in Metavir fibrosis score on the Z-axis.

FIG. 56: For each individual patient (represented by a dot or a “★”), the Ishak fibrosis score (on the X-axis) and the ALT values (on the Y-axis) are given. In panel A (top), the baseline (pre-vaccination) situation is given whereas panel B (bottom) is illustrating the situation at the time of taking of liver biopsies. The seven patients with the highest increase in serum anti-E1 antibody level are represented by a “★”.

FIG. 57: The influence of the age (on the X-axis) of each individual patient (represented by a dot or a “★”) on the Ishak fibrosis score (on the Y-axis) is given. In panel A (top), the baseline (pre-vaccination) situation is given whereas panel B (bottom) is illustrating the situation at the time of taking of liver biopsies. The seven patients with the highest increase in serum anti-E1 antibody level are represented by a “★”.

Table 1: Features of the respective clones and primers used for amplification for constructing the different forms of the E1 protein as despected in Example 1.

Table 2: Summary of Anti-E1 tests

Table 3: Synthetic peptides for competition studies

Table 4: Changes of envelope antibody levels over time.

Table 5: Difference between LTR and NR

Table 6: Competition experiments between murine E2 monoclonal antibodies

Table 7: Primers for construction of E1 glycosylation mutants

Table 8: Analysis of E1 glycosylation mutants by ELISA

Table 9: Profile of adjuvanted E1 Balb/c mice.

Table 10: Humoral responses: No. of immunizations required for different E1-antibodies levels.

Table 11: Chimpanzee antibody titers.

Table 12: Human antibody titers.

Table 13: Human antibody titers (8-28 weeks).

Table 14: Stimulation index (SI) of cultured PBMC, drawn from the individuals four weeks (W16) after the fourth immunization and two weeks (W26) after the fifth immunization in the presence or absence of 3 μg of E1s. A stimulation index of >3 is considered a positive signal.

Table 15: Ishak grading of necro-inflammatory intensities for periportal hepatitis, confluent necrosis, focal inflammation, portal inflammation and the overall total inflammation grading. Scores are indicated as the change from baseline (mean and 95% confidence interval) and the mean baseline- to end-values.

Table 16: Overview of frequencies (given as number of patients) of changes of a given baseline Metavir score (given in top row; Baseline 0 to 4) to a given Metavir score at the end of the second course of therapeutic E1s vaccination (given in left row; EOT 0 to 4). For instance, the “5” marked with a “*” (i.e., “5*”) means that 5 patients had a baseline Metavir score of 1 and a Metavir score of 0 at the end of the second course treatment (EOT=end of treatment).

Table 17: Correlation between serum anti-E1 antibody levels induced by therapeutic E1s vaccination and change in overall Ishak scores. Given are the number of patients corresponding to the possible criteria as outlined in the Table.

### EXAMPLE 1

### Cloning and Expression of the Hepatitis C Virus E1 Protein

1. Construction of Vaccinia Virus Recombination Vectors

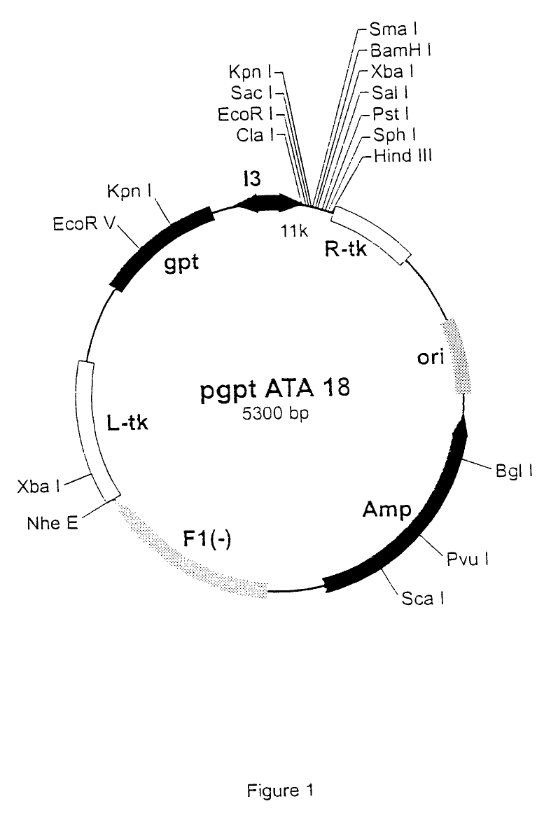

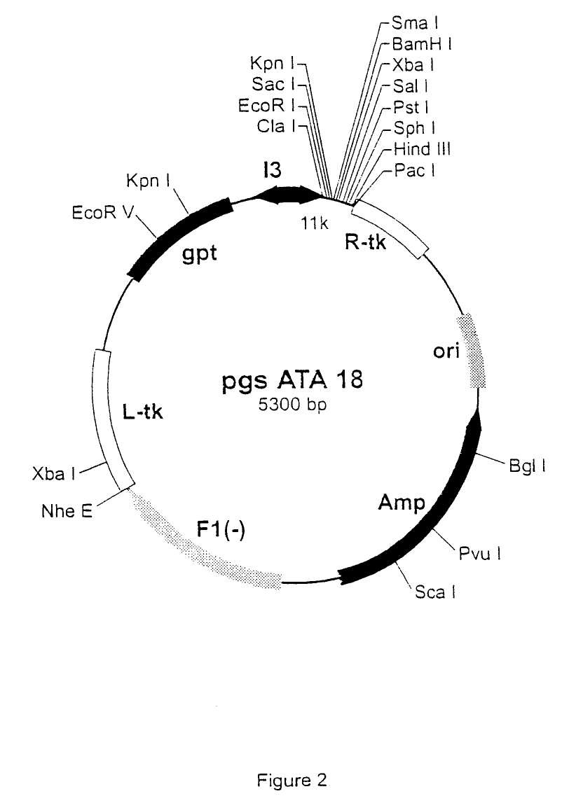

The pgptATA18 vaccinia recombination plasmid is a modified version of pATA18 (Stunnenberg et al, 1988) with an additional insertion containing the E. coli xanthine guanine phosphoribosyl transferase gene under the control of the vaccinia virus 13 intermediate promoter (FIG. 1). The plasmid pgsATA 18 was constructed by inserting an oligonucleotide linker with SEQ ID NO 1/94, containing stop codons in the three reading frames, into the Pst I and HindIII-cut pATA 18 vector. This created an extra Pac I restriction site (FIG. 2). The original HindIII site was not restored.

Oligonucleotide linker with SEQ ID NO 1/94:

| | | | |
|---|---|---|---|
| 5′ | G GCATGC AAGCTT AATTAATT | 3′ | (SEQ ID NO:1) |
| 3′ | ACGTC CGTACG TTCGAA TTAATTAA TCGA | 5′ | (SEQ ID NO:94) |

\overline{PstI } \overline{SphI }H\overline{indIII} {overscore ( Pac I ()}H\overline{indI}II)

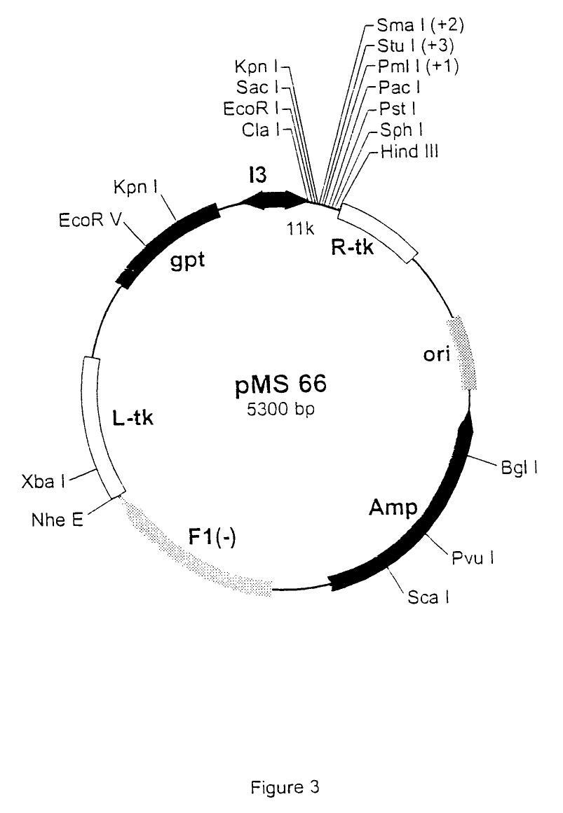

In order to facilitate rapid and efficient purification by means of Ni²⁺ chelation of engineered histidine stretches fused to the recombinant proteins, the vaccinia recombination vector pMS66 was designed to express secreted proteins with an additional carboxy-terminal histidine tag. An oligonucleotide linker with SEQ ID NO 2/95, containing unique sites for 3 restriction enzymes generating blunt ends (Sma I, Stu I and Pml I/Bbr PI) was synthesized in such a way that the carboxy-terminal end of any cDNA could be inserted in frame with a sequence encoding the protease factor Xa cleavage site followed by a nucleotide sequence encoding 6 histidines and 2 stop codons (a new Pac I restriction site was also created downstream the 3′end). This oligonucleotide with SEQ ID NO 2/95 was introduced between the Xma I and Pst I sites of pgptATA18 (FIG. 3).

Oligonucleotide linker with SEQ ID NO 2/95:

| | |
|---|---|
| 5′-CCGGG GAGGCCTGCACGTGATCGAGGGCAGACACCATCACCACCA/ | |
| 3′-C CTCCGGACGTGCACTAGCTCCCGTCTGTGGTAGTGGTGGT/ | |
| XmaI | |
| /TCACTAATAGTTAATTAA CTGCA-3′ | (SEQ ID NO: 2) |
| /AGTGATTATCAATTAATT G-5′ | (SEQ ID NO: 95) |
| PstI | |

Plasmid pgptATA-18 contained within Escherichia coli MC1061 (lambda) has been deposited under the terms of the Budapest Treaty at BCCM/LMBP (Belgian Coordinated Collections of microorganisms/Laboratorium voor Moleculaire Biologie—Plasmidencollectie, Universiteit Gent, K. L. Ledeganckstraat 35, B-9000 Ghent, Belgium), and bears accession number LMBP4486. Said deposit was made on Jan. 9, 2002.

### Example 2

### Construction of HCV Recombinant Plasmids

2.1. Constructs Encoding Different Forms of the E1 Protein

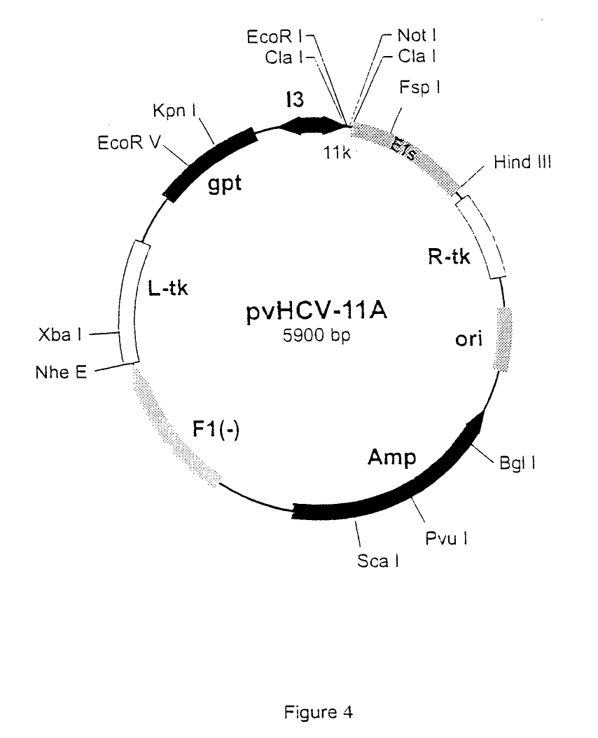

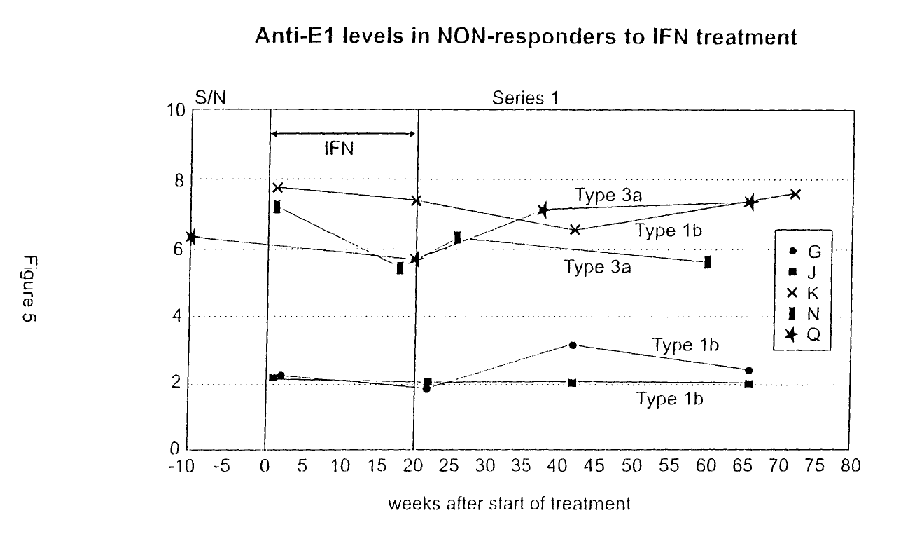

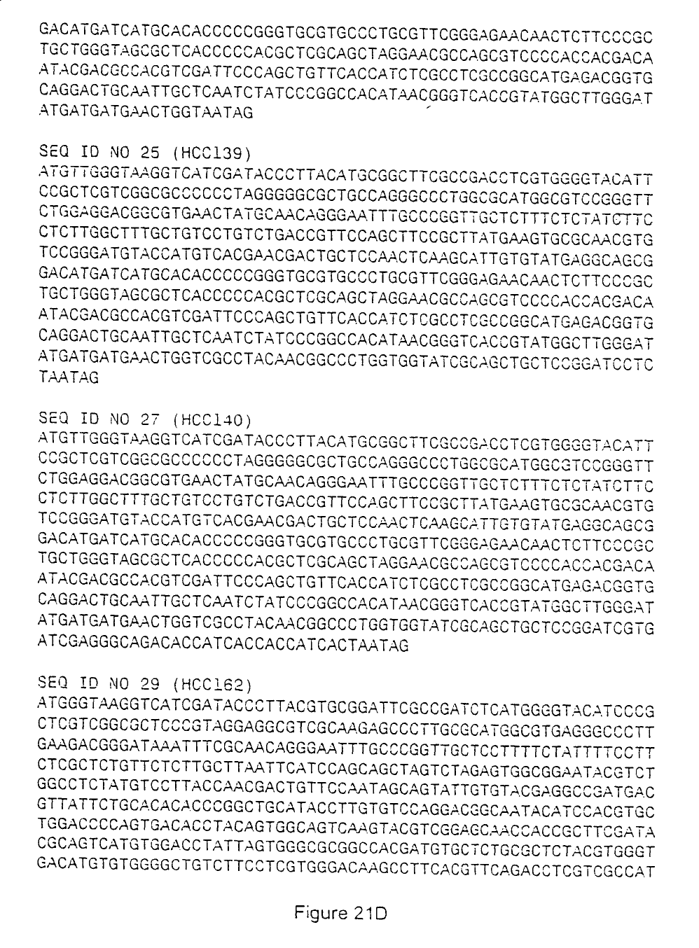

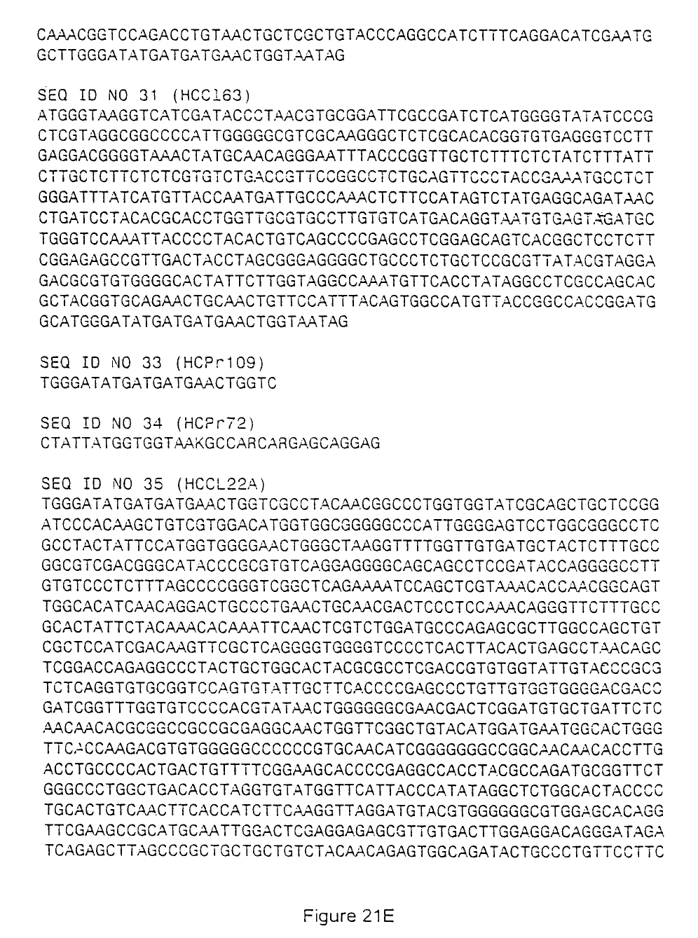

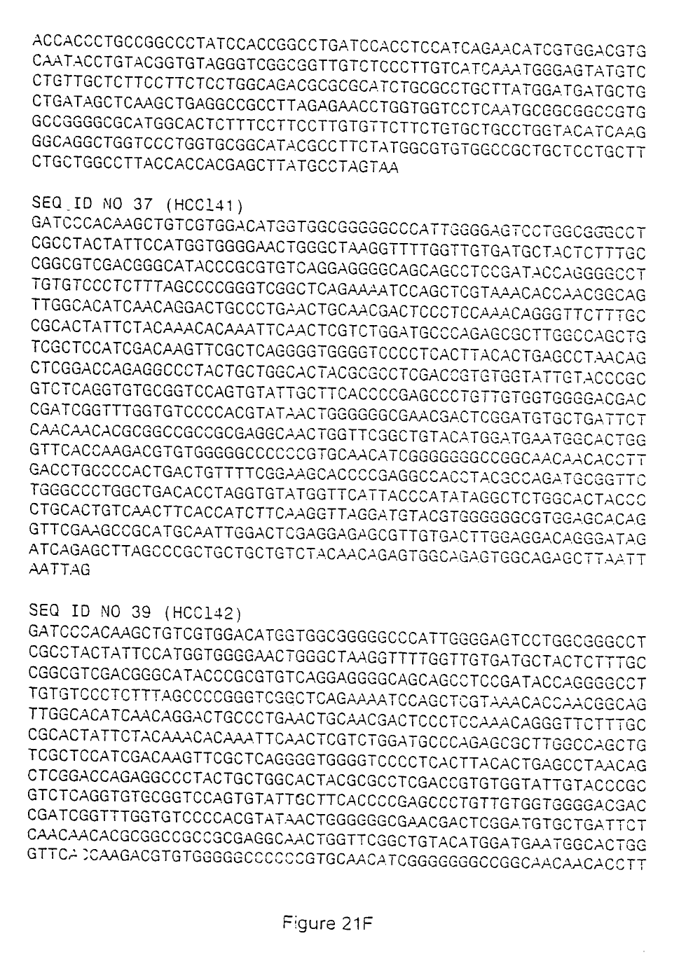

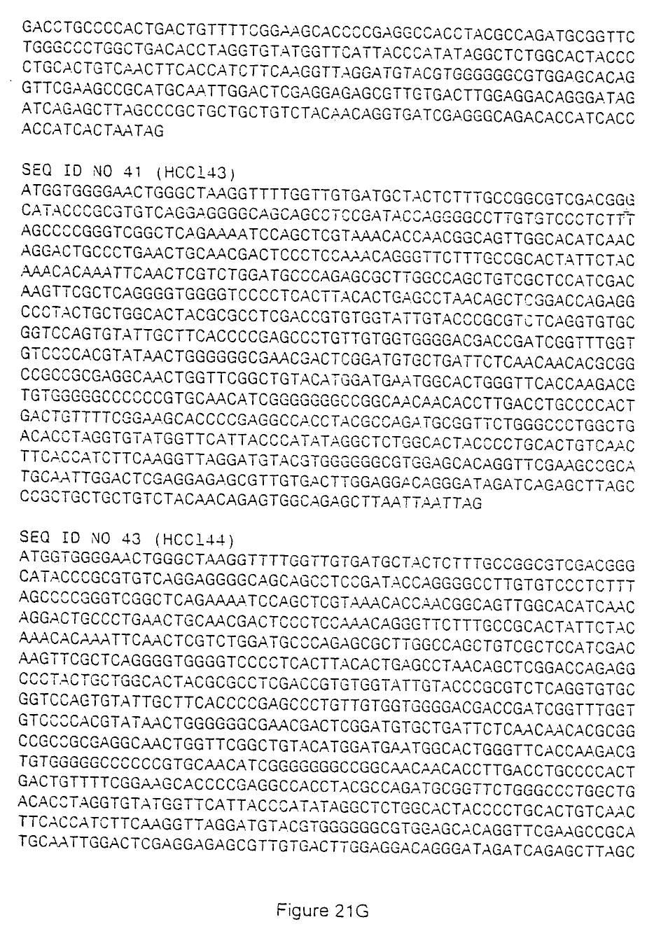

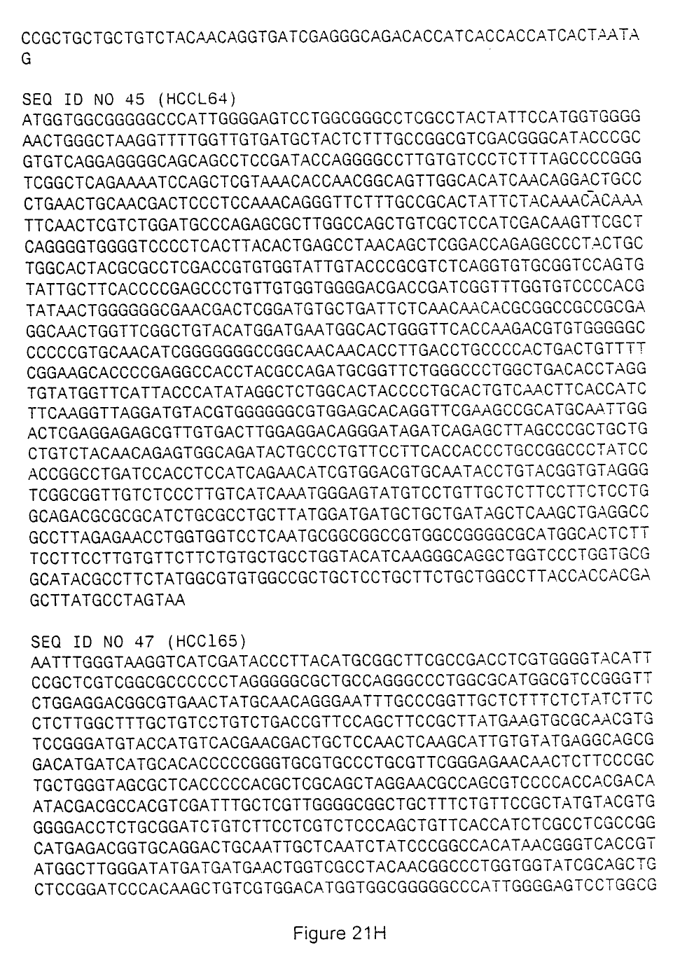

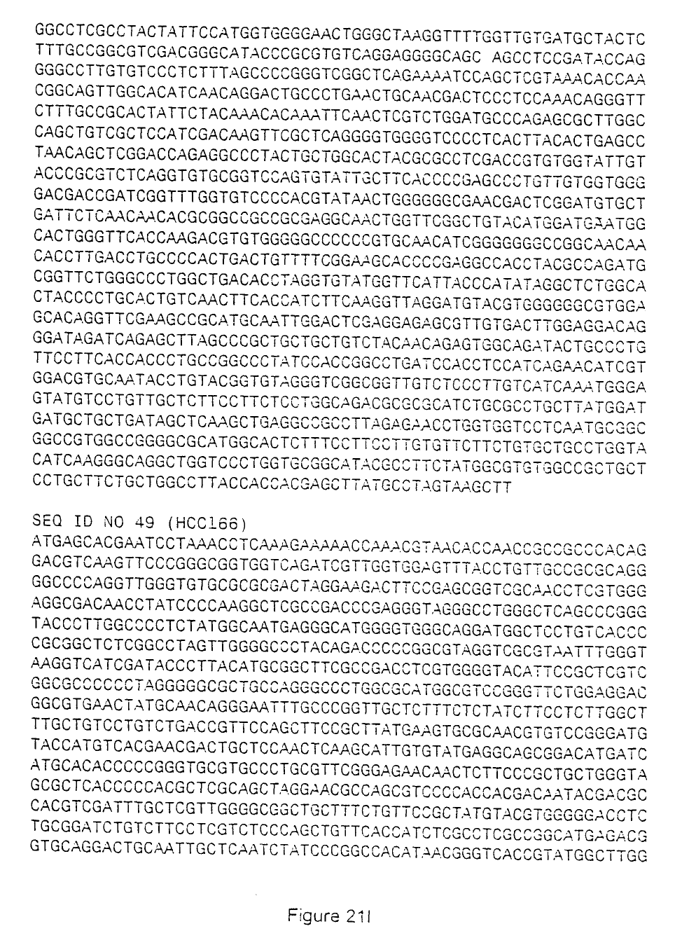

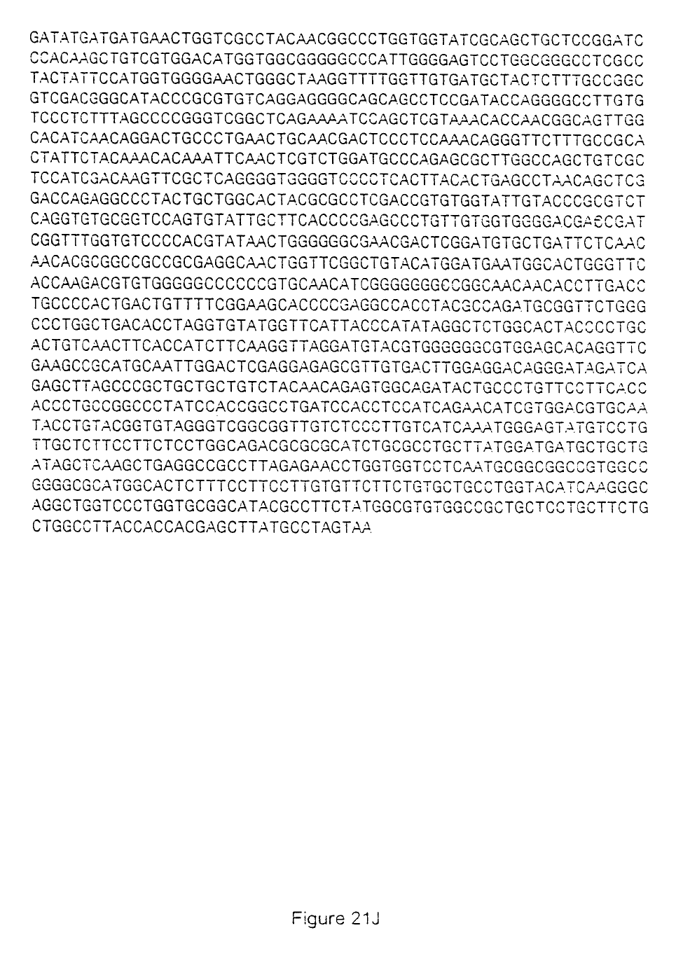

Polymerase Chain Reaction (PCR) products were derived from the serun samples by RNA preparation and subsequent reverse-transcription and PCR as described previously (Stuyver et al., 1993b). Table 1 shows the features of the respective clones and the primers used for amplification. The PCR fragments were cloned into the Sma I-cut pSP72 (Promega) plasmids. The following clones were selected for insertion into vaccinia reombination vectors: HCC19A (SEQ ID NO:3), HCC110A (SEQ ID NO:5), HCC111A (SEQ ID NO:7), HCC112A (SEQ ID NO:9), HCC113A (SEQ ID NO:l 1), and HCC117A (SEQ ID NO:13) as depicted in FIG. 21. cDNA fragments containing the E1-coding regions were cleaved by EcoRI and HindIII restriction from the respective pSP72 plasmids and inserted into the EcoRI/HindIII-cut pgptATA-18 vaccinia recombination vector (described in example 1), downstream of the 11K vaccinia virus late promoter. The respective plasmids were designated pvHCV-9A, pvHCV-10A, pvHCV-11A, pvHCV-12A, pvHCV-13A and pvHCV-17A, of which pvHCV-11A is shown in FIG. 4.

2.2. Hydrophobic Region E1 Deletion Mutants

Clone HCC137, containing a deletion of codons Asp264 to Val287 (nucleotides 790 to 861, region encoding hydrophobic domain I) was generated as follows: 2 PCR fragments were generated from clone HCC110A with primer sets HCPr52 (SEQ ID NO:16)/HCPr107 (SEQ ID NO:19) and HCPr108 (SEQ ID NO:20)/HCPR54 (SEQ ID NO:18). These primers are shown in FIG. 21. The two PCR fragments were purified from agarose gel after electrophoresis and 1 ng of each fragment was used together as template for PCR by means of primers HCPr52 (SEQ ID NO:16) and HCPr54 (SEQ ID NO:18). The resulting fragment was cloned into the Sma I-cut pSP72 vector and clones containing the deletion were readily identified because of the deletion of 24 codons (72 base pairs). Plasmid pSP72HCC137 containing clone HCC137 (SEQ ID 15) was selected. A recombinant vaccinia plasmid containing the full-length E1 cDNA lacking hydrophobic domain I was constructed by inserting the HCV sequence surrounding the deletion (fragment cleaved by Xma I and BamH I from the vector pSP72-HCC137) into the Xma I-Bam H I sites of the vaccinia plasmid pvHCV-10A. The resulting plasmid was named pvHCV-37. After confirmatory sequencing, the amino-terminal region containing the internal deletion was isolated from this vector pvHCV-37 (cleavage by EcoR I and BstE II) and reinserted into the Eco RI and Bst EII-cut pvHCV-11A plasmid. This construct was expected to express an E1 protein with both hydrophobic domains deleted and was named pvHCV-38. The E1-coding region of clone HCC138 is represented by SEQ ID NO:23.

As the hydrophilic region at the E1 carboxyterminus (theoretically extending to around amino acids 337-340) was not completely included in construct pvHCV-38, a larger E1 region lacking hydrophobic domain I was isolated from the pvHCV-37 plasmid by EcoR I/Bam HI cleavage and cloned into an EcoRl/BamHI-cut pgsATA-18 vector. The resulting plasmid was named pvHCV-39 and contained clone HCC139 (SEQ ID NO:25). The same fragment was cleaved from the pvHCV-37 vector by BamH I (of which the sticky ends were filled with Klenow DNA Polymerase I (Boehringer)) and subsequently by EcoR I (5′ cohesive end). This sequence was inserted into the EcoRI and Bbr PI-cut vector pMS-66. This resulted in clone HCC140 (SEQ ID NO:27) in plasmid pvHCV-40, containing a 6 histidine tail at its carboxy-terminal end.

2.3. E1 of Other Genotypes

Clone HCC162 (SEQ ID NO:29) was derived from a type 3a-infected patient with chronic hepatitis C (serum BR36, clone BR36-9-13, SEQ ID NO:19 in WO 94/25601, and see also Stuyver et al. 1993a) and HCC163 (SEQ ID NO:31) was derived from a type 5a-infected child with post-transfusion hepatitis (serum BE95, clone PC-4-1, SEQ ID NO:45 in WO 94/25601).

2.4. E2 Constructs

The HCV E2 PCR fragment 22 was obtained from serum BE11 (genotype 1b) by means of primers HCPr109 (SEQ ID NO:33) and HCPr72 (SEQ ID NO:34) using techniques of RNA preparation, reverse-transcription and PCR, as described in Stuyver et al., 1993b, and the fragment was cloned into the Sma I-cut pSP72 vector. Clone HCC122A (SEQ ID NO:35) was cut with NcoI/AlwNI or by BamHi/AlwNI and the sticky ends of the fragments were blunted (NcoI and BamHI sites with Klenow DNA Polymerase I (Boehringer), and AlwNI with T4 DNA polymerase (Boehringer)). The BamHI/AlwNI cDNA fragment was then inserted into the vaccinia pgsATA-18 vector that had been linearized by EcoR I and Hind III cleavage and of which the cohesive ends had been filled with Klenow DNA Polymerase (Boehringer). The resulting plasmid was named pvHCV-41 and encoded the E2 region from amino acids Met347 to Gln673, including 37 amino acids (from Met347 to Gly383) of the E1 protein that can serve as signal sequence. The same HCV cDNA was inserted into the EcoR I and Bbr PI-cut vector pMS66, that had subsequently been blunt ended with Klenow DNA Polymerase. The resulting plasmid was named pvHCV-42 and also encoded amino acids 347 to 683. The NcoI/AlwNI fragment was inserted in a similar way into the same sites of pgsATA-18 (pvHCV-43) or pMS-66 vaccinia vectors (pvHCV-44). pvHCV-43 and pvHCV-44 encoded amino acids 364 to 673 of the HCV polyprotein, of which amino acids 364 to 383 were derived from the natural carboxyterminal region of the E1 protein encoding the signal sequence for E2, and amino acids 384 to 673 of the mature E2 protein.

2.5. Generation of Recombinant HCV-Vaccinia Viruses

Rabbit kidney RK13 cells (ATCC CCL 37), human osteosarcoma 143B thymidine kinase deficient (TK⁻) (ATCC CRL 8303), HeLa (ATCC CCL 2), and Hep G2 (ATCC HB 8065) cell lines were obtained from the American Type Culture Collection (ATCC, Rockville, Md., USA). The cells were grown in Dulbecco's modified Eagle medium (DMEM) supplemented with 10% foetal calf serum, and with Earle's salts (EMEM) for RK13 and 143 B (TK−), and with glucose (4 g/l) for Hep G2. The vaccinia virus WR strain (Western Reserve, ATTC VR119) was routinely propagated in either 143B or RK13 cells, as described previously (Panicali & Paoletti, 1982; Piccini et al., 1987; Mackett et al., 1982, 1984, and 1986). A confluent monolayer of 143B cells was infected with wild type vaccinia virus at a multiplicity of infection (m.o.i.) of 0.1 (=0.1 plaque forming unit (PFU) per cell). Two hours later, the vaccinia recombination plasmid was transfected into the infected cells in the form of a calcium phosphate coprecipitate containing 500 ng of the plasmid DNA to allow homologous recombination (Graham & van der Eb, 1973; Mackett et al., 1985). Recombinant viruses expressing the E.coli xanthine-guanine phosphoribosyl transferase (gpt) protein were selected on rabbit kidney RK13 cells incubated in selection medium (EMEM containing 25 μg/ml mycophenolic acid (MPA), 250 μg/ml xanthine, and 15 μg/ml hypoxanthine; Falkner and Moss, 1988; Janknecht et al, 1991). Single recombinant viruses were purified on fresh monolayers of RK13 cells under a 0.9% agarose overlay in selection medium. Thymidine kinase deficient (TK⁻) recombinant viruses were selected and then plaque purified on fresh monolayers of human 143B cells (TK−) in the presence of 25 μg/ml 5-bromo-2′-deoxyuridine. Stocks of purified recombinant HCV-vaccinia viruses were prepared by infecting either human 143 B or rabbit RK13 cells at an m.o.i. of 0.05 (Mackett et al, 1988). The insertion of the HCV CDNA fragment in the recombinant vaccinia viruses was confirmed on an aliquot (50 μl) of the cell lysate after the MPA selection by means of PCR with the primers used to clone the respective HCV fragments (see Table 1). The recombinant vaccinia-HCV viruses were named according to the vaccinia recombination plasmid number, e.g. the recombinant vaccinia virus vvHCV-10A was derived from recombining the wild type WR strain with the pvHCV-10A plasmid.

### Example 3

### Infection of Cells with Recombinant Vaccinia Viruses

A confluent monolayer of RK13 cells was infected at a m.o.i. of 3 with the recombinant HCV-vaccinia viruses as described in example 2. For infection, the cell monolayer was washed twice with phosphate-buffered saline pH 7.4 (PBS) and the recombinant vaccinia virus stock was diluted in MEM medium. Two hundred μl of the virus solution was added per 10⁶ cells such that the m.o.i. was 3, and incubated for 45 min at 24° C. The virus solution was aspirated and 2 ml of complete growth medium (see example 2) was added per 10⁶ cells. The cells were incubated for 24 hr at 37° C. during which expression of the HCV proteins took place.

### Example 4

### Analysis of Recombinant Proteins by Means of Western Blotting

The infected cells were washed two times with PBS, directly lysed with lysis buffer (50 mM Tris.HCl pH 7.5, 150 mM NaCl, 1% Triton X-100, 5 mM MgCl₂, 1 μg/ml aprotinin (Sigma, Bomem, Belgium)) or detached from the flasks by incubation in 50 mM Tris.HCL pH 7.5/10 mM EDTA/150 mM NaCl for 5 min, and collected by centrifugation (5 min at 1000 g). The cell pellet was then resuspended in 200 μl lysis buffer (50 mM Tris.HCL pH 8.0, 2 mM EDTA, 150 mM NaCl, 5 mM MgCl₂ aprotinin, 1% Triton X-100) per 10⁶ cells.

The cell lysates were cleared for 5 min at 14,000 rpm in an Eppendorf centrifuge to remove the insoluble debris. Proteins of 20 μl lysate were separated by means of sodium dodecyl sulphate-polyacrylamide gel electrophoresis (SDS-PAGE). The proteins were then electro-transferred from the gel to a nitrocellulose sheet (Amersham) using a Hoefer HSI transfer unit cooled to 4° C. for 2 hr at 100 V constant voltage, in transfer buffer (25 mM Tris.HCl pH 8.0, 192 mM glycine, 20% (v/v) methanol). Nitrocellulose filters were blocked with Blotto (5% (w/v) fat-free instant milk powder in PBS; Johnson et al., 1981) and incubated with primary antibodies diluted in Blotto/0.1% Tween 20. Usually, a human negative control serum or serum of a patient infected with HCV were 200 times diluted and preincubated for 1 hour at room temperature with 200 times diluted wild type vaccinia virus-infected cell lysate in order to decrease the non-specific binding. After washing with Blotto/0.1% Tween 20, the nitrocellulose filters were incubated with alkaline phosphatase substrate solution diluted in Blotto/0.1% Tween 20. After washing with 0.1% Tween 20 in PBS, the filters were incubated with alkaline phosphatase substrate solution (100 mM Tris.HCl pH 9.5, 100 mM NaCl, 5 mM MgCl₂, 0.38 μg/ml nitroblue tetrazolium, 0.165 μg/ml 5-bromo-4-chloro-3-indolylphosphate). All steps, except the electrotransfer, were performed at room temperature.

### Example 5

### Purification of Recombinant E1 or E2 Protein

5.1. Lysis

Infected RK13 cells (carrying E1 or E2 constructs) were washed 2 times with phosphate-buffered saline (PBS) and detached from the culture recipients by incubation in PBS containing 10 mM EDTA. The detached cells were washed twice with PBS and 1 ml of lysis buffer (50 mM Tris.HCl pH 7.5, 150 mM NaCl, 1% Triton X-100, 5 mM MgCl₂, 1 μg/ml aprotinin (Sigma, Bornem, Belgium) containing 2 mM biotinylated N-ethylmaleimide (biotin-NEM) (Sigma) was added per 10⁵ cells at 4° C. This lysate was homogenized with a type B douncer and left at room temperature for 0.5 hours. Another 5 volumes of lysis buffer containing 10 mM N-ethylmaleimide (NEM, Aldrich, Bomem, Belgium) was added to the primary lysate and the mixture was left at room temperature for 15 min. Insoluble cell debris was cleared from the solution by centrifugation in a Beckman JA-14 rotor at 14,000 rpm (30100 g at rₘₐₓ) for 1 hour at 4° C.

5.2. Lectin Chromatography

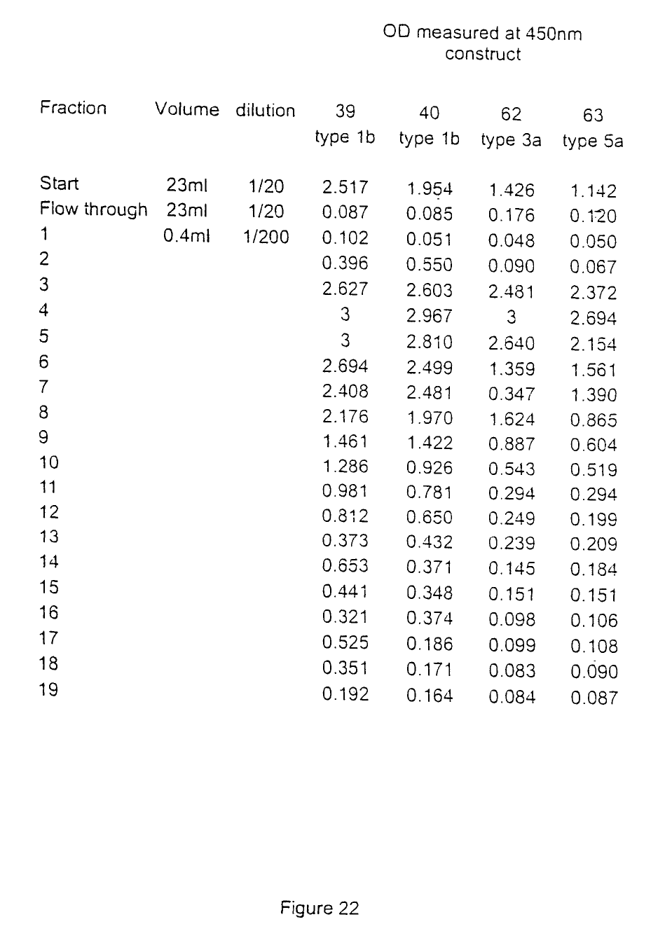

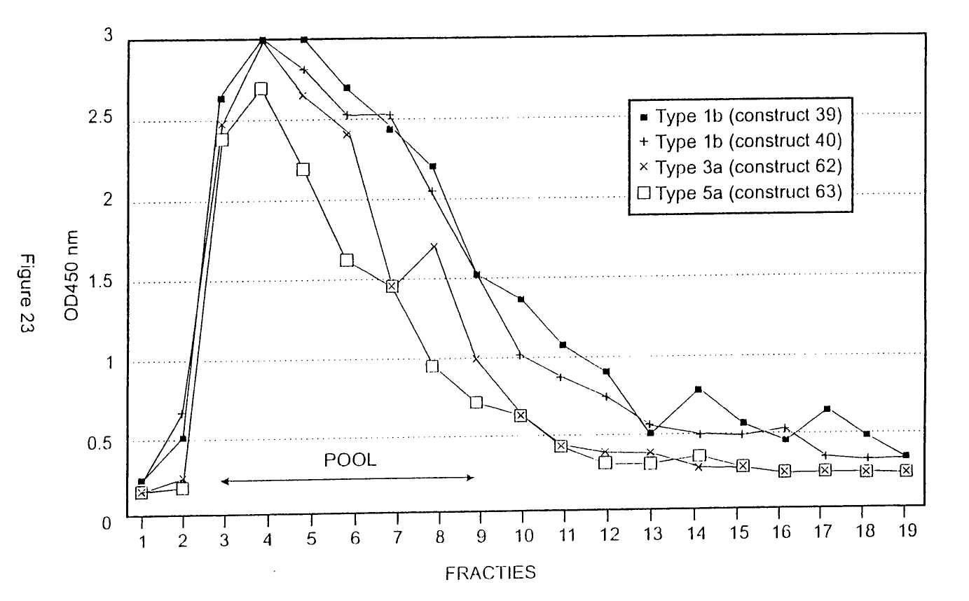

The cleared cell lysate was loaded at a rate of 1 ml/min on a 0.8 by 10 cm Lentil-lectin Sepharose 4B column (Pharmacia) that had been equilibrated with 5 column volumes of lysis buffer at a rate of 1 ml/min. The lentil-lectin column was washed with 5 to 10 column volumes of buffer 1 (0.1M potassium phosphate pH 7.3, 500 mM KCl, 5% glycerol, 1 mM 6-NH₂-hexanoic acid, 1 mM MgCl₂, and 1% DecylPEG (KWANT, Bedum, The Netherlands). In some experiments, the column was subsequently washed with 10 column volumes of buffer 1 containing 0.5% Empigen-BB (Calbiochem, San Diego, Calif., USA) instead of 1% DecylPEG. The bound material was eluted by applying elution buffer (10 mM potassium phosphate pH 7.3, 5% glycerol, 1 mM hexanoic acid, 1 mM MgCl₂, 0.5% Empigen-BB, and 0.5 M α-methyl-mannopyranoside). The eluted material was fractionated and fractions were screened for the presence of E1 or E2 protein by means of ELISA as described in example 6. FIG. 22 shows ELISA results obtained from lentil lectin eluate fractions of 4 different E1 purifications of cell lysates infected with vvHCV39 (type 1b), vvHCV40 (type 1b), vvHCV62 (type 3a), and vvHCV63 (type 5a). FIG. 23 shows the profiles obtained from the values shown in FIG. 22. These results show that the lectin affinity column can be employed for envelope proteins of the different types of HCV.

5.3. Concentration and Partial Reduction

The E1- or E2-positive fractions were pooled and concentrated on a Centricon 30 kDa (Amicon) by centrifugation for 3 hours at 5,000 rpm in a Beckman JA-20 rotor at 4° C. In some experiments the E1- or E2-positive fractions were pooled and concentrated by nitrogen evaporation. An equivalent of 3.10⁸ cells was concentrated to approximately 200 μl. For partial reduction, 30% Empigen-BB (Calbiochem, San Diego, Calif., USA) was added to this 200 μl to a final concentration of 3.5%, and 1M DTT in H₂O was subsequently added to a final concentration of 1.5 to 7.5 mM and incubated for 30 min at 37° C. NEM (1M in dimethylsulphoxide) was subsequently added to a final concentration of 50 mM and left to react for another 30 min at 37° C. to block the free sulphydryl groups.

5.4. Gel Filtration Chromatography

A Superdex-200 HR 10/20 column (Pharmacia) was equilibrated with 3 column volumes PBS/3% Empigen-BB. The reduced mixture was injected in a 500 μl sample loop of the Smart System (Pharmacia) and PBS/3% Empigen-BB buffer was added for gelfiltration.

Fractions of 250 μl were collected from V₀ to Vₜ. The fractions were screened for the presence of E1 or E2 protein as described in example 6.

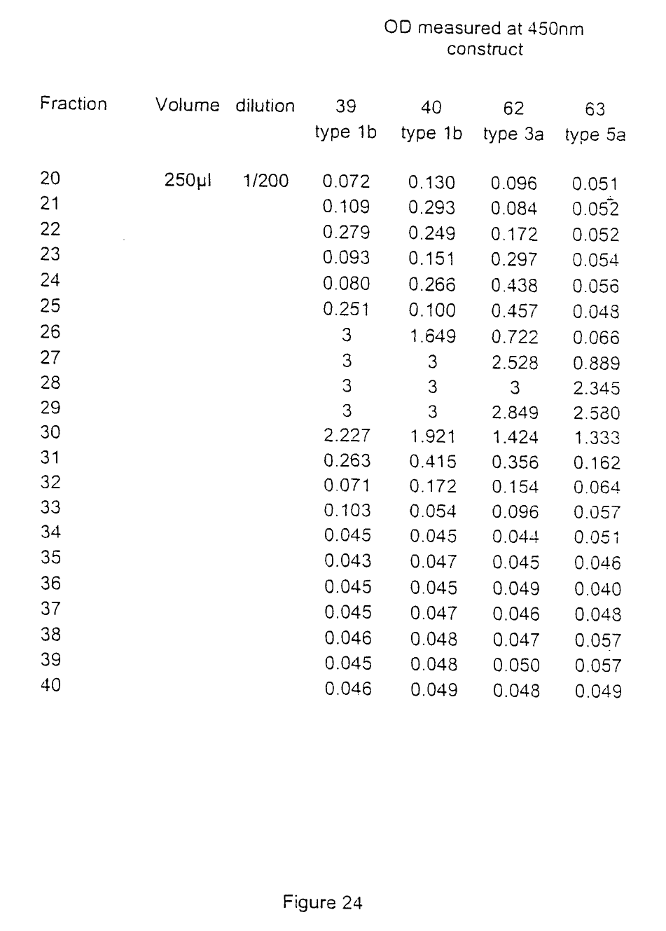

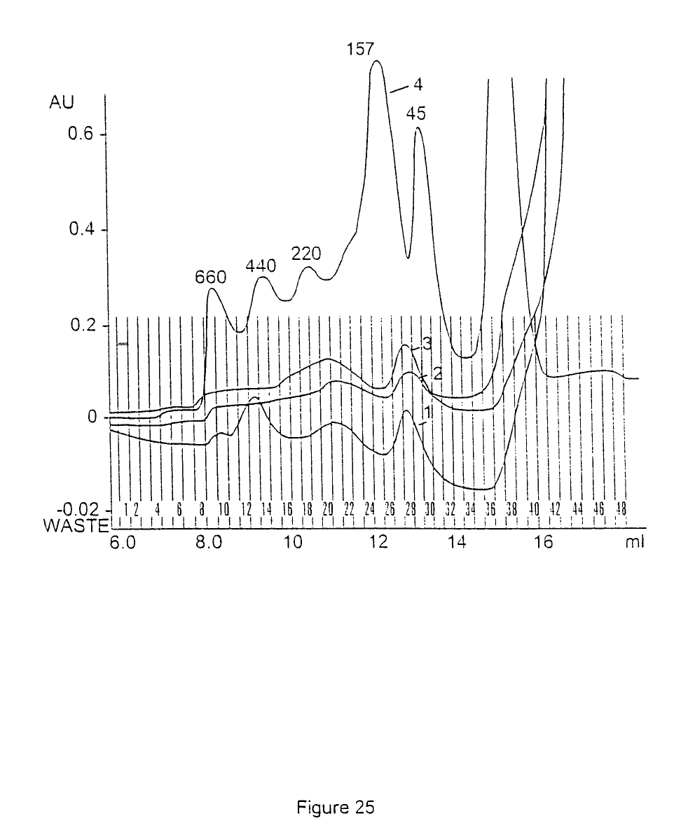

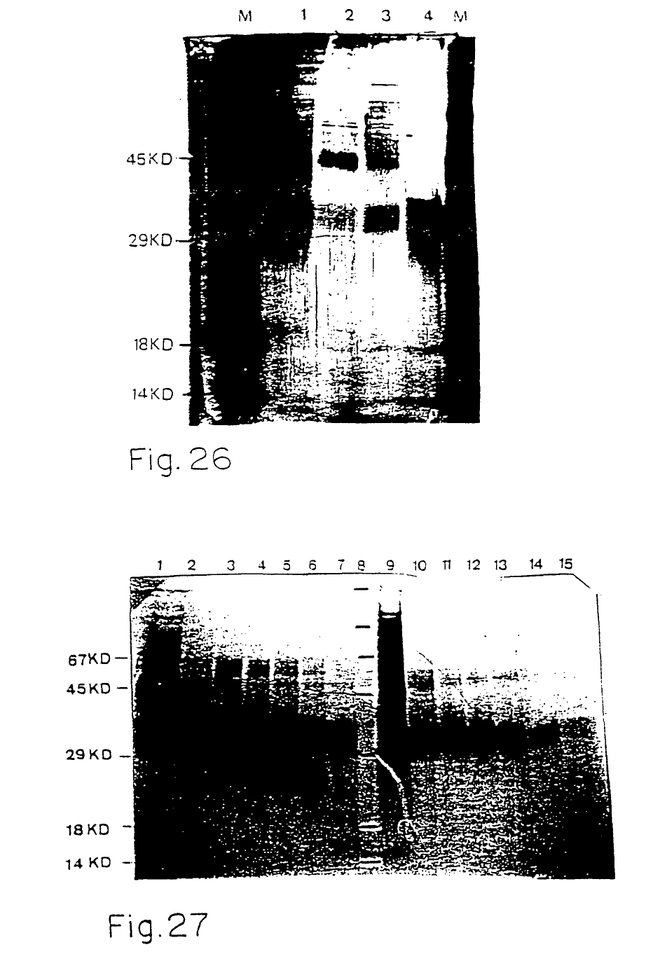

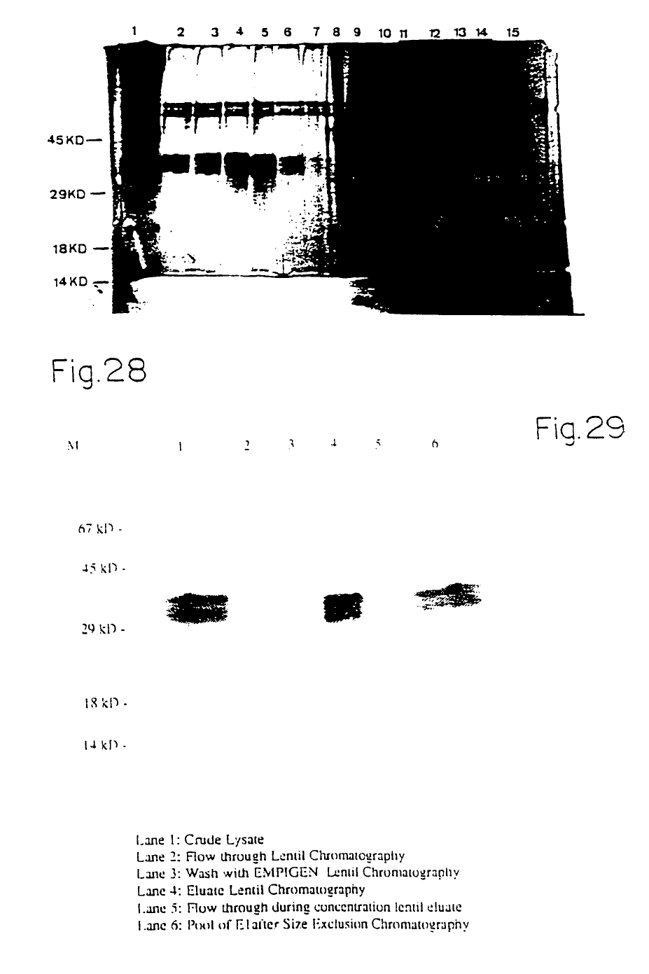

FIG. 24 shows ELISA results obtained from fractions obtained after gelfiltration chromatography of 4 different E1 purifications of cell lysates infected with vvHCV39 (type 1b), vvHCV40 (type 1b), vvHCV62 (type 3a), and vvHCV63 (type 5a). FIG. 25 shows the profiles obtained from purifications of E1 proteins of types 1b, 3a, and 5a (from RK13 cells infected with vvHCV39, wHCV62, and wHCV63, respectively; purified on lentil lectin and reduced as in the previous examples). The peaks indicated with ‘1’, ‘2’, and ‘3’, represent pure E1 protein peaks (E1 reactivity mainly in fractions 26 to 30). These peaks show very similar molecular weights of approximately 70 kDa, corresponding to dimeric E1 protein. Other peaks in the three profiles represent vaccinia virus and/or cellular proteins which could be separated from E1 only because of the reduction step as outlined in example 5.3. and because of the subsequent gelfiltration step in the presence of the proper detergent. As shown in FIG. 26 pool 1 (representing fractions 10 to 17) and pool 2 (representing fractions 18 to 25) contain contaminating proteins not present in the E1 pool (fractions 26 to 30). The E1 peak fractions were ran on SDS/PAGE and blotted as described in example 4. Proteins labelled with NEM-biotin were detected by streptavidin-alkaline phosphatase as shown in FIG. 27. It can be readily observed that, amongst others, the 29 kDa and 45 kDa contaminating proteins present before the gelfiltration chromatography (lane 1) are only present at very low levels in the fractions 26 to 30. The band at approximately 65 kDa represents the E1 dimeric form that could not be entirely disrupted into the monomeric E1 form. Similar results were obtained for the type 3a E1 protein (lanes 10 to 15), which shows a faster mobility on SDS/PAGE because of the presence of only 5 carbohydrates instead of 6. FIG. 28 shows a silver stain of an SDS/PAGE gel run in identical conditions as in FIG. 26. A complete overview of the purification procedure is given in FIG. 29.

The presence of purified E1 protein was further confirmed by means of western blotting as described in example 4. The dimeric E1 protein appeared to be non-aggregated and free of contaminants. The subtype 1b E1 protein purified from vvHCV40-infected cells according to the above scheme was aminoterminally sequenced on an 477 Perkin-Elmer sequencer and appeared to contain a tyrosine as first residue. This confirmed that the E1 protein had been cleaved by the signal peptidase at the correct position (between A191 and Y192) from its signal sequence. This confirms the finding of Hijikata et al. (1991) that the aminoterminus of the mature E1 protein starts at amino acid position 192.

5.5. Purification of the E2 Protein

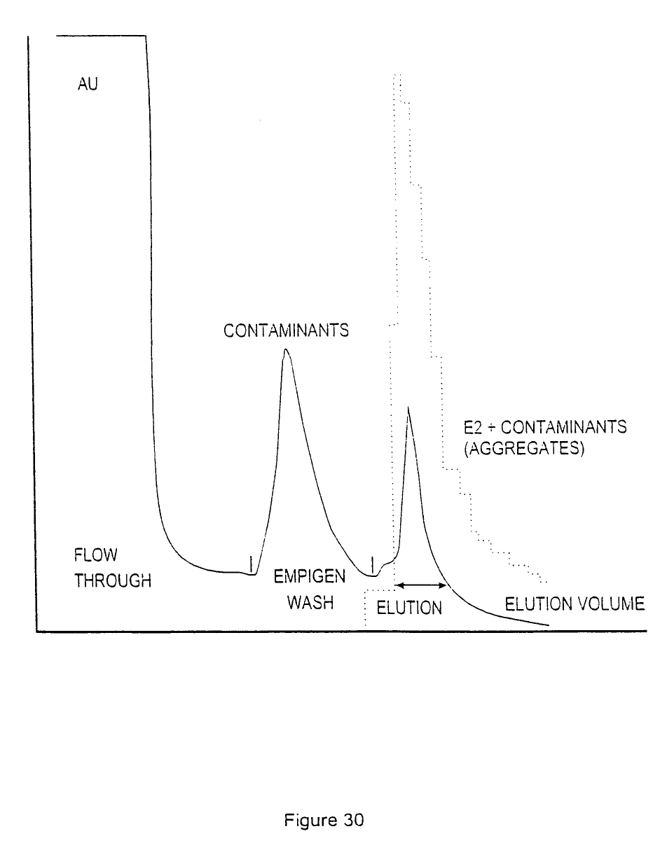

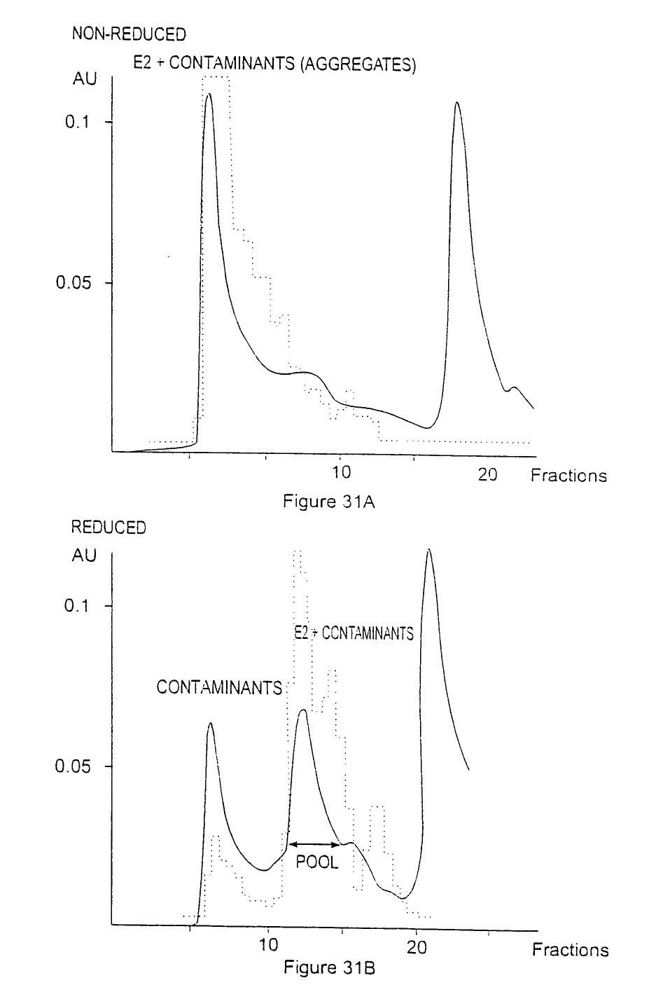

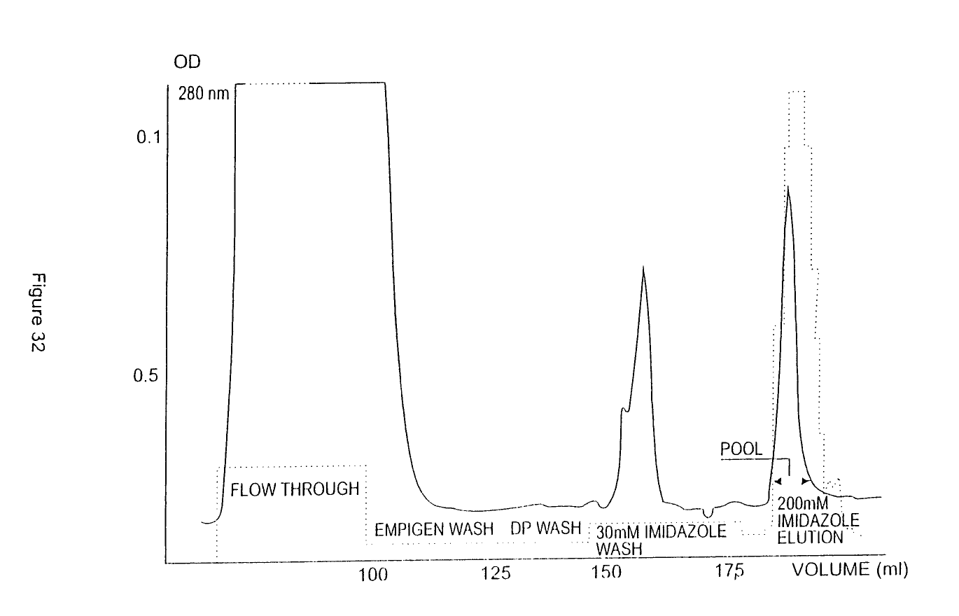

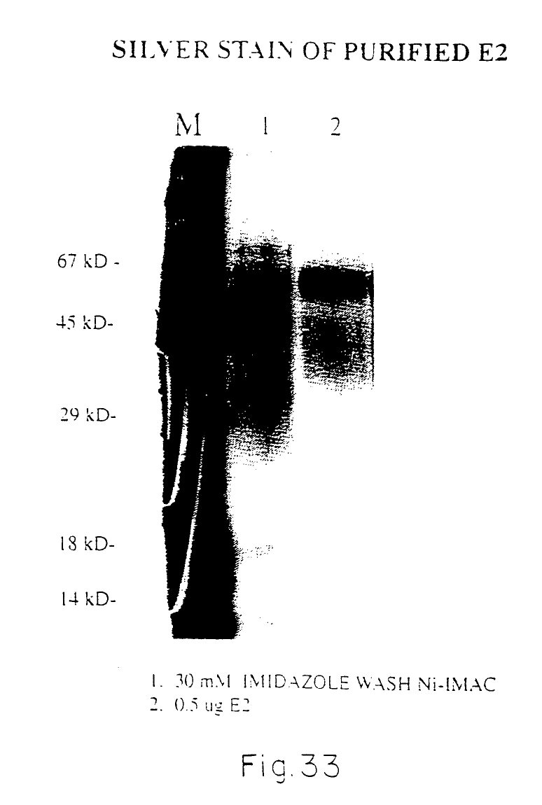

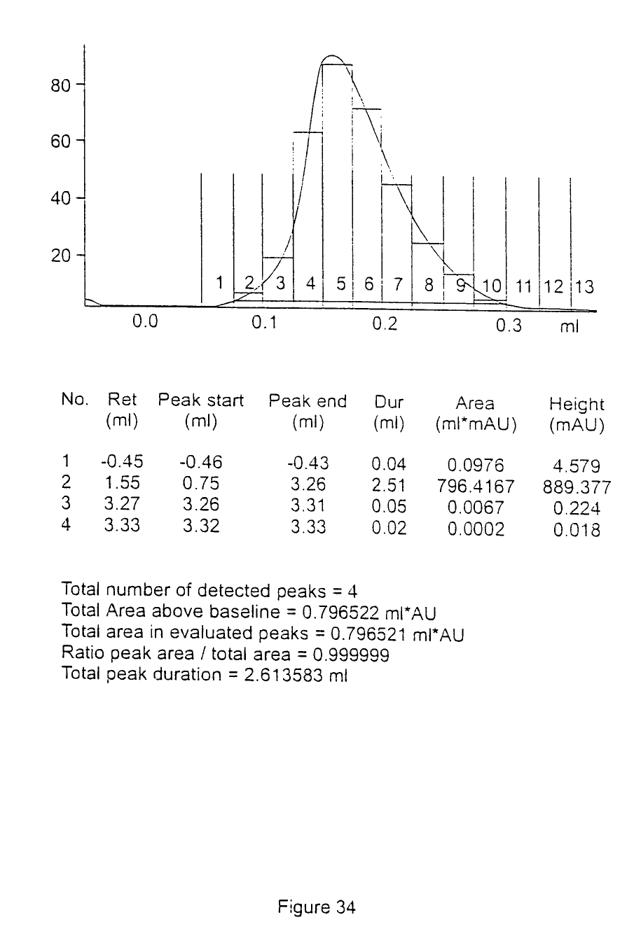

The E2 protein (amino acids 384 to 673) was purified from RK13 cells infected with vvHCV44 as indicated in Examples 5.1 to 5.4. FIG. 30 shows the OD₂₈₀ profile (continuous line) of the lentil lectin chromatography. The dotted line represents the E2 reactivity as detected by ELISA (see example 6). FIG. 31 shows the same profiles obtained from gelfiltration chromatography of the lentil-lectin E2 pool (see FIG. 30), part of which was reduced and blocked according to the methods as set out in example 5.3, and part of which was immediately applied to the column. Both parts of the E2 pool were run on separate gelfiltration columns. It could be demonstrated that E2 forms covalently-linked aggregates with contaminating proteins if no reduction has been performed. After reduction and blocking, the majority of contaminating proteins segregated into the V₀ fraction. Other contaminating proteins copurified with the E2 protein, were not covalently linked to the E2 protein any more because these contaminants could be removed in a subsequent step. FIG. 32 shows an additional Ni²⁺-IMAC purification step carried out for the E2 protein purification. This affinity purification step employs the 6 histidine residues added to the E2 protein as expressed from vvHCV44. Contaminating proteins either run through the column or can be removed by a 30 mM imidazole wash. FIG. 33 shows a silver-stained SDS/PAGE of 0.5 μg of purified E2 protein and a 30 mM imidazole wash. The pure E2 protein could be easily recovered by a 200 mM imidazole elution step. FIG. 34 shows an additional desalting step intended to remove imidazole and to be able to switch to the desired buffer, e.g. PBS, carbonate buffer, saline.

Starting from about 50,000 cm² of RK13 cells infected with vvHCV11A (or vvHCV40) for the production of E1 or vvHCV41, vvHCV42, vvHCV43, or vvHCV44 for production of E2 protein, the procedures described in examples 5.1 to 5.5 allow the purification of approximately 1.3 mg of E1 protein and 0.6 mg of E2 protein.

It should also be remarked that secreted E2 protein (constituting approximately 30-40%, 60-70% being in the intracellular form) is characterized by aggregate formation (contrary to expectations). The same problem is thus posed to purify secreted E2. The secreted E2 can be purified as disclosed above.

### EXAMPLE 6

### ELISA for the Detection of Anti-E1 or Anti-E2 Antibodies or for the Detection of E1 or E2 Proteins

Maxisorb microwell plates (Nunc, Roskilde, Denmark) were coated with 1 volume (e.g. 50 μl or 100 μl or 200 μl) per well of a 5 μg/ml solution of Streptavidin (Boehringer Mannheim) in PBS for 16 hours at 4° C. or for 1 hour at 37° C. Alternatively, the wells were coated with 1 volume of 5 μg/ml of Galanthus nivalis agglutinin (GNA) in 50 mM sodium carbonate buffer pH 9.6 for 16 hours at 4° C. or for 1 hour at 37° C. In the case of coating with GNA, the plates were washed 2 times with 400 μl of Washing Solution of the Innotest HCV Ab III kit (Innogenetics, Belgium). Unbound coating surfaces were blocked with 1.5 to 2 volumes of blocking solution (0.1% casein and 0.1% NaN₃ in PBS) for 1 hour at 37° C. or for 16 hours at 4° C. Blocking solution was aspirated. Purified E1 or E2 was diluted to 100-1000 ng/ml (concentration measured at A=280 nm) or column fractions to be screened for E1 or E2 (see example 5), or E1 or E2 in non-purified cell lysates (example 5.1.) were diluted 20 times in blocking solution, and 1 volume of the E1 or E2 solution was added to each well and incubated for 1 hour at 37° C. on the Streptavidin- or GNA-coated plates. The microwells were washed 3 times with 1 volume of Washing Solution of the Innotest HCV Ab III kit (Innogenetics, Belgium). Serum samples were diluted 20 times or monoclonal anti-E1 or anti-E2 antibodies were diluted to a concentration of 20 ng/ml in Sample Diluent of the Innotest HCV Ab III kit and 1 volume of the solution was left to react with the E1 or E2 protein for 1 hour at 37° C. The microwells were washed 5 times with 400 l of Washing Solution of the Innotest HCV Ab III kit (Innogenetics, Belgium). The bound antibodies were detected by incubating each well for 1 hour at 37° C. with a goat anti-human or anti-mouse IgG, peroxidase-conjugated secondary antibody (DAKO, Glostrup, Denmark) diluted 1/80,000 in 1 volume of Conjugate Diluent of the Innotest HCV Ab III kit (Innogenetics, Belgium), and color development was obtained by addition of substrate of the Innotest HCV Ab III kit (Innogenetics, Belgium) diluted 100 times in 1 volume of Substrate Solution of the Innotest HCV Ab III kit (Innogenetics, Belgium) for 30 min at 24° C. after washing of the plates 3 times with 400 μl of Washing Solution of the Innotest HCV Ab III kit (Innogenetics, Belgium).

### EXAMPLE 7

### Follow Up of Patient Groups with Different Clinical Profiles

7.1. Monitoring of Anti-E1 and Anti-E2 Antibodies

The current hepatitis C virus (HCV) diagnostic assays have been developed for screening and confirmation of the presence of HCV antibodies. Such assays do not seem to provide information useful for monitoring of treatment or for prognosis of the outcome of disease. However, as is the case for hepatitis B, detection and quantification of anti-envelope antibodies may prove more useful in a clinical setting. To investigate the possibility of the use of anti-E1 antibody titer and anti-E2 antibody titer as prognostic markers for outcome of hepatitis C disease, a series of IFN-α treated patients with long-term sustained response (defined as patients with normal transaminase levels and negative HCV-RNA test (PCR in the 5′ non-coding region) in the blood for a period of at least 1 year after treatment) was compared with patients showing no response or showing biochemical response with relapse at the end of treatment.

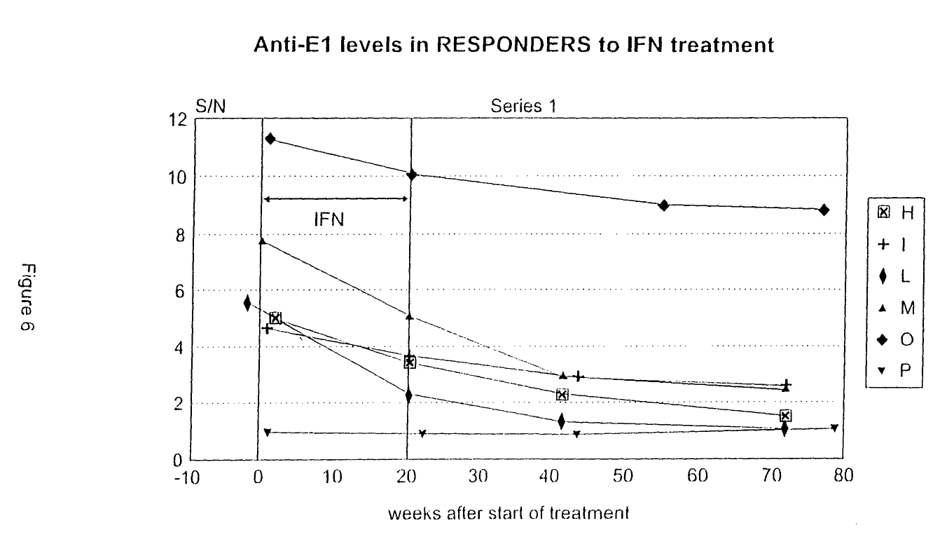

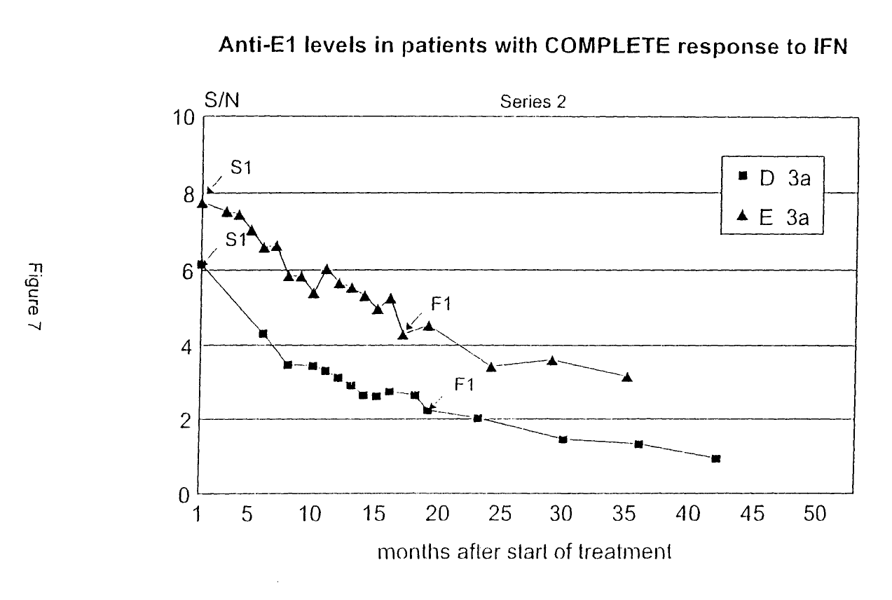

A group of 8 IFN-α treated patients with long-term sustained response (LTR, follow up 1 to 3.5 years, 3 type 3a and 5 type 1b) was compared with 9 patients showing non-complete responses to treatment (NR, follow up 1 to 4 years, 6 type 1b and 3 type 3a). Type 1b (vvHCV-39, see example 2.5.) and 3a E1 (vvHCV-62, see example 2.5.) proteins were expressed by the vaccinia virus system (see examples 3 and 4) and purified to homogeneity (example 5). The samples derived from patients infected with a type 1b hepatitis C virus were tested for reactivity with purified type 1b E1 protein, while samples of a type 3a infection were tested for reactivity of anti-type 3a E1 antibodies in an ELISA as desribed in example 6. The genotypes of hepatitis C viruses infecting the different patients were determined by means of the Inno-LiPA genotyping assay (Innogenetics, Belgium). FIG. 5 shows the anti-E1 signal-to-noise ratios of these patients followed during the course of interferon treatment and during the follow-up period after treatment. LTR cases consistently showed rapidly declining anti-E1 levels (with complete negativation in 3 cases), while anti-E1 levels of NR cases remained approximately constant. Some of the obtained anti-E1 data are shown in Table 2 as average S/N ratios±SD (mean anti-E1 titer). The anti-E1 titer could be deduced from the signal to noise ratio as show in FIGS. 5, 6, 7, and 8.

Already at the end of treatment, marked differences could be observed between the 2 groups. Anti-E1 antibody titers had decreased 6.9 times in LTR but only 1.5 times in NR. At the end of follow up, the anti-E1 titers had declined by a factor of 22.5 in the patients with sustained response and even slightly increased in NR. Therefore, based on these data, decrease of anti-E1 antibody levels during monitoring of IFN-α therapy correlates with long-term, sustained response to treatment. The anti-E1 assay may be very useful for prognosis of long-term response to IFN treatment, or to treatment of the hepatitis C disease in general.

This finding was not expected. On the contrary, the inventors had expected the anti-E1 antibody levels to increase during the course of IFN treatment in patients with long term response. As is the case for hepatitis B, the virus is cleared as a consequence of the seroconversion for anti-HBsAg antibodies. Also in many other virus infections, the virus is eliminated when anti-envelope antibodies are raised. However, in the experiments of the present invention, anti-E1 antibodies clearly decreased in patients with a long-term response to treatment, while the antibody-level remained approximately at the same level in non-responding patients. Although the outcome of these experiments was not expected, this non-obvious finding may be very important and useful for clinical diagnosis of HCV infections.

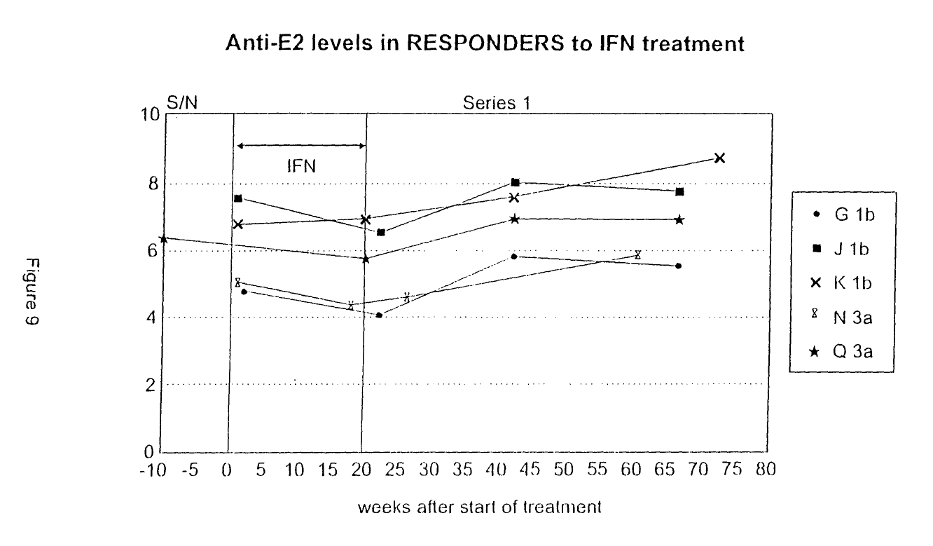

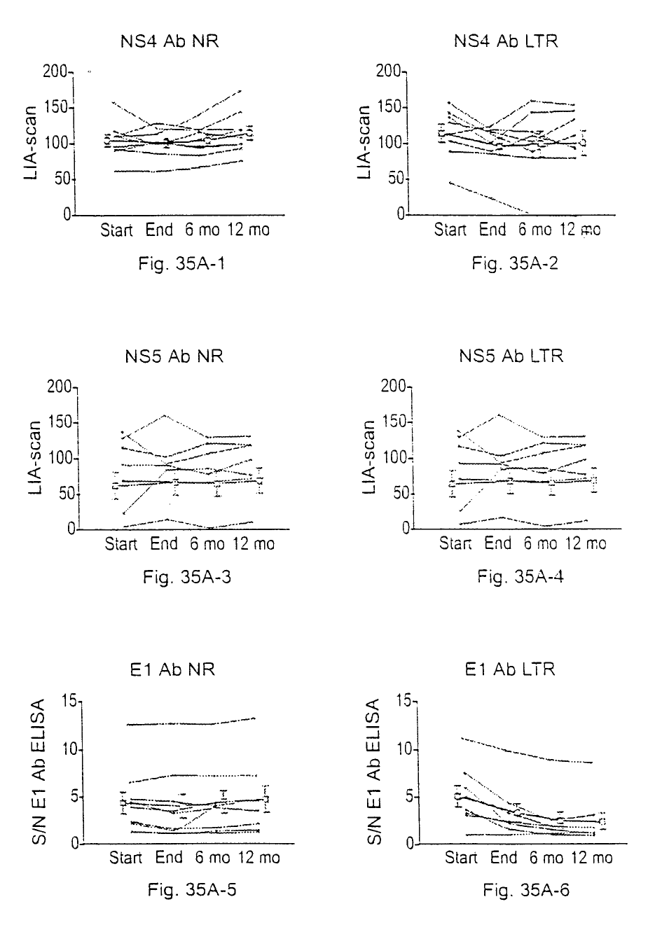

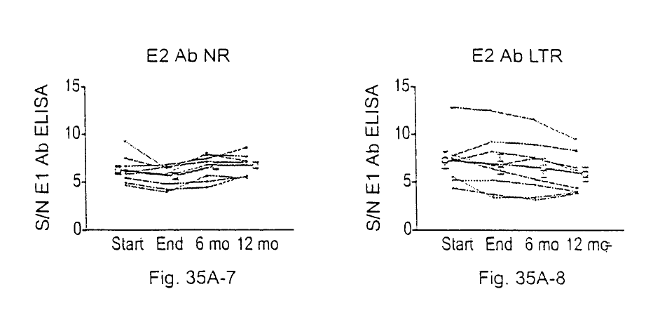

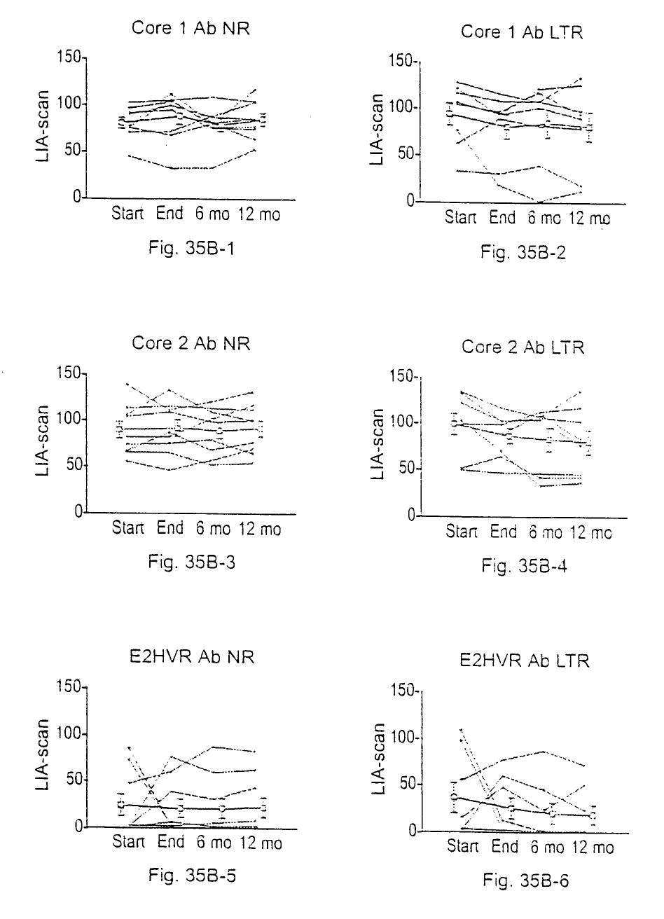

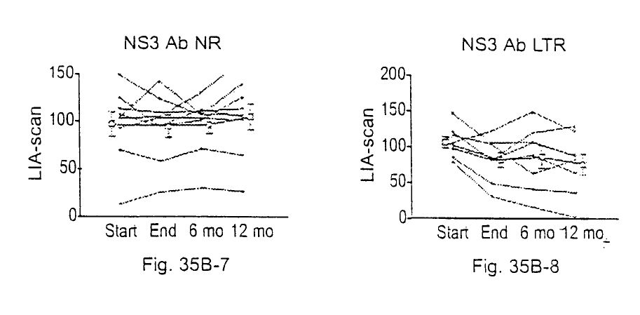

As shown in FIGS. 9, 10, 11, and 12, anti-E2 levels behaved very differently in the same patients studied and no obvious decline in titers was observed as for anti-E1 antibodies. FIG. 35 gives a complete overview of the pilot study.

As can be deduced from Table 2, the anti-E1 titers were on average at least 2 times higher at the start of treatment in long term responders compared with incomplete responders to treatment. Therefore, measuring the titer of anti-E1 antibodies at the start of treatment, or monitoring the patient during the course of infection and measuring the anti-E1 titer, may become a useful marker for clinical diagnosis of hepatitis C. Furthermore, the use of more defined regions of the E1 or E2 proteins may become desirable, as shown in example 7.3.

7.2. Analysis of E1 and E2 Antibodies in a Larger Patient Cohort

The pilot study lead the inventors to conclude that, in case infection was completely cleared, antibodies to the HCV envelope proteins changed more rapidly than antibodies to the more conventionally studied HCV antigens, with E1 antibodies changing most vigorously. We therefore included more type 1b and 3a-infected LTR and further supplemented the cohort with a matched series of NR, such that both groups included 14 patients each. Some partial responders (PR) and responders with relapse (RR) were also analyzed.

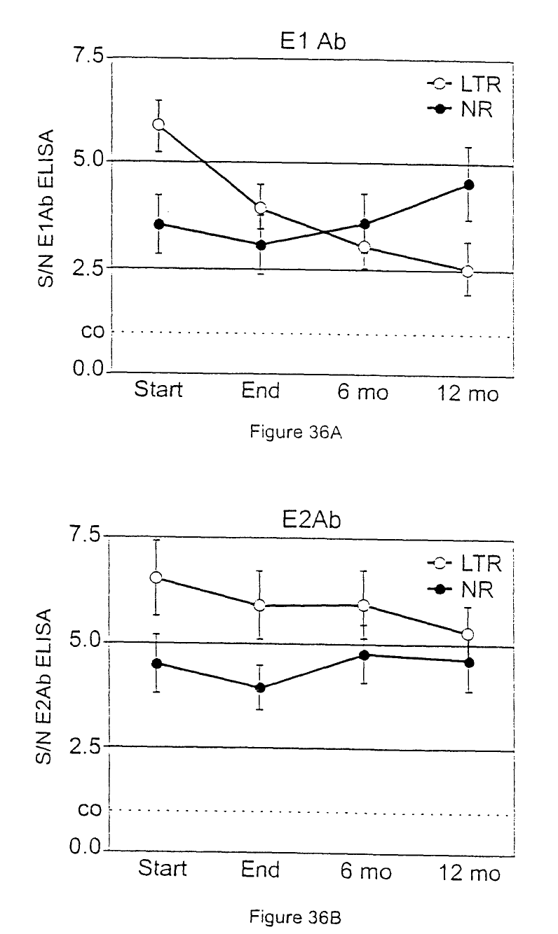

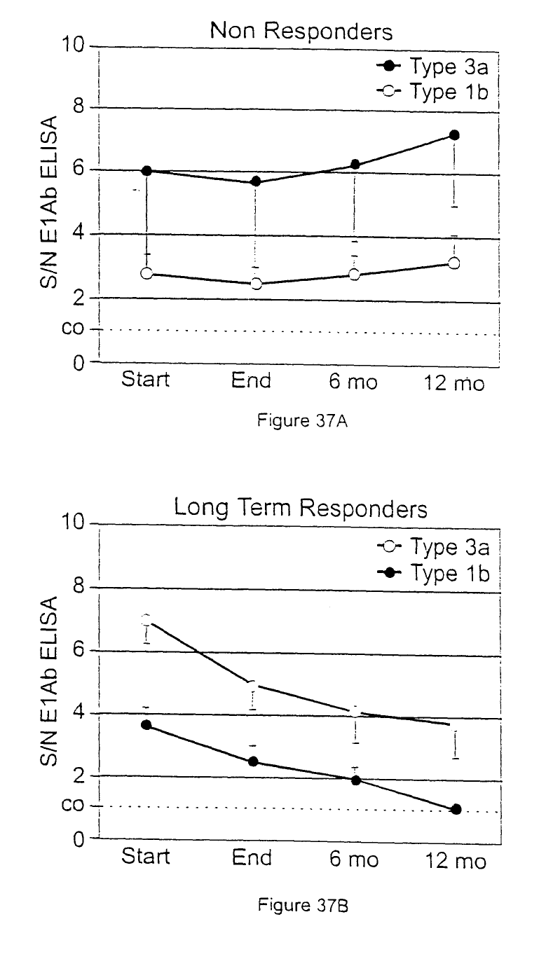

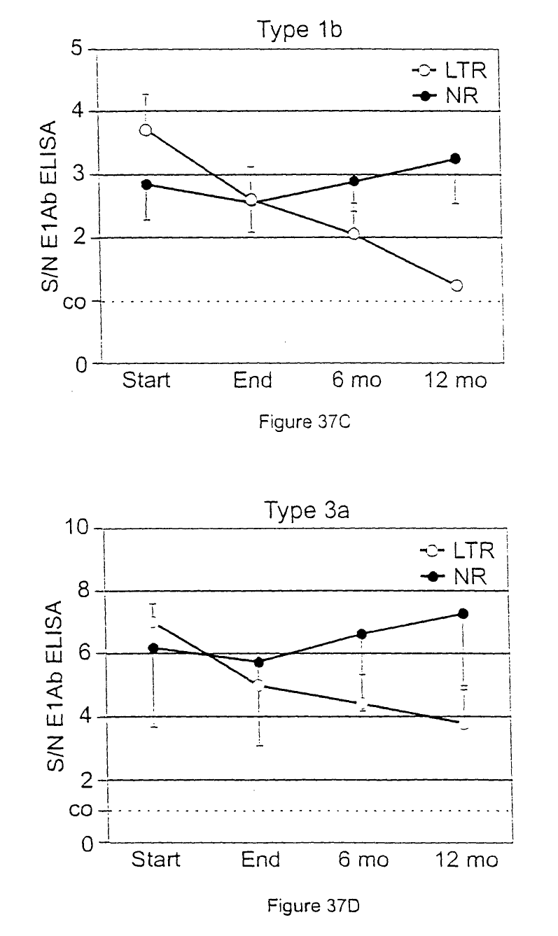

FIG. 36 depicts average E1 antibody (E1Ab) and E2 antibody (E2Ab) levels in the LTR and NR groups and Tables 4 and 5 show the statistical analyses. In this larger cohort, higher E1 antibody levels before IFN-α therapy were associated with LTR (P<0.03). Since much higher E1 antibody levels were observed in type 3a-infected patients compared with type 1b-infected patients (FIG. 37), the genotype was taken into account (Table 4). Within the type 1b-infected group, LTR also had higher E1 antibody levels than NR at the initiation of treatment [P<0.05]; the limited number of type 3a-infected NR did not allow statistical analysis.

Of antibody levels monitored in LTR during the 1.5-year follow up period, only E1 antibodies cleared rapidly compared with levels measured at initiation of treatment [P=0.0058, end of therapy; P=0.0047 and P=0.0051 at 6 and 12 months after therapy, respectively]. This clearance remained significant within type 1- or type 3-infected LTR (average P values<0.05). These data confirmed the initial finding that E1Ab levels decrease rapidly in the early phase of resolvement. This feature seems to be independent of viral genotype. In NR, PR, or RR, no changes in any of the antibodies measured were observed throughout the follow up period. In patients who responded favourably to treatment with normalization of ALT levels and HCV-RNA negative during treatment, there was a marked difference between sustained responders (LTR) and responders with a relapse (RR). In contrast to LTR, RR did not show any decreasing E1 antibody levels, indicating the presence of occult HCV infection that could neither be demonstrated by PCR or other classical techniques for detection of HCV-RNA, nor by raised ALT levels. The minute quantities of viral RNA, still present in the RR group during treatment, seemed to be capable of anti-E1 B cell stimulation. Anti-E1 monitoring may therefore not only be able to discriminate LTR from NR, but also from RR.

7.3. Monitoring of Antibodies of Defined Regions of the E1 Protein

Although the molecular biological approach of identifying HCV antigens resulted in unprecedented breakthrough in the development of viral diagnostics, the method of immune screening of λgt11 libraries predominantly yielded linear epitopes dispersed throughout the core and non-structural regions, and analysis of the envelope regions had to await cloning and expression of the E1/E2 region in mammalian cells. This approach sharply contrasts with many other viral infections of which epitopes to the envelope regions had already been mapped long before the deciphering of the genomic structure. Such epitopes and corresponding antibodies often had neutralizing activity useful for vaccine development and/or allowed the development of diagnostic assays with clinical or prognostic significance (e.g. antibodies to hepatitis B surface antigen).

As no HCV vaccines or tests allowing clinical diagnosis and prognosis of hepatitis C disease are available today, the characterization of viral envelope regions exposed to immune surveillance may significantly contribute to new directions in HCV diagnosis and prophylaxis.

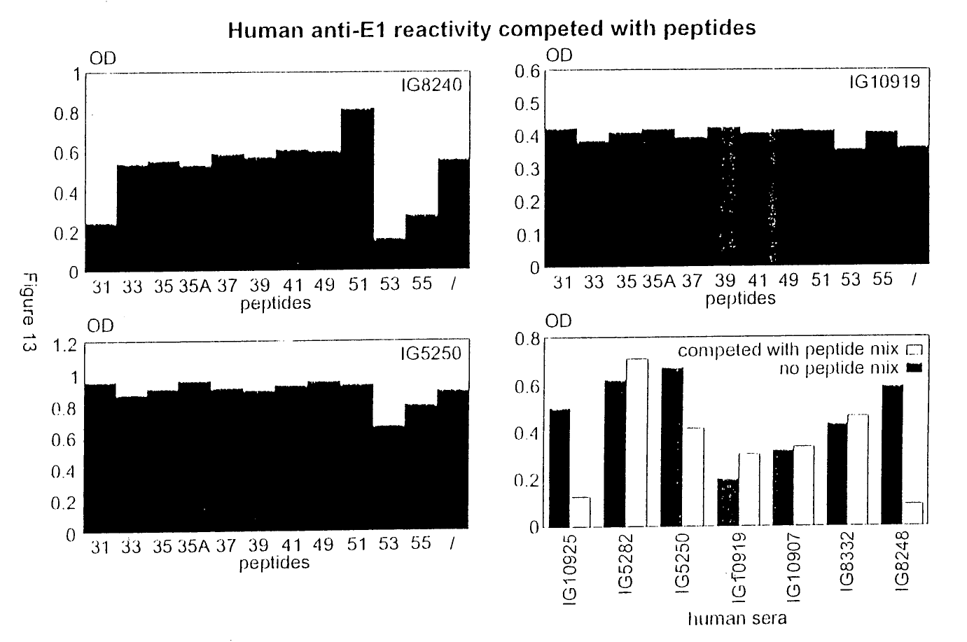

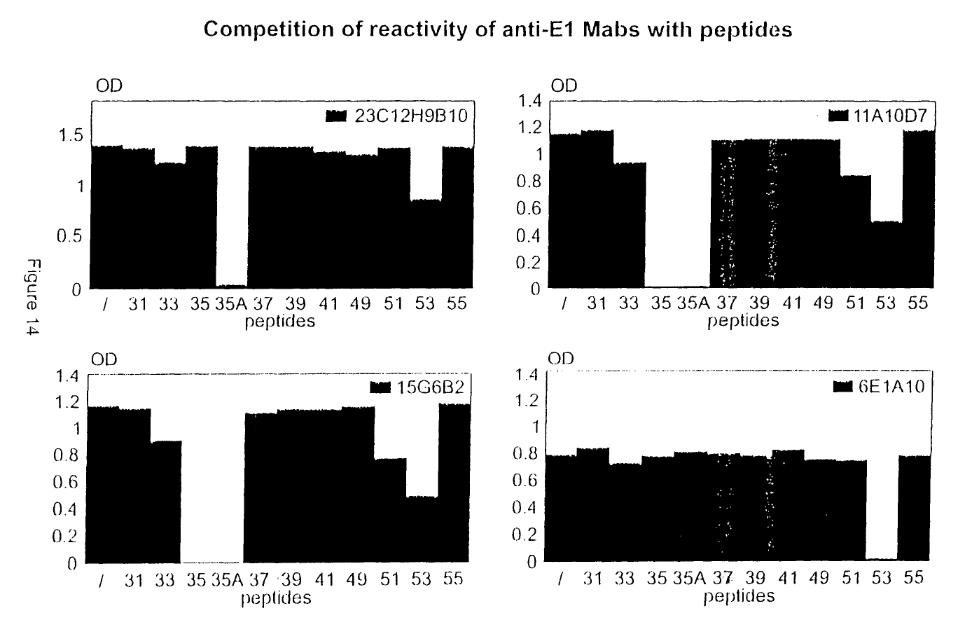

Several 20-mer peptides (Table 3) that overlapped each other by 8 amino acids, were synthesized according to a previously described method (EP-A-0 489 968) based on the HC-J1 sequence (Okamoto et al., 1990). None of these, except peptide env35 (also referred to as E1-35), was able to detect antibodies in sera of approximately 200 HCV cases. Only 2 sera reacted slightly with the env35 peptide. However, by means of the anti-E1 ELISA as described in example 6, it was possible to discover additional epitopes as follows: The anti-E1 ELISA as described in example 6 was modified by mixing 50 μg/ml of E1 peptide with the 1/20 diluted human serum in sample diluent. FIG. 13 shows the results of reactivity of human sera to the recombinant E1 (expressed from vvHCV-40) protein, in the presence of single or of a mixture of E1 peptides. While only 2% of the sera could be detected by means of E1 peptides coated on strips in a Line Iumunoassay format, over half of the sera contained anti-E1 antibodies which could be competed by means of the same peptides, when tested on the recombinant E1 protein. Some of the murine monoclonal antibodies obtained from Balb/C mice after injection with purified E1 protein were subsequently competed for reactivity to E1 with the single peptides (FIG. 14). Clearly, the region of env53 contained the predominant epitope, as the addition of env53 could substantially compete reactivity of several sera with E1, and antibodies to the env31 region were also detected. This finding was surprising, since the env53 and env31 peptides had not shown any reactivity when coated directly to the solid phase.

Therefore peptides were synthesized using technology described by applicant previously (in WO 93/18054). The following peptides were synthesized:

| | |
|---|---|
| peptide env35A-biotm | |
| NH₂-SNSSEAADMIMHTPGCV-GKbiotin | (SEQ ID NO:51) |

spanning amino acids 208 to 227 of the HCV
polyprotein in the E1 region

| | |
|---|---|
| peptide biotin-env53 (‘epitope A’) | |
| biotin-GG-ITGHRMAWDMMMNWSPTTAL-COOH | (SEQ ID NO:52) |

spanning amino acids to 313 of 332 of the
HCV polyprotein in the E1 region

| | |
|---|---|
| peptide 1bE1 (‘epitope B’) | |
| H₂N-YEVRNVSGIYHVTNDCSNSSIVYEAADMIMHT | (SEQ ID NO:53) |
| PGCGK -biotin | |

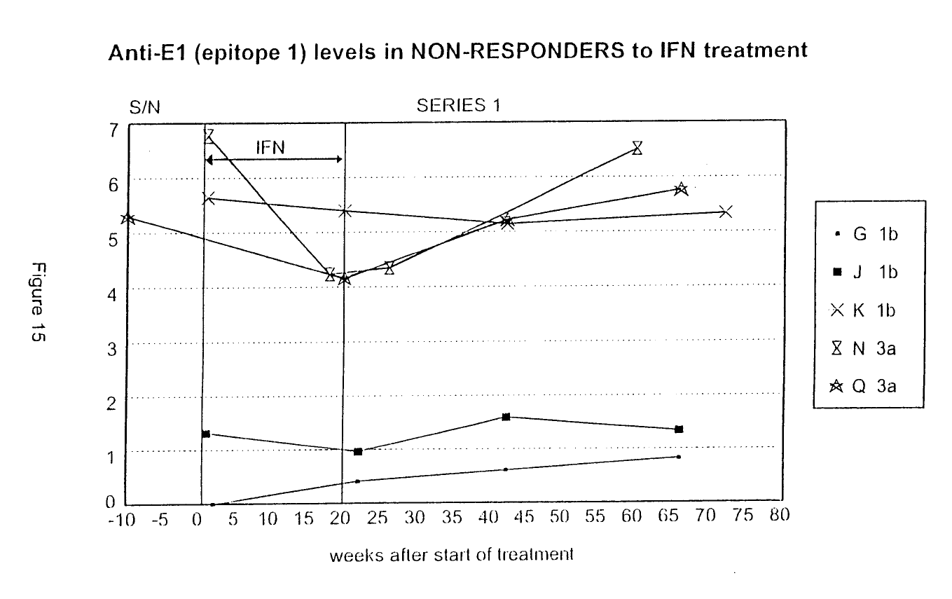

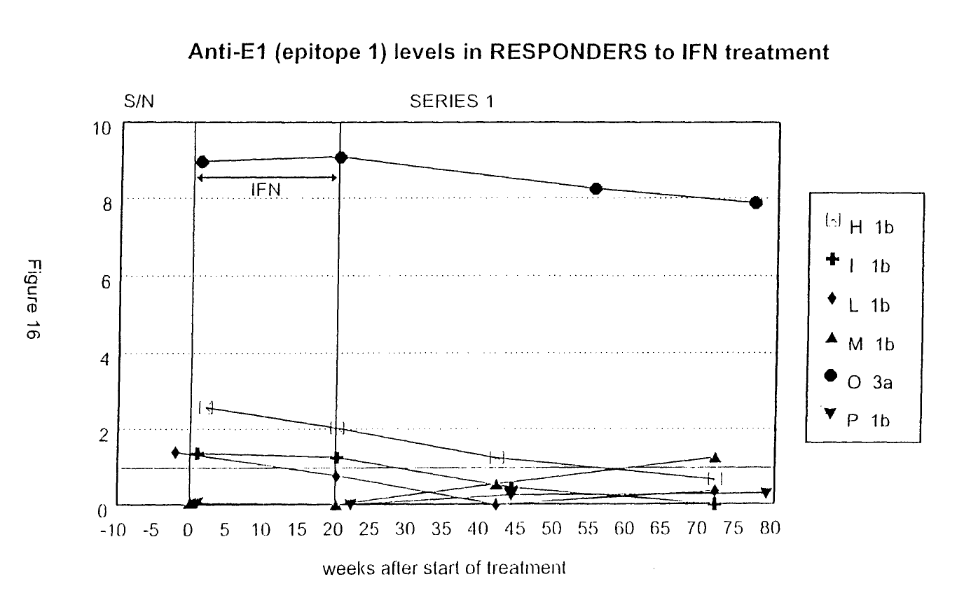

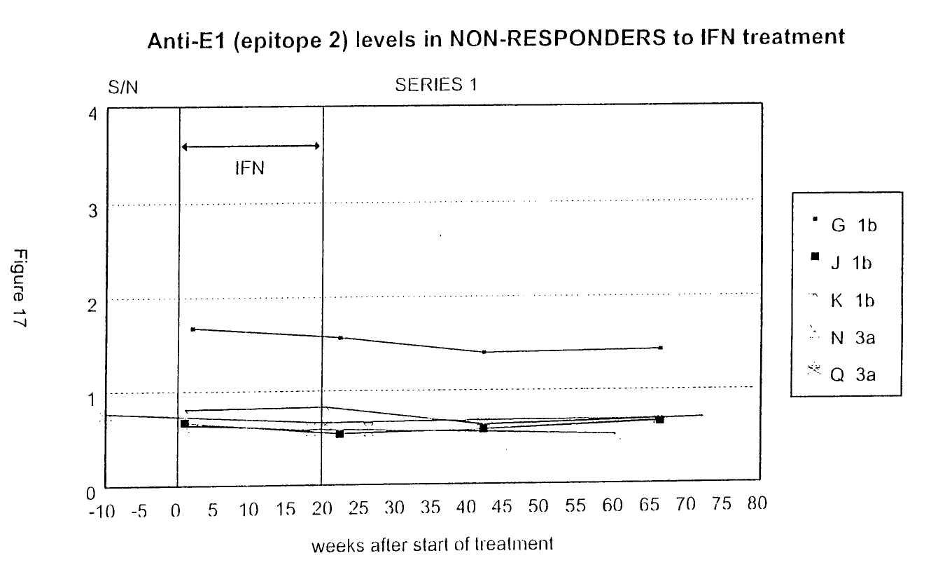

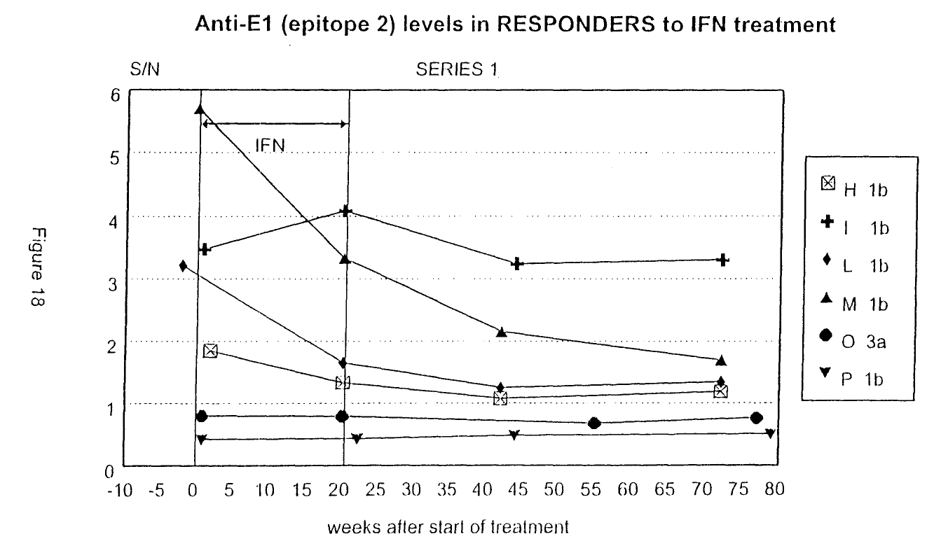

spanning amino acids 192 to 228 of the HCV polyprotein in the E1 region and compared with the reactivities of peptides E1 a-BB (biotin-GG-TPTVATRDGKLPATQLRRHIDLL, SEQ ID NO:54) and E1b-BB (biotin-GG-TPTLAARDASVPTTTIRRHVDLL, SEQ ID NO:55) which are derived from the same region of sequences of genotype 1a and 1b respectively and which have been described at the IXth international virology meeting in Glasgow, 1993 (‘epitope C’). Reactivity of a panel of HCV sera was tested on epitopes A, B and C and epitope B was also compared with env35A (of 47 HCV-positive sera, 8 were positive on epitope B and none reacted with env35A). Reactivity towards epitopes A, B, and C was tested directly to the biotinylated peptides (50 μg/ml) bound to streptavidin-coated plates as described in example 6. Clearly, epitopes A and B were most reactive while epitopes C and env35A-biotin were much less reactive. The same series of patients that had been monitored for their reactivity towards the complete E1 protein (example 7.1.) was tested for reactivity towards epitopes A, B, and C. Little reactivity was seen to epitope C, while as shown in FIGS. 15, 16, 17, and 18, epitopes A and B reacted with the majority of sera. However, antibodies to the most reactive epitope (epitope A) did not seem to predict remission of disease, while the anti-1bE1 antibodies (epitope B) were present almost exclusively in long term responders at the start of WFN treatment. Therefore, anti-1bE1 (epitope B) antibodies and anti-env53 (epitope A) antibodies could be shown to be useful markers for prognosis of hepatitis C disease. The env53 epitope may be advantageously used for the detection of cross-reactive antibodies (antibodies that cross-react between major genotypes) and antibodies to the env53 region may be very useful for universal E1 antigen detection in serum or liver tissue. Monoclonal antibodies that recognized the env53 region were reacted with a random epitope library. In 4 clones that reacted upon immunoscreening with the monoclonal antibody 5E1A10, the sequence -GWD- was present. Because of its analogy with the universal HCV sequence present in all HCV variants in the env53 region, the sequence AWD is thought to contain the essential sequence of the env53 cross-reactive murine epitope. The env31 clearly also contains a variable region which may contain an epitope in the amino terminal sequence -YQVRNSTGL- (SEQ ID NO:93) and may be useful for diagnosis. Env31 or E1-31 as shown in Table 3, is a part of the peptide 1bE1. Peptides E1-33 and E1-51 also reacted to some extent with the murine antibodies, and peptide E1-55 (containing the variable region 6 (V6); spanning amino acid positions 329-336) also reacted with some of the patient sera.

Anti-E2 antibodies clearly followed a different pattern than the anti-E1 antibodies, especially in patients with a long-term response to treatment. Therefore, it is clear that the decrease in anti-envelope antibodies could not be measured as efficiently with an assay employing a recombinant E1/E2 protein as with a single anti-E1 or anti-E2 protein. The anti-E2 response would clearly blur the anti-E1 response in an assay measuring both kinds of antibodies at the same time. Therefore, the ability to test anti-envelope antibodies to the single E1 and E2 proteins, was shown to be useful.

7.4. Mapping of Anti-E2 Antibodies

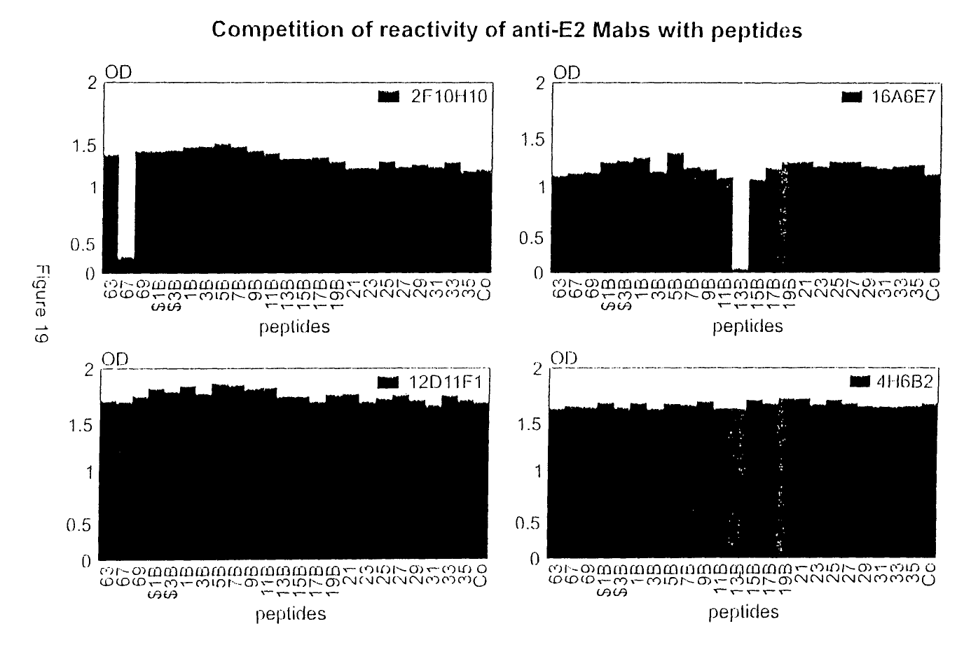

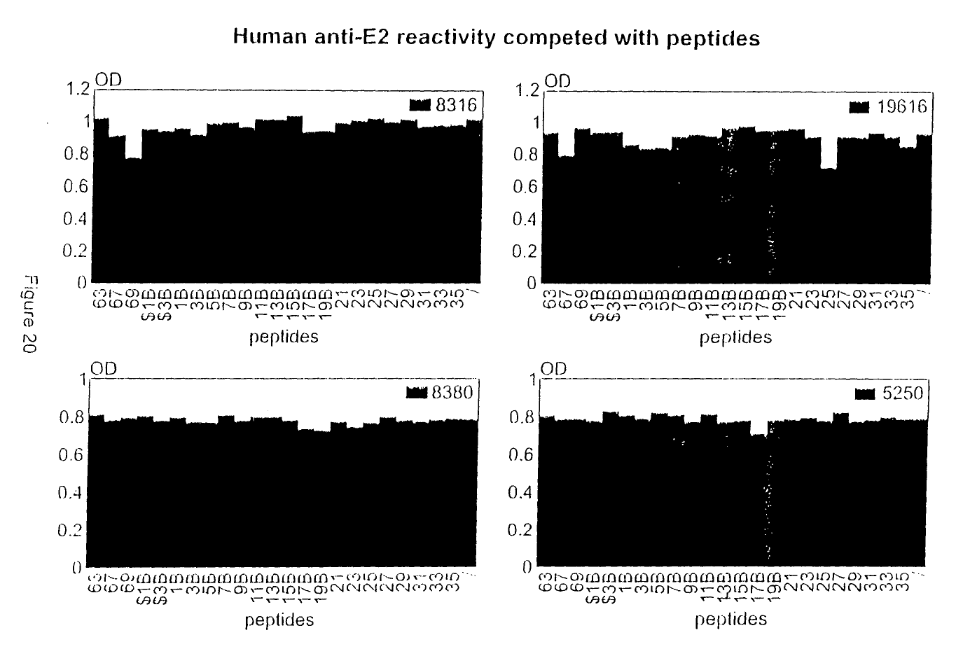

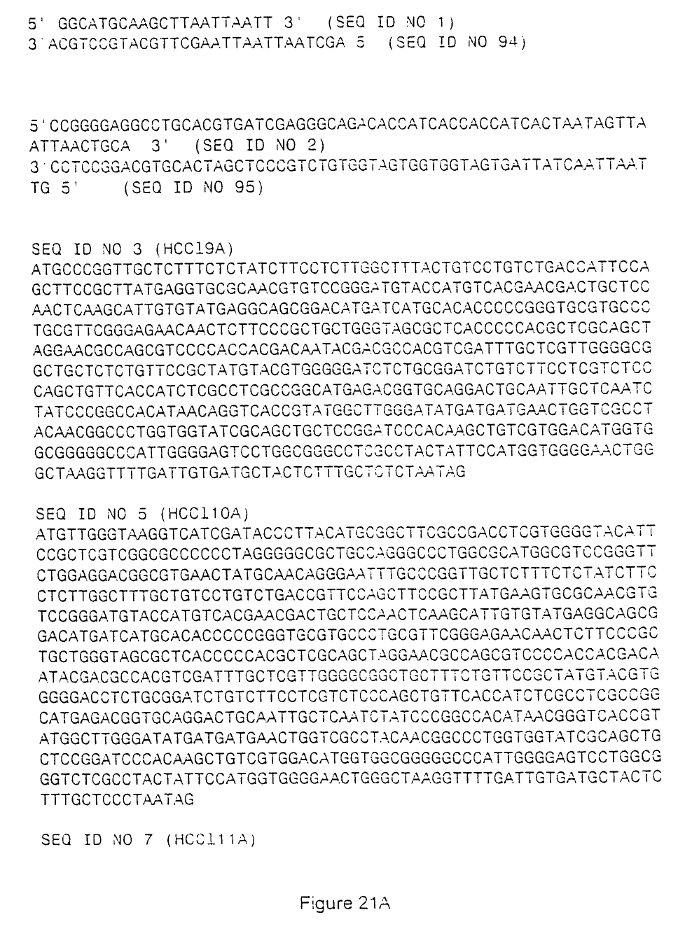

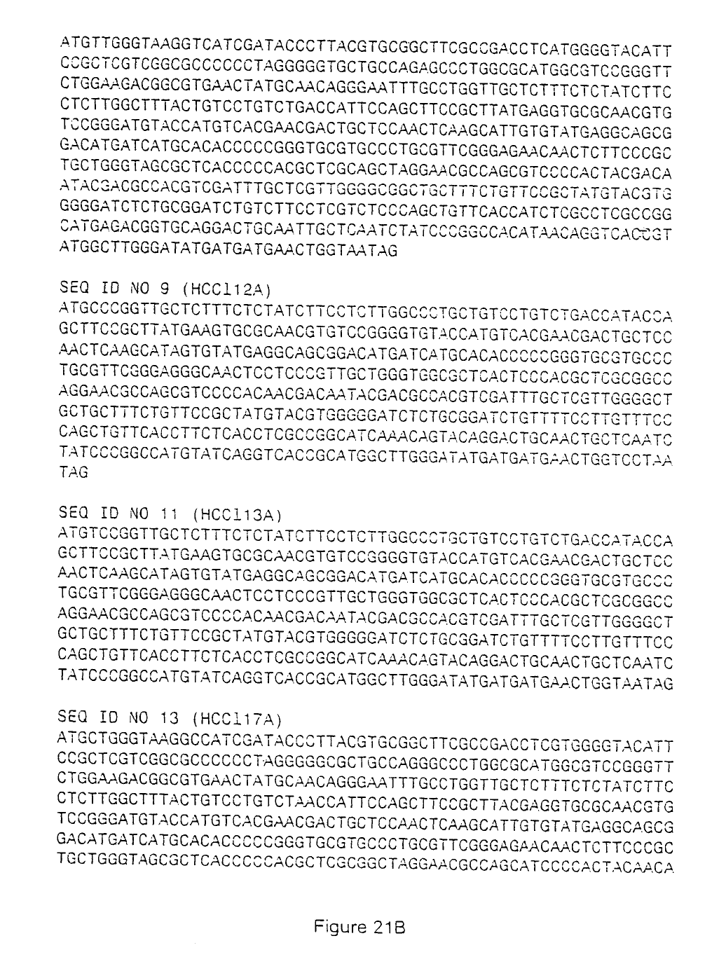

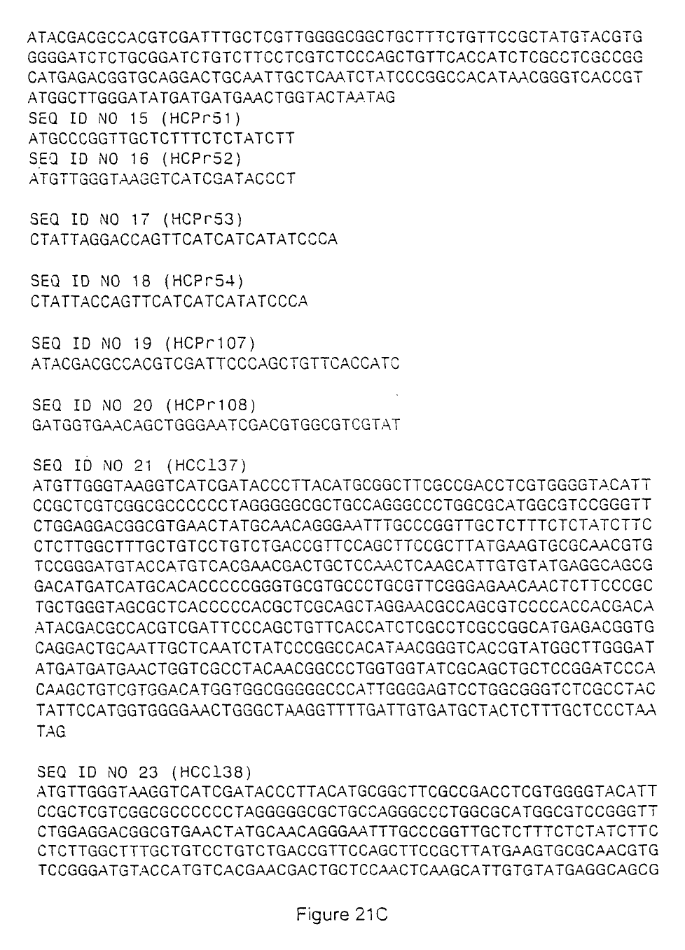

Of the 24 anti-E2 Mabs only three could be competed for reactivity to recombinant E2 by peptides, two of which reacted with the HVRI region (peptides E2-67 and E2-69, designated as epitope A) and one which recognized an epitope competed by peptide E2-13B (epitope C). The majority of murine antibodies recognized conformational anti-E2 epitopes (FIG. 19). A human response to HVRI (epitope A), and to a lesser extent HVRII (epitope B) and a third linear epitope region (competed by peptides E2-23, E2-25 or E2-27, designated epitope E) and a fourth linear epitope region (competed by peptide E2-17B, epitope D) could also frequently be observed, but the majority of sera reacted with conformational epitopes (FIG. 20). These conformational epitopes could be grouped according to their relative positions as follows: the IgG antibodies in the supernatant of hybridomas 15C8C1, 12D11 F1, 9G3E6, 8G10D1H9, 10D3C4, 4H6B2, 17F2C2, 5H6A7, 15B7A2 recognizing conformational epitopes were purified by means of protein A affinity chromatography and 1 mg/ml of the resulting IgG's were biotinylated in borate buffer in the presence of biotin. Biotinylated antibodies were separated from free biotin by means of gelfiltration chromatography. Pooled biotinylated antibody fractions were diluted 100 to 10,000 times. E2 protein bound to the solid phase was detected by the biotinylated IgG in the presence of 100 times the amount of non-biotinylated competing antibody and subsequently detected by alkaline phosphatase labeled streptavidin.

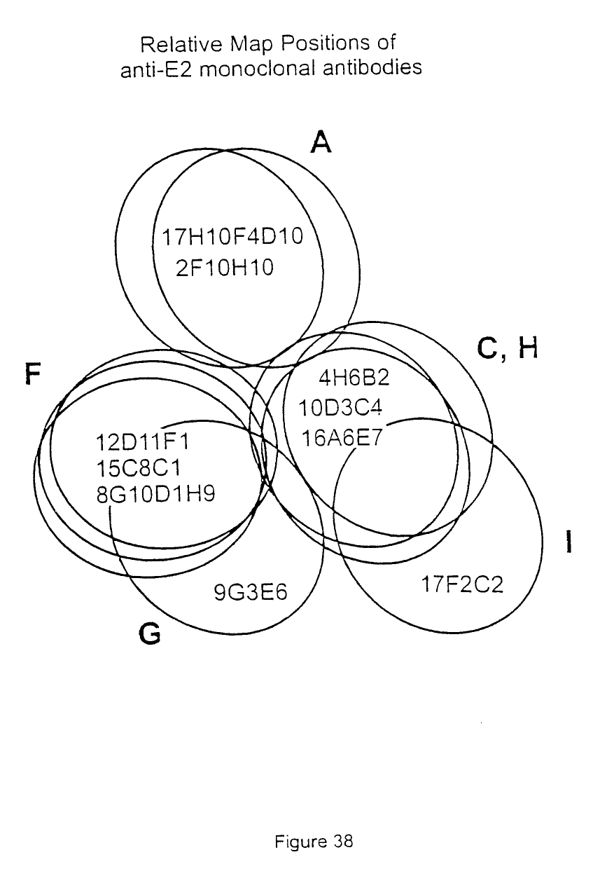

Percentages of competition are given in Table 6. Based on these results, 4 conformational anti-E2 epitope regions (epitopes F, G, H and I) could be delineated (FIG. 38). Alternatively, these Mabs may recognize mutant linear epitopes not represented by the peptides used in this study. Mabs 4H6B2 and 10D3C4 competed reactivity of 16A6E7, but unlike 16A6E7, they did not recognize peptide E2-13B. These Mabs may recognize variants of the same linear epitope (epitope C) or recognize a conformational epitope which is sterically hindered or changes conformation after binding of 16A6E7 to the E2-13B region (epitope H).

### EXAMPLE 8

### E1 Glycosylation Mutants

8.1. Introduction

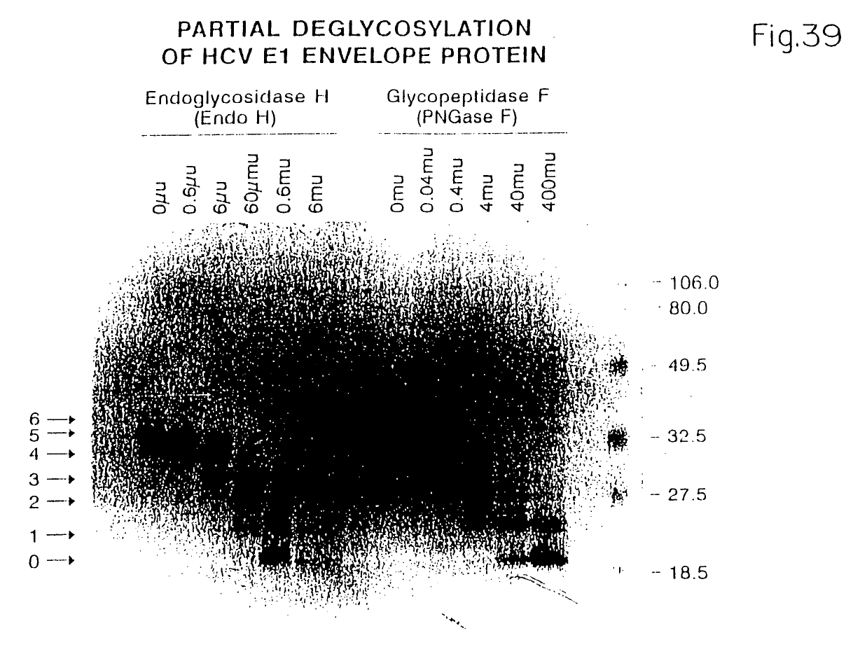

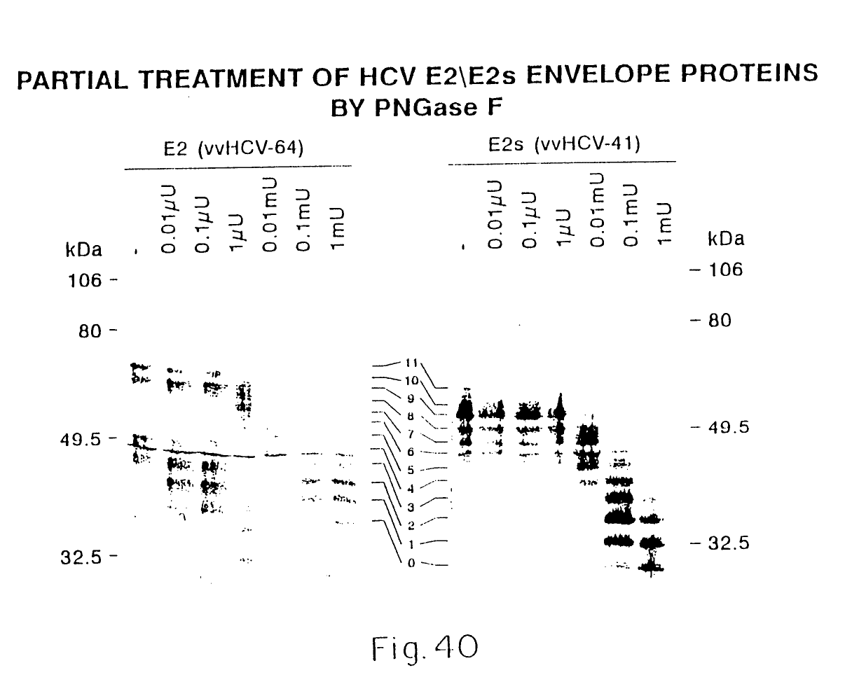

The E1 protein encoded by vvHCV10A, and the E2 protein encoded by vvHCV41 to 44 expressed from manrmalian cells contain 6 and 11 carbohydrate moieties, respectively. This could be shown by incubating the lysate of vvHCV10A-infected or vvHCV44-infected RK13 cells with decreasing concentrations of glycosidases (PNGase F or Endoglycosidase H, (Boehringer Mannhein Biochemica) according to the manufacturer's instructions), such that the proteins in the lysate (including E1) are partially deglycosylated (FIGS. 39 and 40, respectively).

Mutants devoid of some of their glycosylation sites could allow the selection of envelope proteins with improved immunological reactivity. For HIV for example, gp120 proteins lacking certain selected sugar-addition motifs, have been found to be particularly useful for diagnostic or vaccine purpose. The addition of a new oligosaccharide side chain in the hemagglutinin protein of an escape mutant of the A/Hong Kong/3/68 (H3N2) influenza virus prevents reactivity with a neutralizing monoclonal antibody (Skehel et al, 1984). When novel glycosylation sites were introduced into the influenza hemaglutinin protein by site-specific mutagenesis, dramatic antigenic changes were observed, suggesting that the carbohydrates serve as a modulator of antigenicity (Gallagher et al., 1988). In another analysis, the 8 carbohydrate-addition motifs of the surface protein gp70 of the Friend Murine Leukemia Virus were deleted. Although seven of the mutations did not affect virus infectivity, mutation of the fourth glycosylation signal with respect to the amino terminus resulted in a non-infectious phenotype (Kayman et al., 1991). Furthermore, it is known in the art that addition of N-linked carbohydrate chains is important for stabilization of folding intermediates and thus for efficient folding, prevention of malfolding and degradation in the endoplasmic reticulum, oligomerization, biological activity, and transport of glycoproteins (see reviews by Rose et al., 1988; Doms et al., 1993; Helenius, 1994).

After alignment of the different envelope protein sequences of HCV genotypes, it may be inferred that not all 6 glycosylation sites on the HCV subtype 1b E1 protein are required for proper folding and reactivity, since some are absent in certain (sub)types. The fourth carbohydrate motif (on Asn251), present in types 1b, 6a, 7, 8, and 9, is absent in all other types known today. This sugar-addition motif may be mutated to yield‘a type 1b E1 protein with improved reactivity. Also the type 2b sequences show an extra glycosylation site in the V5 region (on Asn299). The isolate S83, belonging to genotype 2c, even lacks the first carbohydrate motif in the V1 region (on Asn), while it is present on all other isolates (Stuyver et al., 1994). However, even among the completely conserved sugar-addition motifs, the presence of the carbohydrate may not be required for folding, but may have a role in evasion of immune surveillance., Therefore, identification of the carbohydrate addition motifs which are not required for proper folding (and reactivity) is not obvious, and each mutant has to be analyzed and tested for reactivity. Mutagenesis of a glycosylation motif (NXS or NXT sequences) can be achieved by either mutating the codons for N, S, or T, in such a way that these codons encode amino acids different from N in the case of N, and/or amino acids different from S or T in the case of S and in the case of T. Alternatively, the X position may be mutated into P, since it is known that NPS or NPT are not frequently modified with carbohydrates. After establishing which carbohydrate-addition motifs are required for folding and/or reactivity and which are not, combinations of such mutations may be made.

8.2. Mutagenesis of the E1 Protein

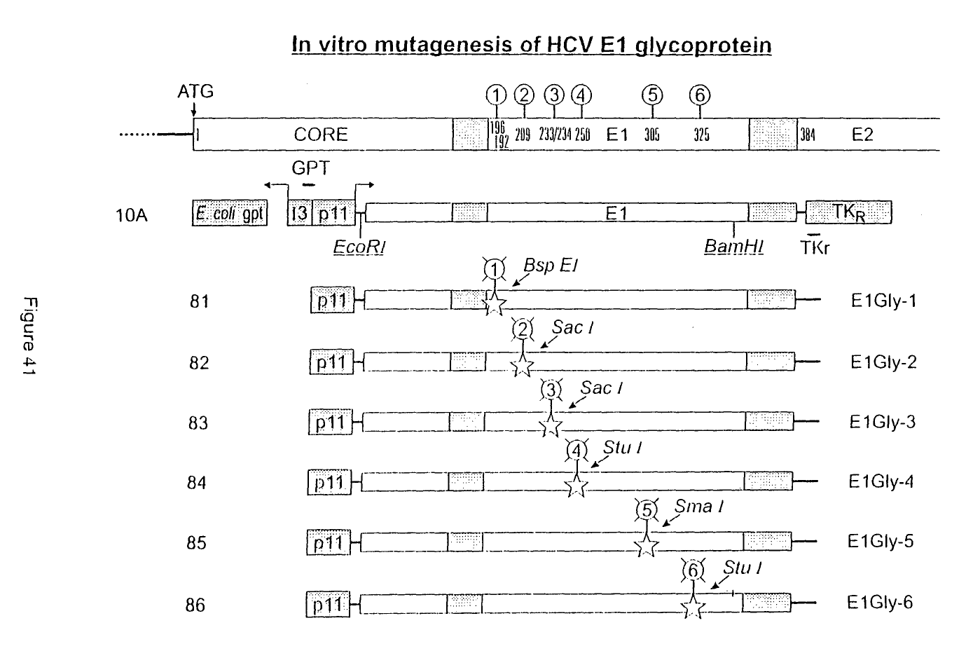

All mutations were performed on the E1 sequence of clone HCC110A (SEQ ID NO:5). The first round of PCR was performed using sense primer ‘GPT’ (see Table 7) targetting the GPT sequence located upstream of the vaccinia 11K late promoter, and an antisense primer (designated GLY#, with # representing the number of the glycosylation site, see FIG. 41) containing the desired base change to obtain the mutagenesis. The six GLY# primers (each specific for a given glycosylation site) were designed such that:

Modification of the codon encoding for the N-glycosylated Asn (AAC or AAT) to a Gln codon (CAA or CAG). Glutamine was chosen because it is very similar to asparagine (both amino acids are neutral and contain non-polar residues, glutamine has a longer side chain (one more —CH₂— group).

The introduction of silent mutations in one or several of the codons downstream of the glycosylation site, in order to create a new unique or rare (e.g. a second Smal site for E1Gly5) restriction enzyme site. Without modifying the amino acid sequence, this mutation will provide a way to distinguish the mutated sequences from the original E1 sequence (pvHCV-10A) or from each other (FIG. 41) . This additional restriction site may also be useful for the construction of new hybrid (double, triple, etc.) glycosylation mutants.

18 nucleotides extend 5’ of the first mismatched nucleotide and 12 to 16 nucleotides extend to the 3′ end. Table 7 depicts the sequences of the six GLY# primers overlapping the sequence of N-linked glycosylation sites.

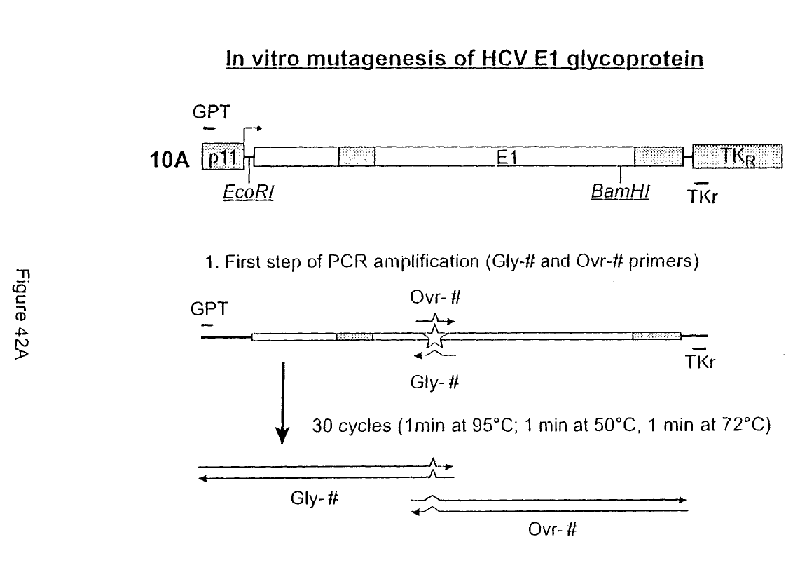

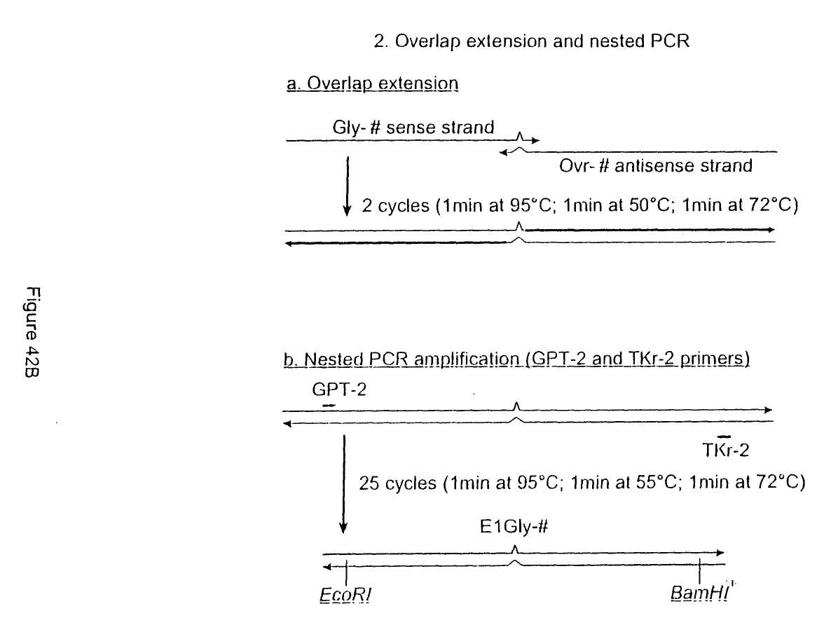

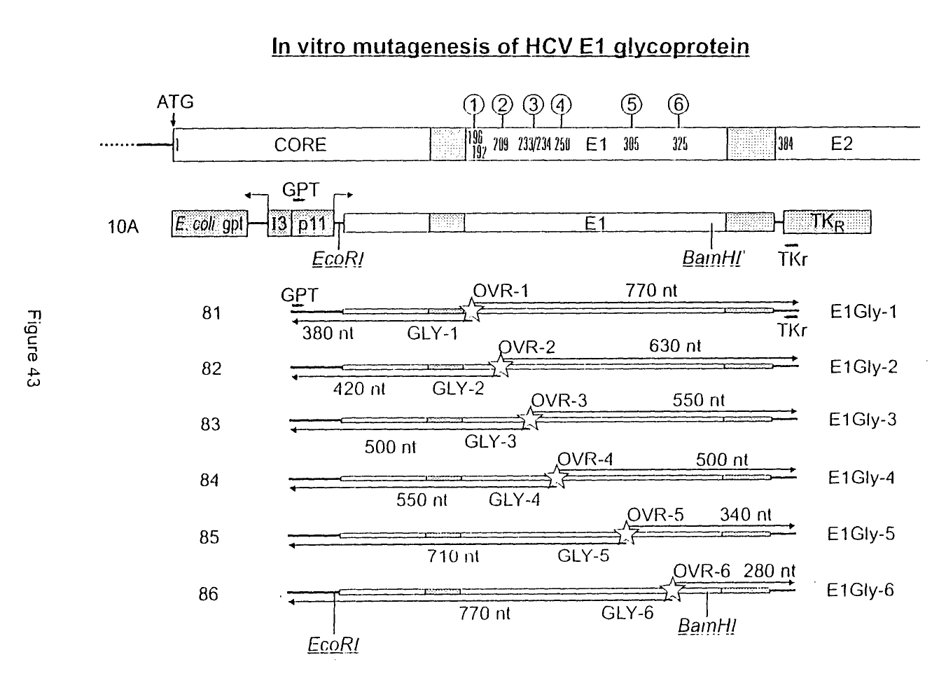

For site-directed mutagenesis, the ‘mispriming’ or ‘overlap extension’ (Horton, 1993) was used. The concept is illustrated in FIGS. 42 and 43. First, two separate fragments were amplified from the target gene for each mutated site. The PCR product obtained from the 5′ end (product GLY#) was amplified with the 5′ sense GPT primer (see Table 7) and with the respective 3′ antisense GLY# primers. The second fragment (product OVR#) was amplified with the 3′ antisense TK_{R} primer and the respective 5′ sense primers (OVR# primers, see Table 7, FIG. 43).

The OVR# primers target part of the GLY# primer sequence. Therefore, the two groups of PCR products share an overlap region of identical sequence. When these intermediate products are mixed (GLY-1 with OVR-1, GLY-2 with OVR-2, etc.), melted at high temperature, and reannealed, the top sense strand of product GLY# can anneal to the antisense strand of product OVR# (and vice versa) in such a way that the two strands act as primers for one another (see FIG. 42.B.). Extension of the annealed overlap by Taq polymerase during two PCR cycles created the full-length mutant molecule E1Gly#, which carries the mutation destroying the glycosylation site number #. Sufficient quantities of the E1GLY# products for cloning were generated in a third PCR by means of a common set of two internal nested primers. These two new primers are respectively overlapping the 3′ end of the vaccinia 11K promoter (sense GPT-2 primer) and the 5′ end of the vaccinia thymidine kinase locus (antisense TK_{R}-2 primer, see Table 7). All PCR conditions were performed as described in Stuyver et al. (1993).

Each of these PCR products was cloned by EcoRI/BamHI cleavage into the EcoRI/BamHI-cut vaccinia vector containing the original E1 sequence (pvHCV-10A).

The selected clones were analyzed for length of insert by EcoRI/BamH I cleavage and for the presence of each new restriction site. The sequences overlapping the mutated sites were confirmed by double-stranded sequencing.

8.3. Analysis of E1 Glycosylation Mutants

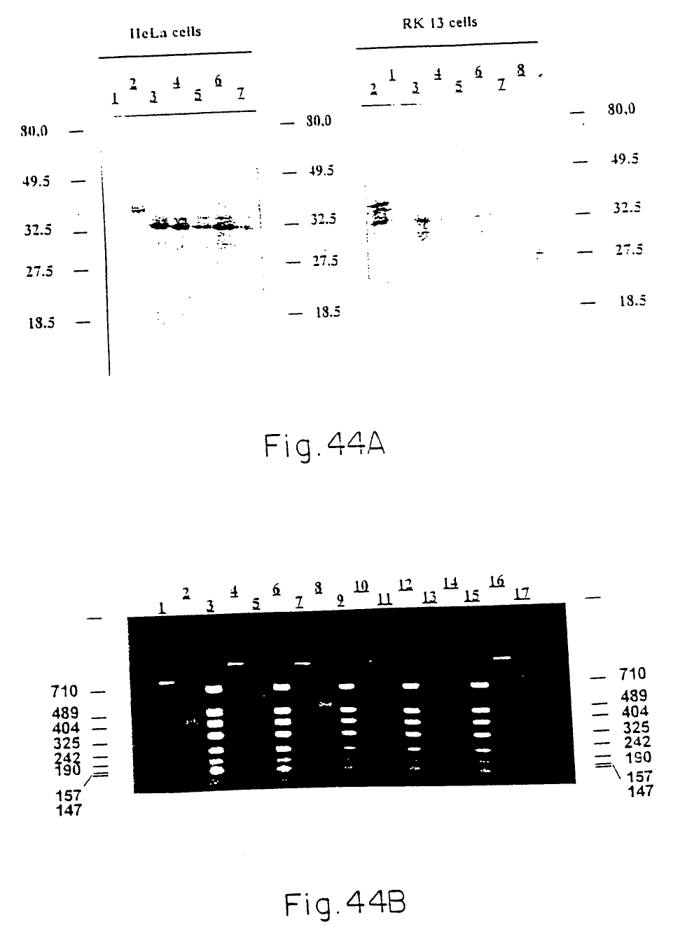

Starting from the 6 plasmids containing the mutant E1 sequences as described in example 8.2, recombinant vaccinia viruses were generated by recombination with wt vaccinia virus as described in example 2.5. Briefly, 175 cm²-flasks of subconfluent RK13 cells were infected with the 6 recombinant vaccinia viruses carrying the mutant E1 sequences, as well as with the vvHCV-10A (carrying the non-mutated E1 sequence) and wt vaccinia viruses. Cells were lysed after 24 hours of infection and analyzed on western blot as described in example 4 (see FIG. 44A). All mutants showed a faster mobility (corresponding to a smaller molecular weight of approximately 2 to 3 kDa) on SDS-PAGE than the original E1 protein; confirming that one carbohydrate moiety was not added. Recombinant viruses were also analyzed by PCR and restriction enzyme analysis to confirm the identity of the different mutants. FIG. 44B shows that all mutants (as shown in FIG. 41) contained the expected additional restriction sites. Another part of the cell lysate was used to test the reactivity of the different mutant by ELISA. The lysates were diluted 20 times and added to microwell plates coated with the lectin GNA as described in example 6. Captured (mutant) E1 glycoproteins were left to react with 20-times diluted sera of 24 HCV-infected patients as described in example 6. Signal to noise (S/N) values (OD of GLY#/OD of wt) for the six mutants and E1 are shown in Table 8. The table also shows the ratios between S/N values of GLY# and E1 proteins. It should be understood that the approach to use cell lysates of the different mutants for comparison of reactivity with patient sera may result in observations that are the consequence of different expression levels rather then reactivity levels. Such difficulties can be overcome by purification of the different mutants as described in example 5, and by testing identical quantities of all the different E1 proteins. However, the results shown in table 5 already indicate that removal of the 1st (GLY1), 3rd (GLY3), and 6th (GLY6) glycosylation motifs reduces reactivity of some sera, while removal of the 2nd and 5th site does not. Removal of GLY4 seems to improve the reactivity of certain sera. These data indicate that different patients react differently to the glycosylation mutants of the present invention. Thus, such mutant E1 proteins may be useful for the diagnosis (screening, confirmation, prognosis, etc.) and prevention of HCV disease.

### EXAMPLE 9

### Expression of HCV E2 Protein in Glycosylation-Deficient Yeasts

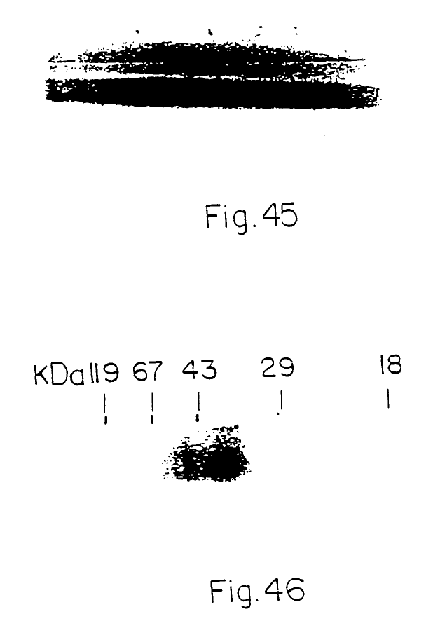

The E2 sequence corresponding to clone HCCl41 was provided with the α-mating factor pre/pro signal sequence, inserted in a yeast expression vector and S. cerevisiae cells transformed with this construct secreted E2 protein into the growth medium. It was observed that most glycosylation sites were modified with high-mannose type glycosylations upon expression of such a construct in S. cerevisiae strains (FIG. 45). This resulted in a too high level of heterogeneity and in shielding of reactivity, which is not desirable for either vaccine or diagnostic purposes. To overcome this problem, S. cerevisiae mutants with modified glycosylation pathways were generated by means of selection of vanadate-resistant clones. Such clones were analyzed for modified glycosylation pathways by analysis of the molecular weight and heterogeneity of the glycoprotein invertase. This allowed us to identify different glycosylation deficient S. cerevisiae mutants. The E2 protein was subsequently expressed in some of the selected mutants and left to react with a monoclonal antibody as described in example 7, on western blot as described in example 4 (FIG. 46).

### EXAMPLE 10

### General Utility

The present results show that not only a good expression system but also a good purification protocol are required to reach a high reactivity of the HCV envelope proteins with human patient sera. This can be obtained using the proper HCV envelope protein expression system and/or purification protocols of the present invention which guarantee the conservation of the natural folding of the protein and the purification protocols of the present invention which guarantee the elimination of contaminating proteins and which preserve the conformation, and thus the reactivity of the HCV envelope proteins. The amounts of purified HCV envelope protein needed for diagnostic screening assays are in the range of grams per year. For vaccine purposes, even higher amounts of envelope protein would be needed. Therefore, the vaccinia virus system may be used for selecting the best expression constructs and for limited upscaling, and large-scale expression and purification of single or specific oligomeric envelope proteins containing high-mannose carbohydrates may be achieved when expressed from several yeast strains. In the case of hepatitis B for example, manufacturing of HBsAg from mammalian cells was much more costly compared with yeast-derived hepatitis B vaccines.

The purification method dislcosed in the present invention may also be used for ‘viral envelope proteins’ in general. Examples are those derived from Flaviviruses, the newly discovered GB-A, GB-B and GB-C Hepatitis viruses, Pestiviruses (such as Bovine viral Diarrhoea Virus (BVDV), Hog Cholera Virus (HCV), Border Disease Virus (BDV)), but also less related virusses such as Hepatitis B Virus (mainly for the purification of HBsAg).

The envelope protein purification method of the present invention may be used for intra- as well as extracellularly expressed proteins in lower or higher eukaryotic cells or in prokaryotes as set out in the detailed description section.

### EXAMPLE 11

### Demonstration of Prophylactic and Therapeutic Utility

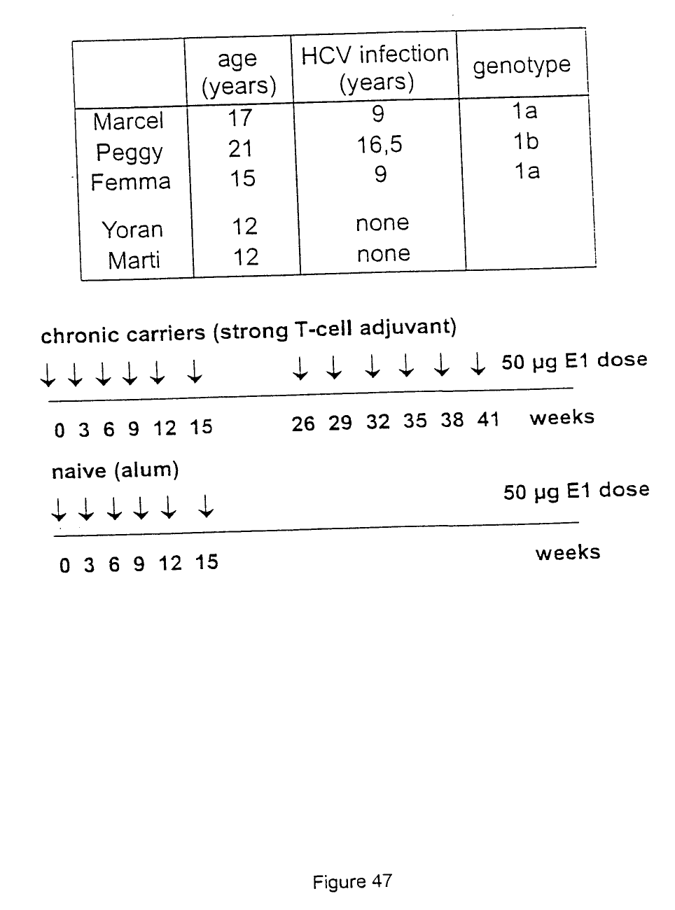

Liver disease in chimpanzees chronically infected with HCV can be reduced by immunization with E1. Multiple immunizations, however, were required in order to reach a significant immune response. One of ordinary skill will appreciate that viral persistence is produced with immune modulation which is either orchestrated by the virus itself or by the host. In order to analyze if such an immune modulation does exist in HCV, the immune responses against E1 and NS3 in naive and chronically infected chimpanzees were compared. Since a lower response in the chronically infected animals was anticipated, this group of animals was selected for a more rigorous immunization schedule including the following: use of an adjuvant proven in mice to be more potent for inducing cellular responses (Table 9) compared to alum, which was the adjuvant used for naive animals; and the immunization schedule for chronically infected animals consisted of 12 immunizations compared to 6 for naive animals (FIG. 47).

Although the number of immunized animals does not allow statistical analysis, the following clear tendency can be detected in the humoral responses (Table 10): the number of immunizations for seroconversion is lower in naive animals; and the magnitude of the immune response is substantially greater in the naive animals, 2/3 infected animals do not reach the level of 10 internal units, even after 12 immunizations.

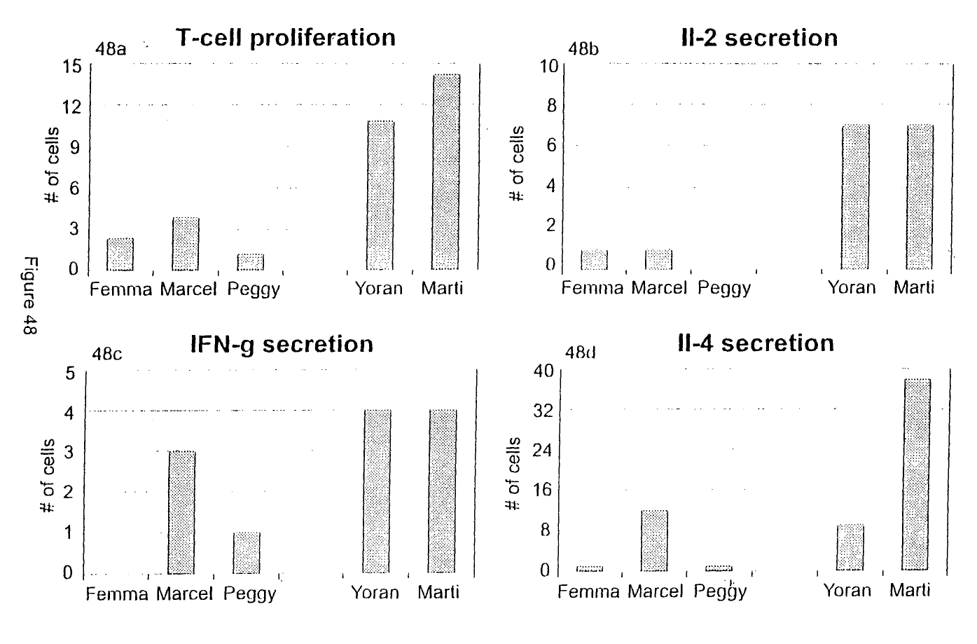

The analysis of the cellular responses, after three immunizations, reveals an even larger difference (FIGS. 48a-d), including the following: E1-specific T-cell proliferation is almost absent in the chronically infected animals, while a clear stimulation can be seen in the naive setting; IL-2 measurements confirmed that the low stimulation of the T-cell compartment in chronic carriers; and, a clear Th2 (IL-4) response in naive animals is induced, as expected for an alum-adjuvant containing vaccine.

This confirms that at least E1 immunization provides a prophylactic effect in naive animals and suggest that E2 and/or combinations of E1 and E2 proteins and/or peptides may provide useful therapeutic and/or prophylactic benefits in naive animals.

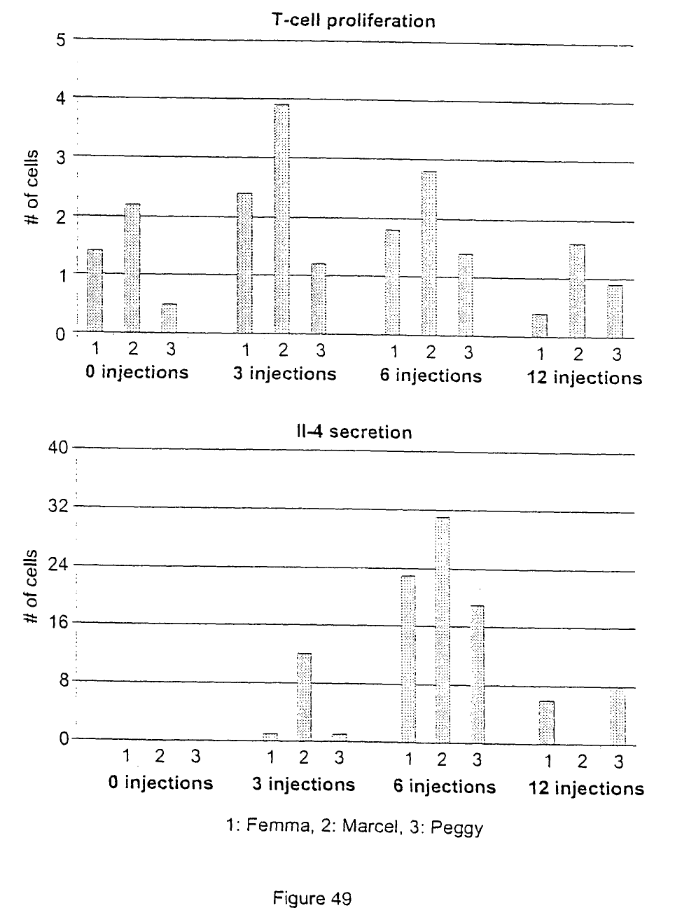

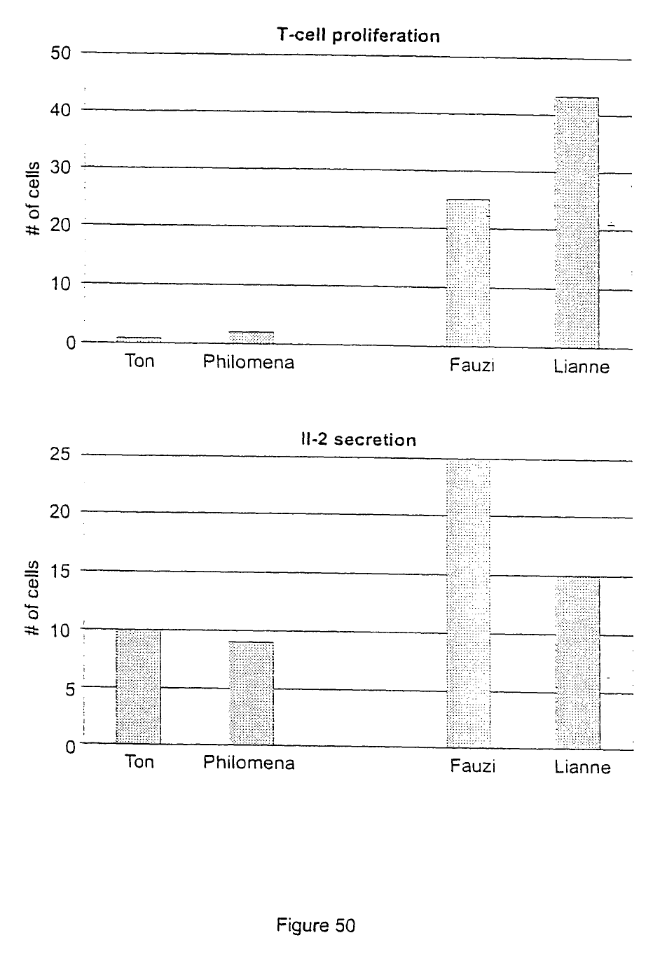

The ‘impairment’ to induce both cellular and humoral responses against an HCV E1 antigen can be only partially overcome by multiple immunizations, as demonstrated by the following results: an increase in antibody titer after each injection was noted but the levels as in naive animals were not reached in 2/3 animals; and the T-cell proliferative responses remain very low (FIG. 49). The ELISPOT results show, however, a minor increase in IL-2 (not shown), no change in IFN-g (not shown) and an increase in IL-4 (FIG. 49) which indicates that Th2 type responses are more readily induced. IL-4 was noted to remain at a low level compared to the level reached after three immunizations in naive animals.

A quite similar observation was made for NS3 immunizations where an even stronger adjuvant (RIBI) was used in the chronic chimpanzee. As compared with an alum formulation in naive animals the following has been noted: the induced antibody titers are comparable in both groups (not shown); and both cytokine secretion and T-cell proliferation are almost absent in the chronic animals compared to the responses in naive animals (FIGS. 49a-b).

Currently there have been some indications that immune responses against HCV in chronic carriers are low or at least insufficient to allow clearance of infection. The above results support the hypothesis that the immune system of HCV chronic carriers may be impaired and that they do not respond to HCV antigens as efficiently as in a naive situation.

In a study by Wiedmann et al., (Hepatology 2000; 31: 230-234), vaccination for HBV was less effective in HCV chronic carriers, which indicates that such an immune impairment is not limited to HCV antigens. De Maria et al. (Hepatology 2000; 32: 444-445) confirmed these data and have proposed adapted vaccine dosing regimens for HCV patients. The data presented herein indicates that increasing the number of immunizations may indeed augment humoral responses but that cellular (especially Th1) responses are difficult to induce, even when powerful adjuvants are used. It may be advantageous to begin immunization at the time of antiviral therapy, when the immune system is more prone to respond.

TABLE 1
Recombinant vaccinia plasmids and viruses

| | HCV cDNA sublcone | | | |
|---|---|---|---|---|
| Plasmid | | | Length | Vector used for |
| Name | Name | Construction | (nt/aa) | insertion |
| pvHCV-13A | E1s | EcoR I - Hind III | 472/157 | pgptATA-18 |
| pvHCV-12A | E1s | EcoR I - Hind III | 472/158 | pgptATA-18 |
| pvHCV-9A | E1 | EcoR I - Hind III | 631/211 | pgptATA-18 |
| pvHCV-11A | E1s | EcoR I - Hind III | 625/207 | pgptATA-18 |
| pvHCV-17A | E1s | EcoR I - Hind III | 625/208 | pgptATA-18 |
| pvHCV-10A | E1 | EcoR I - Hind III | 783/262 | pgptATA-18 |
| pvHCV-18A | COREs | Acc I (Kl) - EcoR I (Kl) | 403/130 | pgptATA-18 |
| pvHCV-34 | CORE | Acc I (Kl) - Fsp I | 595/197 | pgptATA-18 |
| pvHCV-33 | CORE-E1 | Acc I (Kl) | 1150/380 | pgptATA-18 |
| pvHCV-35 | CORE-E1b.his | EcoR I - BamH I (Kl) | 1032/352 | pMS-66 |
| pvHCV-36 | CORE-E1n.his | EcoR I - Nco I (Kl) | 1106/376 | pMS-66 |
| pvHCV-37 | E1Δ | Xma I - BamH I | 711/239 | pvHCV-10A |
| pvHCV-38 | EIΔs | EcoR I - BstE II | 553/183 | pvHCV-11A |
| pvHCV-39 | E1Δb | EcoR I - BamH I | 960/313 | pgsATA-18 |
| pvHCV-40 | E1Δb.his | EcoR I - BamH I (Kl) | 960/323 | pMS-66 |
| pvHCV-41 | E2bs | BamH I (Kl)-AlwN I (T4) | 1005/331 | pgsATA-18 |
| pvHCV-42 | E2bs.his | BamH I (Kl)-AlwN I (T4) | 1005/341 | pMS-66 |
| pvHCV-43 | E2ns | Nco I (Kl) - AlwN I (T4) | 932/314 | pgsATA-18 |
| pvHCV-44 | E2ns.his | Nco I (Kl) - AlwN I (T4) | 932/321 | pMS-66 |
| pvHCV-62 | E1s (type 3a) | EcoR I - Hind III | 625/207 | pgsATA-18 |
| pvHCV-63 | E1s (type 5) | EcoR I - Hind III | 625/207 | pgsATA-18 |
| pvHCV-64 | E2 | BamH I - Hind III | 1410/463 | pgsATA-18 |
| pvHCV-65 | E1-E2 | BamH I - Hind III | 2072/691 | pvHCV-10A |
| pvHCV-66 | CORE-E1-E2 | BamH I - Hind III | 2427/809 | pvHCV-33 |
| pvHCV-81 | E1*-GLY 1 | EcoRI - BamH I | 783/262 | pvHCV-10A |
| pvHCV-82 | E1*-GLY 2 | EcoRI - BamH I | 783/262 | pvHCV-10A |
| pvHCV-83 | El*-GLY 3 | EcoRI - BamH I | 783/262 | pvHCV-10A |
| pvHCV-84 | E1*-GLY 4 | EcoRI - BamH I | 783/262 | pvHCV-10A |
| pvHCV-85 | E1*-GLY 5 | EcoRI - BamH I | 783/262 | pvHCV-10A |
| pvHCV-86 | E1*-GLY 6 | EcoRI - BamH I | 783/262 | pvHCV-10A |

nt: nucleotide;
aa: amino acid;
Kl: Klenow DNA Pol filling;
T4: T4 DNA Pol filling
Position: amino acid position in the HCV polyprotein sequence

TABLE 2
Summary of anti-E1 tests
S/N ± SD (mean anti-E1 titer)

| | Start of treatment | End of treatment | Follow-up |
|---|---|---|---|
| LTR | 6.94 ± 2.29 (1:3946) | 4.48 ± 2.69 (1:568) | 2.99 ± 2.69 |
| | | | (1:175) |
| NR | 5.77 ± 3.77 (1:1607) | 5.29 ± 3.99 (1:1060) | 6.08 ± 3.73 |
| | | | (1:1978) |

LTR: Long-term, sustained response for more than 1 year
NR: No response, response with relapse, or partial response

TABLE 3
Synthetic peptides for competition studies

| | | | | |
|---|---|---|---|---|
| | | | | SEQ |
| | | | | ID |
| PROTEIN | PEPTIDE | AMINO ACID SEQUENCE | POSITION | NO |

| | | | | |
|---|---|---|---|---|
| E1 | E1-31 | LLSCLTVPASAYQVRNSTGL | 181-200 | 56 |
| | E1-33 | QVRNSTGLYHVTNDCPNSSI | 193-212 | 57 |
| | E1-35 | NDCPNSSIVYEAHDAILHTP | 205-224 | 58 |
| | E1-35A | SNSSIVYEAADMIMHTPGCV | 208-227 | 59 |
| | E1-37 | HDAILHTPGCVPCVREGNVS | 217-236 | 60 |
| | E1-39 | CVREGNVSRCWVAMTPTVAT | 229-248 | 61 |
| | E1-41 | AMTPTVATRDGKLPATQLRR | 241-260 | 62 |
| | E1-43 | LPATQLRRHIDLLVGSATLC | 253-272 | 63 |
| | E1-45 | LVGSATLCSALYVGDLCGSV | 265-284 | 64 |
| | E1-49 | QLFTFSPRRHWTTQGCNCSI | 289-308 | 65 |
| | E1-51 | TQGCNCSIYPGHITGHRMAW | 301-320 | 66 |
| | E1-53 | ITGHRMAWDMMMNWSPTAAL | 313-332 | 67 |
| | E1-55 | NWSPTAALVMAQLLRIPQAI | 325-344 | 68 |
| | E1-57 | LLRIPQAILDMIAGAHWGVL | 337-356 | 69 |
| | E1-59 | AGAHWGVLAGIAYFSMVGNM | 349-368 | 70 |
| | E1-63 | VVLLLFAGVDAETIVSGGQA | 373-392 | 71 |
| E2 | E2-67 | SGLVSLFTPGAKQNIQLINT | 397-416 | 72 |
| | E2-69 | QNIQLINTNGSWHINSTALN | 409-428 | 73 |
| | E2-$3B | LNCNESLNTGWWLAGLIYQHK | 427-446 | 74 |
| | E2-$1B | AGLIYQHKFNSSGCPERLAS | 439-458 | 75 |
| | E2-1B | GCPERLASCRPLTDFDQGWG | 451-470 | 76 |
| | E2-3B | TDFDQGWGPISYANGSGPDQ | 463-482 | 77 |
| | E2-5B | ANGSGPDQRPYCWHYPPKPC | 475-494 | 78 |
| | E2-7B | WHYPPKPCGIVPAKSVCGPV | 487-506 | 79 |
| | E2-9B | AKSVCGPVYCFTPSPVVVGT | 499-518 | 80 |
| | E2-11B | PSPVVVGTTDRSGAPTYSWG | 511-530 | 81 |
| | E2-13B | GAPTYSWGENDTDVFVLNNT | 523-542 | 82 |
| | E2-17B | GNWFGCTWMNSTGFTKVCGA | 547-566 | 83 |
| | E2-19B | GFTKVCGAPPVCIGGAGNNT | 559-578 | 84 |
| | E2-21 | IGGAGNNTLHCPTDCFRKHP | 571-590 | 85 |
| | E2-23 | TDCFRKHPDATYSRCGSGPW | 583-602 | 86 |
| | E2-25 | SRCGSGPWITPRCLVDYPYR | 595-614 | 87 |
| | E2-27 | CLVDYPYRLWHYPCTINYTI | 607-626 | 88 |
| | E2-29 | PCTINYTIFKIRMYVGGVEH | 619-638 | 89 |
| | E2-31 | MYVGGVEHRLEAACNWTPGE | 631-650 | 90 |
| | E2-33 | ACNWTPGERCDLEDRDRSEL | 643-662 | 91 |
| | E2-35 | EDRDRSELSPLLLTTTQWQV | 655-674 | 92 |

TABLE 4
Change of Envelope Antibody levels over time (complete study, 28 patients)

| Wilcoxon Signed | | E1Ab NR | | E1Ab LTR | E1Ab LTR | E1Ab LTR | | E1Ab LTR | | |
|---|---|---|---|---|---|---|---|---|---|---|
| Rank test (P values) | E1Ab NR | All | E1Ab NR | type 1b | type 3a | All | E2Ab NR | type 1b | type 3a | All |
| End of therapy* | 0.1167 | 0.2604 | 0.285 | | 0.0058** | 0.043** | 0.0499** | 0.0186** | 0.0640 | |
| 6 months follow up* | 0.86 | | 0.7213 | 0.5930 | 0.0047** | 0.043** | 0.063 | | 0.04326 | 0.0464** |
| 12 months follow up* | 0.7989 | 0.3105 | 1 | | 0.0051** | 0.0679 | 0.0277** | 0.0869 | 0.0058** | |

*Data were compared with values obtained at initiation of therapy
**P values < 0.05

TABLE 5
Difference between LTR and NR (complete study)

| | | E1Ab | | | E2Ab |
|---|---|---|---|---|---|
| Mann-Withney | E1Ab S/N | titers | E1Ab S/N | E1Ab S/N | S/N |
| U test (P values) | All | All | type 1b | type 3a | All |
| Initiation of therapy | 0.0257* | 0.05* | 0.68 | 0.1078 | |
| End of therapy | 0.1742 | | | 0.1295 | |
| 6 months follow up | 1 | 0.6099 | 0.425 | 0.3081 | |
| 12 months follow up | 0.67 | 0.23 | 0.4386 | 0.6629 | |

*P values < 0.05

TABLE 6
Competition experiments between murine E2 monoclonal antibodies

| | Decrease (%) of anti-E2 reactivity of biotinylated anti-E2 mabs | | | | | | | | | |
|---|---|---|---|---|---|---|---|---|---|---|
| competitor | 17H10F4D10 | 2F10H10 | 16A6E7 | 10D3C4 | 4H6B2 | 17C2F2 | 9G3E6 | 12D11F1 | 15C8C1 | 8G10D1H9 |
| 17H10F4D10 | — | 62 | 10 | ND | 11 | ND | 5 | 6 | 30 | ND |
| 2F10H10 | 90 | — | 1 | ND | 30 | ND | 0 | 4 | 12 | ND |
| 16A6E7 | ND | ND | — | ND | ND | ND | ND | ND | ND | ND |
| 10D3C4 | 11 | 50 | 92 | — | 94 | 26 | 28 | 43 | 53 | 30 |
| 4H6B2 | ND | ND | 82 | ND | — | ND | ND | ND | ND | ND |
| 17C2F2 | 2 | ND | 75 | ND | 56 | — | 11 | 10 | 0 | 0 |
| 9G3E6 | ND | ND | 68 | ND | 11 | ND | — | 60 | 76 | ND |
| 12D11F1 | ND | ND | 26 | ND | 13 | ND | ND | — | 88 | ND |
| 15C8C1 | ND | ND | 18 | ND | 10 | ND | ND | ND | — | ND |
| 8G10D1H9 | 2 | 2 | 11 | ND | 15 | ND | 67 | 082 | 81 | — |
| competitor controls | | | | | | | | | | |
| 15B7A2 | 0 | 0 | 9 | 15 | 10 | 9 | 0 | 0 | 0 | 5 |
| 5H6A7 | 0 | 2 | 0 | 12 | 8 | 0 | 0 | 4 | 0 | 0 |
| 23C12H9 | ND | ND | 2 | 12 | ND | 4 | ND | ND | ND | 2 |

ND, not done

TABLE 7
Primers

| | | |
|---|---|---|
| SEQ ID NO:96 | GPT | 5′-GTTTAACCACTGCATGATG-3′ |
| SEQ ID NO:97 | TK_{R} | 5′-GTCCCATCGAGTGCGGCTAC-3′ |
| SEQ ID NO:98 | GLY1 | 5′-CGTGACATGGTACATTCCGGACACTTGGCGCACTTCATAAGCGGA-3′ |
| SEQ ID NO:99 | GLY2 | 5′-TGCCTCATACACAATGGAGCTCTGGGACGAGTCGTTCGTGAC-3′ |
| SEQ ID NO:100 | GLY3 | 5′-TACCCAGCAGCGGGAGCTCTGTTGCTCCCGAACGCAGGGCAC-3′ |
| SEQ ID NO:101 | GLY4 | 5′-TGTCGTGGTGGGGACGGAGGCCTGCCTAGCTGCGAGCGTGGG-3′ |
| SEQ ID NO:102 | GLY5 | 5′-CGTTATGTGGCCCGGGTAGATTGAGCACTGGCAGTCCTGCACCGTCTC-3′ |
| SEQ ID NO:103 | GLY6 | 5′-CAGGGCCGTTGTAGGCCTCCACTGCATCATCATATCCCAAGC-3′ |
| SEQ ID NO:104 | OVR1 | 5′-CCGGAATGTACCATGTCACGAACGAC-3′ |
| SEQ ID NO:105 | OVR2 | 5′-GCTCCATTGTGTATGAGGCAGCGG-3′ |
| SEQ ID NO:106 | OVR3 | 5′-GAGCTCCCGCTGCTGGGTAGCGC-3′ |
| SEQ ID NO:107 | OVR4 | 5′-CCTCCGTCCCCACCACGACAATACG-3′ |
| SEQ ID NO:108 | OVR5 | 5′-CTACCCGGGCCACATAACGGGTCACCG-3′ |
| SEQ ID NO:109 | OVR6 | 5′-GGAGGCCTACAACGGCCCTGGTGG-3′ |
| SEQ ID NO:110 | GPT-2 | 5′-TTCTATCGATTAAATAGAATTC-3′ |
| SEQ ID NO:111 | TK_{R}-2 | 5′-GCCATACGCTCACAGCCGATCCC-3′ |

nucleotides underlined represent additional restriction site
nucleotides in bold represent mutations with respect to the original HCC110A sequence

TABLE 8
Analysis of E1 glycosylation mutants by ELISA
SERUM

| | 1 | 2 | 3 | 4 | 5 | 6 | 7 | 8 | 9 |
|---|---|---|---|---|---|---|---|---|---|
| SN GLY1 | 1.802462 | 2.120971 | 1.403871 | 1.205597 | 2.120191 | 2.866913 | 1.950345 | 1.866183 | 1.730193 |
| SN GLY2 | 2.400795 | 1.76818 | 2.325495 | 2.639308 | 2.459019 | 5.043993 | 2.146302 | 1.595477 | 1.688973 |
| SN GLY3 | 1.642718 | 1.715477 | 2.261646 | 2.354748 | 1.591818 | 4.833742 | 1.96692 | 1.482099 | 1.602222 |
| SN GLY4 | 2.578154 | 3.824038 | 3.874605 | 1.499387 | 3.15 | 4.71302 | 4.198751 | 3.959542 | 3.710507 |
| SN GLY5 | 2.482051 | 1.793761 | 2.409344 | 2.627358 | 1.715311 | 4.964765 | 2.13912 | 1.576336 | 1.708937 |
| SN GLY6 | 2.031487 | 1.495737 | 2.131613 | 2.527925 | 2.494833 | 4.784027 | 2.02069 | 1.496489 | 1.704976 |
| SN E1 | 2.828205 | 2.227036 | 2.512792 | 2.790881 | 3.131579 | 4.869128 | 2.287753 | 1.954198 | 1.805556 |
| | 10 | 11 | 12 | 13 | 14 | 15 | 16 | 17 | 18 |
| SN GLY1 | 2.468162 | 1.220654 | 1.629403 | 5.685561 | 3.233604 | 3.763498 | 1.985105 | 2.317721 | 6.675179 |
| SN GLY2 | 2.482212 | 1.467582 | 2.070524 | 7.556682 | 2.567613 | 3.621928 | 3.055649 | 2.933792 | 7.65433 |
| SN GLY3 | 2.191558 | 1.464216 | 1.721164 | 7.930538 | 2.763055 | 3.016099 | 2.945628 | 2.515305 | 5.775357 |
| SN GLY4 | 5.170841 | 4.250784 | 3.955153 | 8.176816 | 6.561122 | 5.707668 | 5.684498 | 5.604813 | 6.4125 |
| SN GLY5 | 3.021807 | 1.562092 | 2.07278 | 8.883408 | 2.940334 | 3.125561 | 3.338912 | 2.654224 | 5.424107 |
| SN GLY6 | 2.677757 | 1.529608 | 1.744221 | 8.005561 | 2.499952 | 2.621704 | 2.572385 | 2.363301 | 5.194107 |
| SN E1 | 2.616822 | 1.55719 | 2.593886 | 8.825112 | 3.183771 | 3.067265 | 3.280335 | 2.980354 | 7.191964 |
| | | | | | | | SUM | AVERAGE | |
| | 19 | 20 | 21 | 22 | 23 | 24 | S/N | S/N | |
| SN GLY1 | 1.93476 | 2.47171 | 4.378633 | 1.188748 | 2.158889 | 1.706992 | 59.88534 | 2.495223 | |
| SN GLY2 | 2.127712 | 2.921288 | 4.680101 | 1.150781 | 1.661914 | 1.632785 | 69.65243 | 2.902185 | |
| SN GLY3 | 1.980185 | 2.557384 | 4.268633 | 0.97767 | 1.336775 | 1.20376 | 62.09872 | 2.587447 | |
| SN GLY4 | 3.813321 | 3.002535 | 4.293038 | 2.393011 | 3.68213 | 2.481585 | 102.6978 | 4.279076 | |
| SN GLY5 | 2.442804 | 3.126761 | 4.64557 | 1.153656 | 1.817901 | 1.638211 | 69.26511 | 2.886046 | |
| SN GLY6 | 1.506716 | 2.665433 | 2.781063 | 1.280743 | 1.475062 | 1.716423 | 61.32181 | 2.555075 | |
| SN E1 | 2.771218 | 3.678068 | 5.35443 | 1.167286 | 2.083333 | 1.78252 | 76.54068 | 3.189195 | |
| | 1 | 2 | 3 | 4 | 5 | 6 | 7 | 8 | 9 |
| GLY1/E1 | 0.637316 | 0.952374 | 0.55869 | 0.431977 | 0.677036 | 0.588794 | 0.852516 | 0.954961 | 0.958261 |
| GLY2/E1 | 0.848876 | 0.793961 | 0.925463 | 0.94569 | 0.785233 | 1.035913 | 0.93817 | 0.816436 | 0.935431 |
| GLY3/E1 | 0.580834 | 0.770296 | 0.900053 | 0.84373 | 0.508312 | 0.992733 | 0.859761 | 0.758418 | 0.887385 |
| GLY4/E1 | 0.911587 | 1.717097 | 1.541952 | 0.537245 | 1.005882 | 0.967939 | 1.835317 | 2.026172 | 2.05505 |
| GLY5/E1 | 0.877607 | 0.805447 | 0.958831 | 0.941408 | 0.547746 | 1.019642 | 0.935031 | 0.806641 | 0.946488 |
| GLY6/E1 | 0.718296 | 0.671626 | 0.848305 | 0.90578 | 0.796669 | 0.982522 | 0.883264 | 0.765781 | 0.944294 |
| | 10 | 11 | 12 | 13 | 14 | 15 | 16 | 17 | 18 |
| GLY1/E1 | 0.94319 | 0.783882 | 0.628171 | 0.644248 | 1.015652 | 1.226988 | 0.605153 | 0.777666 | 0.928144 |
| GLY2/E1 | 0.94856 | 0.942455 | 0.798232 | 0.85627 | 0.806469 | 1.180833 | 0.931505 | 0.984377 | 1.064289 |
| GLY3/E1 | 0.837488 | 0.940294 | 0.663547 | 0.898633 | 0.867856 | 0.983319 | 0.897966 | 0.843962 | 0.803029 |
| GLY4/E1 | 1.976 | 2.72978 | 1.524798 | 0.92654 | 2.060802 | 1.860833 | 1.732902 | 1.880587 | 0.89162 |
| GLY5/E1 | 1.154762 | 1.003148 | 0.799102 | 1.006606 | 0.923538 | 1.019006 | 1.017857 | 0.890574 | 0.75419 |
| GLY6/E1 | 1.023286 | 0.982288 | 0.672435 | 0.907134 | 0.785217 | 0.854737 | 0.784184 | 0.79296 | 0.72221 |
| | | | | | | | SUM | AVERAGE | |
| | 19 | 20 | 21 | 22 | 23 | 24 | E1/GLY# | E1/GLY# | |
| GLY1/E1 | 0.698162 | 0.672013 | 0.817759 | 1.018386 | 1.036267 | 0.9576281 | 9.36524 | 0.806885 | |
| GLY2/E1 | 0.76779 | 0.794245 | 0.874061 | 0.98586 | 0.797719 | 0.9159982 | 1.67384 | 0.903077 | |
| GLY3/E1 | 0.714554 | 0.695306 | 0.797215 | 0.837558 | 0.641652 | 0.6753141 | 9.19921 | 0.799967 | |
| GLY4/E1 | 1.376045 | 0.816335 | 0.801773 | 2.050064 | 1.767422 | 1.3921783 | 6.38592 | 1.51608 | |
| GLY5/E1 | 0.881491 | 0.850109 | 0.867612 | 0.988323 | 0.872593 | 0.9190422 | 1.78679 | 0.907783 | |
| GLY6/E1 | 0.543702 | 0.724683 | 0.519395 | 1.097197 | 0.70803 | 0.9629191 | 9.59691 | 0.816538 | |

TABLE 9
Profile of adjuvated E1 in Balb/c mice

| | alum | T-cell adjuvant | RBI |
|---|---|---|---|
| antibody titre | 96000 ± 101000 | 62000 ± 60000 | 176000 ± 149000 |
| (mean ± SD, | | | |
| n = 6) | | | |
| antibody isotypes | IgG1 | IgG1/2b | IgG1/2a |
| T-cell | 11750 (2/3) | 48300 (3/3) | 26000 (3/3) |
| preliferation | | | |
| in spleen¹ | | | |
| (n = 23) | | | |
| T-cell | no specific | 4000 | 8000 |
| proliferation | stimulation | | |
| in lymph node² | | | |
| cytokine profile | Il-4 | IFN-g/Il-4 | IFN-g/Il-4 |
| (spleen) | | | |

¹after three s.c./i.m. immunizations, 3 randomly selected mice were analyzed individually, the result is expressed as the mean specific cpm obtained after 4 days of E1 stimulation (1 μg/ml), the number in brackets refers to the number of mice with specific stimulation above background
²after one single intra footpath immunization (n = 2), the result is expressed as the mean specific cpm obtained after 5 days of E1 stimulation (1 μg/ml)

TABLE 10
Humoral Responses: No. of immunizations required
for different E-1 antibodies levels

| Animal | status | seroconversion¹ | >1 U/ml² | >10 U/ml |
|---|---|---|---|---|
| Marcel | chronic | 3 | 4 | 5 |
| Peggy | chronic | 3 | 5 | >12 |
| Femma | chronic | 4 | 5 | >12 |
| Yoran | naive | 3 | 4 | 5 |
| Marti | naive | 2 | 3 | 5 |

¹defined as ELISA signal higher than cut-off level if no E1-antibodies were present prior to immunization, in the other cases the observation of a titer higher than the 3 individual time points of pre-immunization titers was considered as the point of seroconversion.
²the unit is defined as follows: the level of E1 antibodies in human chronic carriers prior to interferon therapy and infected with genotype 1 b is <0.1 U/ml for 50% of the patients, between 0.1 to 1 U/ml for 25% of the patients and >1 U/ml in the remaining 25% of patients, n = 58

### EXAMPLE 12

### Immunization of a Chimpanzee Chronically Infected with HCV Subtype 1b

A chimpanzee (Phil) already infected for over 13 years (5015 days before immunization) with an HCV subtype Ib strain was vaccinated with E1 (aa 192-326) which was derived from a different strain of genotype 1 b, with a 95.1% identity on the amino acid level (see also Table 2 of WO 99/67285 the whole of which is incorporated herein by reference), and which was prepared as described in examples 1-3 of WO 99/97285. The chimpanzee received in total 6 intramuscular immunizations of each 50 μg E1 in PBS/0.05% CHAPS mixed with RIBI R-730 (MPLA+TDM+CWS) according to the manufacturer's protocol (Ribi Inc. Hamilton, Mont.). The 6 immunizations were given in two series of three shots with a three week interval and with a lag period of 6 weeks between the two series. Starting 150 days prior to immunization, during the immunization period and until 1 year post immunization (but see below and WO 99/67285) the chimpanzee was continuously monitored for various parameters indicative for the activity.of the HCV induced disease. These parameters included blood chemistry, ALT ,AST, gammaGT, blood chemistry, viral load in the serum, viral load in the liver and liver histology. In addition, the immune answer to the immunization was monitored both on the humoral and cellular level. During this period the animal was also monitored for any adverse effects of the immunization, such as change in behaviour, clinical symptoms, body weight, temperature and local reactions (redness, swelling, indurations). Such effects were not detected.

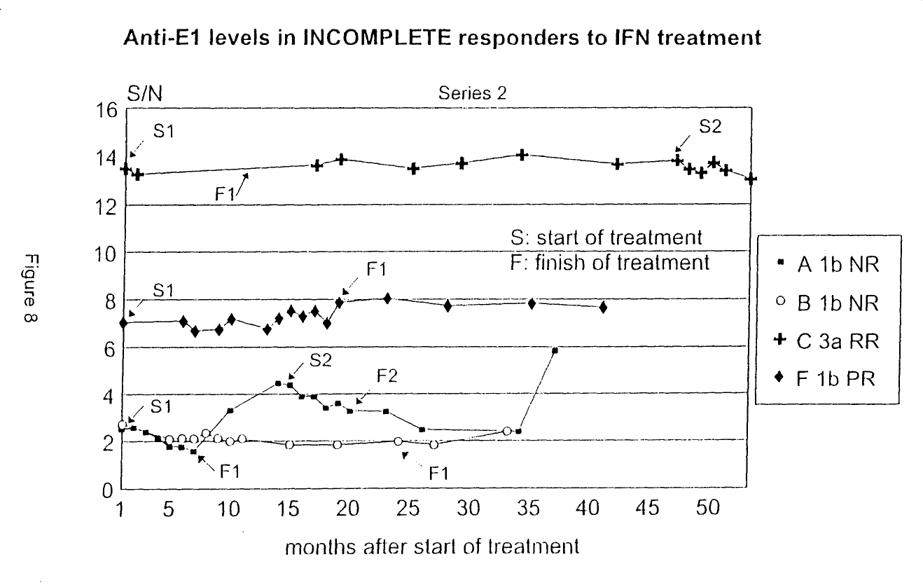

Clearly, ALT (and especially gammaGT, data not shown) levels decreased as soon as the antibody level against E1 reached its maximum (see, FIG. 8 of WO 99/67285). ALT rebounded rather rapidly as soon as the antibody levels started to decline, but gammaGT remained at a lower level as long as anti-E1 remained detectable.

E2 antigen in the liver decreased to almost undetectable levels during the period in which anti-E1 was detectable and the E2 antigen rebounded shortly after the disappearance of these antibodies. Together with the Core and E2 antigen becoming undetectable in the liver, the inflammation of the liver markedly decreased (see also Table 3 of WO 99/67285). This is a major proof that the vaccine induces a reduction of the liver damage, probably by clearing, at least partially, the viral antigens from its major target organ, the liver . The viraemia level, as measured by Amplicor HCV Monitor (Roche, Basel, Switzerland), remained approximately unchanged in the serum during the whole study period.

More detailed analyses of the humoral response revealed that the maximum end-point titer reached 14.5×10³ (after the sixth immunization) and that this titer dropped to undetectable 1 year post immunization (FIG. 8 of WO 99/67285). FIG. 9 of WO 99/67285 shows that the main epitopes, which can be mimicked by peptides, recognized by the B-cells are located at the N-terminal region of E2 (peptides V1V2 and V2V3, for details on the peptides used see Table 4 of WO 99/67285). Since the reactivity against the recombinant E1 is higher and longer lasting, it can also be deduced from this figure, that the antibodies recognizing these peptides represent only part of the total antibody population against E1. The remaining part is directed against epitopes which cannot be mimicked by peptides, i.e discontinuous epitopes. Such epitopes are only present on the complete E1 molecule or even only on the particle-like structure. Such an immune response against E1 is unique, at least compared to what is normally observed in human chronic HCV carriers (WO 96/13590 to Maertens et al.) and in chimpanzees (van Doom et al., 1996), who raise anti-E1 antibodies in their natural course of infection. In those patients, anti-E1 is in part also directed to discontinuous epitopes but a large proportion is directed against the C4 epitope (±50% of the patient sera), a minor proportion against V1V2 (ranging from 2-70% depending on the genotype), and reactivity against V2V3 was only exceptionally recorded (Maertens et al., 1997).

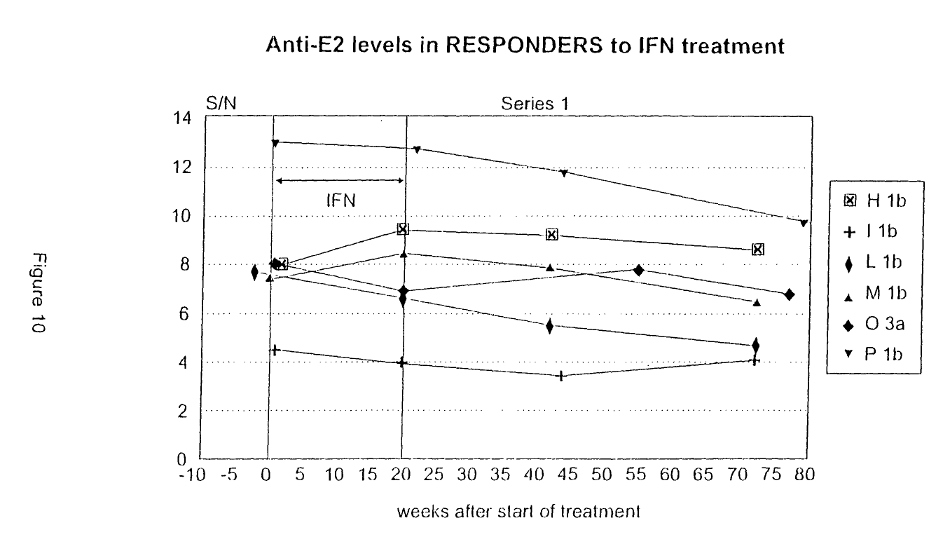

Analysis of the T-cell reactivity indicated that also this compartment of the immune system is stimulated by the vaccine in a specific way, as the stimulation index of these T-cells rises from 1 to 2.5, and remains somewhat elevated during the follow up period (FIG. 10 of WO 99/67285). It is this T cell reactivity that is only seen in Long term responders to interferon therapy (see: PCT/EP 94/03555 to Leroux-Roels et al.; Leroux-Roels et al., 1996).

### EXAMPLE 13

### Immunization of a Chronic HCV Carrier with Different Subtype

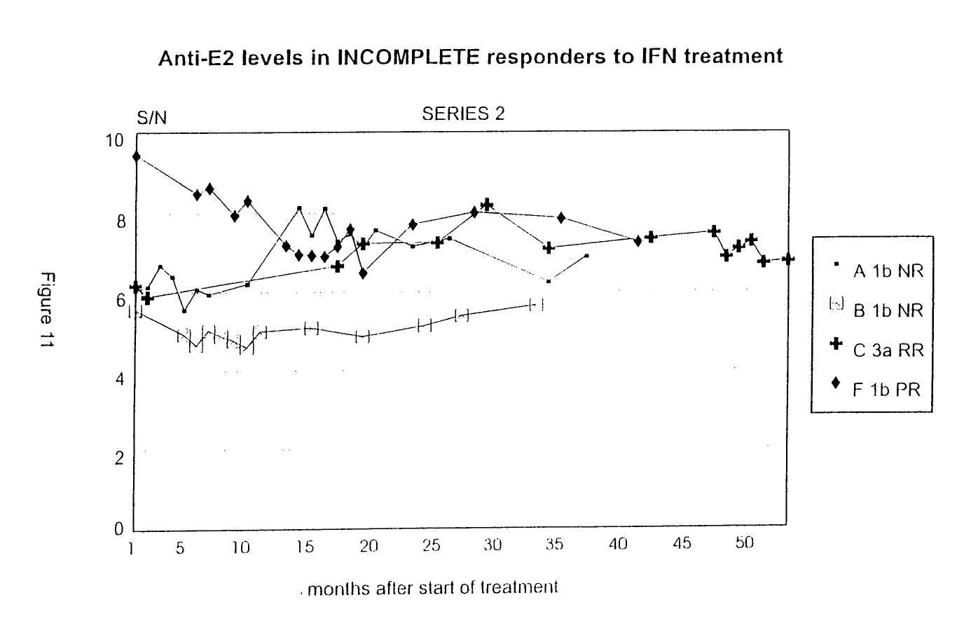

A chimpanzee (Ton) already infected for over 10 years (3809 days before immunization) with HCV from genotype 1a was vaccinated with E1 from genotype 1b, with only a 79.3% identity on the amino acid level (see also Table 2 of WO 99/67285), and prepared as described in the previous examples. The chimpanzee received a total of 6 intramuscular immunizations of 50 μg E1 in PBS/0.05% CHAPS each mixed with RIBI R-730 according to the manufacturer's protocol (Ribi Inc. Hamilton, Mont.). The 6 immunizations were given in two series of three shots with a three week interval and with a lag period of 4 weeks between the two series. Starting 250 days prior to immunization, during the immunization period and until 9 months (but see below and WO 99/67285) post immunization the chimpanzee was continuously monitored for various parameters indicative for the activity of the HCV induced disease. These parameters included blood chemistry, ALT, AST, gammaGT, viral load in the serum, viral load in the liver and liver histology. In addition, the immune answer to the immunization was monitored both on the humoral and cellular level. During this period the animal was also monitored for any adverse effects of the immunization, such as change in behaviour, clinical symptoms, body weight, temperature and local reactions (rednessi swelling, indurations). Such-effects were not detected. Clearly, ALT levels (and gammaGT levels, data not shown) decreased as soon as the antibody level against E1 reached its maximum (FIG. 11 of WO 99/67285). ALT and gammaGT rebounded as soon as the antibody levels started to decline, but ALT and gammaGT remained at a lower level during the complete follow up period. ALT levels were even significantly reduced after vaccination (62±6 U/l) as compared to the period before vaccination (85±11 U/l). Since less markers of tissue damage were recovered in the serum, these findings were a first indication that the vaccination induced an improvement of the liver disease.

E2 antigen levels became undetectable in the period in which anti-E1 remained above a titer of 1.0×10³, but became detectable again at the time of lower E1 antibody levels. Together with the disappearance of HCV antigens, the inflammation of the liver markedly decreased from moderate chronic active hepatitis to minimal forms of chronic persistent hepatitis (Table 3 of WO 99/67285). This is another major proof that the vaccine induces a reduction of the liver damage, probably by clearing, at least partially, the virus from its major target organ, the liver .

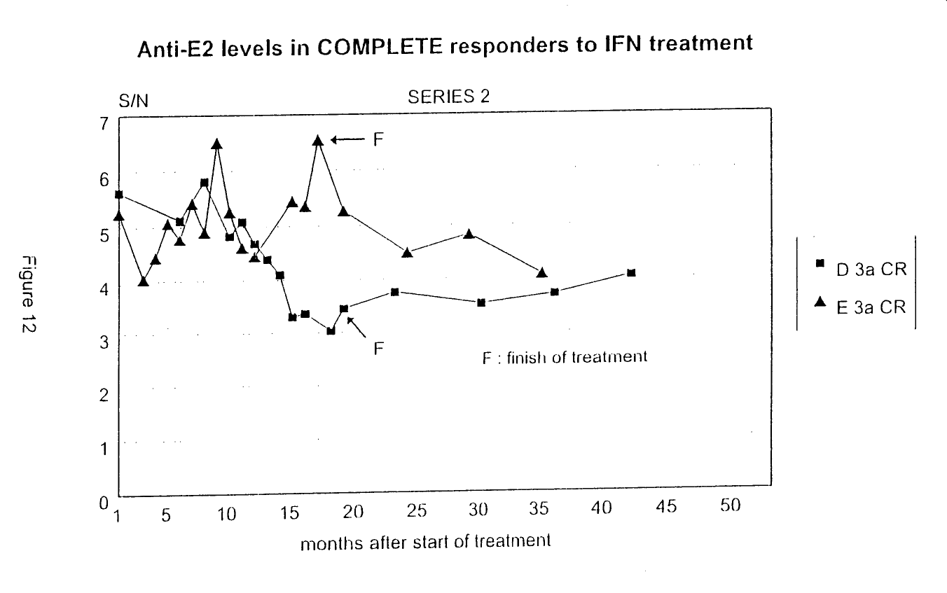

The viraemia level, as measured by Amplicor HCV Monitor (Roche, Basel, Switzerland), in the serum remained at approximately similar levels during the whole study period. More detailed analysis of the humoral response revealed that the maximum end-point titer reached was 30×10³ (after the sixth immunization) and that this titer dropped to 0.5×10³ nine months after immunization (FIG. 11 of WO 99/67285). FIG. 12 of WO 99/67285 shows that the main epitopes, which can be mimicked by peptides and are recognized by the B-cells, are located at the N-terminal region (peptides V1V2 and V2V3, for details on the peptides used see Table 4 of WO 99/67285). Since the reactivity against the recombinant E1 is higher and longer lasting, it can also be deduced from this figure, that the antibodies recognizing these peptides represent only part of the total antibody population against E1. The remaining part is most likely directed against epitopes which cannot be mimicked by peptides, i.e. discontinuous epitopes. Such epitopes are probably only present on the complete E1 molecule or even only on the particle-like structure. Such an immune response against E1 is unique, at least compared to what is normally observed in human chronic HCV carriers, which have detectable anti-E1. In those patients, anti-E1 is in part also discontinuous, but a large proportion is directed against he C4 epitope (50% of the patient sera), a minor proportion against V1V2 (ranging from 2-70% depending on the genotype) and exceptionally reactivity against V2V3 was recorded (Maertens et al., 1997). As this chimpanzee is infected with an 1a isolate the antibody response was also evaluated for cross-reactivity towards a E1-1a antigen. As can be seen in FIG. 13 of WO 99/67285, such cross-reactive antibodies are indeed generated, although, they form only part of the total antibody population. Remarkable is the correlation between the reappearance of viral antigen in the liver and the disappearance of detectable anti-1a E1 antibodies in the serum.

Analysis of the T-cell reactivity indicated that also this compartment of the immune system is stimulated by the vaccine in a specific way, as the stimulation index of these T-cells rises from 0.5 to 5, and remains elevated during the follow up period (FIG. 14 of WO 99/67285).

### EXAMPLE 14

### Reboosting of HCV Chronic Carriers with E1

As the E1 antibody titers as observed in examples 12 and 13 were not stable and declined over time, even to undetectable levels for the 1b infected chimp, it was investigated if this antibody response could be increased again by additional boosting. Both chimpanzees were immunized again with three consecutive intramuscular immunization with a three week interval (50 μg E1 mixed with RIBI adjuvant). As can be judged from FIGS. 8 and 11 of WO 99/67285, the anti-E1 response could indeed be boosted, once again the viral antigen in the liver decreased below detection limit. The viral load in the serum remained constant although in Ton (FIG. 11 of WO 99/67285). A viremia level of <10⁵ genome equivalents per ml was measured for the first time during the follow up period.

Notable is the finding that, as was already the case for the first series of immunizations, the chimpanzee infected with the subtype 1b HCV strain (Phil) responds with lower anti-E1 titers, than the chimpanzee infected with subtype 1a HCV strain (maximum titer in the first round 14.5×10³ versus 30×10³ for Ton and after additional boosting only 1.2×10³ for Phil versus 40×10³ for Ton). Although for both animals the beneficial effect seems to be similar, it could be concluded from this experiment that immunization of a chronic carrier with an E1 protein derived from another subtype or genotype may be especially beneficial to reach higher titers, maybe circumventing a preexisting and specific immune suppression existing in the host and induced by the infecting subtype or genotype. Alternatively, the lower titers observed in the homologous setting (1b vaccine+1b infection) may indicate binding of the bulk of the antibodies to virus. Therefore, the induced antibodies may possess neutralizing capacity.

### EXAMPLE 15

### Demonstration of Prophylactic Utility of E1-Vaccination in Chimpanzee

The HCV E1s protein (amino acids 192-326) was expressed in Vero cells using recombinant vaccinia virus HCV11B. This vaccinia virus is essentially identical to vvHCV11A (as described in U.S. Pat. No. 6,150,134, the entire contents of which is hereby incorporated by reference) but has been passaged from RK13 to Vero cells. The protein was purified (by means of lentil chromatography, reduction-alkylation and size exclusion chromatography) essentially as described in example 9 of PCT/E99/04342 (WO 99/67285), making use of iodoacetamide as alkylating agent for the cysteines. After purification the 3% empigen-BB was exchanged to 3% betain by size exclusion chromatography as described in example 1 of PCT/E99/04342 this process allows to recover E1s as a particle. Finally the material was desalted to PBS containing 0.5% betain and an E1s concentration of 500 μg/ml. This E1 was mixed with an equal volume of Alhydrogel 1.3% (Superfos, Denmark) and finally further diluted with 8 volumes of 0.9% NaCl to yield alum-adjuvanted E1 at a concentration of 50 μg E1/ml and 0.13% of Alhydrogel.

The HCV E2deltaHVRI (amino acids 412-715) was expressed in and purified from Vero essentially as described for E1 using recombinant vaccinia virus HCV101 which has been recombined from pvHCV-101 described in Example 8 of PCT/E99/04342 and wild type vaccinia virus. Also E2deltaHVRI behaves as a particle (measured by dynamic light scattering) after exchange of empigen to betain.

Five chimpanzees were selected which tested negative for HCV-RNA and HCV-antibodies. One of the animals (Huub) was not immunized, 2 animals received 6 immunizations with 50 μg E1 adjuvanted with alum (Marti and Yoran) while the remaining 2 animals received 6 immunizations with 50 μg E2deltaHVRI adjuvanted with alum (Joost and Karlien). All immunizations were administered intramuscularly with a 3 week interval. Humoral and cellular immune responses were assessed in each animal against the antigen with which they where immunized and in each animal both type of responses was detected as shown in Table 11.

All chimpanzees became HCV-RNA positive (determined with Monitor HCV, Roche, Basel, Switzerland) on day 7 post challenge and a first ALT and gammaGT peak was measured between days 35 and 63. This evidences that all chimps developed acute hepatitis. Remarkably, both E1 immunized animals resolved their infection while the E2deltaHVRI and the control animal did not. This is evidenced by the fact that the E1 immunized animals lost HCV-RNA (determined with Monitor HCV, Roche, Basel, Switzerland) at day 98 (Yoran) and 133 (Marti) and remained negative so far until day 273 with monthly testing. All the other animals stayed RNA-positive during the entire follow up period of 273 days so far with ALT and gammaGT values not returning to normal as for the E1 immunized chimpanzees but gradually increasing.

TABLE 11
antibody titers were determined by ELISA two weeks after
the 6^{th} immunization. A serial dilution of the sample was
compared to an in house standard (this in house standard defined
as having 1000 mU/ml of E1 or anti-E2deltaHVR I antibody is
a mixture of three sera from HCV chronic carriers selected based
on a high anti-envelope titer). The stimulation index, which
reflects the cellular immune response, was obtained by culturing
PBMC, drawn from the animals two weeks after the third
immunization, in the presence or absence of envelope antigen
and determining the amount of tritiated thymidine incorporated
in these cells during a pulse of 18 hours after 5 days of culture.
The stimulation index is the ratio of thymidine incorporated
in the cells cultured with envelope antigen versus the ones
cultured without antigen. A stimulation index of >3 is
considered a positive signal.

| | Anti-E1 response | | Anti-E2deltaHVRI response | |
|---|---|---|---|---|
| | | Stimulation | Antibody | Stimulation |
| Chimpanzee | Antibody titer | index | titer | index |
| Yoran | 14110 | 10.9 | | |
| Marti | 5630 | 14.2 | | |
| Joost | | | 3210 | 8.5 |
| Karlien | | | 1770 | 11.2 |

Three weeks after the last of 6 immunizations all animals including the control were challenged with 100 CID (chimpanzee infectious doses) of a genotype 1b inoculum (J4.91, kindly provided by Dr. J. Bukh, NIH, Bethesda, Md.). The amino acid sequence divergence between the vaccine proteins and the J4.91 isolate (of which the sequence information is available under accession number BAA01583) is 7% (9 out of 135 amino acids) for E1s and 11% (32 out of 304 amino acids) for E2delta HVRI; Consequently this challenge is considered heterologous and reflects a real life challenge.

In conclusion we have shown that E1-immunization changes the natural history of HCV infection by preventing evolution to a chronic infection, which is the major health problem related with HCV.

### EXAMPLE 16

### Similar E1 Responses which Allowed Clearing of Infection in Chimpanzee Can be Induced in Man

In order to obtain a prophylactic effect of E1 immunization in man it is required that similar immune responses can be induced in man compared to chimpanzee. Therefore we vaccinated 20 male human volunteers, in which no anti-E1 responses (humoral or cellular) could be detected, with 3 doses of 20 μg E1s formulated on 0.13% Alhydrogel in 0.5 ml. All immunizations were given intramuscularly with a 3 week interval. As evidenced in Table 12, 17 out of 20 volunteers indeed mounted a significant humoral and cellular immune response against E1 and this without serious adverse events. Only 1 volunteer (subject 021) should be considered as a non-responder since neither humoral nor cellular responses were above the cut-off level after 3 E1 immunizations. The observation that the humoral response is lower compared to chimpanzee relates to the fact that only 3 immunizations with 20 μg were given and not 6 with 50 μg.

TABLE 12
antibody titers were determined by ELISA two weeks after the
third immunization. A serial dilution of the sample was compared
to an in house standards (this in house standard defined
as having 1000 mU/ml of E1 or anti-E2deltaHVR I antibody
is a mixture of three sera from HCV chronic carriers selected
based on a high anti-envelope titer). The stimulation index (cellular
immune response) was obtained by culturing PBMC, drawn
from the individuals two weeks after the third immunization, in the
presence or absence of 1 μg of E1s and determining the amount
of tritiated thymidine incorporated in these cells during a pulse of 18
hours after 5 days of culture. The stimulation index is the ratio of
thymidine incorporated in the cells cultured with envelope antigen
versus the ones cultured without antigen. A stimulation index
of >3 is considered a positive signal.

| Subject | Antibody | Stimulation |
|---|---|---|
| no | titer | index |
| 002 | 1370 | 30.9 |
| 003 | 717 | 13.2 |
| 004 | 800 | 9.1 |
| 007 | 680 | 3.8 |
| 008 | 1026 | 3.9 |
| 009 | 325 | 4.6 |
| 010 | 898 | 7.7 |
| 011 | 284 | 4.1 |
| 012 | 181 | 3.6 |
| 013 | <20 | 3.5 |
| 014 | 49 | 4.6 |
| 015 | 228 | 3.8 |
| 016 | 324 | 4.1 |
| 017 | <20* | 6.2 |
| 018 | <20 | 6.7 |
| 019 | 624 | 3.1 |
| 020 | 84 | 5.5 |
| 021 | <20 | 2.1 |
| 022 | 226 | 2.7 |
| 023 | 163 | 7.6 |

*this individual is considered anti-E1 positive after immunization since a significant increase in ELISA signal was seen between the preimmune sample and the sample after three immunization, the titer however is very low and does not allow accurate determination.

### EXAMPLE 17

### Boosting of E1 Responses in Vaccinated Healthy Volunteers

19 out of the 20 human volunteers of example 16 were boosted once more with 20 μg E1 s formulated on 0.13% Alhydrogel in 0.5 ml at week 26 (i.e. 20 weeks after the third immunization). Again antibody titers and cellular immune responses were determined 2 weeks after this additional immunization. In all individuals the antibody titer had decreased during the 20 week interval but could easily be boosted by this additional immunization to a level equal or higher of that observed at week 8. On average the antibody titer was double as high after this boost compared to the week 8 titer, and 7 times as high compared to the week 26 titer (Table 13).

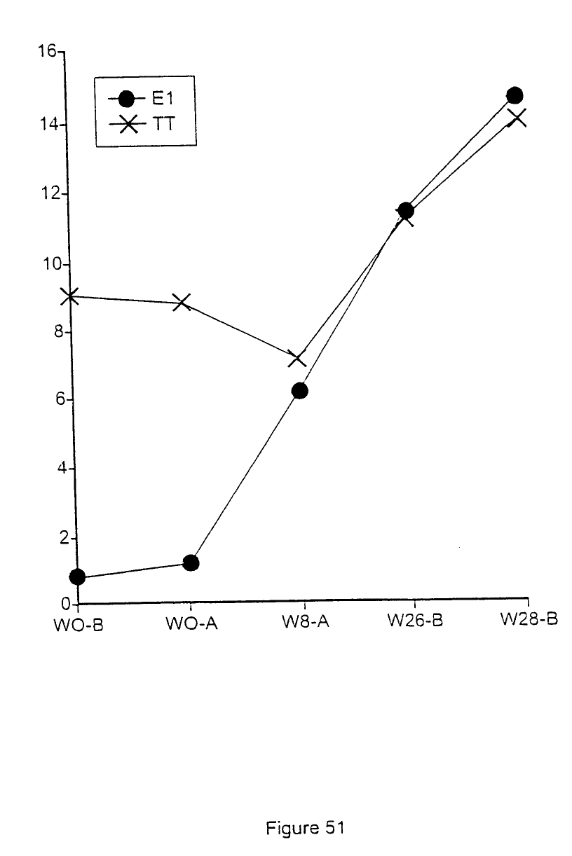

Remarkably the T-cell responses were for the majority of individuals still high after the 20 week interval. Taking in account a normalization to the tetanos response, which is present in most individuals as a consequence of previous vaccinations, there is no change in the geomeatric mean of the stimulation index. After the additional boost, taking in account a normalization to the tetanos response, no change is noted (FIG. 51). This confirms that a strong T-help response was induced after 3 E1 immunizations and indicates that these immunizations induced already a very good T-help memory which requires, at least for a period of 6 months, no further boosting.

TABLE 13
antibody titers were determined by ELISA two
weeks (= week 8) and 20 weeks (= week 26)
after the third immunization and finally also 2 weeks
after the boost (= Week 28). A serial dilution of the
sample was compared to an in house standards
(this in house standard defined as having 1000 mU/ml
of E1 antibody, is a mixture of three sera from HCV
chronic carriers selected based on a high
anti-envelope titer). For accurate comparison the
determination of the titer at week 8 was repeated
within the same assay as for the week 26 and
28 samples, which explains the differences with
table 12 of example 16

Antibody titer

| Subject no | Week 8 | Week 26 | Week 28 |
|---|---|---|---|
| 002 | 1471 | 443 | 3119 |
| 003 | 963 | 95 | 2355 |
| 004 | 1006 | 409 | 2043 |
| 007 | 630 | 65 | 541 |
| 008 | 926 | 81 | 819 |
| 009 | 704 | 77 | 269 |
| 010 | 1296 | 657 | 3773 |
| 011 | 253 | 65 | 368 |
| 012 | 254 | 148 | 760 |
| 013 | 36 | <20 | 166 |
| 014 | 53 | 40 | 123 |
| 016 | 159 | 45 | 231 |
| 017 | 109 | 39 | 568 |
| 018 | 43 | 23 | 50 |
| 019 | 425 | 157 | 1894 |
| 020 | 73 | 33 | 113 |
| 021 | 25 | <20 | 26 |
| 022 | 280 | 150 | 357 |
| 024 | 177 | 81 | 184 |
| average | 467 | 138 | 936 |

Legend to FIG. 51: The stimulation index (cellular immune response) was obtained by culturing PBMC (10⁵ cells), drawn from the individuals before immunization (week 0), two weeks after the third immunization (week 8), before the booster immunization (week 26) and two weeks after the booster immunization (week 28), in the presence or absence of 3 μg of recombinant E1s or 2 μg tetanos toxoid and determining the amount of tritiated thymidine incorporated in these cells during a pulse of 18 hours after 5 days of culture. The stimulation index is the ratio of thymidine incorporated in the cells cultured with envelope antigen versus the ones cultured without antigen. Samples of week 0 and 8 were determined in a first assay (A), while the samples of week 26 and 28 were determined in a second assay (B) in which the samples of week 0 were reanalyzed. Results are expressed as the geometric mean stimulation index of all 20 (A, experiment) or 19 (B, experiment) volunteers.

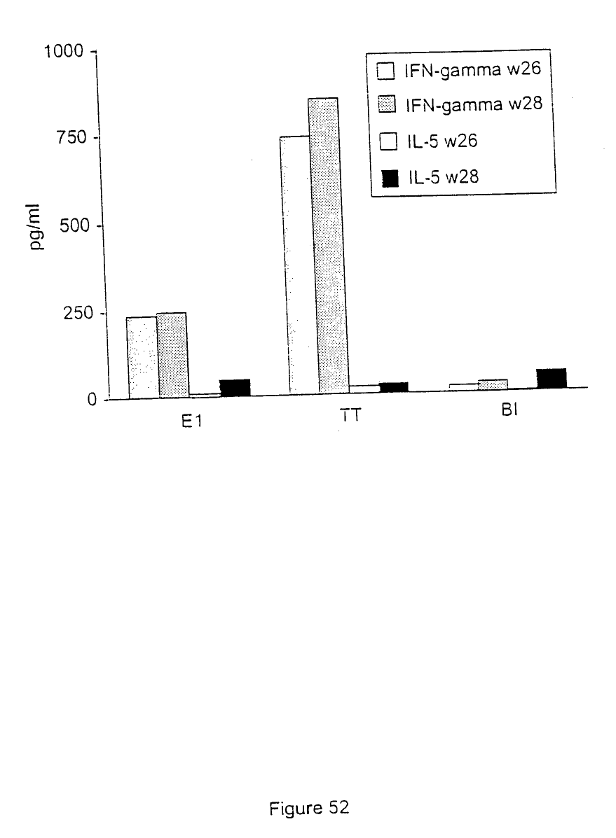

In addition the TH1 cytokine interferon-gamma and Th2 cytokine interleukin-5 were measured in the supernatants of the PBMC cultures of samples taken at week 26 and 28 and restimulated with E1. As can be judged from FIG. 52 the predominant cytokine secreted by the E1 stimulated PBMC is interferon-gamma. It is highly surprising to see that a strong Th1 biased response is observed with an alum adjuvanted E1, since alum is known to be a Th2 inducer. Once more the results confirm that a good T-cell memory response is induced, as prior to the final boost (week 26) already a very strong response is observed. The interferon-gamma secretion was found to be specific as in an additional experiment we saw no difference in interferon-gamma secretion between E1 stimulated cell cultures and non-stimulated cell cultures of these volunteers using samples drawn at week 0.

Legend to FIG. 52: PBMC (10⁵ cells), drawn from the individuals before the booster immunization (week 26) and two weeks after the booster immunization (week 28), were cultured in the presence of 3 μg of recombinant E1s (E1) or 2 μg of tetanos toxoid (TT) or no antigen (B1). Cytokines were measured in the supernatant taken after 24 hours (interleukin-5) or after 120 hours (interferon-gamma) by means of ELISA. The stimulation index is the ratio of cytokine measured in the supernatants of cells cultured with envelope antigen versus the ones cultured without antigen. Results are expressed as the geometric mean of pg cytokine/ml secreted of all 19 volunteers. Samples with a cytokine amount below detection limit were assigned the value of the detection limit. Similarly samples with extremely high concentrations of cytokine out of the linear range of the assay were assigned the value of the limit of the linear range of the assay.

### EXAMPLE 18

### Fine Mapping of Cellular Response Against E1 in Vaccinated Healthy Volunteers

In order to map the E1 specific responses a series of 20-mer peptides was synthesized, using standard Fmoc chemistry, with 8 amino acids overlap and covering the entire sequence of E1s. All peptides were C-terminally amidated and N-terminally acetylated, with the exception of IGP 1626 which has a free amino-terminus.

| | |
|---|---|
| IGP1626: YEVRNVSGIYHVTNDCSNSS (amino acid 192-211) | (SEQ ID NO:112) |
| IGP1627: TNDCSNSSIVYEAADMIMHT (amino acid 204-223) | (SLQ ID NO:113) |
| IGP1628: AADMIMHTPGCVPCVRENNS (amino acid 216-235) | (SEQ ID NO:114) |
| IGP1629: PCVRENNSSRCWVALTPTLA (amino acid 228-247) | (SEQ ID NO:115) |
| IGP1630: VALTPTLAARNASVPTTTIR (amino acid 240-259) | (SEQ ID NO:116) |
| IGP1631: SVPTTTIRRHVDLLVGAAAF (amino acid 252-271) | (SEQ ID NO:117) |
| IGP1632: LLVGAAAFCSAMYVGDLCGS (amino acid 264-283) | (SEQ ID NO:118) |
| IGP1633: YVGDLCGSVFLVSQLFTISP (amino acid 276-295) | (SEQ ID NO:119) |
| IGP1634: SQLFTISPRRHETVQDCNCS (amino acid 288-307) | (SEQ ID NO:120) |
| IGP1635: TVQDCNCSIYPGHITGHRMA (amino acid 300-319) | (SEQ ID NO:121) |
| IGP1636: HITGHRMAWDMMMNWSPTTA (amino acid 312-331) | (SEQ ID NO:122) |

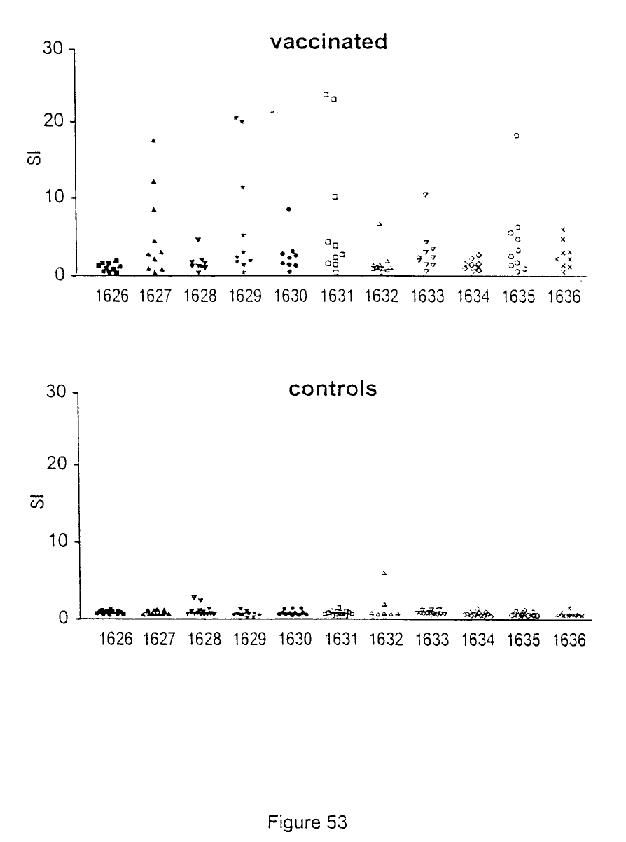

PBMC from 14 different healthy donors not vaccinated with E1s or 10 donors vaccinated with E1s were cultured in the presence of 25 μg/ml (non vaccinated persons) or 10 μg/ml (vaccinated persons, samples taken after the third or booster injection) of each peptide separately. As can be judged from FIG. 53 the peptides IGP 1627, 1629, 1630, 1631, 1633, 1635 and 1635 all induced significantly higher responses in vaccinated persons compared to non-vaccinated persons. Using a stimulation index of 3 as cut-off the peptides IGP 1627, 1629, 1631 and 1635 were the most frequently recognized (i.e. recognized by at least half of the vaccinated persons tested).

This experiment proofs that the T-cell responses induced by E1s derived from mammalian cell culture are specific against E1 since these responses can not only be recalled by the same E1s derived from mammalian cell culture but also by synthetic peptides. In addition this experiment delineates the most immunogenic T-cell domains in E1 are located between amino acids 204-223, 228-271, 276-295, 300-331 and more particularly even between amino acids 204-223, 228-247, 252-271 and 300-319.

Legend to FIG. 53: The stimulation index (cellular immune response) was obtained by culturing PBMC (3×10⁵ cells), in the presence or absence of peptides and determining the amount of tritiated thymidine incorporated in these cells during a pulse after 5-6 days of culture. The stimulation index is the ratio of thymidine incorporated in the cells, cultured with peptide versus the ones cultured without peptide. Results are expressed as individual values for vaccinated persons (top panel) or non vaccinated or controls (lower panel).

The present invention also provides therefor, the following E1 peptides, proteins, compisitions and kits containing the same, nucleic acid sequences coding for these peptides and proteins containing the same, and methods of their manufacture and use, as are generally described herein for other E1 and related peptides of the present invention.

IGP 1626 spanning positions 192-211 of the E1 region (SEQ ID NO:112),

IGP 1627 spanning positions 204-223 of the E1 region (SEQ ID NO:113),

IGP 1628 spanning positions 216-235 of the E1 region (SEQ ID NO:114),

IGP 1629 spanning positions 228-247 of the E1 region (SEQ ID NO:115),

IGP 1630 spanning positions 240-259 of the E1 region (SEQ ID NO:116),

IGP 1631 spanning positions 252-271 of the E1 region (SEQ ID NO:117),

IGP 1632 spanning positions 264-283 of the E1 region (SEQ ID NO:118),

IGP 1633 spanning positions 276-295 of the E1 region (SEQ ID NO:119),

IGP 1634 spanning positions 288-307 of the E1 region (SEQ ID NO:120),

IGP 1635 spanning positions 300-319 of the E1 region (SEQ ID NO:121),

IGP 1636 spanning positions 312-331 of the E1 region (SEQ ID NO:122).

### EXAMPLE 19

### Exemplification of Therapeutic Utility of E1-Vaccination in Man

The HCV E1s protein (amino acids 192-326 (SEQ 10 NO: 123: YEVRNVSGMYHVTNDCSNSSIVYEAADMIMHTPGCVPCVRENNSSRCWVALTPT LAARNASVPTTTIRRHVDLLVGAAAFCSAMYVGDLCGSVFLVSQLFTISPRRHETV QDCNCSIYPGHITGHRMAWDMMMNW)) was expressed in Vero cells using recombinant vaccinia virus HCV11B. This vaccinia virus is essentially identical to vvHCV11A (described in, for example, PCT/EP95/03031 and U.S. Pat. No. 6,510,134, the entire contents of each of which are incorporated herein by reference) but has been passaged from RK13 to Vero cells. The protein was purified (by means of lentil chromatography, reduction-alkylation and size exclusion chromatography) essentially as described in Example 9 of PCT/E99/04342 (the entire contents of each of which are incorporated herein by reference), making use of iodoacetamide as alkylating agent for the cysteines. After purification the 3% Empigen-BB (N-Dodecyl-N,N-dimethylglycine) was exchanged to 3% betain by size exclusion chromatography as described in Example 1 of PCT/E99/04342 this process allows to recover E1s as a particle. Finally the material was desalted to PBS containing 0.5% betain and an E1s concentration ot 400 μg/mL. This E1 was mixed with an equal volume of Alhydrogel 1.3% (Superfos, Denmark) and finally further diluted with 8 volumes of 0.9% NaCl to yield alum-adjuvanted E1 at a concentration of 40 μg E1/mL and 0.13% of Alhydrogel.

In order to demonstrate a therapeutic effect of E1 immunization in man, an immune response was induced in chronically infected patients. This immune response was both quantitatively and qualitatively different from the base line response against E1 which is present in such patients.

Twenty-six chronically genotype 1 infected HCV patients were vaccinated with 5 doses ot 20 μg E1s formulated on 0.13% Alhydrogel in 0.5 mL. All immunizations were given intramuscularly. Immunizations were performed at week 0, 4, 8, 12 and 24. In addition, 9 patients received an identical number of placebo injections consisting of alum only.

As evidenced in the following Table 14, a T -cell response is normally absent in chronic HCV carriers (only 4%, 1 out ot 26, have a detectable T -cell reactivity). Upon immunization this increases to about 70% (18 out of 26) already after 4 immunizations. This observation demonstrates that the immune response has been qualitatively changed from a T -cell non-responsiveness to a clear response. Furthermore this immune response can be sustained, by boosting with larger intervals since after the fifth immunization 3 monhts later, the cellular immune response is still of the same level or slightly increased. No significant changes were observed in the placebo group.

TABLE 14
The stimulation index (SI; cellular immune response)
was obtained by culturing PBMC, drawn from the
individuals four weeks (W16) after the fourth
immunization and two weeks (W26) after the fifth
immunizationt in the presence or absence of 3 μg of
E1s and determining the amount of tritiated thymidine
incorporated in these cells during a pulse of 18 hours
after 5 days of culture. The stimulation index is the
ratio of thymidine incorporated in the cells cultured
with envelope antigen versus the ones cultured
without antigen. A stimulation index of >3 is
considered a positive signal.

T-cell response

| | W0 | W16 | W26 |
|---|---|---|---|
| SI > 3 | 1/26 | 18/26 | 7/25 |
| Mean SI | 1.5 | 13.7 | 19.3 |

In addition the antibody titers were determined by ELISA prior to the first immunization (this was done for each patient on three samples taken at different time points before immunization) and after the fifth immunization (this was done for each patient on two samples: W26 and W28). A serial dilution of the sample was compared to an in-house standard (this in-house standard is, as previously described, defined as having 1000 mU/mL of E1 antibody is a mixture of three sera from HCV chronic carriers selected based on a high anti-envelope titer). From this analysis it was concluded that, on average, the titer doubled from 331 to 715 mU/mL. This result demonstrates that the humoral arm of the immune response has at least been quantitatively altered.

An E1 vaccine formulated on alum significantly changes both the qualitative and quantitative immune response against E1. Such an E1 based vaccine and/or any of the vaccines described herein may be additionally useful if administered in conjunction with antiviral therapy such but not limited to interferon and alternatively with its combination with ribavirin (i.e., prior to, after or with a composition of the present invention). The following as well as all previously and subsequently identified text, references, patents, publication, etc., are hereby incorporated herein in their entirety by reference.

### EXAMPLE 20

### Effects of Therapeutic E1-Vaccination in Chronically Infected Patients

In a first course of this study, 26 patients received 5 doses of 20 μg E1s as described in Example 19. In a second course, 25 of said 26 patients received a further 6 intramuscular doses of 20 μg E1s formulated on 0.13% Alhydrogel in 0.5 mL (as in Example 19). The immunizations of this second series were administered with 3-week intervals and the first injection was given at week 50. Four weeks after the last injection of the second course, a liver biopsy was performed in 24 out of the 25 patients who completed the two courses of therapeutic E1 s vaccination. A liver biopsy was also performed in all patients prior to the onset of the therapeutic vaccine treatment (i.e. prior to course 1 vaccination scheme). The mean time elapsed between pre-treatment and post-treatment biopsies was 17 months. Liver histology slides thus obtained before and after therapeutic vaccination were scored centrally in a blind way by two expert pathologists for fibrosis and inflammation, this according to the Ishak and Metavir scoring systems (Ishak et al. 1995 which is a modification of the scoring system of Knodell et al. 1981; Bedossa and Poynard 1996), as well as for anti-E2 immunostain using the IGH222 murine anti-E2 HVRI monoclonal antibody as described in International Patent Application W099/50301. Perisinusoidal fibrosis was assessed based on staining of collagen with Sirius Red (on liver histology slides).

The Ishak scores range from 0 to 18 for grading of inflammation and from 0 to 6 for staging of fibrosis/cirrhosis. The sum of the Ishak inflammation and fibrosis scores comes closest to the Histological Activity Index (HAI; Knodell et al. 1981) which has been widely used. The change in Ishak fibrosis score in the 24 patients who followed the two courses of therapeutic E1s vaccination is −0.04 (with a 95% confidence interval of −0.60 to +0.68) and with a change from baseline- to end-value from 2.54 (baseline) to 2.50 (end).

An overview of the different assessed necro-inflammatory intensities (Ishak scoring) is given in Table 15.

The Ishak equivalent of the HAI scores for the treated patients showed a mean absolute change from baseline of −0.17 (with a 95% confidence interval of −1.36 to 1.03) and with a change from baseline- to end-value from 8.88 (baseline) to 8.71 (end). Furthermore, nine (9) out of the 24 patients (38%) improved 2 points or more on the sum of Ishak inflammation- and Ishak fibrosis-scores whereas ten (10) out of the 24 patients remained stable (no change or a change of +1 or −1) and five (5) patients evolved to a worse condition (worsening of 2 points or more).

The Metavir scores range from 0 to 3 for grading of inflammation and from 0 to 4 for staging of fibrosis/cirrhosis. The overall progression rate of the Metavir score in an untreated patient is estimated to be 0.133 per year (Poynard et al. 1997).

For the patients of the current study, the average progression over the 17-month period based on the linear extension of the baseline score and the estimated duration of the infection (which is reported for 19 out of the 24 patients who followed the two courses of therapeutic E1s vaccination) would be 0.20. This correlates well with the published overall rate of progression which would be 0.19 for the 17 month period.

The change in Metavir score observed for the treated patients is, however, 0.00 (with a 95% confidence interval of −0.43 to +0.43) and with a mean baseline- an end-score of 1.67. An overview of Metavir scores at baseline and at the end of the second course treatment is given in Table 16. Unblinded comparison of pre- and post treatment slides revealed that 10 patients had diminished perisinusoidal fibrosis based on Sirius Red staining.

The anti-E2 immunostain scores range from 0 to 4, with 0=no E2 antigen detectable, 1=occasional cell with detectable E2 antigen, 2=clusters of E2 antigen positive cells but less than 25% of cells positive, 3=clusters of E2 antigen positive cells and 25-50% of cells positive, 4=clusters of E2 antigen positive cells and more than 50% of cells positive. The anti-E2 immunostain scores for the treated patients showed a mean absolute change from baseline of −0.75 (with a 95% confidence interval of −1.64 to 0.14) and with a change from baseline- to end-value from 2.54 (baseline) to 1.79 (end). The reduction seen for anti-E2 immoreactivity in the liver using blind scoring was confirmed by unblinded comparison of paired slides per patient. Eleven patients showed negativation or a marked reduction in HCV E2 immunostaining, while only three patients had a stronger immunostain after treatment. Of these 11 patients, 3 also showed a reduction in steatosis.

Serum HCV RNA levels were determined using the Amplicor HCV Monitor kit (Roche, Basel, Switzerland). The serum HCV RNA levels remained unchanged or did not change more than one log from baseline except in 1 patient. This concerned a 37-year old treatment-naive female patient, infected with a genotype 1a virus and with flu-like symptoms at baseline. HCV-RNA dropped 3 logs from week 8 (after two injections with E1) to reach levels below 3000 IU/ml at weeks 16 and 20, in parallel ALT dropped from a single peak value of 400 U/ml at week 8 to normal ALT values at weeks 16, 20 and 24. This was accompanied by disappearance of the flu-like symptoms. After this period both ALT and HCV-RNA increased again, and symptoms re-appeared. The sequence of the virus present during the emerging viral load increase was compared with the baseline sequence. No evidence for the presence of escape mutant virus was found. Serum ALT (alanine aminotransferase activity) levels in the treated patients decreased on average 22% (with a 95% confidence interval of −15% to −30%) as compared to the baseline levels. A positive correlation between % change from baseline in ALT levels and absolute change from baseline in fibrosis scores was noticed: a Spearman rank of p=0.0007 for Ishak fibrosis score and of p=0.002 for Metavir fibrosis score were obtained as correlations between the change from baseline in serum ALT and the absolute change from baseline Ishak and Metavir fibrosis scores, respectively.

A further outcome of the current study is the predictive character of the immune response (in terms of increase in serum anti-E1s antibody levels) to treatment for improvement in histological scores (fibrosis and overall) as well as serum ALT levels.

Antibody titers were determined by ELISA. A serial dilution of a serum sample was compared to an in house standard (this in house standard defined as having 1000 mU/mL of E1s antibody is a mixture of three sera from HCV chronic carriers selected based on a high anti-envelope titer). The detection limit for this assay is 5 mU/mL.

A Spearman rank of p=0.007 was obtained as correlation between the change from baseline in serum anti-E1 antibody level and the absolute change from baseline in total Ishak (inflammation plus fibrosis) score. Pearson p-values of p=0.06 and of p=0.009 were obtained as correlation between the change from baseline in serum anti-E1 antibody level and the absolute (p=0.06) and relative (p=0.009) change from baseline in Ishak fibrosis score, respectively. A Pearson p-value of p=0.01 was obtained as correlation between the change from baseline in serum anti-E1 antibody level and the relative change from baseline in ALT value.

These associations remain after correction for baseline liver histology scores, ALT, serum HCV-RNA viral load, age and sex, and IFN exposure and thus as add to the likelihood of a real positive effect induced by the therapeutic E1s vaccination treatment.

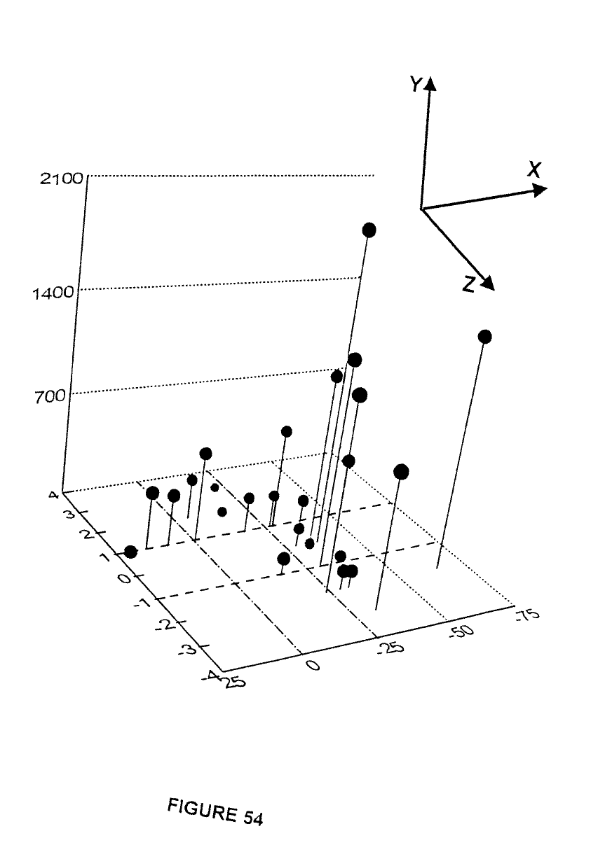

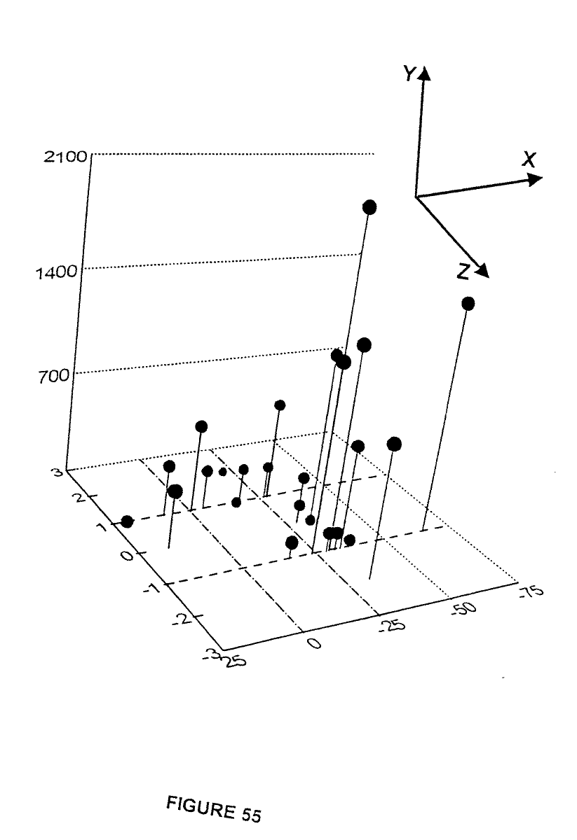

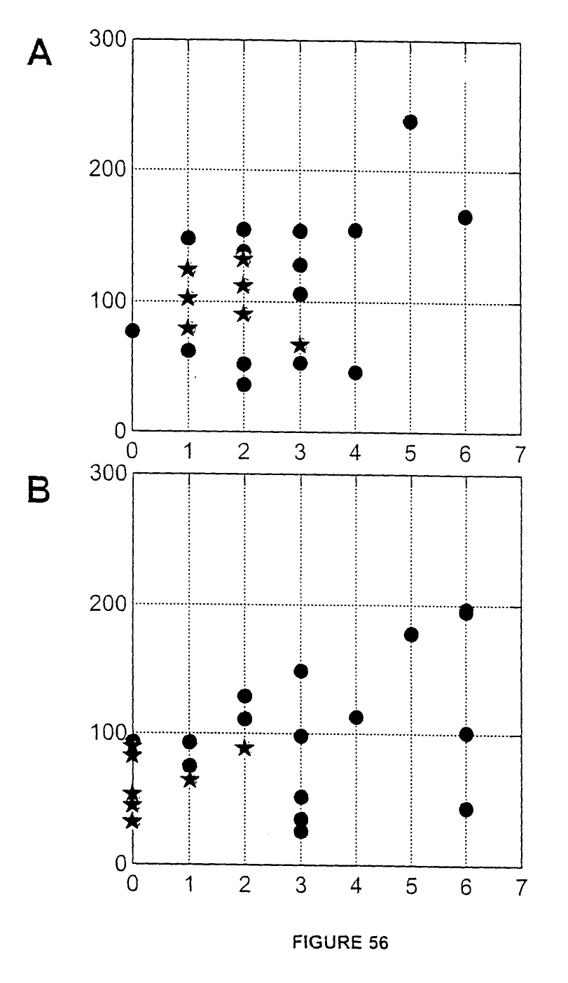

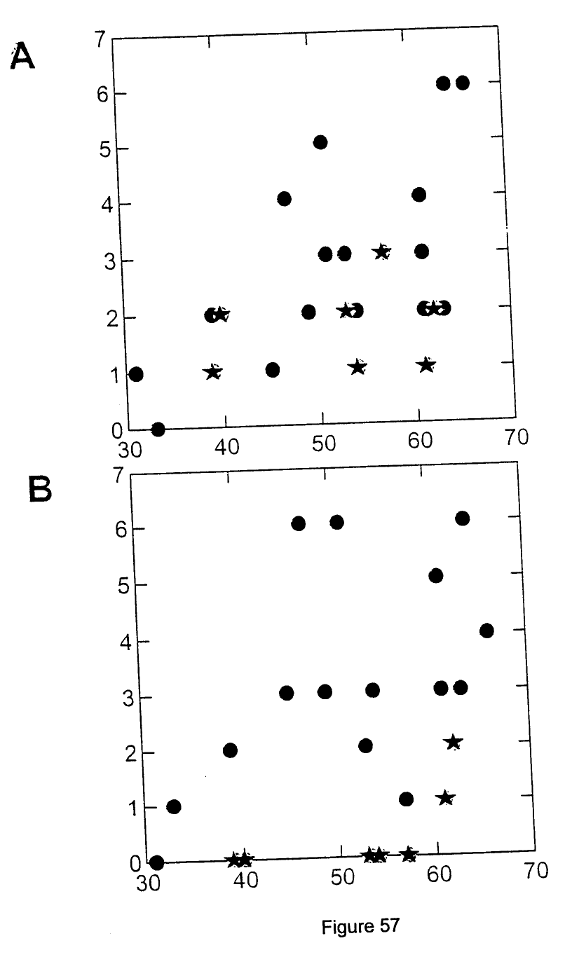

The seven patients with the highest increase in serum antibody levels in response to the therapeutic vaccine candidate (i.e., patients with an increase in anti-E1 antibody level of at least 700 mU/mL) had on average a significant decrease of −0.9 points (with a 95% confidence interval of −0.2 to −1.5) on Metavir liver fibrosis score. In this subgroup, serum ALT on average decreased 37% (with a 95% confidence interval of −25 to −49%). Table 17 provides an overview of the correlation between immune response (in terms of anti-E1s antibody levels induced by treatment) and overall change (relative to baseline) in Ishak scores. FIG. 54 illustrates the correlation between changes in Ishak fibrosis score, changes in ALT-levels and changes in anti-E1s antibody levels. FIG. 55 illustrates the correlation between changes in Metavir fibrosis score, changes in ALT-levels and changes in anti-E1s antibody levels. FIG. 56 illustrates Ishak fibrosis score versus ALT levels both at the start of the first treatment course (panel A) and at the end of the second treatment course (panel B). FIG. 57 illustrates patient's age versus Ishak fibrosis score both at the start of the first treatment course (panel A) and at the end of the second treatment course (panel B). Both FIGS. 56 and 57 further indicate the seven patients with the highest increase in anti-E1s antibody levels induced by the therapeutic vaccination treatment.

A positive correlation between serum anti-E1s antibody levels and T cell proliferation index values at the end of the second treatment course was observed, with a Pearson p-value of p=0.009.

Concludingly, the increase in antibody levels to the E1s vaccination significantly predicted improvement in liver fibrosis (Metavir and Ishak scores), sum of inflammation and fibrosis scores (Ishak), and in ALT levels, even after correcting for any other baseline prognostic variables. The increase in anti-E1 antibodies after vaccination was also significantly correlated with an increase in T cell proliferation index to E1.

This study is clearly supportive for a E1-based therapeutic vaccination strategy to have the potential to halt disease progression towards liver cirrhosis.

TABLE 15
Ishak grading of necro-inflammatory intensities
for periportal hepatitis, confluent necrosis,
focal inflammation, portal inflammation and the
overall total inflammation grading. Scores are
indicated as the change from baseline (mean
and 95% confidence interval) and the mean
baseline- to end-values.

| | Change of | |
|---|---|---|
| | score from | Change of score |
| | baseline | from |
| | mean (95% confidence | baseline to end |
| Score for | interval) | (mean) |
| periportal hepatitis | −0.21 (−0.62 to 0.20) | 1.42 to 1.21 |
| confluent necrosis | 0.38 (−0.17 to 0.92) | 0.00 to 0.38 |
| focal inflammation | −0.17 (−0.59 to 0.26) | 2.63 to 2.46 |
| portal inflammation | −0.13 (−0.51 to 0.26) | 2.29 to 2.17 |
| total inflammation grading | −0.13 (−1.18 to 0.93) | 6.33 to 6.21 |

TABLE 16
Overview of frequencies (given as number of patients) of changes of
a given baseline Metavir score (given in top row; Baseline 0 to 4)
to a given Metavir score at the end of the second course of
therapeutic E1s vaccination (given in left row; EOT 0 to 4).
For instance, the “5” marked with a “*” (i.e., “5*”) means
that 5 patients had a baseline Metavir score of 1 and a Metavir score
of 0 at the end of the second course treatment (EOT = end of
treatment).

| | Base- | | | | | |
|---|---|---|---|---|---|---|
| | line 0 | Baseline 1 | Baseline 2 | Baseline 3 | Baseline 4 | Total |
| EOT 0 | 0 | 5* | 1 | 0 | 0 | 6 |
| EOT 1 | 1 | 3 | 2 | 0 | 0 | 6 |
| EOT 2 | 0 | 5 | 1 | 0 | 0 | 6 |
| EOT 3 | 0 | 0 | 0 | 1 | 1 | 2 |
| EOT 4 | 0 | 0 | 1 | 2 | 1 | 4 |
| Total | 1 | 13 | 5 | 3 | 2 | 24 |

TABLE 17
Correlation between serum anti-E1 antibody
levels induced by therapeutic E1s vaccination
and change in overall Ishak scores. Given are
the number of patients corresponding to the
possible criteria as outlined in the Table.

| | | | |
|---|---|---|---|
| Overall change of Isbak score (change | <−1 | −1, 0, or +1 | >+1 |
| from baseline) | | | |
| High anti-E1s antibody response | 5 | 2 | 0 |
| Low anti-E1s antibod response | 4 | 8 | 5 |

### EXAMPLE 21

### Effects of Therapeutic E1-Vaccination Regimen

In a first course of this study, 26 patients received 5 doses of 20 μg E1s (“E1s patients”) and 9 patients received an identical number of placebo injections (“placebo patients”) consisting of alum only, as described in Example 19. In a second course, 25 of said 26 patients that received E1s during the first course and 9 of said 9 patients that received a placebo were all immunized with a further 6 intramuscular doses of 20 μg E1s formulated on 0.13% Alhydrogel in 0.5 mL (as in Example 19), thus giving rise to two groups of patients: the “E1s/E1s patients” and “placebo/E1s patients”. The immunizations of this second series, i.e. course 2, were administered with 3-week intervals (compared to 4-week intervals during the first series, i.e. course 1) and the first injection was given at week 50. The median values for anti-E1 antibodies after 4 E1s injections were 195 mU/mL for 4-week intervals (as deduced from the E1s patients) and 274 mU/mL for 3-week intervals (as deduced from the placebo/E1s patients), thus indicating that a 3-week interval regimen induces a stronger humoral immune response in a shorter period of time.

In placebo/E1s patients E1s-specific T cell proliferation increased from 0/9 patients at baseline to 9/9 patients after 6 injections with 3-week intervals at week 69.

If the humoral and cellular immune response to E1 s in healthy male volunteers (see Examples 16 and 17) are compared with those in the patient group, a somewhat slower increase in anti-E1 antibody levels and T cell response is observed. The booster effect seen in healthy volunteers was almost absent in patients (median titer of 165 mU/mL after the fifth immunization administered to the E1s/E1s patients as a booster 12 weeks after the 4^{th} immunization), thus suggesting a somewhat impaired build up of immunological memory to E1s in patients. Repeated intramuscular injections at 3-week intervals seem to overcome this impairment (median titer of 530 mU/mL after 6 consecutive immunizations with a 3-week interval in the placebo/E1s patients), thus indicating another advantage of an E1s vaccination regimen with 3-week intervals and/or administering more than 4 doses in the first series of immunizations.

### References

Alric L, Duffaut M, Selves J, Sandre K, Mularczyck M, Izopet J, Desmorat H, Bureau C, Chaouche N, Dalbergue B, Vinel J P (2001) J Hepatol 35(2):272-278.

Asseline U, Delarue M, Lancelot G, Toulme F, Thuong N (1984) Proc. Natl. Acad. Sci. USA 81:3297-301.

Bailey, J. and Cole, R. (1959) J. Biol. Chem. 234, 1733-1739.

Ballou, L., Hitzeman, R., Lewis, M. & Ballou, C. (1991) PNAS 88, 3209-3212.

Bedossa P, Poynard T. (1996) Hepatology 24(2):289-293.

Benesch, R., Benesch, R. E., Gutcho, M. & Lanfer, L. (1956) Science 123, 981.

Cavins, J. & Friedman. (1970) Anal. Biochem. 35, 489.

Cleland, W. (1964) Biochemistry 3, 480

Creighton, E. (1988) BioEssays 8, 57

Darbre, A., John Wiley & Sons Ltd. (1987) Practical Protein Chemistry—A Handbook.

Darbre, A., John Wiley & Sons Ltd. (1987) Practical Proteinchemistry p.69-79.

Doms et al, (1993), Virology 193, 545-562.

Ellman, G. (1959) Arch. Biochem. Biohys. 82, 70.

Falck-Ytter Y, Kale H, Mullen K D, Sarbah S A, Sorescu L, McCullough A J (2002) Ann Intern Med 136(4):288-292.

Falkner, F. & Moss, B. (1988) J. Virol. 62, 1849-1854.

Friedman, M. & Krull. (1969) Biochem. Biophys. Res. Commun. 37, 630.

Gallagher J. (1988) J. Cell Biol. 107, 2059-2073.

Glazer, A., Delange, R., Sigman, D. (1975) North Holland publishing company, Elsevier, Biomedical. Part: Modification of protein (p. 116).

Graham, F. & van der E b, A. (1973) Virology 52, 456-467.

Grakoui et al. (1993) Journal of Virology 67:1385-1395.

Grassetti, D. & Murray, J. (1969) Analyt. Chim. Acta. 46, 139.

Grassetti, D. & Murray, J. (1967) Arch. Biochem Biophys. 119, 41.

Helenius, Mol. Biol. Cell (1994), 5: 253-265.

Hijikata, M., Kato, N., Ootsuyama, Y., Nakagawa, M. & Shimotohno, K. (1991) Proc. Natl. Acad. Sci. U.S.A. 88(13):5547-51.

Hochuli, E., Bannwarth, W., Dobeli, H., Gentz, R., Stuiber, D. (1988) Biochemistry 88, 8976.

Hsu, H., Donets, M., Greenberg, H. & Feinstone, S. (1993) Hepatology 17:763-771.

Inoue, Y., Suzuki, R., Matsuura, Y., Harada, S., Chiba, J., Watanabe, Y., Saito, I. & Miyamura, T. (1992) J. Gen. Virol. 73:2151-2154.

Ishak K, Baptista A, Bianchi L, Callea F, De Groote J, Gudat F, Denk H, Desmet V, Korb G, MacSween R N, et al. (1995) J Hepatol 22(6):696-699.

Janknecht, R., de Martynoff, G. et al., (1991) Proc. Natl. Acad. Sci. USA 88, 8972-8976.

Kayman (1991) J. Virology 65, 5323-5332.

Kato, N., Oostutyama, Y., Tanaka, T., Nakagawa, M., Muraiso, K., Ohkoshi, S., Hijikata, M., Shimitohno, K. (1992) Virus Res. 22:107-123.

Kniskern, P., Hagopian, A., Burke, P., Schultz, L., Monμgomery, D., Hurni, W., Yu Ip, C., Schulman, C., Maigetter, R., Wampler, D., Kubek, D., Sitrin, R., West, D., Ellis, R., Miller, W. (1994) Vaccine 12:1021-1025.

Kohara, M., Tsukiyama-Kohara, K., Maki, N., Asano, K., Yoshizawa, K., Miki, K., Tanaka, S., Hattori, N., Matsuura, Y., Saito, I., Miyamura, T. & Nomoto, A. (1992) J. Gen. Virol. 73:2313-2318.

Knodell R G, Ishak K G, Black W C, Chen T S, Craig R, Kaplowitz N, Kiernan T W, Wollman J. (1981) Hepatology 1(5):431-435.

Lauer G M, Walker B D (2001) N Engl J Med 345(l):41-52.

Lefkowitch J H (1997) Viral Hepatitis Reviews 3(3):155-166.

Mackett, M., Smith, G. & Moss, B. (1985) In: ‘DNA cloning: a practical approach’ (Ed. Glover, D.) IRL Press, Oxford.

Mackett, M., & Smith, G. (1986) J. Gen. Virol. 67, 2067-2082.

Mackett, M., Smith, G. & Moss, B. (1984) J. Virol. 49, 857-864.

Mackett, M., Smith, G. & Moss, B. (1984) Proc. Natl. Acad. Sci. USA 79, 7415-7419.

Matsukura M, Shinozuka K, Zon G, Mitsuya H, Reitz M, Cohen J, Broder S (1987) Proc. Natl. Acad. Sci. USA 84:7706-10.

Means, G. (1971) Holden Day, Inc.

Means, G. & Feeney, R. (1971) Holden Day p.105 & p. 217.

Miller P, Yano J, Yano E, Carroll C, Jayaram K, Ts'o P (1979) Biochemistry 18:5134-43.

Mita, E., Hayashi, N., Ueda, K., Kasahara, A., Fusamoto, H., Takamizawa, A., Matsubara, K., Okayama, H. & Kamada T. (1992) Biochem. Biophys. Res. Comm. 183:925-930.

Moore, S. (1963) J. Biol. Chem. 238, 235-237.

Nielsen P, Egholm M, Berg R, Buchardt O (1991) Science 254:1497-500.

Nielsen P, Egholm M, Berg R, Buchardt O (1993) Nucleic-Acids-Res. 21:197-200.

Okamoto, H., Okada, S., Sugiyama, Y., Yotsumoto, S., Tanaka, T., Yoshizawa, H., Tsuda, F., Miyakawa, Y. & Mayumi, M. (1990) Jpn. J. Exp. Med. 60:167-177.

Panicali & Paoletti (1982) Proc. Natl. Acad. Sci. USA 79, 4927-4931.

Piccini, A., Perkus, M. & Paoletti, E. (1987) Meth. Enzymol. 153, 545-563.

Poynard T, Bedossa P, Opolon P. (1997) Lancet 349(9055):825-832.

Poynard T, McHutchison J, Manns M, Trepo C, Lindsay K, Goodman Z, Ling M H, Albrecht J (2002) Gastroenterology 122(5):1303-1313.

Reichard O, Glaumann H, Fryden A, Norkrans G, Wejstal R, Weiland O (1999) J Hepatol 30(5):783-787.

Rose (1988) Annu. Rev. Cell Biol. 1988, 4: 257-288; Ruegg, V. and Rudinger, J. (1977) Methods Enzymol. 47, 111-116.

Schvarcz R, Glaumann H, Reichard O, Weiland O (1999) J Viral Hepat 6(3):237-242. Shan, S. & Wong (1993) CRC-press p. 30-33.

Shiffman M L (1999) Viral Hepatitis Reviews 5(1):27-43.

Spaete, R., Alexander, D., Rugroden, M., Choo, Q., Berger, K., Crawford, K., Kuo, C., Leng, S., Lee, C., Ralston, R., et al. (1992) Virology 188(2):819-30.

Skehel, J., (1984) Proc. Natl. Acad. Sci. USA 81, 1179-1783.

Stunnenberg, H., Lange, H., Philipson, L., Miltenburg, R. & van der Vliet, R. (1988) Nucl. Acids Res. 16, 2431-2444.

Stuyver, L., Van Arnhem, W., Wyseur, A., DeLeys, R. & Maertens, G. (1993a) Biochem. Biophys. Res. Commun. 192, 635-641.

Stuyver, L., Rossau, R., Wyseur, A., Duhamel, M., Vanderborght, B., Van Heuverswyn, H., & Maertens, G. (1993b) J. Gen. Virol. 74, 1093-1102.

Stuyver, L., Van Arnhem, W., Wyseur, A., Hernandez, F., Delaporte, E., Maertens, G. (1994) Proc. Natl. Acad. Sci. USA 91, 10134-10138.

Takimoto M, Ohkoshi S, Ichida T, Takeda Y, Nomoto M, Asakura H, Naito A, Mori S, Hata K, Igarashi K, Hara H, Ohta H, Soga K, Watanabe T, Kamimura T (2002) Dig Dis Sci. 47(1):170-176.

Weil, L. & Seibler, S. (1961) Arch. Biochem. Biophys. 95, 470.

Yokosuka, O., Ito, Y., Imazeki, F., Ohto, M. & Omata, M. (1992) Biochem. Biophys. Res.

We claim:

1. A method to reduce liver disease in a chronic HCV-infected mammal comprising administering a therapeutic HCV vaccine composition to said mammal.

2. A method to reduce liver fibrosis progression in a chronic HCV-infected mammal comprising administering a therapeutic HCV vaccine composition to said mammal.

3. A method to reduce liver fibrosis in a chronic HCV-infected mammal comprising administering a therapeutic HCV vaccine composition to said mammal.

4. A method to reduce liver disease by at least 2 points according to the overall Ishak score in a chronic HCV-infected mammal comprising administering a therapeutic HCV vaccine composition to said mammal.

5. A method to reduce liver disease by at least 1 point according to the Ishak fibrosis score in a chronic HCV-infected mammal comprising administering a therapeutic HCV vaccine composition to said mammal.

6. A method to reduce serum ALT levels in a chronic HCV-infected mammal comprising administering a therapeutic HCV vaccine composition to said mammal.

7. A method to reduce steatosis in a chronic HCV-infected mammal comprising administering a therapeutic HCV vaccine composition to said mammal.

8. A method to reduce anti-E2 immunoreactivity in the liver of a chronic HCV-infected mammal comprising administering a therapeutic HCV vaccine composition to said mammal.

9. A method of treating a chronic HCV-infected mammal comprising a plurality of administrations of a therapeutic HCV vaccine composition to said mammal wherein said administrations are separated by a time interval of 4 weeks or less.

10. The method of claim 9 wherein said time interval is 3 weeks.

11. The method according to claim 9 wherein said plurality of administrations consists of a first series of at least 5 administrations followed by an administration-free period of at least 12 weeks followed by a second series of at least 3 administrations.

12. The method according to any of claims 1 to 9 wherein said therapeutic HCV vaccine composition comprises a HCV antigen and, optionally, a pharmaceutically acceptable adjuvant.

13. The method according to claim 12 wherein said HCV antigen is an E1 or E2 antigen, or an immunogenic part of an E1 or E2 antigen.

14. The method according to claim 12 wherein said HCV antigen is an E1s antigen.

15. The method according to claim 12 wherein said HCV antigen is the E1s antigen as defined by SEQ ID NO:123.

16. The method according to claim 12 wherein said pharmaceutically acceptable adjuvant is alum.

17. A method to predict changes in liver disease in a chronic HCV-infected mammal comprising
(i) determining the level of serum anti-E1 antibody level prior to therapeutic vaccination with a HCV vaccine composition comprising an E1 antigen;
(ii) determining the level of serum anti-E1 antibody level after therapeutic vaccination with a HCV vaccine composition comprising an E1 antigen;
(iii) inferring the difference in level of serum anti-E1 antibody level determined in (i) and (ii) and, therefrom, predicting the change in liver disease.

18. A therapeutic HCV vaccine composition comprising a therapeutically effective amount of at least one purified HCV single or specific oligomeric recombinant envelope protein selected from the group consisting of an E1 protein, an E2 protein, a part of said E1 and E2 proteins, an E1/E2 protein complex formed from purified HCV single or specific oligomeric recombinant E1 or E2 proteins or parts thereof; and optionally a pharmaceutically acceptable adjuvant.

19. A therapeutic HCV vaccine composition comprising a therapeutically effective amount of a combination of at least two purified HCV single or specific oligomeric recombinant envelope proteins selected from the group consisting of E1 proteins derived from different HCV genotypes or subtypes, E2 proteins derived from different HCV genotypes or subtypes, parts of said E1 and E2 proteins, and E1/E2 protein complexes formed from purified HCV single or specific oligomeric recombinant E1 or E2 proteins, or parts thereof, derived from different HCV genotypes or subtypes; and optionally a pharmaceutically acceptable adjuvant.

20. A therapeutic HCV vaccine composition comprising a therapeutically effective amount of at least one of the following E1 and E2 peptides:
E1-31 spanning amino acids 181 to 200 of the Core/E1 V1 region (SEQ ID NO:56),
E1-33 spanning amino acids 193 to 212 of the E1 region (SEQ ID NO:57),
E1-35 spanning amino acids 205 to 224 of the E1 V2 region (SEQ ID NO:58),
E1-35A spanning amino acids 208 to 227 of the E1 V2 region (SEQ ID NO:59),
1bE1 spanning amino acids 192 to 228 of E1 regions V1, C1, and V2 regions (SEQ ID NO:53),
E1-51 spanning amino acids 301 to 320 of the E1 region (SEQ ID NO:66),
E1-53 spanning amino acids 313 to 332 of the E1 C4 region (SEQ ID NO:67),
E1-55 spanning amino acids 325 to 344 of the E1 region (SEQ ID NO:68),
Env 67 or E2-67 spanning amino acid positions 397 to 418 of the E2 region (SEQ ID NO:72),
Env 69 or E2-69 spanning amino acid positions 409 to 428 of the E2 region (SEQ ID NO:73),
Env 23 or E2-23 spanning positions 583 to 602 of the E2 region (SEQ ID NO:86),
Env 25 or E2-25 spanning positions 595 to 614 of the E2 region (SEQ ID NO:87),
Env 27 or E2-27 spanning positions 607 to 626 of the E2 region (SEQ ID NO:88),
Env 17B or E2-17B spanning positions 547 to 586 of the E2 region (SEQ ID NO:83),
Env 13B or E2-13B spanning positions 523 to 542 of the E2 region (SEQ ID NO:82),
IGP 1626 spanning positions 192-211 of the E1 region (SEQ ID NO:112),
IGP 1627 spanning positions 204-223 of the E1 region (SEQ ID NO:113),
IGP 1628 spanning positions 216-235 of the E1 region (SEQ ID NO:114),
IGP 1629 spanning positions 228-247 of the E1 region (SEQ ID NO:115),
IGP 1630 spanning positions 240-259 of the E1 region (SEQ ID NO:116),
IGP 1631 spanning positions 252-271 of the E1 region (SEQ ID NO:117),
IGP 1632 spanning positions 264-283 of the E1 region (SEQ ID NO:118),
IGP 1633 spanning positions 276-295 of the E1 region (SEQ ID NO:119),
IGP 1634 spanning positions 288-307 of the E1 region (SEQ ID NO:120),
IGP 1635 spanning positions 300-319 of the E1 region (SEQ ID NO:121) and
IGP 1636 spanning positions 312-331 of the E1 region (SEQ ID NO:122);
and, optionally, a pharmaceutically acceptable adjuvant.

21. A therapeutic HCV composition comprising a therapeutically effective amount of at least one purified HCV single or specific oligomeric recombinant envelope protein selected from the group consisting of an E1 protein, an E2 protein, a part of said E1 and E2 protein, and an E1/E2 protein complex formed from purified HCV single or specific oligomeric recombinant E1 or E2 proteins or parts thereof, and optionally a pharmaceutically acceptable adjuvant.

22. A therapeutic HCV composition comprising a therapeutically effective amount of a combination of at least two purified HCV single or specific oligomeric recombinant envelope proteins selected from the group consisting of E1 proteins derived from different HCV genotypes or subtypes, E2 proteins derived from different HCV genotypes or subtypes, parts of said E1 and E2 proteins, and E1/E2 protein complexes formed from purified HCV single or specific oligomeric recombinant E1 or E2 proteins, or parts thereof, derived from different HCV genotypes or subtypes; and optionally a pharmaceutically acceptable adjuvant.

23. A therapeutic HCV composition comprising a therapeutically effective amount of at least one of the following E1 and E2 peptides:
E1-31 spanning amino acids 181 to 200 of the Core/E1 V1 region (SEQ ID NO:56),
E1-33 spanning amino acids 193 to 212 of the E1 region (SEQ ID NO:57),
E1-35 spanning amino acids 205 to 224 of the E1 V2 region (SEQ ID NO:58),
E1-35A spanning amino acids 208 to 227 of the E1 V2 region (SEQ ID NO:59),
1bE1 spanning amino acids 192 to 228 of E1 regions V1, C1, and V2 regions (SEQ ID
NO:53),
E1-51 spanning amino acids 301 to 320 of the E1 region (SEQ ID NO:66),
E1-53 spanning amino acids 313 to 332 of the E1 C4 region (SEQ ID NO:67),
E1-55 spanning amino acids 325 to 344 of the E1 region (SEQ ID NO:68),
Env 67 or E2-67 spanning amino acid positions 397 to 418 of the E2 region (SEQ ID
NO:72),
Env 69 or E2-69 spanning amino acid positions 409 to 428 of the E2 region (SEQ ID
NO:73),
Env 23 or E2-23 spanning positions 583 to 602 of the E2 region (SEQ ID NO:86),
Env 25 or E2-25 spanning positions 595 to 614 of the E2 region (SEQ ID NO:87),
Env 27 or E2-27 spanning positions 607 to 626 of the E2 region (SEQ ID NO:88),
Env 17B or E2-17B spanning positions 547 to 586 of the E2 region (SEQ ID NO:83),
Env 13B or E2-13B spanning positions 523 to 542 of the E2 region (SEQ ID NO:82),
IGP 1626 spanning positions 192-211 of the E1 region (SEQ ID NO:112),
IGP 1627 spanning positions 204-223 of the E1 region (SEQ ID NO:113),
IGP 1628 spanning positions 216-235 of the E1 region (SEQ ID NO:114),
IGP 1629 spanning positions 228-247 of the E1 region (SEQ ID NO:115),
IGP 1630 spanning positions 240-259 of the E1 region (SEQ ID NO:116),
IGP 1631 spanning positions 252-271 of the E1 region (SEQ ID NO:117),
IGP 1632 spanning positions 264-283 of the E1 region (SEQ ID NO:118),
IGP 1633 spanning positions 276-295 of the E1 region (SEQ ID NO:119),
IGP 1634 spanning positions 288-307 of the E1 region (SEQ ID NO:120),
IGP 1635 spanning positions 300-319 of the E1 region (SEQ ID NO:121) and
IGP 1636 spanning positions 312-331 of the E1 region (SEQ ID NO:122);
and, optionally, a pharmaceutically acceptable adjuvant.

24. The therapeutic HCV composition according to any of claims 18 to 23 which is therapeutically effective in a HCV carrier infected with a HCV genotype different from the HCV genotype or HCV genotypes from which said E1, E2, or E1/E2 protein complexes are derived.

25. A composition comprising at least one of the following E1 and E2 peptides:
IGP 1626 spanning positions 192-211 of the E1 region (SEQ ID NO:112),
IGP 1627 spanning positions 204-223 of the E1 region (SEQ ID NO:113),
IGP 1628 spanning positions 216-235 of the E1 region (SEQ ID NO:114),
IGP 1629 spanning positions 228-247 of the E1 region (SEQ ID NO:115),
IGP 1630 spanning positions 240-259 of the E1 region (SEQ ID NO:116),
IGP 1631 spanning positions 252-271 of the E1 region (SEQ ID NO:117),
IGP 1632 spanning positions 264-283 of the E1 region (SEQ ID NO:118),
IGP 1633 spanning positions 276-295 of the E1 region (SEQ ID NO:119),
IGP 1634 spanning positions 288-307 of the E1 region (SEQ ID NO:120),
IGP 1635 spanning positions 300-319 of the E1 region (SEQ ID NO:121) and
IGP 1636 spanning positions 312-331 of the E1 region (SEQ ID NO:122);
and, optionally, a pharmaceutically acceptable adjuvant.

26. The composition according to any of claims 18, 19, 21, or 22 wherein the cysteines of said recombinant HCV envelope proteins are blocked, or according to any of claims 20, 23 and 25 wherein the cysteines of said E1 and E2 peptides are blocked.

27. The composition according to any of claims 18, 19, 21, or 22 wherein said recombinant HCV envelope proteins are added as viral-like particles.

28. The composition according to any of claims 18, 19, 21, or 22 wherein said recombinant HCV E1 envelope protein is an E1s protein.

29. The composition according to claim 28 wherein said E1s protein is defined by SEQ ID NO:123.

30. The composition according to any of claims 18, 19, 21, or 22 wherein said recombinant HCV envelope proteins are produced by recombinant mammalian cells, by recombinant yeast cells or by a recombinant virus.

31. The composition according to any of claims 20, 23 and 25 wherein said peptides are recombinant peptides or synthetic peptides.

32. An immunogenic HCV composition comprising a recombinant virus allowing expression of at least one HCV recombinant envelope protein chosen from an E1 protein and/or an E2 protein, and parts of said E1 and E2 proteins; and, optionally, a pharmaceutically acceptable adjuvant.

33. A HCV vaccine composition comprising a recombinant virus allowing expression of at least one HCV recombinant envelope protein chosen from an E1 protein and/or an E2 protein, and parts of said E1 and E2 proteins; and, optionally, a pharmaceutically acceptable adjuvant.

34. The composition according to claim 32 or 33 which is effective against a HCV genotype or subtype different from the HCV genotype or subytpe from which said E1 protein and/or E2 protein, or said parts thereof, are derived.

35. The composition according to claim 32 or 33 wherein said recombinant virus is a recombinant vaccinia virus, a recombinant avipox virus or a recombinant Ankara Modified Virus.

36. A method of treating a mammal infected with HCV comprising administering to said mammal an effective amount of a composition according to any one of claims 18 to 23, 25, 32 and 33.

37. The method according to any of claims 1 to 9 and 17 wherein said mammal is a human.

38. The method of claim 36 wherein said mammal is a human.
